(19) United States
(12) Patent Application Publication
(10) Pub. No.: US 2026/0215771 A1
(43) Pub. Date: Jul. 30, 2026

(54) APPARATUS AND METHOD FOR LOCKING SUTURES

(71) Applicant: Edwards Lifesciences Corporation, Irvine, CA (US)

(72) Inventors: Haim Brauon, Beit Dagan (IL); Gidon Pesach, Kfar Vitkin (IL); Gal Aviram, Kiryat Tiv'on (IL); Hadas Hoshen Cohen, Yoqneam (IL); Rodolfo Rodriguez, San Luis Obispo, CA (US); Steven M. Ford, Laguna Beach, CA (US); Da-Yu Chang, Irvine, CA (US); Ofry Efraim Yossef, Tel-Aviv (IL); Aviv Galon, Tel Aviv (IL); Ido Halabi, Givatayim (IL)

(21) Appl. No.: 19/629,657

(22) Filed: Mar. 26, 2026

Related U.S. Application Data
(63) Continuation of application No. PCT/US2024/049212, filed on Sep. 30, 2024.
(60) Provisional application No. 63/680,238, filed on Aug. 7, 2024.
(60) Provisional application No. 63/587,402, filed on Oct. 2, 2023.

(51) Int. Cl. A61B 17/04; A61B 17/00

(52) U.S. Cl. CPC A61B 17/0469; A61B 2017/00358; A61B 2017/00367; A61B 2017/00438; A61B 2017/0488

(57) ABSTRACT

Implementations described herein are directed to a tool (e.g., a delivery tool) for surgically delivering, applying, and locking multiple suture locks – e.g., to secure multiple sutures. Each suture lock comprises a first lock piece and a second lock piece. Each suture lock can be transported individually from a storage zone at the tool’s proximal portion, via a multi-shaft system, to the tool’s distal portion, where the suture locks are sequentially secured to respective sutures. In some implementations, each suture lock’s first and second lock pieces are advanced distally to the distal portion, where the suture lock is secured to the suture by engaging the first lock piece with the second lock piece. Other embodiments are also described.

### CROSS-REFERENCE TO RELATED APPLICATIONS

The present application is a continuation of International Patent Application PCT/US2024/049212, filed September 30, 2024, which claims the benefit of U.S. Patent Application No. 63/587,402, filed October 2, 2023, and of U.S. Patent Application No. 63/680,238, filed August 7, 2024, the entire disclosures all of which are incorporated by reference for all purposes.

### TECHNICAL FIELD

Some implementations disclosed herein relate in general to securing suture. More specifically, some implementations relate to surgical use of suture locks to lock sutures.

### BACKGROUND

Sutures are used for a variety of surgical purposes, such as approximation of tissue and ligation of tissue. When placing sutures, the strand of suture material to be used typically has a needle affixed to one end which is passed (looped) through the tissue to be approximated or ligated, forming a stitch. The stitch is then tensioned appropriately, and the two free ends of the suture loop, the needle end and the non-needle end, are knotted to retain the desired tension in the stitch. Forming knots in suture during open surgery is a simple matter, though time-consuming, but forming knots in sutures during endoscopic surgery can require two surgeons to cooperate in a multi-step process which is performed with multiple instruments to pass the needle and suture back and forth to tie the suture knot.

Suture locking devices that eliminate the need to tie knots in order to speed up surgical procedures are known. Suture retainers or locks are used in place of suture knots to prevent passage of a suture end into and through tissue and to maintain the tension applied to the suture material during a suturing procedure.

When using a method that employs a suture lock to secure sutures, the suture lock can be delivered by advancing the suture lock over and along the suture lines to the area of interest, and then engaging the suture lock to the sutures such that the suture lock secures the sutures in place. With the suture lock thus secured, the excess sutures can be cut and removed from the patient. However, deployment of several suture locks during a procedure can be very time consuming, difficult to accomplish without error, and prone to inconsistent tensioning from one lock to the next. In light of the foregoing, there is presently a need for improved systems for securing sutures with suture locks.

### SUMMARY OF THE INVENTION

There is further provided, in accordance with some implementations, a system for use with a suture, the system including: multiple suture locks, each suture lock including: a first lock piece, and/or a second lock piece; and/or a tool: including: at a proximal portion of the tool, a storage zone in which the multiple suture locks are disposed, and/or a multi-shaft system, extending from the proximal portion of the tool to a distal portion of the tool, and/or operable to, for each of the multiple suture locks, sequentially: advance the first lock piece and the second lock piece distally from the storage zone to the distal portion, and/or within the distal portion, secure the suture lock to the suture by engaging the first lock piece with the second lock piece.

For some implementations, at least one of the tool and the suture locks is sterile.

For some implementations, the tool is operable to, for each of the multiple suture locks, secure the suture lock to the suture by sandwiching the suture between first lock piece and the second lock piece.

For some implementations, the tool is operable to, for each of the multiple suture locks, sequentially, within the distal portion: thread the suture through the first lock piece, and/or secure the suture lock to the suture by engaging the first lock piece and the suture with the second lock piece.

For some implementations, the tool further includes a trigger, the trigger operable to, upon application of an actuation force to the trigger, actuate the multi-shaft system to, while a length of suture is threaded through the first lock piece: secure the suture lock to the suture by engaging the first lock piece with the second lock piece, and/or trim the suture.

For some implementations, the trigger is configured to, upon application of the actuation force to the trigger, actuate the multi-shaft system to trim the suture while engaging the first lock piece with the second lock piece.

For some implementations, the trigger is operable such that: upon ceasing application of the actuation force to the trigger, subsequently to securing the suture lock to the suture by engaging the first lock piece with the second lock piece, and/or prior to actuation of the multi-shaft system to trim the suture; the multi-shaft system continues to engage the first lock piece with the second lock piece.

For some implementations: the suture lock is a first suture lock of the multiple suture locks, the trigger is operable to, upon application of the actuation force to the trigger, cyclically actuate the multi-shaft system such that trimming the suture advances the tool toward a ready state in which the tool is actuatable to: engage a second suture lock of the multiple suture locks, and/or advance the second suture lock to the distal portion of the tool.

For some implementations, the tool includes: a snare, operable to thread a length of the suture through the first lock piece and into the multi-shaft system, and/or a suture-exposing mechanism, operable to remove the length of suture from the multi-shaft system.

For some implementations, the suture-exposing mechanism is operable to remove the length of suture from the multi-shaft system while the length of suture remains threaded through the first lock piece.

For some implementations, the snare is operable to: extend distally through the first lock piece, and/or retract distally, drawing the length of suture proximally through the first lock piece.

For some implementations, the suture-exposing mechanism includes a cantilever that is pivotable between: an aligned state in which the cantilever is aligned along the multi-shaft system, and/or an oblique state in which the cantilever is angled away from the multi-shaft system.

For some implementations: the snare is operable to thread the suture through the first lock piece and through a portion of the cantilever, while the cantilever is in the aligned state, and/or pivoting the cantilever away from the system, while the length of suture is threaded through the portion, exposes the suture from the system.

For some implementations: the first lock piece includes a tube, and/or the second lock piece includes a plug.

For some implementations: the tube has: a distal face, and/or an outer surface that defines: at a first portion of the tube, a first width of the tube, at a second portion of the tube, a second width of the tube, the second width being smaller than the first width, and/or a shoulder between the first portion and the second portion, the second portion extending axially between the distal face and the shoulder; and/or wherein the tool is configured to hold the tube in the distal portion of the tool by engaging the shoulder, such that the distal face of the tube protrudes distally from a distal aperture of the tool.

For some implementations: the first portion of the tube is a proximal portion of the tube, and/or the second portion of the tube is a distal portion of the tube having a distal outer diameter.

For some implementations: the shoulder of the tube is wider than a distal surface of the tube, and/or the tool is configured to hold the tube in the distal portion of the tool by the shoulder of the tube, such that the distal surface of the tube protrudes medially and distally from the tool.

For some implementations, the tool is the tool is operable such that, for each of the multiple suture locks, following the securing of the suture lock to the suture, the suture lock remains within the distal portion of the tool until an external force is applied to remove the suture lock from the tool.

For some implementations, the tool is operable such that, for each of the multiple suture locks, following the securing of the suture lock to the suture, the suture lock remains within the distal portion of the tool, until an external force between 50–100 g is applied to remove the suture lock from the tool.

For some implementations, the tool further includes a shuttle: mounted to be repeatedly shuttled, each shuttling including (i) an outbound leg in which the shuttle is slid distally over and along the multi-shaft system from the proximal portion of the tool to the distal portion of the tool, and (ii) a return leg in which the shuttle is subsequently slid proximally over and along the multi-shaft system to return from the distal portion of the tool to the proximal portion of the tool; and/or operable such that, during each shuttling: the outbound leg slides one of the suture locks distally from the storage zone to the distal portion, and/or the return leg leaves the suture lock at the distal portion.

For some implementations, the tool is configured such that, upon completion of an outbound leg of the shuttling, the shuttle is inhibited from being shuttled on a second outbound leg of the shuttling until the tool has been actuated to secure the suture lock to the suture within the distal portion of the tool.

For some implementations: the shuttle is configured to perform a subsequent outbound leg of the shuttling after completion of a return leg of the shuttling, and/or the tool is configured to inhibit completion of the return leg of the shuttling until the tool has been actuated to secure the suture lock to the suture within the distal portion of the tool.

For some implementations, the shuttle is operable such that during each shuttling, the respective outbound leg unlocks the suture lock by separating the first lock piece from the second lock piece, such that the tool assumes a ready state in which the tool holds the first lock piece and the second lock piece, separated from each other, within the distal portion of the tool.

For some implementations: the tool further includes a trigger, operable to secure the suture lock to the suture within the distal portion of the tool by actuating the multi-shaft system to engage the first lock piece with the second lock piece, and/or the tool is configured such that the respective return leg of each shuttling facilitates operation of the trigger.

For some implementations: the proximal portion of the tool includes an interlock, the interlock: lockable such that the interlock inhibits operation of the trigger, and/or unlockable such that the interlock facilitates operation of the trigger; and/or the respective return leg of each shuttling facilitates operation of the trigger by unlocking the interlock.

For some implementations: the suture lock is a first suture lock, and/or the tool is configured such that securing the first suture lock to the suture by operating the trigger resets the tool, thereby facilitating the shuttle, during a subsequent outbound leg of the shuttling, sliding a second suture lock distally from the storage zone to the distal portion.

For some implementations, the tool is configured such that securing the first suture lock to the suture by operating the trigger resets the tool such that the multi-shaft system inhibits operation of the trigger until the shuttle, during the subsequent outbound leg of the shuttling, slides the second suture lock distally from the storage zone to the distal portion.

For some implementations, the proximal portion of the tool includes a shuttle adapter, the shuttle adapter configured to: engage the shuttle upon the shuttle completing a return leg of the shuttling in a manner that inhibits the shuttle from engaging the second suture lock, and/or upon the tool securing the first suture lock to the suture by operating the trigger, facilitate the shuttle, during the subsequent outbound leg of the shuttling, sliding the second suture lock distally from the storage zone to the distal portion.

For some implementations: the tool further includes a snare, housed by the shuttle; and/or the shuttle is operable to, while the tool is in the ready state: release the snare from the shuttle distally through the first lock piece, such that the snare snares a length of suture, and/or draw the snared suture proximally back through the first lock piece by proximally retracting the snare.

For some implementations, the tool is configured such that: distally releasing the snare inhibits operation of the tool to secure the suture lock to the suture, and/or drawing the snare proximally facilitates operation of the tool to secure the suture lock to the suture.

For some implementations: the multi-shaft system includes a plurality of coaxial shafts; and/or the tool is configured to, for each of the multiple suture locks: upon the tool distally releasing the snare, inhibit operation of the tool to secure the suture lock to the suture by locking the shafts of the multi-shaft system with respect to each other, and/or upon the tool drawing the snare proximally, facilitate operation of the tool to secure the suture lock to the suture by unlocking the shafts of the multi-shaft system with respect to each other.

For some implementations, the shuttle includes a carriage and a snare pump, and the snare pump is operable such that, while the tool is in the ready state, sliding the snare pump with respect to the carriage releases the snare from the shuttle distally through the first lock piece, such that the snare snares a length of suture.

For some implementations, the shuttle is operable such that sliding the snare pump along a given length of the carriage, releases twice the given length of snare from the shuttle, distally through the first lock piece.

For some implementations, the tool is configured to inhibit operation of the snare pump until the tool assumes the ready state.

For some implementations, the tool includes a pump lock, configured to obstruct sliding of the snare pump with respect to the carriage, until the tool assumes the ready state.

For some implementations, the shuttle includes a pusher, reversibly engageable to the suture lock and configured to: while supporting the suture lock within the distal portion of the tool, facilitate sliding of the snare pump with respect to the carriage, and/or while disengaged from the suture lock, obstruct sliding of the snare pump with respect to the carriage.

For some implementations: the multi-shaft system includes a lock shaft, a distal portion of the lock shaft defining a holding zone; and/or the tool is operable to, during each outbound leg of the shuttling: transport the suture lock from the storage zone toward the holding zone, and/or unlock the suture lock by separating the first lock piece from the second lock piece, such that the tool assumes the ready state.

For some implementations, the tool is operable to, during each outbound leg of the shuttling, unlock the suture lock by distally advancing the first lock piece away from the second lock piece and into the holding zone.

For some implementations, the tool is operable to, during each outbound leg of the shuttling, unlock the suture lock by distally advancing the first lock piece away from the second lock piece such that the first lock piece is friction-fit within the holding zone.

For some implementations, the tool is configured such that, during each outbound leg of the shuttling, the shuttle unlocks the suture lock such that the first lock piece protrudes distally from the tool.

For some implementations, the tool is operable to, during each outbound leg of the shuttling, unlock the suture lock by distally advancing the first lock piece away from the second lock piece such that the lock shaft hugs the first lock piece laterally and proximally.

For some implementations, the tool is operable to, during each outbound leg of the shuttling, unlock the suture lock such that distally advancing the first lock piece away from the second lock piece radially expands the holding zone.

For some implementations: the distal portion of the lock shaft defines a receiving zone, proximally adjacent to the holding zone, and/or the shuttle is operable to, during each outbound leg of the shuttling, unlock the suture lock such that the receiving zone guides the first lock piece into the holding zone.

For some implementations, the tool further includes: a force applicator at the distal portion of the tool; and/or a force regulator, proximal from the force applicator, the force regulator defining a force threshold, and being configured to, upon application of an actuation force to the trigger, transfer the actuation force to the force applicator in a manner that: while the actuation force remains below the force threshold, secures the suture lock to the suture by engaging the first lock piece with the second lock piece, and/or upon the actuation force reaching the force threshold, trims the suture.

For some implementations, the force threshold is between 2.5–3.5 kg of force.

For some implementations, the force threshold is between 2.75–3.25 kg of force.

For some implementations, the force threshold is 3 kg of force.

For some implementations: the multi-shaft system includes: a first shaft defining a lumen, a distal portion of the first shaft defining a holding zone, a second shaft, configured to, by sliding distally, secure the suture lock to the suture by engaging the first lock piece with the second lock piece at the holding zone, and/or a third shaft defining a blade at a distal portion of the third shaft, the third shaft configured to trim the suture by sliding distally; and/or wherein: while the actuation force remains below the force threshold, the second shaft defines the force applicator, and/or upon the actuation force reaching the force threshold, the third shaft defines the force applicator.

For some implementations, the tool is configured such that: application of the actuation force, below the force threshold, to the trigger advances the second shaft with respect to the first shaft, thereby securing the suture lock to the suture by engaging the first lock piece with the second lock piece at the holding zone, and/or application of the actuation force beyond the force threshold to the trigger advances the third shaft with respect to the first shaft, thereby trimming the suture.

For some implementations, the shuttle is operable such that, during each shuttling: the outbound leg slides one of the suture locks distally through the lumen of the first shaft, toward the holding zone, and/or the force regulator is configured to, upon application of the actuation force below the force threshold to the trigger, transfer the actuation force to the second shaft such that the second shaft secures the suture lock to the suture.

For some implementations, the tool is operable to, upon application of the actuation force below the force threshold to the trigger, secure the suture lock to the suture by pushing distally upon the second lock piece.

For some implementations, the tool is operable to, upon application of the actuation force below the force threshold to the trigger, secure the suture lock to the suture by engaging the first lock piece with the second lock piece.

For some implementations: the multi-shaft system includes an inner shaft, the suture lock defines a suture lock-lumen, and/or the shuttle is operable such that, during each shuttling, the outbound leg slides one of the suture locks distally from the storage zone toward the holding zone by sliding the suture lock over and along the inner shaft while the inner shaft is disposed in the suture lock-lumen.

For some implementations, the shuttle is operable such that, during each shuttling, the outbound leg slides one of the suture locks distally from the storage zone toward the holding zone by sliding the suture lock over and along the inner shaft while the suture lock is disposed within the lumen of the first shaft.

For some implementations, the first shaft defines support arms that are configured to, while the shuttle slides the suture lock distally from the storage zone toward the holding zone: maintain a radial position of the inner shaft within the lumen of the first shaft, and/or deflect radially to facilitate distal movement of the suture lock between the support arms.

For some implementations, the second shaft defines pusher arms that are configured to, while the shuttle slides the suture lock distally from the storage zone toward the holding zone: maintain a radial position of the inner shaft within the lumen of the first shaft, and/or deflect radially to facilitate distal movement of the suture lock between the pusher arms.

For some implementations, the tool is configured to, upon application of the actuation force below the force threshold to the trigger, secure the suture lock to the suture by engaging the first lock piece with the second lock piece while the inner shaft is disposed in the suture lock-lumen.

For some implementations, the tool is configured to, upon application of the actuation force beyond the force threshold to the trigger, trim the suture while the inner shaft is disposed in the suture lock-lumen.

For some implementations, the shuttle is operable such that, during each shuttling, the outbound leg slides one of the suture locks distally from the storage zone toward the holding zone, up to a separation point at which sliding the shuttle further distally: separates the first lock piece from the second lock piece, and/or seats the first lock piece in the holding zone.

For some implementations, the tool is operable such that: sliding the shuttle distally to the separation point pushes the suture lock over and along the inner shaft, while the inner shaft is disposed within the suture lock-lumen, and/or sliding the shuttle further distally from the separation point pushes the first lock piece distally off of the inner shaft and into the holding zone.

For some implementations, the tool is operable such that sliding the shuttle further distally from the separation point separates the first lock piece from the second lock piece such that the first lock piece defines a first lock-piece lumen and the second lock piece defines a second lock-piece lumen.

For some implementations, the tool is operable to, for each of the multiple suture locks, thread the suture through the first lock-piece lumen while: the first lock piece is seated in the holding zone, and/or the inner shaft is disposed within the second lock-piece lumen.

For some implementations, the tool is operable to, for each of the multiple suture locks, thread the suture through the first lock-piece lumen, without threading the suture through the second lock-piece.

For some implementations, the tool is operable to, for each of the multiple suture locks, thread the suture through the first lock-piece lumen while the inner shaft is not disposed within the first lock-piece lumen.

For some implementations, the tool is operable to, for each of the multiple suture locks, secure the suture lock to the suture by engaging the first lock-piece with the second lock-piece while the suture is disposed within the first lock-piece lumen.

For some implementations, the tool is operable to, for each of the multiple suture locks, secure the suture lock to the suture by engaging the first lock-piece with the second lock-piece while the suture is disposed outside of the second lock-piece lumen.

For some implementations, the tool is operable to, for each of the multiple suture locks, trim the suture while: the suture is disposed within the first lock-piece lumen, and/or the inner shaft is disposed within the suture-lock lumen.

For some implementations, the tool is operable to, for each of the multiple suture locks, trim the suture while the suture is disposed outside of the second lock-piece lumen.

For some implementations, upon application of the actuation force below the force threshold to the trigger, the actuation force pushes the second lock piece distally such that the first lock piece engages with the second lock piece in the holding zone.

For some implementations, the inner shaft is configured to longitudinally restrain the second lock piece while the shuttle separates the first lock piece from the second lock piece.

For some implementations, a distal portion of the inner shaft defines a detent, the detent configured to inhibit distal sliding of the second lock piece past the detent while the shuttle separates the first lock piece from the second lock piece.

For some implementations, the tool is configured such that application of the actuation force below the force threshold to the trigger engages the first lock piece with the second lock piece in the holding zone by pushing the second lock piece from the separation point toward the first lock piece.

For some implementations, the tool includes an inner shaft spring, the inner shaft spring configured to, upon application of the actuation force below the force threshold to the trigger, engage the first lock piece with the second lock piece by pushing the inner shaft and the second lock piece from the separation point toward the first lock piece.

For some implementations, the tool is configured such that: while the actuation force is not applied to the trigger, the second shaft inhibits the inner shaft spring’s pushing the inner shaft and the second lock piece from the separation point toward the first lock piece, and/or upon application of the actuation force below the force threshold to the trigger, the second shaft facilitates the inner shaft spring’s pushing the inner shaft and the second lock piece from the separation point toward the first lock piece.

There is further provided, in accordance with some implementations, a system for use with a suture, the system including: multiple suture locks, each suture lock including a plug and a tube; and/or a tool: including: at a proximal portion of the tool, a storage zone in which the multiple suture locks are disposed, and/or a multi-shaft system, extending from the proximal portion of the tool to a distal portion of the tool, and/or operable to, for each of the multiple suture locks, sequentially: advance the suture lock distally from the storage zone to the distal portion, and/or within the distal portion, secure the suture lock to the suture by pushing the plug into the tube.

For some implementations, at least one of the tool and the suture locks is sterile.

For some implementations, for each of the suture locks disposed in the storage zone, the plug is disposed in the tube.

For some implementations, the tool is configured to advance each of the suture locks distally from the storage zone while the plug is disposed at least partly within the tube.

For some implementations, the tool is configured to separate the tube from the plug prior to pushing the plug into the tube.

For some implementations, the tool is configured to: advance each of the suture locks from the storage zone to the distal portion of the tool while the plug is at least partly disposed within the tube, and/or within the distal portion, separate the tube from the plug.

For some implementations, the tool is configured to separate the tube from the plug by advancing the tube, within the distal portion of the tool, distally from the plug.

For some implementations, the tool is configured such that, following the securing of the suture lock to the suture, the suture lock remains within the distal portion of the tool, until an external force is applied to remove the suture lock from the tool.

For some implementations, the tool is configured such that, following the securing of the suture lock to the suture, the suture lock remains within the distal portion of the tool, until an external force of 50–100 g is applied to remove the suture lock from the tool.

For some implementations: the multi-shaft system defines a holding zone at the distal portion of the tool; and/or the tool is configured to, while holding the tube in the holding zone: thread the suture through the tube, secure the suture lock to the suture by pushing the plug into the tube such that a portion of the suture becomes sandwiched between an outer surface of the plug and an inner surface of the tube, and/or trim the suture.

For some implementations: the distal portion of the multi-shaft system defines a holding arm bordering the holding zone, and/or the tool is configured to fit the tube into the holding zone by changing an orientation of the holding arm.

For some implementations, the tool is configured such that, after the tube fits into the holding zone, the holding arm supports the tube in the holding zone.

For some implementations: the tube has: a distal face, and/or an outer surface that defines: at a first portion of the tube, a first width of the tube, at a second portion of the tube, a second width of the tube, the second width being smaller than the first width, and/or a shoulder between the first portion and the second portion, the second portion extending axially between the distal face and the shoulder; and/or wherein the tool is configured to hold the tube in the holding zone by engaging the shoulder, such that the distal face of the tube protrudes distally from a distal aperture of the tool.

For some implementations: the first portion of the tube is a proximal portion of the tube, and/or the second portion of the tube is a distal portion of the tube having a distal outer diameter.

For some implementations: the shoulder of the tube is wider than a distal surface of the tube, and/or the tool is configured to hold the tube in the holding zone by the shoulder of the tube, such that the distal surface of the tube protrudes medially and distally from the tool.

For some implementations, the tool further includes a shuttle: mounted to be repeatedly shuttled, each shuttling including (i) an outbound leg in which the shuttle is slid distally over and along the multi-shaft system from the proximal portion of the tool to the distal portion of the tool, and (ii) a return leg in which the shuttle is subsequently slid proximally over and along the multi-shaft system to return from the distal portion of the tool to the proximal portion of the tool; and/or configured such that, during each shuttling: the outbound leg slides one of the suture locks distally from the storage zone to the distal portion, and/or the return leg leaves the suture lock at the distal portion.

For some implementations, the shuttle is configured such that the respective outbound leg of each shuttling: transports the suture lock from the storage zone to the distal portion of the tool, and/or unlocks the suture lock by separating the plug from the tube, such that the tool assumes a ready state in which the tool holds the plug and the tube, separated from each other, within the distal portion of the tool.

For some implementations: the tool further includes a snare, housed by the shuttle; and/or the shuttle is configured to, while the tool is in the ready state: release the snare from the shuttle distally through the tube, such that the snare snares a length of suture, and/or draw the snared suture proximally back through the tube by proximally retracting the snare.

For some implementations: the multi-shaft system includes a lock shaft, a distal portion of the lock shaft defining a distal holding zone; and/or the shuttle is configured to, by sliding distally along the lock shaft while the shuttle is engaged with the tube: transport the suture lock from the storage zone toward the holding zone, and/or unlock the suture lock by separating the plug from the tube, such that the tool assumes the ready state.

For some implementations, the shuttle is configured to, by sliding distally along the lock shaft while the shuttle is engaged with the tube: transport the suture lock from the storage zone toward the holding zone, and/or unlock the suture lock by advancing the tube away from the plug and into the holding zone, such that the tool assumes the ready state.

For some implementations, the shuttle is configured to, by sliding distally along the lock shaft while the shuttle is engaged with the tube: transport the suture lock from the storage zone toward the holding zone, and/or unlock the suture lock by advancing the tube away from the plug and into the holding zone, such that the tube protrudes distally from the tool and the tool assumes the ready state.

For some implementations, the shuttle is configured to, by sliding distally along the lock shaft while the shuttle is engaged with the tube and the tool is in the ready state, compress the holding zone around the tube.

For some implementations: the tool includes an outer shaft slidably housing the lock shaft, and/or the shuttle is configured to, by sliding distally along the lock shaft while the shuttle is engaged with the tube and the tool is in the ready state, compress the holding zone around the tube by passing the outer shaft over and along the holding zone.

For some implementations: the distal portion of the lock shaft is biased to expand radially outward; and/or the shuttle is configured to, by sliding proximally along the lock shaft while the shuttle is: engaged to the outer shaft, and/or disengaged from the tube; allow the distal portion of the lock shaft to expand radially outward, thereby releasing the suture lock from the holding zone.

For some implementations: the multi-shaft system includes an inner shaft, the plug defines a plug-lumen, and/or the shuttle is configured to, by sliding distally along the lock shaft while the shuttle is engaged with the tube, transport the suture lock from the storage zone toward the holding zone by sliding the plug-lumen over and over and along the inner shaft while the inner shaft is disposed in the plug-lumen.

For some implementations, the inner shaft is configured to longitudinally restrain the plug while the shuttle unlocks the suture lock.

For some implementations, a distal portion of the inner shaft defines a detent, the detent configured to inhibit distal sliding of the plug past the detent while the shuttle unlocks the suture lock.

For some implementations: the tube defines a tube-lumen, and/or the multi-shaft system includes a plug shaft, and is actuatable to lock the suture lock by pushing the plug shaft to push the plug distally into the tube-lumen.

For some implementations, the multi-shaft system is actuatable to lock the suture lock by pushing the plug shaft to push the plug distally into the tube-lumen such that a portion of the suture becomes sandwiched between an outer surface of the plug and an inner surface of the tube, laterally from the plug-lumen.

For some implementations, the inner shaft is configured to be pulled distally by the plug as the plug is pushed distally into the tube-lumen, such that the inner shaft accompanies the plug in a manner that maintains the plug aligned with the tube-lumen.

For some implementations: the multi-shaft system includes an inner wire along which the inner shaft is slidably disposed; a distal portion of the inner shaft defines a compressible detent; and/or the multi-shaft system is configured such that: while the distal portion of the inner shaft is disposed over and along the inner wire, the inner wire inhibits radial compression of the detent, thereby inhibiting distal sliding of the plug past the detent while the shuttle unlocks the suture lock, and/or advancing the distal portion of the inner shaft distally beyond the inner wire increases compressibility of the detent, thereby facilitating withdrawal of the inner shaft from within the plug, while the plug is disposed withing the tube-lumen.

For some implementations: tool further includes a trigger, operable to secure the suture lock to the suture within the distal portion by actuating the multi-shaft system to push the plug into the tube, and/or the shuttle is configured such that the respective return leg of each shuttling facilitates operation of the trigger.

For some implementations: the proximal portion of the tool includes an interlock, the interlock: lockable such that the interlock inhibits operation of the trigger, and/or unlockable such that the interlock facilitates operation of the trigger; and/or the respective return leg of each shuttling facilitates operation of the trigger by unlocking the interlock.

For some implementations, the tool further includes: a force applicator at the distal portion of the tool; and/or a force regulator, proximal from the force applicator, the force regulator defining a force threshold, and being configured to, upon application of an actuation force to the trigger, transfer the actuation force to the force applicator in a manner that: secures the suture lock to the suture by pushing the plug into the tube while the actuation force remains below the force threshold, and/or upon the actuation force reaching the force threshold, causes the tool to trim the suture.

For some implementations: the multi-shaft system includes: a plug shaft, configured to push the plug distally into the tube by the plug shaft sliding distally, and/or a cutter shaft defining a blade at a distal portion of the cutter shaft, the cutter shaft configured to trim the suture by sliding distally; and/or wherein: while the actuation force remains below the force threshold, the plug shaft defines the force applicator, and/or upon the actuation force reaching the force threshold, the cutter shaft defines the force applicator.

For some implementations, the force regulator is configured to, upon continued application of the actuation force to the trigger after the tool having trimmed the suture, transfer the actuation force to the force applicator in a manner that causes the tool to release the suture lock.

For some implementations, the force threshold is between 1.5–2.5 kg of force.

For some implementations, the force threshold is between 1.75–2.25 kg of force.

For some implementations, the force threshold is 2 kg of force.

There is further provided, in accordance with some implementations, a system for locking a suture, the system including: a suture lock including: a plug, and/or a tube defining a tube-lumen; and/or a tool: having a ready state in which the tool holds the plug and the tube, separated from each other, within a distal portion of the tool, and/or including: a snare at the distal portion of the tool, the snare configured to draw the suture through the tube-lumen while the tool is in the ready state, a force applicator at the distal portion of the tool, and/or a force regulator, proximal from the force applicator, the force regulator defining a force threshold, and being configured to, upon application of an actuation force to a proximal portion of the tool while the tool is in the ready state and the suture is disposed through the tube, transfer the actuation force to the force applicator in a manner that: secures the suture lock to the suture by inserting the plug into the tube-lumen while the actuation force remains below the force threshold, and/or upon the actuation force reaching the force threshold, causes the tool to trim the suture.

For some implementations, at least one of the plug, the tube, and the tool is sterile.

For some implementations, the force regulator is configured to, upon continued application of the actuation force to the proximal portion of the tool after the tool having trimmed the suture, transfer the actuation force to the force applicator in a manner that causes the tool to release the suture lock.

For some implementations, the force threshold is between 1.5–2.5 kg of force.

For some implementations, the force threshold is between 1.75–2.25 kg of force.

For some implementations, the force threshold is 2 kg of force.

For some implementations, the tool includes a multi-shaft system connecting the force regulator to the distal portion of the tool, a distal portion of the multi-shaft system defining the force applicator.

For some implementations: while the actuation force remains below the force threshold, a first member of the multi-shaft system defines the force applicator, and/or upon the actuation force reaching the force threshold, a second member of the multi-shaft system defines the force applicator.

For some implementations, the tool further includes a shuttle configured to: transport the suture lock from a proximal portion of the tool to the distal portion of the tool, and/or unlock the suture lock by separating the plug from the tube, such that the tool assumes the ready state.

For some implementations: the shuttle houses the snare; and/or the shuttle is configured to, while the tool is in the ready state: release the snare from the shuttle and distally through the tube-lumen, such that the snare snares a length of suture, and/or draw the snared suture proximally back through the tube-lumen by proximally retracting the snare.

For some implementations: the tool further includes a multi-shaft system including a lock shaft that defines a proximal storage zone and a distal holding zone; and/or the shuttle is configured to, by sliding distally along the lock shaft while the shuttle is engaged with the tube: transport the suture lock from the storage zone toward the holding zone, and/or unlock the suture lock by separating the plug from the tube, such that the tool assumes the ready state.

For some implementations, the shuttle is configured to, by sliding distally along the lock shaft while the shuttle is engaged with the tube: transport the suture lock from the storage zone toward the holding zone, and/or unlock the suture lock by advancing the tube away from the plug and into the holding zone, such that the tool assumes the ready state.

For some implementations, the multi-shaft system includes an inner shaft, configured to longitudinally restrain the plug while the shuttle unlocks the suture lock.

For some implementations, the multi-shaft system includes a plug shaft that slidably fits within the lock shaft, the plug shaft configured to lock the suture lock by pushing the plug distally into the tube-lumen at the holding zone.

For some implementations, the tool is configured such that: the lock shaft holds the tube in the holding zone such that the tube protrudes distally from the tool, and/or while the plug locks the suture lock by pushing the plug distally into the tube at the holding zone.

For some implementations: the multi-shaft system includes an inner shaft, the plug is shaped to define a plug-lumen, the shuttle is configured to transport the suture lock from the storage zone toward the holding zone by sliding the suture lock over and along the inner shaft, while the inner shaft is disposed in the plug-lumen, and/or the plug shaft is configured to lock the suture lock by pushing the plug distally into the tube at the holding zone, such that the inner shaft is pulled distally along with the plug.

For some implementations, the shuttle is configured to compress the holding zone around the tube by sliding distally over and along the lock shaft while the tool is in the ready state.

For some implementations: the tool includes an outer shaft slidably housing the lock shaft; and/or the tool is configured such that, while: the tool is in the ready state, and/or the shuttle is engaged with the outer shaft, sliding the shuttle distally over and along the lock shaft compresses the holding zone around the tube by passing the outer shaft over and along the holding zone.

There is further provided, in accordance with some implementations, a method, including: within a distal portion of a tool: unlocking a suture lock, while the suture lock remains unlocked, threading a suture through the suture lock, and/or while the suture remains threaded through the suture lock, securing the suture lock to the suture by locking the suture lock; and/or releasing, from the tool, the suture lock secured to the suture.

For some implementations, the method further includes sterilizing the suture lock.

For some implementations: the suture lock includes a tube and a plug, the step of unlocking includes pushing the tube distally away from the plug, and/or the step of securing includes locking the suture lock by pushing the plug distally into the tube.

For some implementations: the step of unlocking includes pushing the tube distally away from the plug, such that the tube protrudes distally from the tool, and/or the step of locking includes pushing the plug distally into the tube, while the tube protrudes distally from the tool.

For some implementations: the tool includes a shuttle and a multi-shaft system extending from a proximal portion of the tool to a distal portion of the tool, the multi-shaft system defining a proximal storage zone and a distal holding zone; and/or the method further includes, prior to the step of unlocking, distally advancing the suture lock from the storage zone toward the holding zone by: engaging the suture lock with the shuttle, and/or sliding the shuttle distally over and along the multi-shaft system.

For some implementations, the step of locking does not require sliding the shuttle over and along the multi-shaft system.

For some implementations: the multi-shaft system includes an inner shaft, and/or the step of distally advancing the suture lock includes sliding the suture lock from the storage zone toward the holding zone by sliding the suture lock over and along the inner shaft.

For some implementations: the step of engaging includes engaging the tube of the suture lock with the shuttle, and/or the step of unlocking includes, while the tube is engaged with the shuttle, pushing the tube distally away from the plug and into the holding zone by sliding the shuttle distally over and along the multi-shaft system.

For some implementations, the step of unlocking includes, while the suture lock is engaged with the shuttle, pushing the tube distally away from the plug and the inner shaft, while the plug remains disposed over and along the inner shaft.

For some implementations: the inner shaft defines a detent at a distal portion of the inner shaft, and/or the step of unlocking includes, using the detent, longitudinally restraining the plug from sliding distally past the detent.

For some implementations: the tube defines a tube-lumen, the multi-shaft system includes a plug shaft that slidably houses the inner shaft, and/or the step of locking includes, using the plug shaft, pushing the plug distally into the tube-lumen at the holding zone.

For some implementations: the plug is shaped to define a plug-lumen; the inner shaft defines a detent at a distal portion of the inner shaft; and/or the step of locking includes, while the inner shaft is disposed in the plug-lumen, using the plug shaft to: distally slide the plug over and along the inner shaft, such that the plug abuts the detent, and/or slide the inner shaft distally, along with the plug, such that the inner shaft aligns the plug with the tube-lumen.

For some implementations: the multi-shaft system includes an inner wire along which the inner shaft is slidably disposed, the step of locking includes, using the plug shaft, sliding the distal portion of the inner shaft distally beyond the inner wire, and/or the step of releasing includes sliding the inner shaft proximally, thereby radially compressing the detent and withdrawing the inner shaft proximally from the suture lock, through the plug-lumen.

For some implementations: the tool further includes a snare, housed within the shuttle; and/or the step of threading includes threading the suture through the tube by: releasing the snare from the shuttle, distally through the tube, threading the suture through the snare, and/or retracting the suture and the snare proximally through the tube.

For some implementations: the suture lock is a first suture lock; the step of threading includes threading the suture through the tube of the first suture lock by sliding the shuttle proximally over and along the multi-shaft system; and/or the method further includes advancing a second suture lock from the storage zone to the distal portion of the multi-shaft system by sliding the shuttle distally over and along the multi-shaft system.

For some implementations: the step of unlocking includes pushing the tube distally away from the plug and into the holding zone by: engaging the tube with the shuttle, and/or sliding the shuttle distally over and along the multi-shaft system; and/or the method further includes compressing the holding zone around the tube such that the tube is seated at the holding zone.

For some implementations: the multi-shaft system includes: a lock shaft, a distal portion of the lock shaft defining the holding zone, and/or an outer shaft slidably housing the lock shaft; and/or the step of compressing includes: engaging the outer shaft with the shuttle, and/or compressing the holding zone around the tube by passing the outer shaft over and along the lock shaft.

For some implementations: the tool includes: a force applicator at the distal portion of the tool, and/or a force regulator, proximal from the force applicator, the force regulator defining a force threshold; and/or the step of locking includes locking the suture lock by applying an actuation force to a proximal portion of the tool while the suture remains threaded through the suture lock, such that the force regulator transfers the actuation force to the force applicator in a manner that: locks the suture lock while the actuation force remains below the force threshold, and/or trims the suture upon the actuation force reaching the force threshold.

For some implementations, the method further includes, subsequently to the step of locking, releasing the suture lock from the tool by continuing to apply the actuation force, exceeding the force threshold, to the proximal portion of the tool.

There is further provided, in accordance with some implementations, apparatus for use with a suture, the apparatus including a suture lock that includes: a tube having an inner surface defining a tube-lumen, the tube having a tube axis along which the tube-lumen is oriented; and/or a plug having a plug axis along which the plug is oriented; wherein: the suture lock is configured to be secured to the suture by sandwiching a portion of the suture between an outer surface of the plug and the inner surface of the tube, and/or the suture lock is transitionable between: an unlocked state in which the plug is disposed outside of the tube-lumen, and/or a locked state in which the plug is partially disposed within the tube-lumen, such that: the plug protrudes distally from the tube, thereby defining a distal protrusion of the suture lock, and/or the plug is recessed within a proximal portion of the tube, thereby defining a proximal recess of the suture lock.

For some implementations, at least one of the plug and the tube is sterile.

For some implementations, an axial depth of the proximal recess is greater than an axial length of the distal protrusion.

For some implementations: the locked state is an unloaded locked state in which the tube houses the plug, in absence of the suture; and/or while the portion of the suture is threaded through the tube-lumen, the suture lock is transitionable between: a loaded unlocked state in which the plug is disposed outside of the tube-lumen, and/or a loaded locked state in which the plug is partially disposed within the tube-lumen, such that: the portion of the suture is between the outer surface of the plug and the inner surface of the tube, the plug protrudes proximally from the tube, thereby defining a proximal protrusion of the suture lock, and/or the plug is recessed within a distal portion of the tube, thereby defining a distal recess of the suture lock.

For some implementations, while the suture lock is in the unloaded locked state, the plug axis and the tube axis are colinear, thereby defining a lock axis.

For some implementations, while the suture lock is in the loaded locked state, the plug axis and the tube axis are coaxial.

For some implementations, while the suture lock is in the loaded locked state, the plug axis and the tube axis intersect.

For some implementations, while the suture lock is in the unloaded locked state, the inner surface of the tube is parallel to the outer surface of the plug.

For some implementations, the inner surface of the tube is nonparallel to the tube axis.

For some implementations, the outer surface of the plug is nonparallel to the plug axis.

For some implementations: the plug has an inner surface, defining a plug-lumen, oriented along the plug axis; and/or the suture lock is configured to be secured to the suture by insertion of the plug into the tube-lumen such that: the plug axis becomes coaxial with the tube axis, and/or the portion of the suture becomes sandwiched between the outer surface of the plug and the inner surface of the tube, laterally from the plug-lumen.

For some implementations: the tube axis is a central axis of the tube, and/or the plug axis is a central axis of the plug.

For some implementations, the inner surface of the plug is parallel to the plug axis.

For some implementations, the distal portion of the tube is narrower than the proximal portion of the tube.

For some implementations, the outer surface of the tube defines a distally-facing shoulder between a wide portion of the tube and a narrow portion of the tube.

For some implementations, the narrow portion of the tube extends axially from the shoulder to a distal face of the plug.

For some implementations, the system further includes a tool: extending from a proximal portion of the tool to a distal portion of the tool, and/or defining a holding zone at the distal portion of the tool; wherein the tool is configured to thread the suture through the suture lock by pulling the suture proximally through the tube-lumen while holding the tube at the holding zone.

For some implementations, the tool further includes a shuttle, the shuttle configured to: slide the suture lock from the proximal portion of the tool to the distal portion of the tool, and/or unlock the suture lock by sliding the tube away from the plug and into the holding zone.

For some implementations, the tool includes an inner shaft, configured to longitudinally restrain the plug while the shuttle unlocks the suture lock.

For some implementations, the shuttle is configured to slide the suture lock from the proximal portion of the tool to the distal portion of the tool by sliding the suture lock over and along the inner shaft, while the inner shaft is disposed in the plug-lumen.

For some implementations, the tool includes a plug shaft, configured to secure the suture lock to the suture by: pushing the plug distally into the tube-lumen, and/or pulling the inner shaft distally.

There is further provided, in accordance with some implementations, apparatus for use with a suture, the apparatus including a suture lock that includes: a tube having a frustoconical inner surface defining a tube-lumen, the tube having a tube axis along which the tube-lumen is oriented; and/or a plug having: an inner surface defining a plug-lumen, the plug having a plug axis along which the plug-lumen is oriented, and/or a frustoconical outer surface; wherein the suture lock is securable to the suture by insertion of the plug into the tube-lumen such that: a portion of the suture becomes sandwiched between the outer surface of the plug and the inner surface of the tube, laterally from the plug-lumen, and/or the outer surface of the plug grips the inner surface of the tube.

For some implementations, at least one of the plug and the tube is sterile.

For some implementations, the suture lock is securable to the suture at a continuum of depths of the plug within the tube-lumen, dependent on characteristics of the suture.

For some implementations, the suture lock is securable to the suture by insertion of the plug into the tube-lumen such that: the portion of the suture becomes sandwiched between the outer surface of the plug and the inner surface of the tube at a first lateral side of the plug, and/or the outer surface of the plug grips the inner surface of the tube at a second lateral side of the plug, opposite the first lateral side of the plug.

For some implementations: the tube axis is a central axis of the tube, and/or the plug axis is a central axis of the plug.

For some implementations, the suture lock is configured such that, in the absence of the suture, insertion of the plug into the tube-lumen positions the plug axis to be colinear with the tube axis.

For some implementations, the plug is configured to be inserted into the tube, in absence of the suture, such that the inner surface of the tube is parallel to the outer surface of the plug.

For some implementations, the inner surface of the tube is nonparallel to the tube axis.

For some implementations, the outer surface of the plug is nonparallel to the plug axis.

For some implementations, the system further includes a tool, the tool: having a proximal portion and a distal portion, defining a holding zone at the distal portion of the tool, and/or configured to thread the suture through the suture lock by pulling the suture proximally through the tube-lumen while holding the tube at the holding zone.

For some implementations, the tool includes: a multi-shaft system that extends from the proximal portion to the distal portion; and/or a shuttle, slidable distally over and along the multi-shaft system in a manner that slides the suture lock distally toward the distal portion of the tool.

For some implementations, the tool is configured such that, at the distal portion of the tool, the sliding of the shuttle distally slides the tube away from the plug and into the holding zone.

For some implementations, the tool includes an inner shaft, configured to restrain the plug while the shuttle slides the tube away from the plug and into the holding zone.

For some implementations, the tool is configured such that the sliding of the shuttle distally slides the suture lock from the proximal portion of the tool to the distal portion of the tool by sliding the plug-lumen over and over and along the inner shaft.

For some implementations, a distal portion of the inner shaft defines a detent, the detent configured to inhibit distal sliding of the plug past the detent.

For some implementations, the tool includes a plug shaft, and is actuatable to secure the suture lock to the suture by pushing the plug shaft to push the plug distally into the tube-lumen.

For some implementations, the inner shaft is configured to be pulled distally by the plug as the plug is pushed distally into the tube-lumen, such that the inner shaft accompanies the plug in a manner that maintains the plug aligned with the tube-lumen.

For some implementations: the multi-shaft system includes an inner wire along which the inner shaft is slidably disposed, a distal portion of the inner shaft defines a compressible detent, and/or the multi-shaft system is configured such that: while the distal portion of the inner shaft is disposed over and along the inner wire, the inner wire inhibits radial compression of the detent, thereby inhibiting distal sliding of the plug past the detent while the shuttle slides the tube away from the plug and into the holding zone, and/or advancing the distal portion of the inner shaft distally beyond the inner wire increases compressibility of the detent, thereby facilitating withdrawal of the inner shaft from within the plug, while the plug is disposed withing the tube-lumen.

There is further provided, in accordance with some implementations, a system for use with a suture, the system including: a suture lock that includes: a tube: defining a tube-lumen that defines a proximal-distal axis of the suture lock, having a distal face, and/or having an outer surface that defines: at a first portion of the tube, a first width of the tube, at a second portion of the tube, a second width of the tube, the second width being smaller than the first width, and/or a shoulder between the first portion and the second portion, the second portion extending axially between the distal face and the shoulder, and/or a plug; and/or a tool configured to: hold the tube in a distal holding zone by engaging the shoulder, such that the distal face of the tube protrudes distally from a distal aperture of the tool, while continuing to hold the suture lock in the holding zone, lock the suture lock by inserting the plug into the tube-lumen, and/or subsequently release the suture lock out of the distal aperture.

For some implementations, at least one of the plug, the tube, and the tool is sterile.

For some implementations, the tool is configured to, while holding the tube in the holding zone: thread the suture through the tube, secure the suture lock to the suture by inserting the plug into the tube-lumen such that a portion of the suture becomes sandwiched between an outer surface of the plug and an inner surface of the tube, and/or trim the suture.

For some implementations: the first portion of the tube is a proximal portion of the tube, and/or the second portion of the tube is a distal portion of the tube having a distal outer diameter.

For some implementations: the shoulder of the tube is wider than a distal surface of the tube, and/or the tool is configured to hold the tube in the holding zone by the shoulder of the tube, such that the distal surface of the tube protrudes medially and distally from the tool.

For some implementations: the plug is shaped to define a plug-lumen, the tool includes an inner shaft, and/or the tool is configured to advance the suture lock from a proximal portion of the tool to a distal portion of the tool by sliding the suture lock over and along the inner shaft, while the inner shaft is disposed in the plug-lumen.

For some implementations, the tool is configured to lock the suture lock by inserting the plug into the tube-lumen such that a portion of the suture becomes sandwiched between an outer surface of the plug and an inner surface of the tube, laterally from the plug-lumen.

For some implementations, the tool further includes a plug shaft, and the tool is configured to secure the suture lock to the suture lock by advancing the plug shaft distally in a manner that: pushes the plug distally into the tube-lumen, and/or pulls the inner shaft distally.

For some implementations: the tool has a ready state in which the tool holds: the tube in the holding zone, such that the distal face of the tube protrudes distally from the distal aperture of the tool, and/or the plug, separated from the tube, within a distal portion of the tool; and/or the tool further includes: a snare at the distal portion of the tool, the snare configured to draw the suture through the tube-lumen while the tool is in the ready state, a force applicator at the distal portion of the tool, and/or a force regulator, proximal from the force applicator, the force regulator defining a force threshold, and being configured to, upon application of an actuation force to a proximal portion of the tool while the tool is in the ready state and the suture is disposed through the tube, transfer the actuation force to the force applicator in a manner that: locks the suture lock to the suture by inserting the plug into the tube-lumen while the actuation force remains below the force threshold, and/or upon the actuation force reaching the force threshold, causes the tool to trim the suture.

For some implementations, the force regulator is configured to, upon continued application of the actuation force to the proximal portion of the tool after the tool having trimmed the suture, transfer the actuation force to the force applicator in a manner that causes the tool to release the suture lock.

For some implementations, the force threshold is between 1.5–2.5 kg of force.

For some implementations, the force threshold is between 1.75–2.25 kg of force.

For some implementations, the force threshold is 2 kg of force.

For some implementations, the tool includes a multi-shaft system connecting the force regulator to the distal portion of the tool, a distal portion of the multi-shaft system defining the force applicator.

For some implementations: while the actuation force remains below the force threshold, a first member of the multi-shaft system defines the force applicator, and/or upon the actuation force reaching the force threshold, a second member of the multi-shaft system defines the force applicator.

For some implementations: the tool further includes: a lock shaft that defines a proximal storage zone and the distal holding zone, and/or a shuttle, slidable distally over and along the lock shaft in a manner that slides the suture lock distally toward the distal portion of the tool; and/or wherein the shuttle is configured to, by sliding distally over and along the lock shaft while the shuttle is engaged with the tube: transport the suture lock from the storage zone toward the holding zone, and/or unlock the suture lock by separating the plug from the tube, such that the tool assumes the ready state.

For some implementations: the shuttle houses the snare; and/or the shuttle is configured to, while the tool is in the ready state: release the snare from the shuttle and distally through the tube-lumen, such that the snare snares a length of suture, and/or draw the snared suture proximally back through the tube-lumen by proximally retracting the snare.

For some implementations: the distal portion of the lock shaft defines a holding arm bordering the holding zone, and/or the shuttle is configured to fit the tube into the holding zone by changing an orientation of the holding arm.

For some implementations, the distal portion of the lock shaft is configured such that, after the tube fits into the holding zone, the holding arm supports the tube in the holding zone.

For some implementations, the shuttle is configured to, by sliding distally over and along the lock shaft while the shuttle is engaged with the tube: transport the suture lock from the storage zone toward the holding zone, and/or unlock the suture lock by advancing the tube away from the plug and into the holding zone, such that the tool assumes the ready state.

For some implementations, the tool includes an inner shaft, configured to longitudinally restrain the plug while the shuttle unlocks the suture lock.

For some implementations: the tool further includes a plug shaft that slidably fits within the lock shaft, and/or the plug shaft is configured to lock the suture lock by pushing the plug distally into the tube at the holding zone.

For some implementations: the plug is shaped to define a plug-lumen, the shuttle is configured to transport the suture lock from the storage zone toward the holding zone by sliding the suture lock over and along the inner shaft, while the inner shaft is disposed in the plug-lumen, and/or the plug shaft is configured to lock the suture lock by pushing the plug distally into the tube at the holding zone, such that the inner shaft is pulled distally along with the plug.

For some implementations, the shuttle is configured to compress the holding zone around the tube by sliding distally over and along the lock shaft while the tool is in the ready state.

For some implementations: the tool further includes an outer shaft slidably housing the lock shaft; and/or the tool is configured such that, while: the tool is in the ready state, and/or the shuttle is engaged with the outer shaft, sliding the shuttle distally over and along the lock shaft compresses the holding zone around the tube by passing the outer shaft over and along the holding zone.

There is further provided, in accordance with some implementations, a system for use with a suture, the system including: a suture lock that has an outer surface that defines a shoulder; and/or a tool configured to: advance the suture lock from a proximal portion of the tool to a holding zone at a distal portion of the tool, hold at least a part of the suture lock in the holding zone by engaging the shoulder, such that a distal face of the suture lock protrudes distally from a distal aperture of the tool, secure the suture lock to the suture while continuing to hold the suture lock in the holding zone, and/or subsequently release the suture lock out of the distal aperture.

For some implementations, at least one of the suture lock and the tool is sterile.

For some implementations: the shoulder of the suture lock is wider than a distal surface of the suture lock, and/or the tool is configured to hold at least the part of the suture lock in the holding zone by the shoulder of the suture lock, such that the distal surface of the suture lock protrudes medially and distally from the tool.

For some implementations: the tool further includes: a snare at the distal portion of the tool, the snare configured to draw the suture through at least the part of the suture lock; a force applicator at the distal portion of the tool; and/or a force regulator, proximal from the force applicator, the force regulator defining a force threshold, and being configured to, upon application of an actuation force to a proximal portion of the tool while the suture is disposed through at least the part of the suture lock, transfer the actuation force to the force applicator in a manner that: secures the suture lock to the suture while the actuation force remains below the force threshold, and/or upon the actuation force reaching the force threshold, causes the tool to trim the suture.

For some implementations, the force regulator is configured to, upon continued application of the actuation force to the proximal portion of the tool after the tool having trimmed the suture, transfer the actuation force to the force applicator in a manner that causes the tool to release the suture lock.

For some implementations, the force threshold is between 1.5–2.5 kg of force.

For some implementations, the force threshold is between 1.75–2.25 kg of force.

For some implementations, the force threshold is 2 kg of force.

For some implementations, the tool includes a multi-shaft system connecting the force regulator to the distal portion of the tool, a distal portion of the multi-shaft system defining the force applicator.

For some implementations: while the actuation force remains below the force threshold, a first member of the multi-shaft system defines the force applicator, and/or upon the actuation force reaching the force threshold, a second member of the multi-shaft system defines the force applicator.

For some implementations, the tool further includes a shuttle configured to, by sliding distally over and along the multi-shaft system: transport the suture lock from the proximal portion of the tool to the distal portion of the tool, and/or unlock the suture lock, such that the tool assumes a ready state in which at least the part of the unlocked suture lock is disposed in the holding zone.

For some implementations: the shuttle houses the snare; and/or the shuttle is configured to, while the tool is in the ready state: release the snare from the shuttle and distally through at least the part of the suture lock, such that the snare snares a length of suture, and/or draw the snared suture proximally back through at least the part of the suture lock by proximally retracting the snare.

For some implementations, the multi-shaft system includes an inner shaft, configured to longitudinally restrain the suture lock while the shuttle unlocks the suture lock.

For some implementations: the distal portion of the tool defines a holding arm bordering the holding zone, and/or the shuttle is configured to fit at least the part of the suture lock into the holding zone by changing an orientation of the holding arm.

For some implementations, the distal portion of the tool is configured such that, after at least the part of the suture lock fits into the holding zone, the holding arm supports at least the part of the suture lock in the holding zone.

For some implementations, the shuttle is configured to compress the holding zone around the suture lock by sliding distally over and along the multi-shaft system while the tool is in the ready state.

For some implementations: the multi-shaft system further includes an outer shaft; and/or the tool is configured such that, while: the tool is in the ready state, and/or the shuttle is engaged with the outer shaft, sliding the shuttle distally over and along the distal portion of the tool compresses the holding zone around at least the part of the unlocked suture lock by passing the outer shaft over and along the holding zone.

There is further provided, in accordance with some implementations, a system for locking a suture, the system including: a suture lock including: a plug, and/or a tube defining a tube-lumen; and/or a tool including: a lock shaft: extending from a proximal portion of the tool to a distal portion of the tool, and/or defining a holding zone at a distal portion of the lock shaft, and/or a shuttle, configured to: slide the suture lock from a proximal portion of the lock shaft to the distal portion of the lock shaft, and/or unlock the suture lock by sliding the tube away from the plug and into the holding zone.

For some implementations, at least one of the plug, the tube, and the tool is sterile.

For some implementations, the tool is configured to thread a suture through the suture lock by pulling the suture proximally through the tube-lumen while the tube is seated at the holding zone.

For some implementations, the tool further includes an outer shaft slidably housing the lock shaft, and the shuttle is configured to compress the holding zone by drawing the outer shaft over the holding zone.

For some implementations: the tool further includes a snare, housed within the shuttle; and/or the tool is configured such that, while the suture lock is unlocked and the tube is seated in the holding zone, the shuttle is operatable to: release the snare from the shuttle and distally through the tube-lumen, and/or retract the snare proximally through the tube and into the shuttle.

For some implementations, the tool is configured such that distally sliding the shuttle over and along the holding zone fits the tube into the holding zone.

For some implementations: the distal portion of the lock shaft defines a holding arm bordering the holding zone, and/or the tool is configured such that distally sliding the shuttle over and along the holding zone fits the tube into the holding zone by changing an orientation of the holding arm.

For some implementations, the distal portion of the lock shaft is configured such that, after the tube fits into the holding zone, the holding arm supports the tube in the holding zone.

For some implementations, the tool is configured such that distally sliding the shuttle over and along the holding zone compresses the holding zone around the tube.

For some implementations: the tool further includes an outer shaft, and/or the tool is configured such that, while the shuttle is engaged with the outer shaft, sliding the shuttle distally over and along the distal portion of the tool compresses the holding zone around the tube by passing the outer shaft over and along the holding zone.

For some implementations: the tool further includes a snare, housed within the shuttle; and/or the tool is configured such that, while the suture lock is unlocked and the tube is disposed at the holding zone, the shuttle is operatable to: advance the snare from the shuttle and distally through the tube, and/or retract the snare proximally out of the tube and into the shuttle.

For some implementations, the tool is configured to thread a suture through the tube by pulling the suture proximally through the tube while at least a part of the unlocked suture lock is disposed at the holding zone.

For some implementations, the tool includes an inner shaft, configured to longitudinally restrain the plug while the shuttle unlocks the suture lock.

For some implementations: the plug is shaped to define a plug-lumen, and/or the shuttle is configured to slide the suture lock from the proximal portion of the lock shaft to the distal portion of the lock shaft by sliding the suture lock over and along the inner shaft, while the inner shaft is disposed in the plug-lumen.

For some implementations, the tool includes a plug shaft, configured to lock the suture lock by: pushing the plug distally into the tube-lumen, and/or sliding the inner shaft distally.

There is further provided, in accordance with some implementations, a system for locking a suture, the system including: multiple suture locks; and/or a tool including: at a proximal portion of the tool, a storage zone in which the multiple suture locks are disposed, a lock shaft: extending from the proximal portion of the tool to a distal portion of the tool, and/or defining a holding zone at a distal portion of the lock shaft, and/or a shuttle, mounted such that sliding of the shuttle distally over and along the lock shaft slides one of the suture locks distally from the storage zone into the holding zone, and compresses the holding zone in a manner that traps the suture lock in the holding zone.

For some implementations, at least one of the tool and the suture locks is sterile.

For some implementations, the tool further includes an outer shaft slidably housing the lock shaft, and the shuttle is configured to compress the holding zone by drawing the outer shaft over the holding zone.

There is further provided, in accordance with some implementations, a system for locking a suture, the system including: a suture lock; and/or a tool including: at a proximal portion of the tool, a storage zone in which the suture lock are disposed, a lock shaft, extending from the proximal portion of the tool to a distal portion of the tool, at the distal portion of the tool, a holding zone, and/or a shuttle, mounted such that sliding of the shuttle distally over and along the lock shaft: slides the suture lock distally out of the storage zone, unlocks the suture lock, and/or advances at least a part of the unlocked suture lock into the holding zone.

For some implementations, at least one of the suture lock and the tool is sterile.

For some implementations: the tool further includes a snare, housed within the shuttle; and/or the tool is configured such that, while the suture lock is unlocked and at least the part of the unlocked the suture lock is disposed at the holding zone, the shuttle is operatable to: advance the snare from the shuttle and distally through at least the part of the unlocked suture lock, and/or retract the snare proximally out of at least the part of the unlocked suture lock and into the shuttle.

For some implementations, the tool is configured to thread a suture through at least the part of the unlocked suture lock by pulling the suture proximally through at least the part of the unlocked suture lock while at least the part of the unlocked suture lock is disposed at the holding zone.

For some implementations: the suture lock is shaped to define a lumen, and/or the shuttle is configured to thread the suture through at least the part of the unlocked suture lock within the holding zone by pulling the snare proximally through the lumen while at least the part of the unlocked suture lock is seated at the holding zone.

For some implementations, the shuttle includes: a snare, housed within the shuttle; a snare pump, operatable to: release the snare from the shuttle, and/or thread the suture through at least the part of the suture lock by pulling the suture proximally through the lumen while at least the part of the suture lock is seated at the holding zone; and/or a pusher, reversibly engageable to at least the part of the suture lock and configured to: support at least the part of the suture lock while at least the part of the suture lock is seated at the holding zone, and/or inhibit operation of the snare pump while the pusher is disengaged from the suture lock.

For some implementations: the snare pump is operatable by longitudinally sliding the snare pump with respect to the holding zone; and/or the pusher is configured such that: while the pusher engages at least the part of the suture lock, the pusher assumes a first conformation in which the snare pump is slidable with respect to the pusher, and/or while the pusher is disengaged from the suture lock, the pusher assumes a second conformation in which the pusher inhibits sliding the snare pump with respect to the holding zone.

For some implementations, the shuttle includes: a snare, housed within a snare tube; a snare pump, operatable to: release the snare from the snare tube, and/or thread the suture through at least the part of the suture lock by pulling the suture proximally through the lumen while at least the part of the suture lock is seated at the holding zone; and/or a snare interlock coupled to the snare tube, the snare interlock configured to: facilitate release of the snare from the snare tube while a distal opening of the snare tube faces the holding zone, and/or inhibit release of the snare from the snare tube while the distal opening of the snare tube faces away from the holding zone.

For some implementations: the snare pump is operatable by longitudinally sliding the snare pump with respect to the holding zone; and/or the snare interlock includes a snare interlock lever, the snare interlock lever: coupled to the snare tube at a first portion of the snare interlock lever, shaped to define a brake at a second portion of the snare interlock lever, and/or pivotable such that: while the distal opening of the snare tube faces away from the holding zone, the brake inhibits sliding the snare pump with respect to the holding zone, and/or while the distal opening of the snare tube faces the holding zone, the brake allows sliding the snare pump with respect to the holding zone.

There is further provided, in accordance with some implementations, a system, including: multiple suture locks, each suture lock including: a plug, and/or a tube; and/or a tool including: at a proximal portion of the tool, a storage zone in which the multiple suture locks are disposed, a multi-shaft system, extending from the proximal portion of the tool to a distal portion of the tool, a shuttle: mounted to be repeatedly shuttled, each shuttling including (i) an outbound leg in which the shuttle is slid distally over and along the multi-shaft system from the proximal portion of the tool to the distal portion of the tool, and (ii) a return leg in which the shuttle is subsequently slid proximally over and along the multi-shaft system to return from the distal portion of the tool to the proximal portion of the tool, and/or configured such that, during each shuttling: the outbound leg slides one of the suture locks distally from the storage zone to the distal portion, and/or the return leg leaves the suture lock at the distal portion, and/or a trigger, operable to lock the suture lock within the distal portion by actuating the multi-shaft system to push the plug into the tube.

There is further provided, in accordance with some implementations, a system for securing a suture, the system including a suture lock that includes: a plug; and/or a tube, configured to receive the plug, and including a wall that: circumscribes and defines a tube axis and a tube-lumen along the tube axis, and/or has a set of axial slits therein, distributed circumferentially around the tube axis to provide the wall with compliance that facilitates insertion of the plug into the tube and retention of the plug by the tube.

For some implementations, the suture lock is sterile.

For some implementations, the plug is configured to be inserted into the tube by being advanced along the tube axis and into the tube-lumen by a locking force that is lesser than a locking force that would be required in absence of the set of axial slits.

For some implementations, the plug is configured to be retained within the tube-lumen by interference fit.

For some implementations, a proximal portion of the tube has a compliance that is greater than a compliance of a distal portion of the tube.

For some implementations, the set of axial slits is configured to provide the wall with compliance by each axial slit changing shape upon insertion of the plug into the tube.

For some implementations, the set of axial slits is configured to provide the wall with compliance by each axial slit expanding upon insertion of the plug into the tube.

For some implementations, the set of axial slits are a set of proximal slits cut from a proximal end of the tube such that the proximal slits define a set of proximal tabs therebetween.

For some implementations, the proximal tabs are configured to provide the wall with compliance by expanding radially outward as the plug enters the tube-lumen.

For some implementations: the set of slits is a first set of axial slits, and/or the tube has a second set of axial slits therein, distributed circumferentially around the tube axis and extending further distally than the first set of slits.

For some implementations, the second set of axial slits are circumferentially offset from the first set of axial slits.

For some implementations, the first set of axial slits are configured to provide a proximal portion of the wall with a greater compliance than the second set of axial slits provide to a distal portion of the wall.

For some implementations, the first set of axial slits are configured to provide the proximal portion of the wall with the greater compliance by changing shape, upon insertion of the plug into the tube, to a greater degree than the second set of axial slits.

For some implementations, the tube has a distal slit therein, the distal slit configured to resist advancing the plug distally through the tube, past the distal slit.

For some implementations, the distal slit is perpendicular to the tube axis.

For some implementations, the plug is shaped to define a ridge that facilitates retention of the plug by the tube.

For some implementations, the ridge is a proximal ridge, configured to inhibit advancing the plug distally through the tube such that the proximal ridge enters the tube.

For some implementations: the ridge is a first ridge, the plug is shaped to further define a second ridge, and/or the tube is configured to snap into place between the first ridge and the second ridge.

For some implementations, the ridge is a distal ridge, configured to, after insertion of the plug into the tube, inhibit removing the plug from the tube.

For some implementations, the distal ridge has: a distal shoulder that facilitates insertion of the plug into the tube, and/or a proximal step that inhibits withdrawing the plug through the tube.

For some implementations, the suture lock has: an unlocked state in which the plug is disposed outside of the tube-lumen, and/or a locked state in which the plug is at least partially disposed within the tube-lumen.

For some implementations, while the suture lock is in the unlocked state, the plug has an external diameter that is greater than an internal diameter of the tube.

For some implementations, the tube has a greater elasticity than the plug.

For some implementations, the tube includes nitinol.

For some implementations, the plug includes cobalt-chrome.

There is further provided, in accordance with some implementations, a system for securing a suture, the system including a suture lock that includes: a plug; and/or a tube, configured to receive the plug, and including a wall that: circumscribes and defines a tube axis and a tube-lumen along the tube axis, and/or has a set of slits therein, distributed circumferentially around the tube axis and aligned with the tube axis.

For some implementations, the suture lock is sterile.

For some implementations, the plug is configured to be inserted into the tube by being advanced along the tube axis and into the tube-lumen by a locking force that is lesser than a locking force that would be required in absence of the set of slits.

For some implementations, the plug is configured to be retained within the tube-lumen by interference fit.

For some implementations, a proximal portion of the tube has a compliance that is greater than a compliance of a distal portion of the tube.

For some implementations, the set of slits is configured to provide the wall with compliance by each slit changing shape upon insertion of the plug into the tube.

For some implementations, the set of slits is configured to provide the wall with compliance by each slit expanding upon insertion of the plug into the tube.

For some implementations, the set of slits are a set of proximal slits cut from a proximal end of the tube such that the proximal slits define a set of proximal tabs therebetween.

For some implementations, the proximal tabs are configured to provide the wall with compliance by expanding radially outward as the plug enters the tube-lumen.

For some implementations: the set of slits is a first set of slits, and/or the tube has a second set of slits therein, distributed circumferentially around the tube axis and extending further distally than the first set of slits.

For some implementations, the second set of slits are circumferentially offset from the first set of slits.

For some implementations, the first set of slits are configured to provide a proximal portion of the wall with a greater compliance than the second set of slits provide to a distal portion of the wall.

For some implementations, the first set of slits are configured to provide the proximal portion of the wall with the greater compliance by changing shape, upon insertion of the plug into the tube, to a greater degree than the second set of slits.

For some implementations, the tube has a distal slit therein, the distal slit configured to resist advancing the plug distally through the tube, past the distal slip.

For some implementations, the distal slit is perpendicular to the tube axis.

For some implementations, the plug is shaped to define a ridge that facilitates retention of the plug by the tube.

For some implementations, the ridge is a proximal ridge, configured to inhibit advancing the plug distally through the tube such that the proximal ridge enters the tube.

For some implementations: the ridge is a first ridge, the plug is shaped to further define a second ridge, and/or the tube is configured to snap into place between the first ridge and the second ridge.

For some implementations, the ridge is a distal ridge, configured to, after insertion of the plug into the tube, inhibit removing the plug from the tube.

For some implementations, the distal ridge has: a distal shoulder that facilitates insertion of the plug into the tube, and/or a proximal step that inhibits withdrawing the plug through the tube.

For some implementations, the suture lock has: an unlocked state in which the plug is disposed outside of the tube-lumen, and/or a locked state in which the plug is at least partially disposed within the tube-lumen.

For some implementations, while the suture lock is in the unlocked state, the plug has an external diameter that is greater than an internal diameter of the tube.

For some implementations, the tube has a greater elasticity than the plug.

For some implementations, the tube includes nitinol.

For some implementations, the plug includes cobalt-chrome.

There is further provided, in accordance with some implementations, a system for securing a suture, the system including a suture lock that includes: a plug, formed from cobalt-chrome; and/or a tube, formed from nitinol, and configured to receive the plug.

For some implementations, the suture lock is sterile.

There is further provided, in accordance with some implementations, a system for use with a suture, the system including: multiple suture locks, each suture lock including: a first lock piece, and/or a second lock piece; and/or a tool: including: at a proximal portion of the tool, a storage zone in which the multiple suture locks are disposed, and/or operable to, for each of the multiple suture locks, sequentially: advance the first lock piece and the second lock piece distally from the storage zone to a distal portion of the tool, thread the suture through the first lock piece, and/or while the suture remains disposed through the first lock piece: secure the suture lock to the suture by engaging the first lock piece with the second lock piece, and/or trim the suture.

For some implementations, at least one of the tool and the suture locks is sterile.

For some implementations, for each of the suture locks disposed in the storage zone, the second lock piece is engaged with the first lock piece.

For some implementations, the tool is configured to advance each of the suture locks distally from the storage zone while the second lock piece is engaged with the first lock piece.

For some implementations, the tool is operable to secure each of the suture locks to the suture by sandwiching the suture between first lock piece and the second lock piece.

For some implementations, the tool is configured to separate the first lock piece from the second lock piece, prior to engaging the first lock piece with the second lock piece.

For some implementations, the tool is configured to: advance each of the suture locks from the storage zone to the distal portion while the second lock piece is engaged with the first lock piece, and/or within the distal portion of the tool, separate the first lock piece from the second lock piece.

For some implementations, the tool is configured to separate the first lock piece from the second lock piece by advancing the first lock piece, within the distal portion of the tool, distally from the second lock piece.

For some implementations, the tool further includes a trigger, configured to, upon application of an actuation force to the trigger, actuate the tool to trim the suture while engaging the first lock piece with the second lock piece.

For some implementations: the first lock piece and the second lock piece include a first suture lock of the multiple suture locks, the trigger is operable to, upon application of the actuation force to the trigger, cyclically actuate the tool such that trimming the suture advances the tool toward a ready state in which the tool is actuatable to advance a first lock piece and a second lock piece of a second suture lock to the distal portion of the tool.

For some implementations, subsequently to securing the suture lock to the suture by engaging the first lock piece with the second lock piece, and prior to actuation of the tool to trim the suture, the tool continues to engage the first lock piece with the second lock piece, in absence of the actuation force.

There is further provided, in accordance with some implementations, a system for use with a suture, the system including: multiple suture locks, each suture lock including: a first lock piece, and/or a second lock piece; and/or a tool, including: at a proximal portion of the tool, a storage zone in which the multiple suture locks are disposed, a snare, operable to, for each of the multiple suture locks, sequentially thread the suture through the first lock piece and into a distal portion of the tool, and/or a multi-shaft system: extending from the proximal portion of the tool to the distal portion of the tool, and/or operable to, for each of the multiple suture locks, sequentially: advance the first lock piece and the second lock piece distally from the storage zone to the distal portion, and/or while the suture remains disposed through the first lock piece, secure the suture lock to the suture by engaging the first lock piece with the second lock piece, and/or a suture-exposing mechanism, operable to expose part of the suture laterally from the tool.

For some implementations, at least one of the tool and the suture locks is sterile.

For some implementations, the snare is operable to, for each of the multiple suture locks, sequentially thread the suture through the first lock piece and into a distal portion of the multi-shaft system.

For some implementations, the suture-exposing mechanism is operable to expose part of the suture laterally from the multi-shaft system and into the distal portion of the multi-shaft system while the suture remains disposed through the first lock piece.

For some implementations, the snare is operable to: extend distally through the first lock piece, while the snare is extended distally from the first lock piece, snare a length of the suture and/or while holding the snared suture, retract proximally, thereby threading the suture through the first lock piece.

For some implementations, the suture-exposing mechanism includes a cantilever that is pivotable between: an aligned state in which the cantilever is aligned along the multi-shaft system, and/or an oblique state in which the cantilever is angled laterally away from the multi-shaft system.

For some implementations: the snare is operable to thread the suture through the first lock piece and through a portion of the cantilever, while the cantilever is in the aligned state, and/or the cantilever is pivotable toward the oblique state while the suture remains threaded through the portion of the cantilever, thereby exposing part of the suture from the multi-shaft system, while the suture remains threaded through the first lock piece.

The present disclosure will be more fully understood from the following detailed description of implementations thereof, taken together with the drawings, in which:

### BRIEF DESCRIPTION OF THE DRAWINGS

FIG. 1A is a schematic illustration showing a tool for use with a suture and a suture lock, including aesthetic features thereof, in accordance with some implementations;

FIGS. 1B–F are schematic illustrations showing a suture lock comprising a tube and a plug, including aesthetic features thereof, in accordance with some implementations;

FIGS. 2-3 and FIGS. 4A-B are schematic illustrations showing a handle of the tool, including aesthetic features thereof, in accordance with some implementations;

FIGS. 5-7 are schematic illustrations showing a shuttle of the tool, including aesthetic features thereof, in accordance with some implementations;

FIGS. 8A-B and FIGS. 9-10 are schematic illustrations showing a multi-shaft system of the tool, including aesthetic features thereof, in accordance with some implementations;

FIGS. 11A-H are schematic illustrations showing use of the tool to advance the suture lock to a distal portion of the multi-shaft system, and to thread a length of suture through the suture lock, including aesthetic features thereof, in accordance with some implementations;

FIGS. 11I-P, FIGS. 12A-E and FIG. 13 are schematic illustrations showing use of the tool to lock the suture lock onto a length of suture at an implant, including aesthetic features thereof, in accordance with some implementations;

FIG. 14 is a schematic illustration showing a plurality of suture locks having been applied to respective sutures at the implant, including aesthetic features thereof, in accordance with some implementations;

FIG. 15 shows schematic illustrations of a tool for use with the suture and the suture lock, in accordance with some implementations;

FIGS. 16-17 and FIGS. 18A-B are schematic illustrations showing a handle of the tool, in accordance with some implementations;

FIGS. 19 and 20A–B are schematic illustrations showing a shuttle of the tool, in accordance with some implementations;

FIGS. 21A-B and FIGS. 22-23 are schematic illustrations showing a multi-shaft system of the tool, in accordance with some implementations;

FIG. 24 is a schematic illustration showing the delivery tool in a pre-loading state, in accordance with some implementations;

FIGS. 25A-L are schematic illustrations showing use of the tool to advance the suture lock a distal portion of the system, and to thread a length of suture through the suture lock, in accordance with some implementations;

FIGS. 25M-R and FIGS. 26A-F are schematic illustrations showing use of the tool to lock the suture lock onto a length of suture at an implant, in accordance with some implementations;

FIGS. 27A-C, FIGS. 28A-B, 29A-B and FIG. 30 are schematic illustrations showing suture locks, in accordance with some implementations; and

FIGS. 31, 32A-F and FIGS. 33A-D are schematic illustrations showing delivery tools, in accordance with some implementations.

### DETAILED DESCRIPTION

The present disclosure includes different variants of some elements. Variants of a given element typically have the same structure and/or function as each other except for any differences described. For any given element for which different variants are disclosed, the identical name is used for each variant, in order to denote that they are, in fact, variants of the same given element. Unless stated otherwise, implementations of the devices, systems, and techniques described herein may include any arrangement in which one variant of an element is substituted with another identically-named variant of that element. Furthermore, throughout the figures, suffixes are used to denote different variants of the same element. Unless stated otherwise, such variants may be substituted with each other, mutatis mutandis. That is, unless stated otherwise, any element having a given reference numeral may be substituted with any other element (e.g., any other variant of the element) having the same reference numeral, independent of any suffix.

The disclosure of numerical ranges should be understood as referring to each discrete point within the range, inclusive of endpoints, unless otherwise noted. Unless otherwise indicated, all numbers expressing quantities, magnitudes, percentages, temperatures, times, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods as known to those of ordinary skill in the art. When directly and explicitly distinguishing examples from discussed references, the example numbers are not approximate unless the word “about” is recited.

In order to avoid undue clutter from having too many reference numbers and lead lines on a particular drawing, some elements are introduced via one or more drawings and not explicitly identified in every other drawing that contains that element.

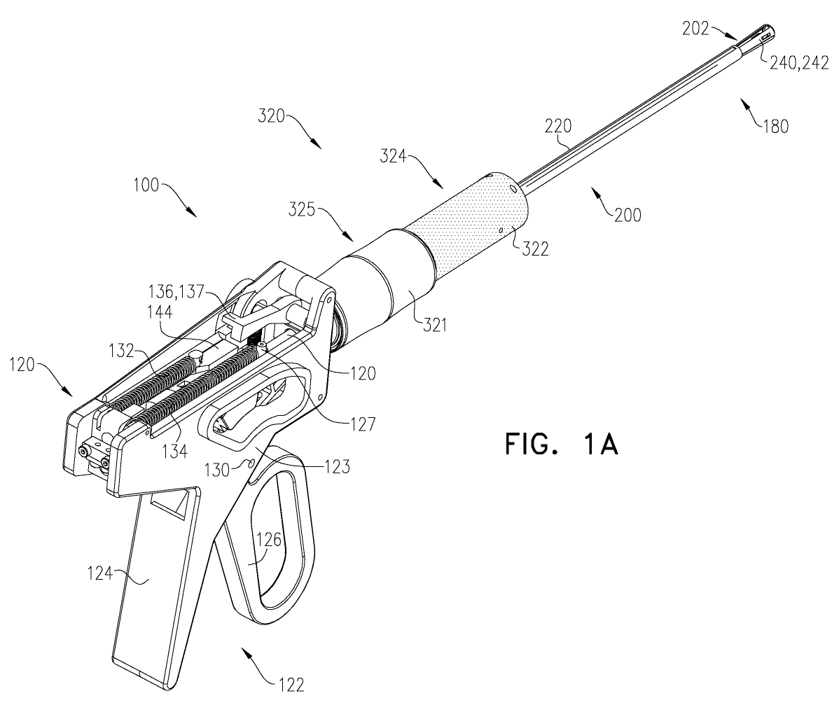

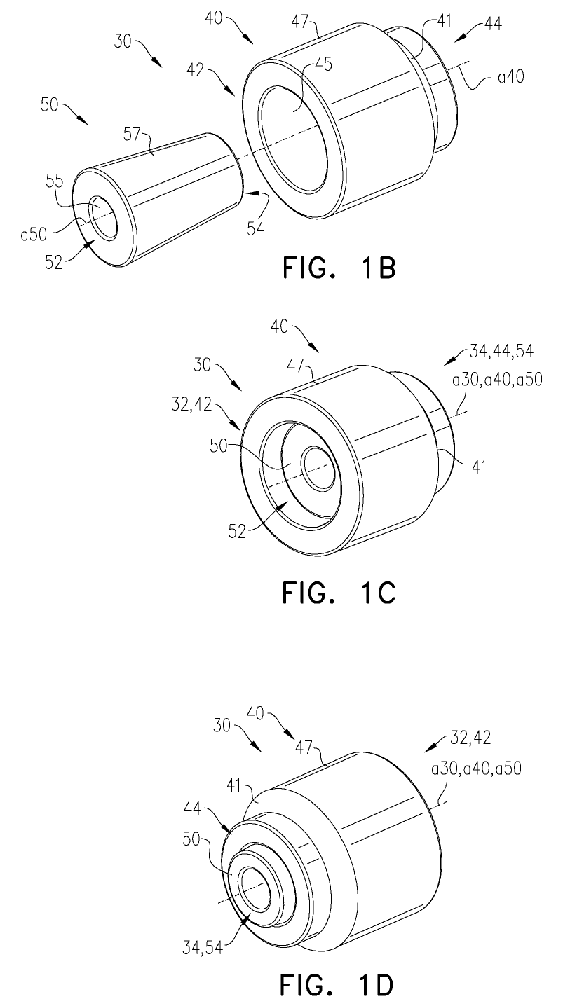

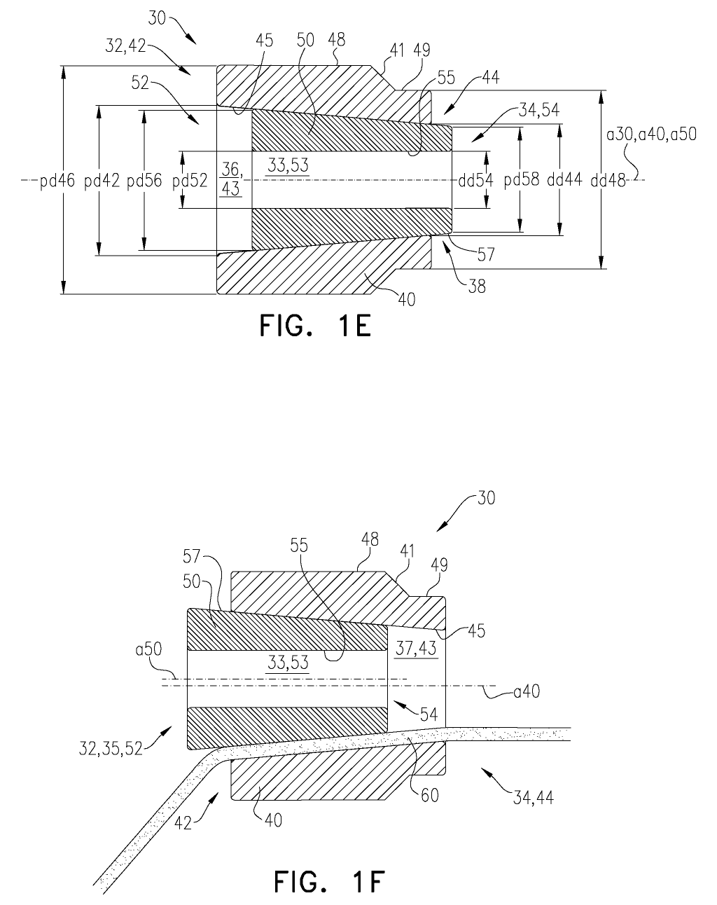

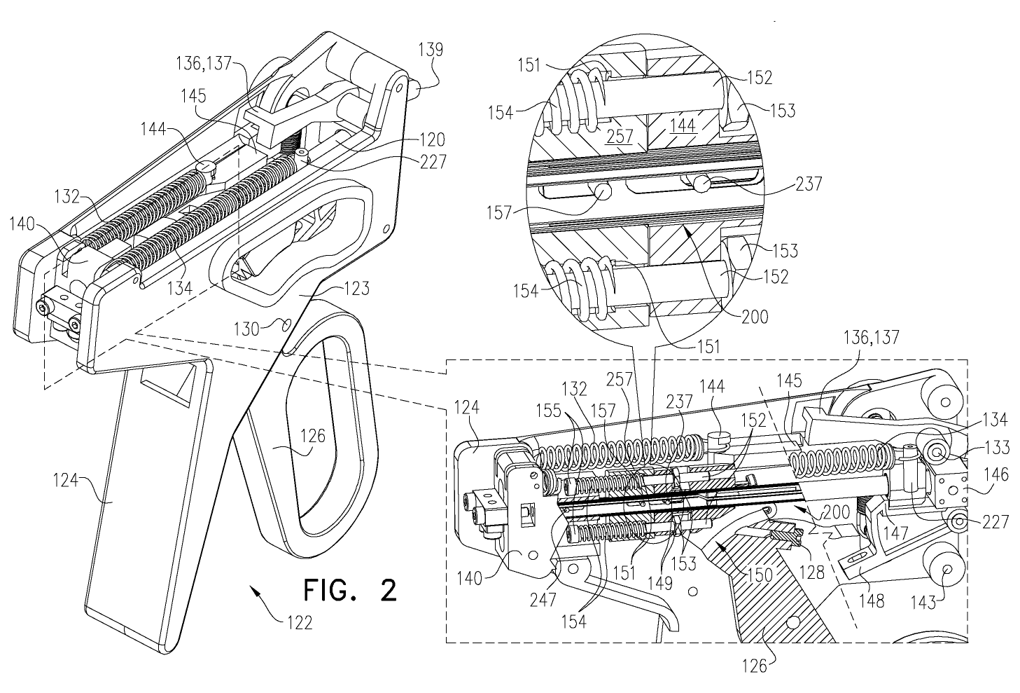

Reference is made to FIG. 1A, which is a schematic illustration showing a tool 100 (e.g., a delivery tool) for use with a suture 60 and a suture lock 30, in accordance with some implementations.

In some implementations, and as shown, tool 100 has a handle 122 at the tool’s proximal portion 120, a multi-shaft system 200 that extends from the handle to the tool’s distal portion 180, and a shuttle 320 that is slidable over and along the multi-shaft system, between the handle and a distal portion 202 of the multi-shaft system.

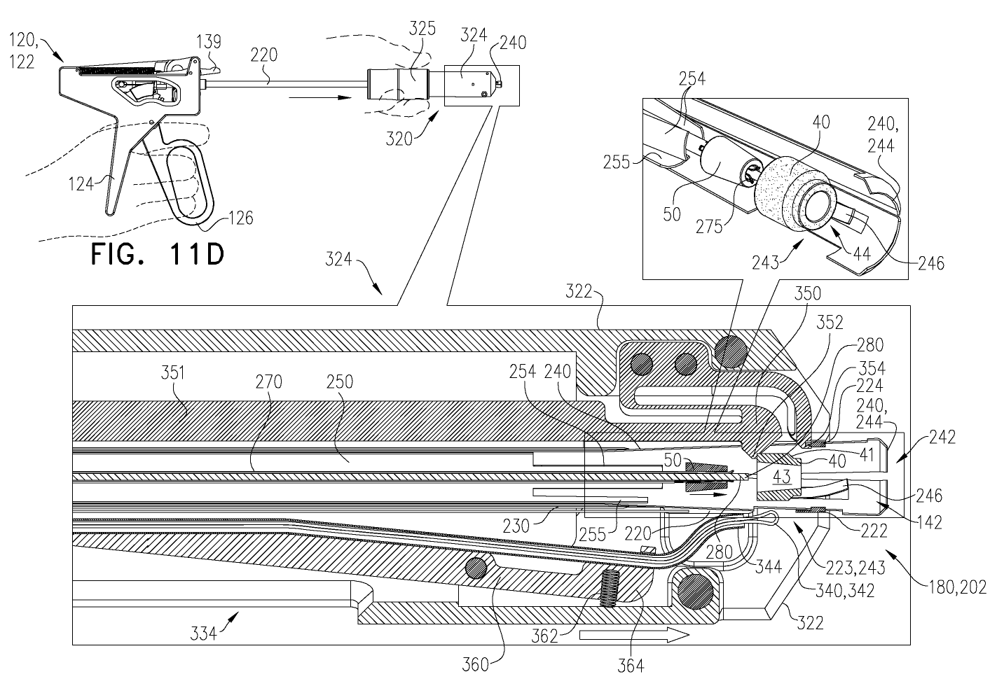

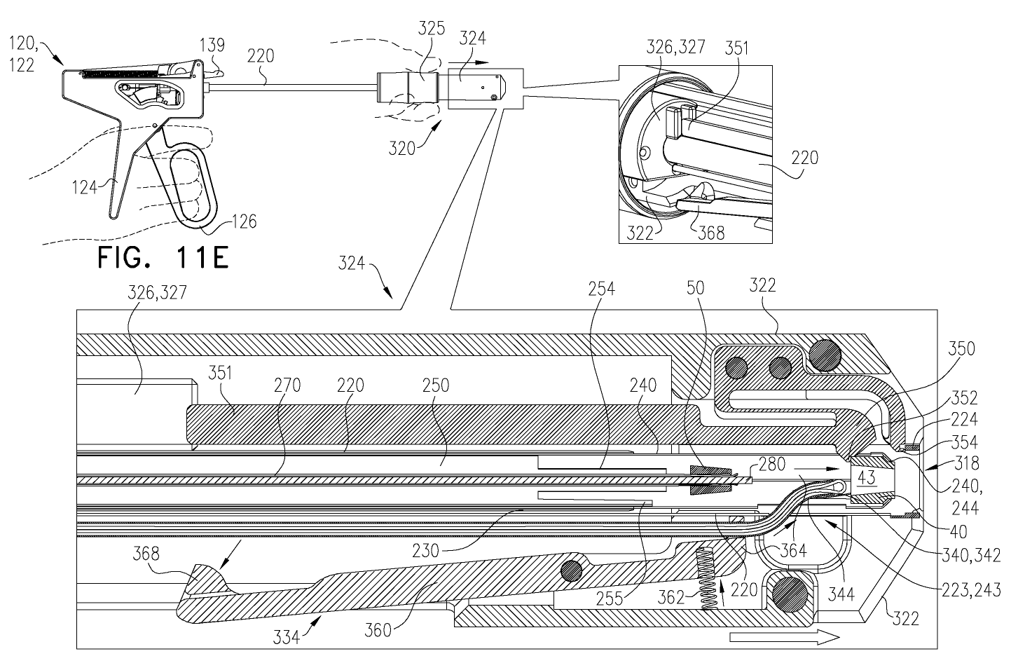

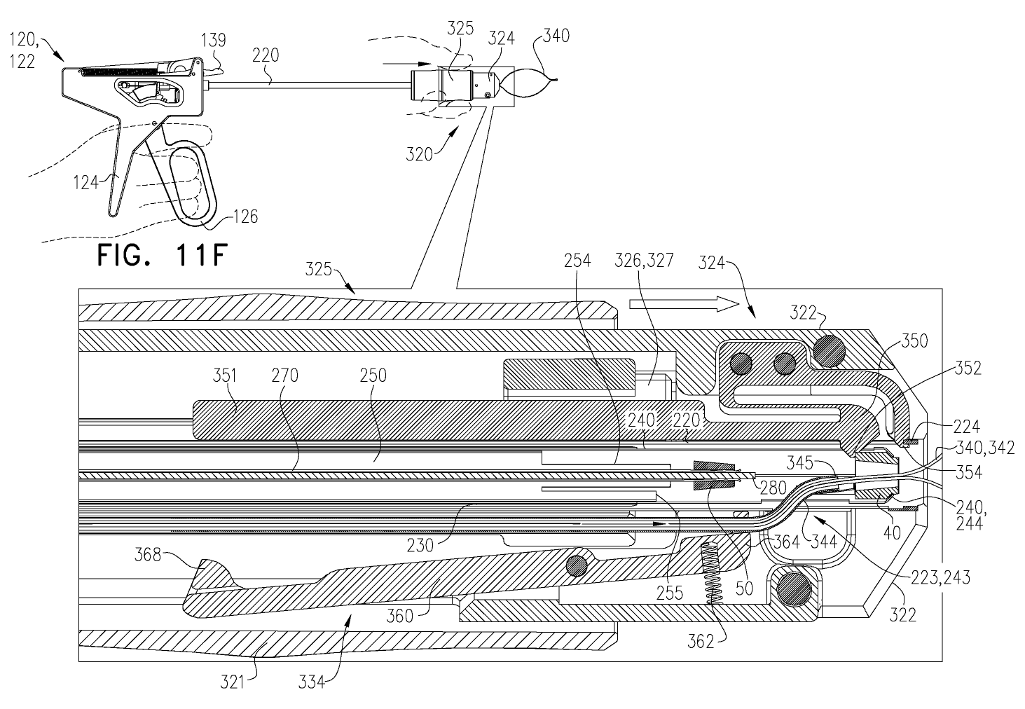

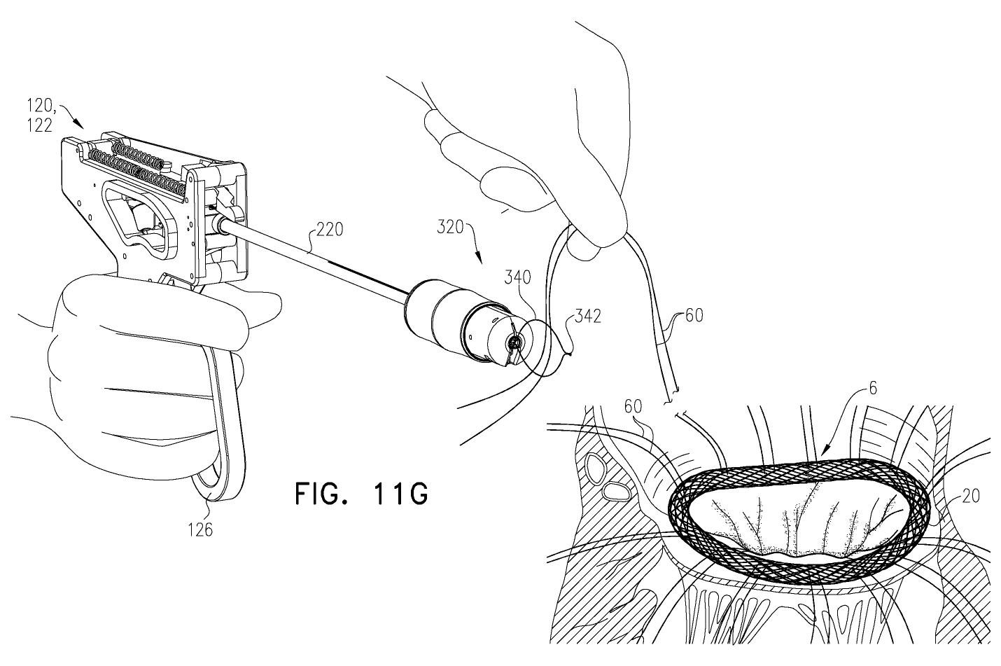

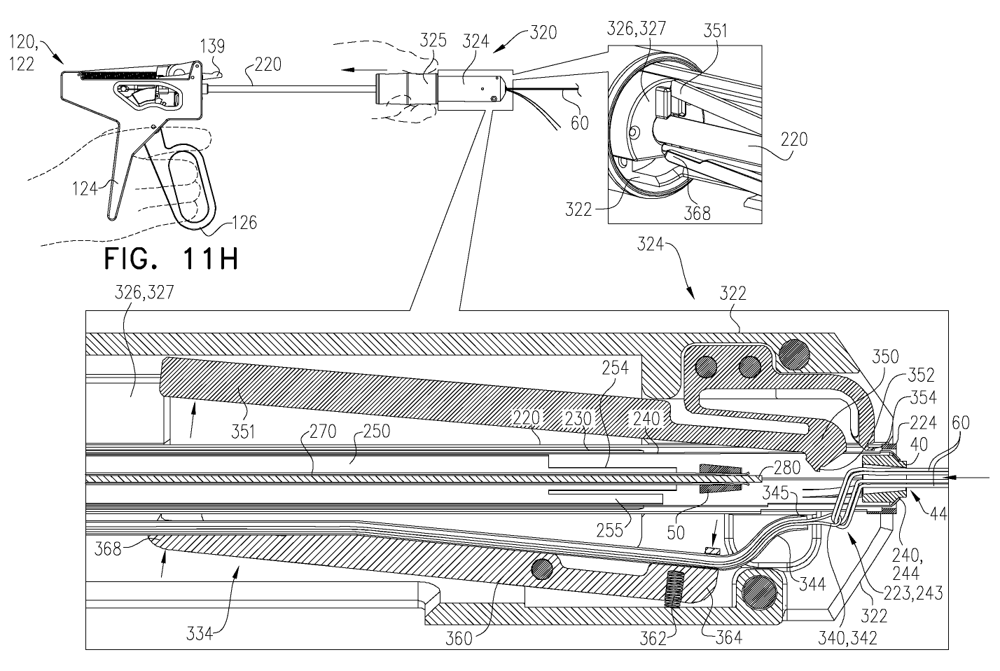

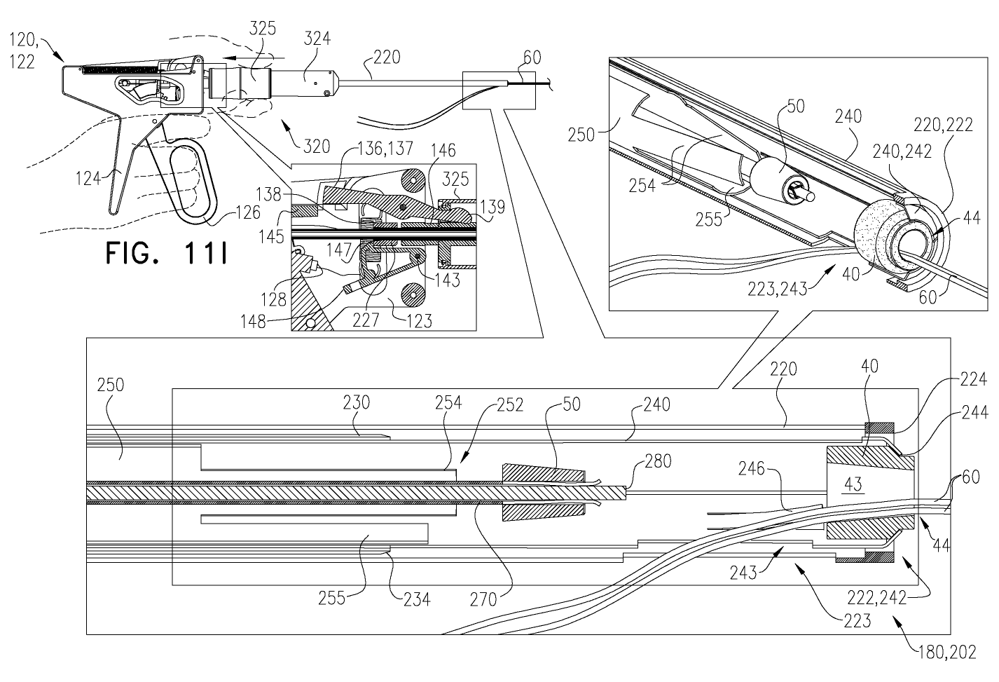

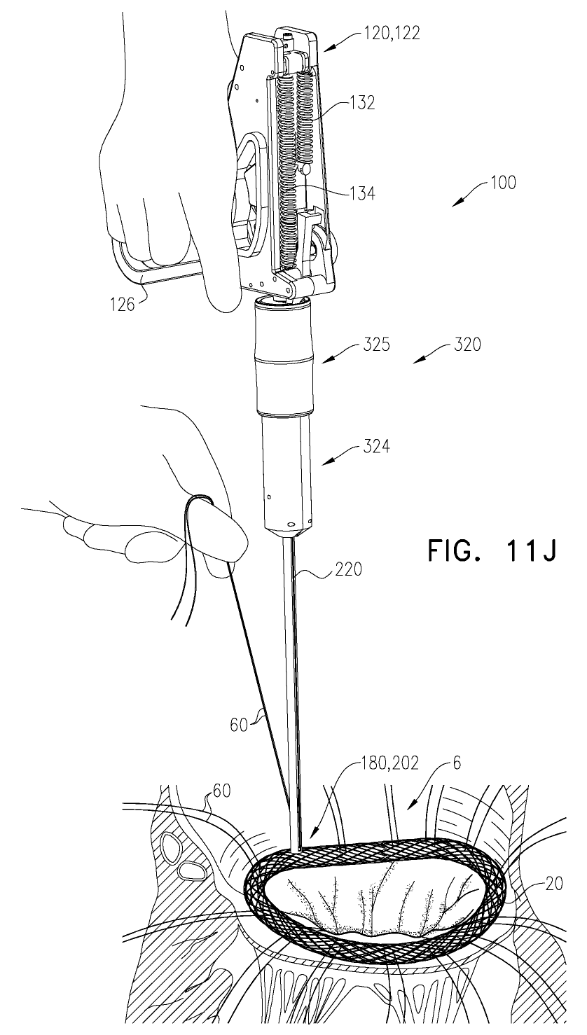

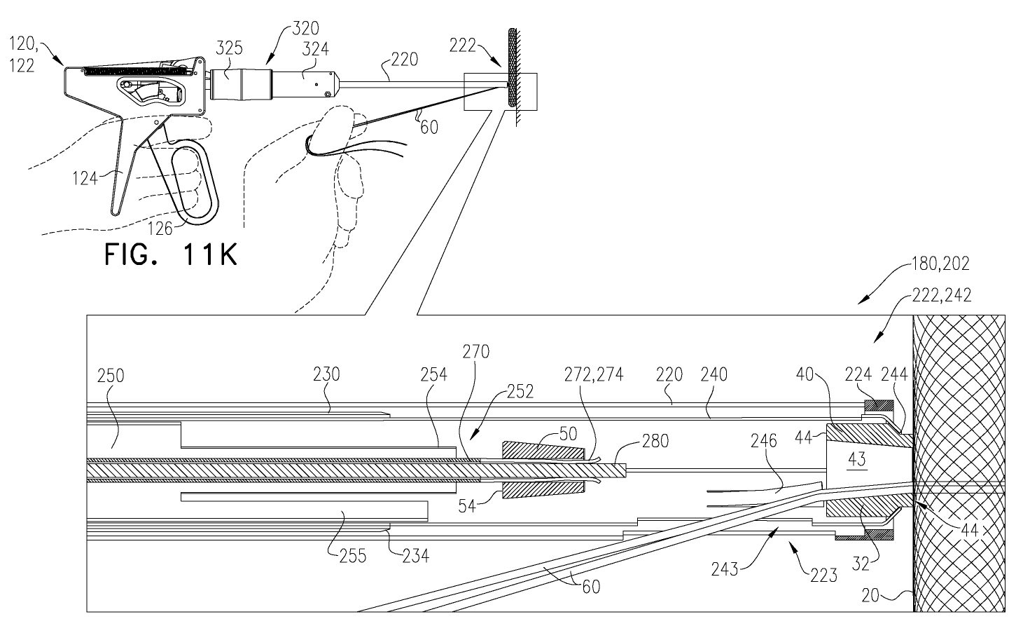

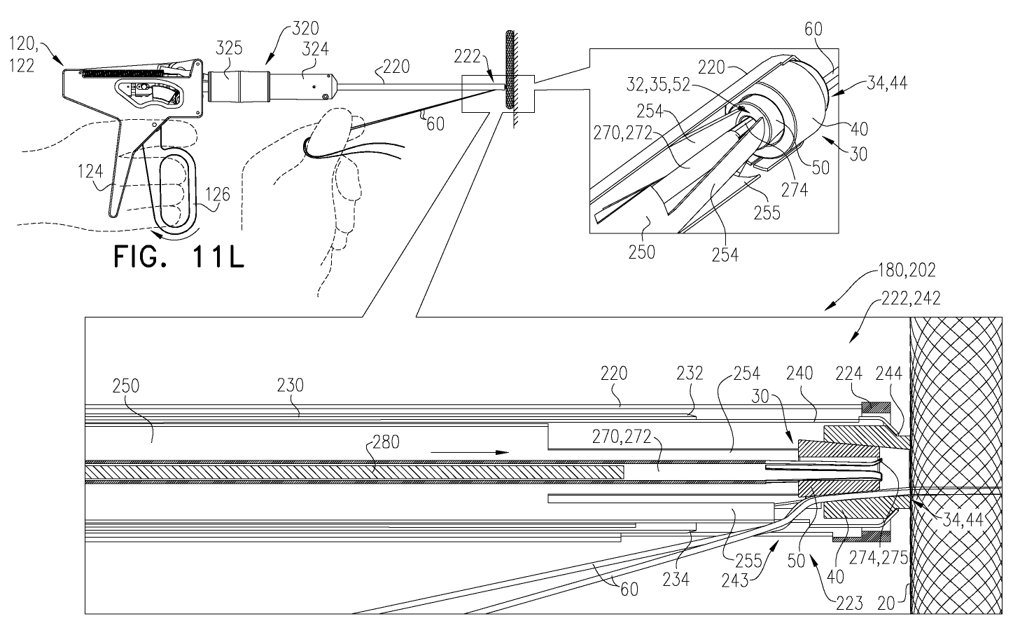

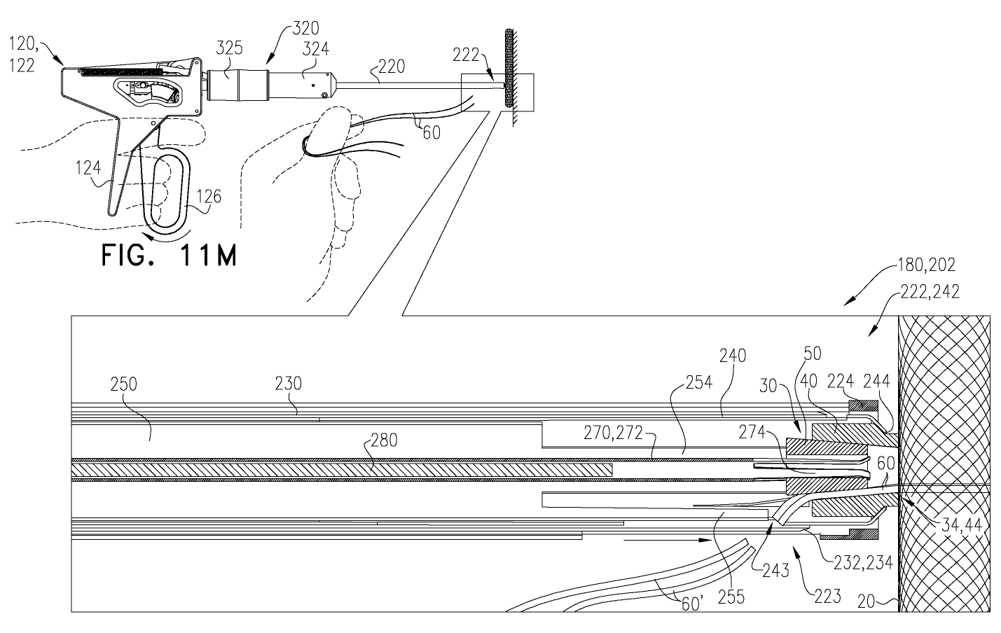

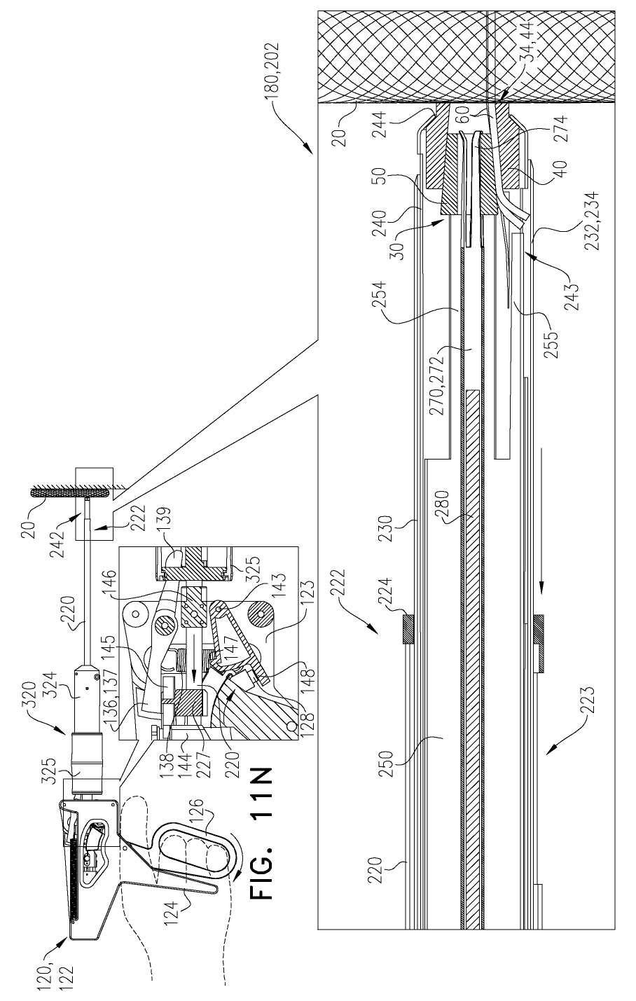

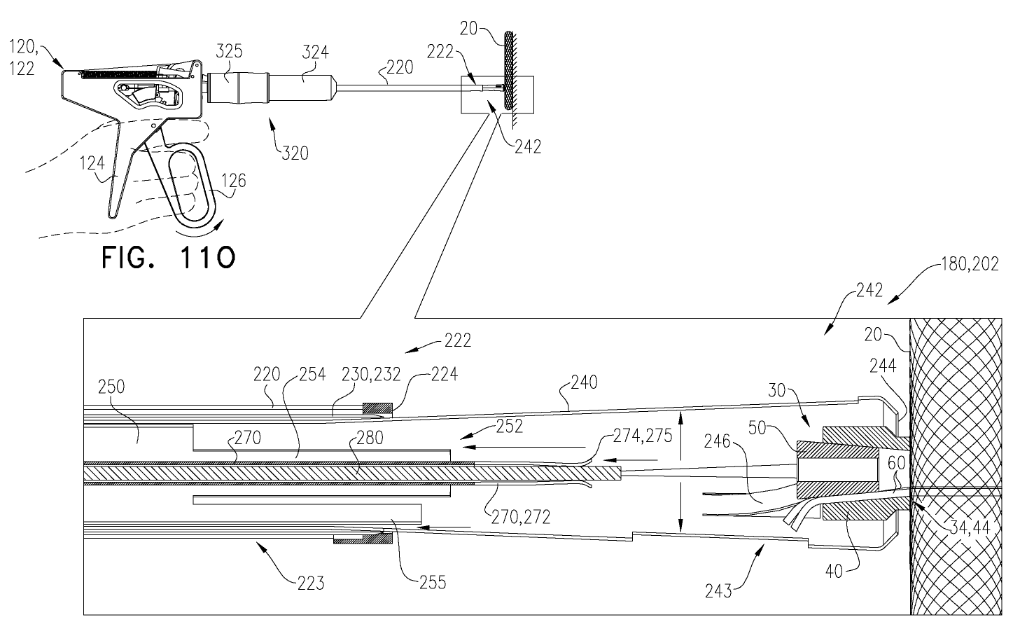

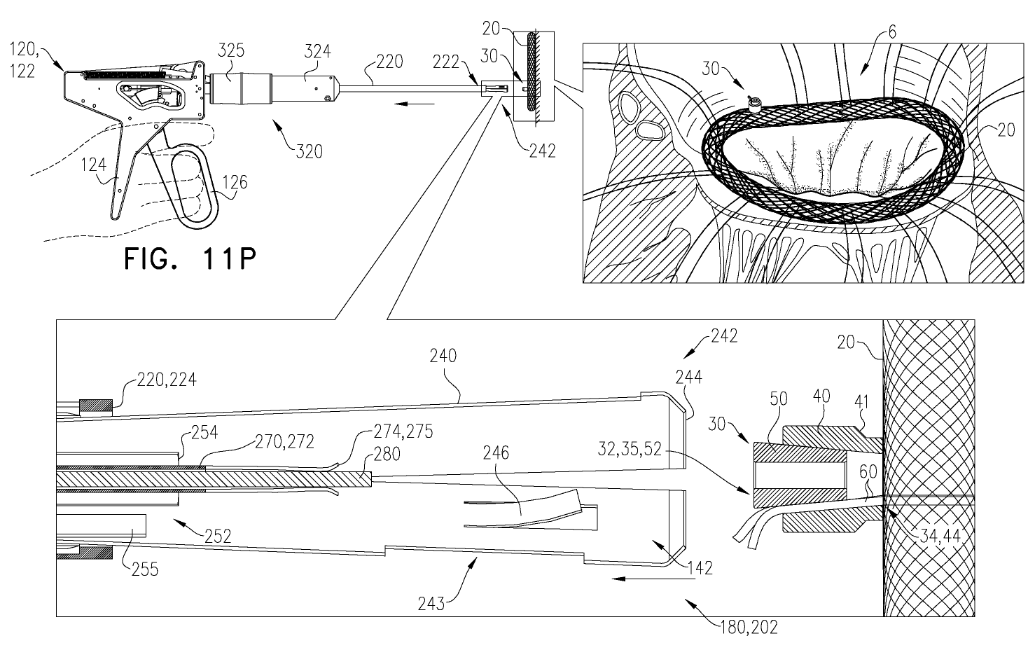

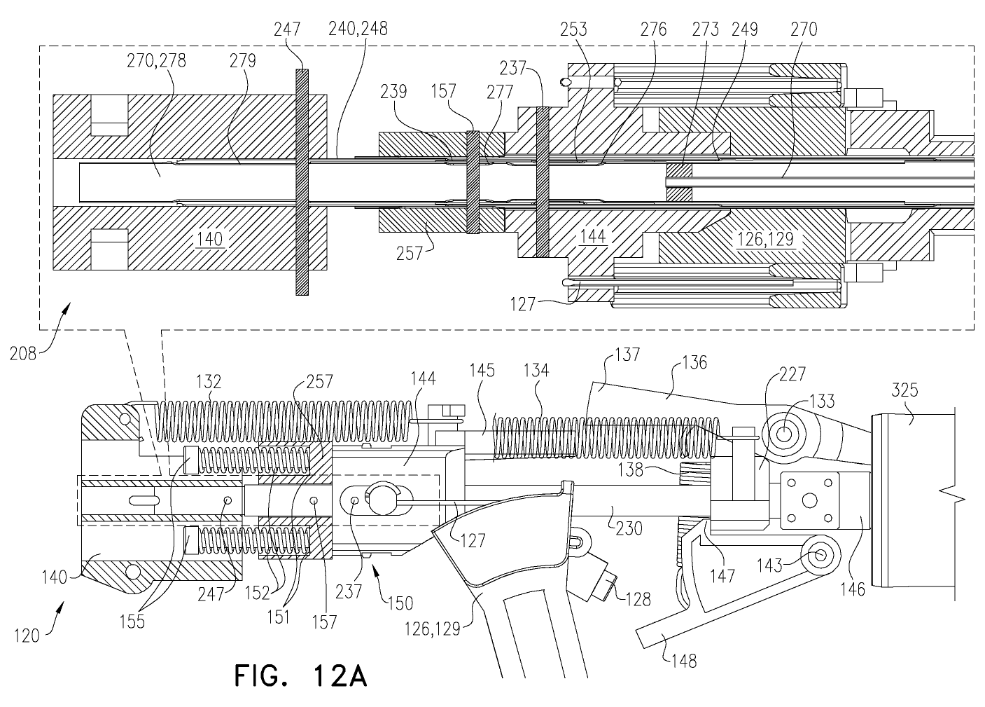

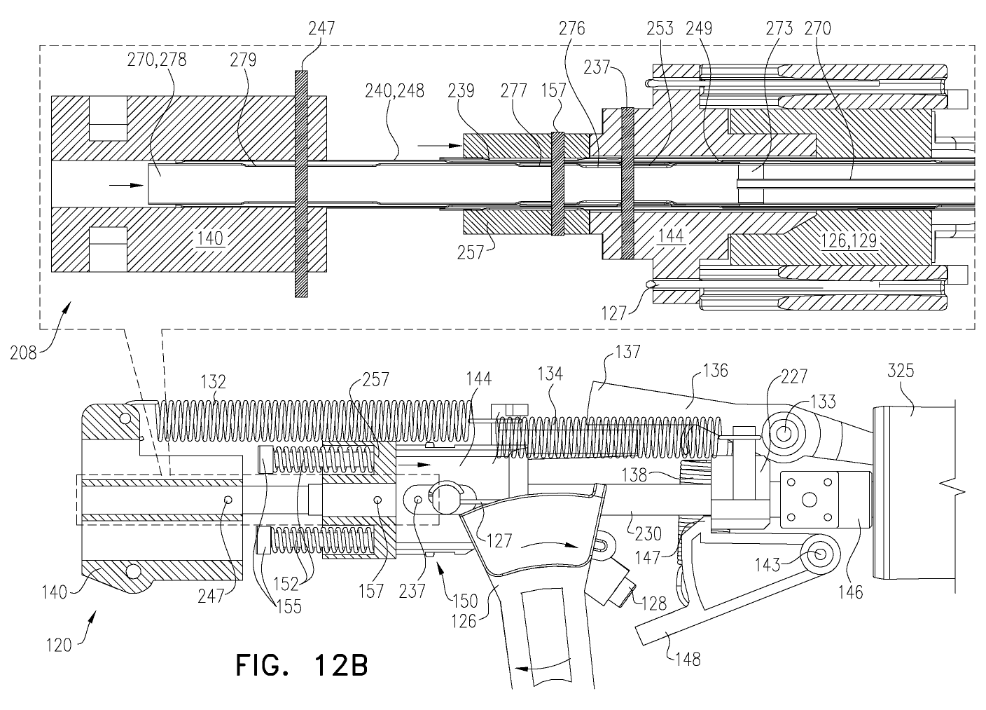

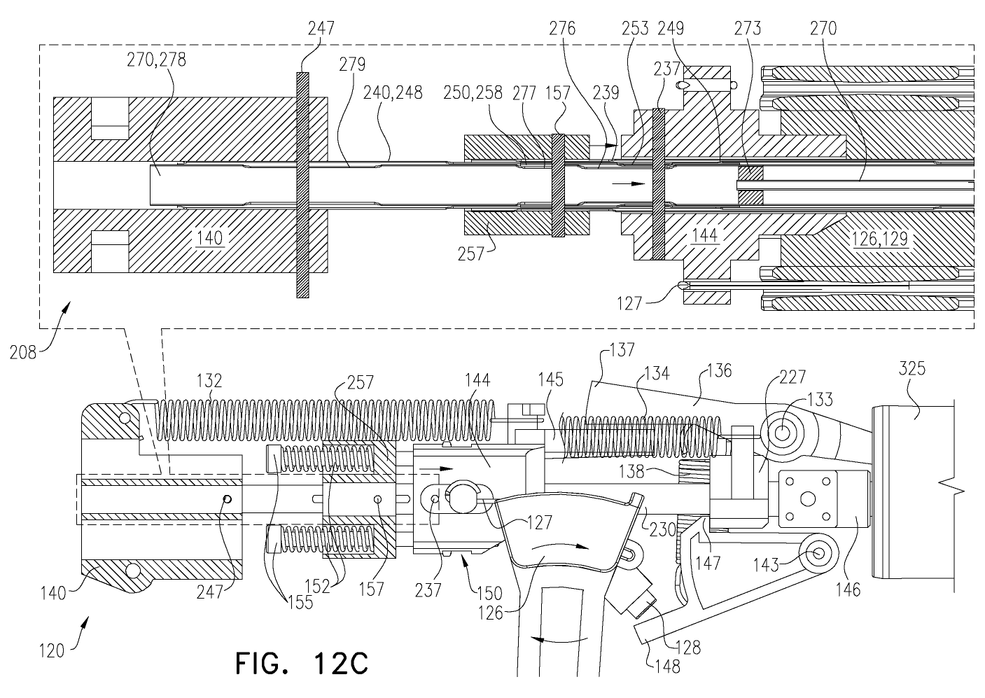

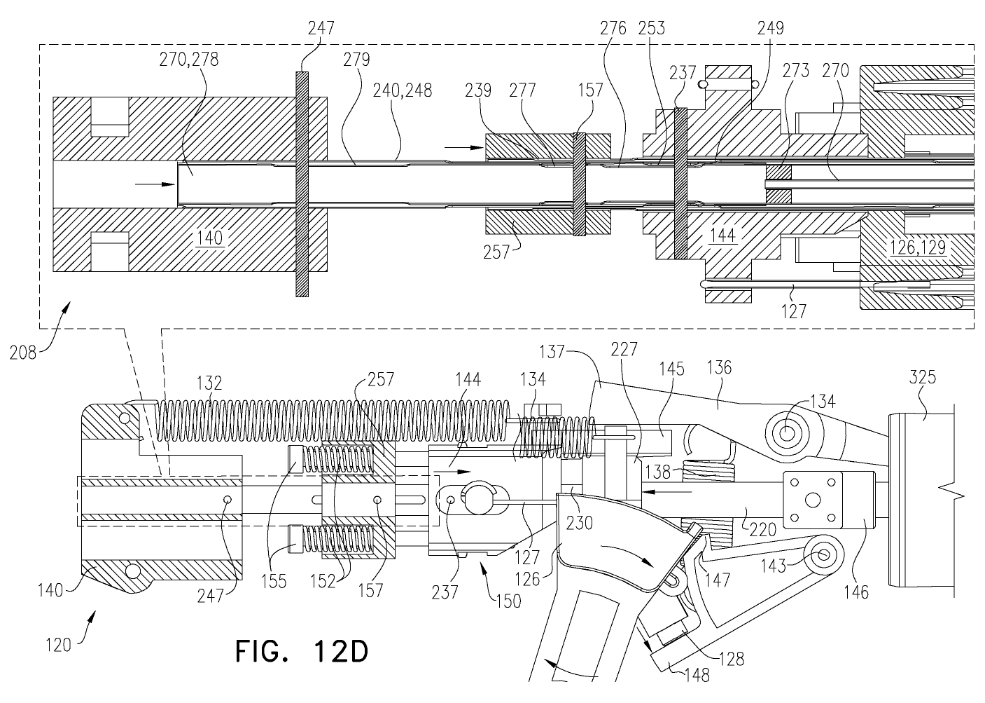

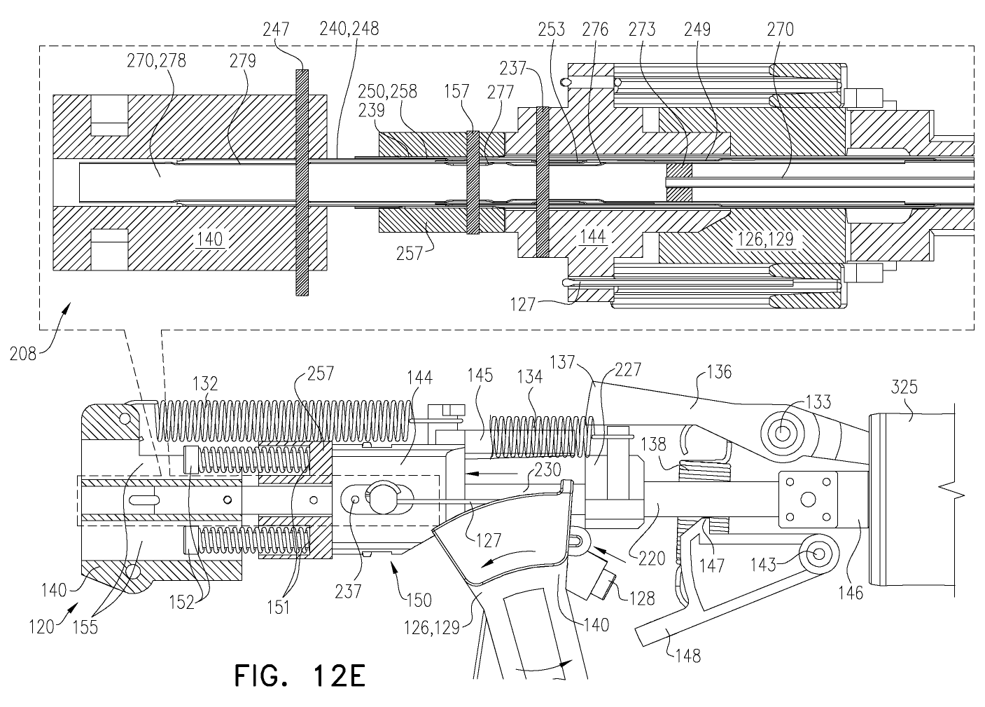

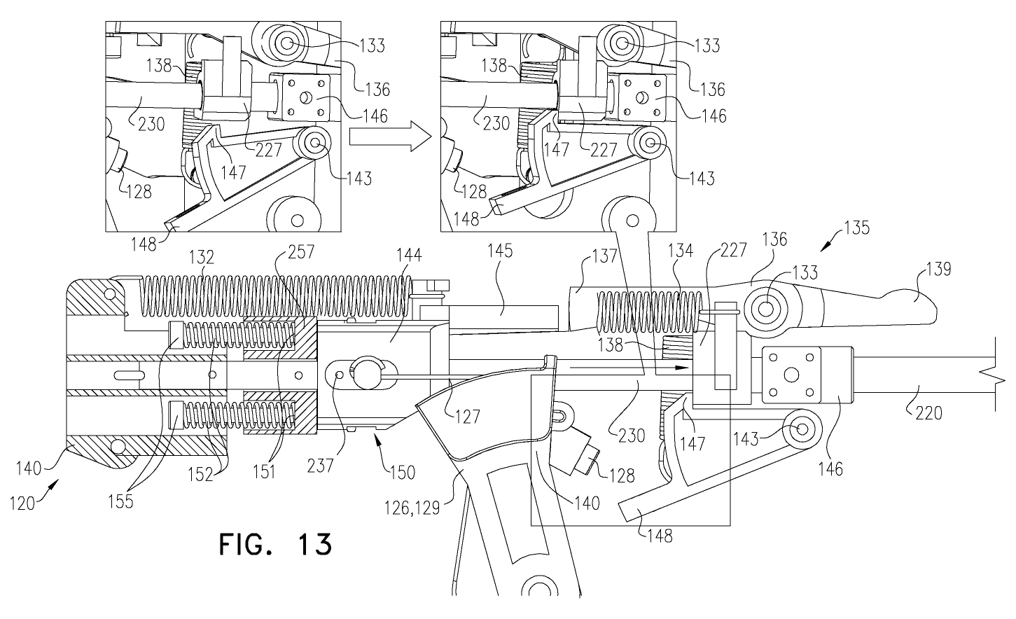

In some implementations, tool 100 is operated by applying an actuation force to proximal portion 120 (e.g., to handle 122 thereof), which transfers the actuation force to a force applicator at the tool’s distal portion 180, as described hereinbelow. In some such implementations, and as described hereinbelow with reference to FIGS. 11L–N, the actuation force pivots the handle’s trigger 126 (e.g., about a hinge 130) toward the handle’s base 124.

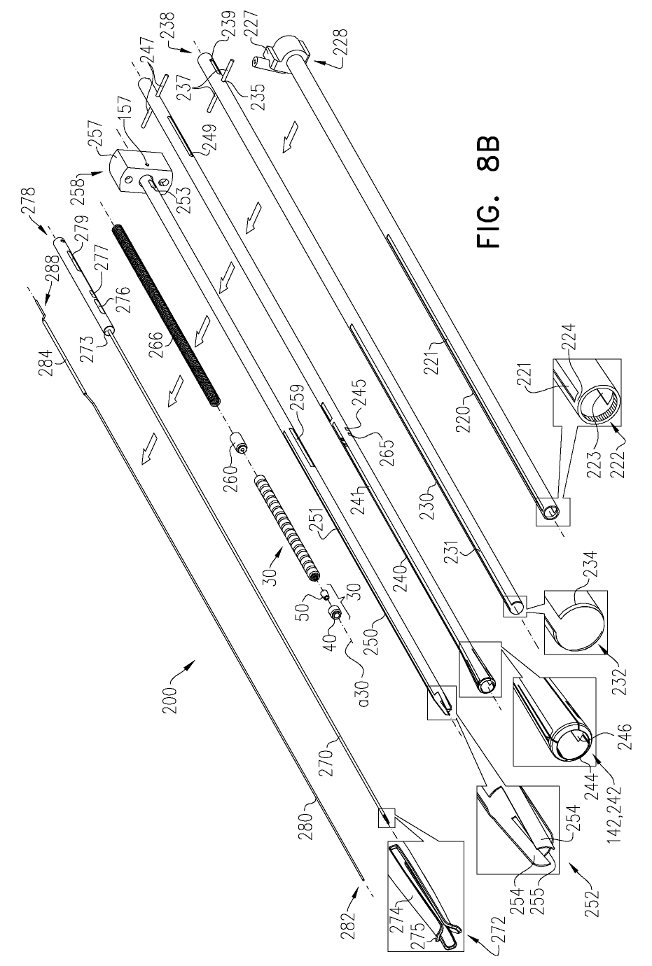

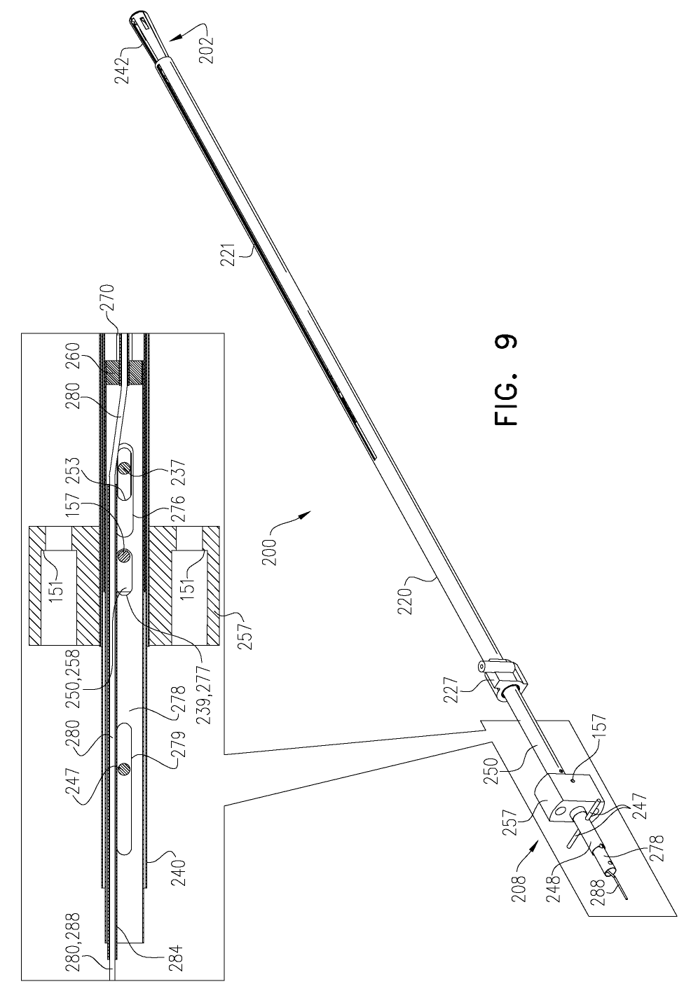

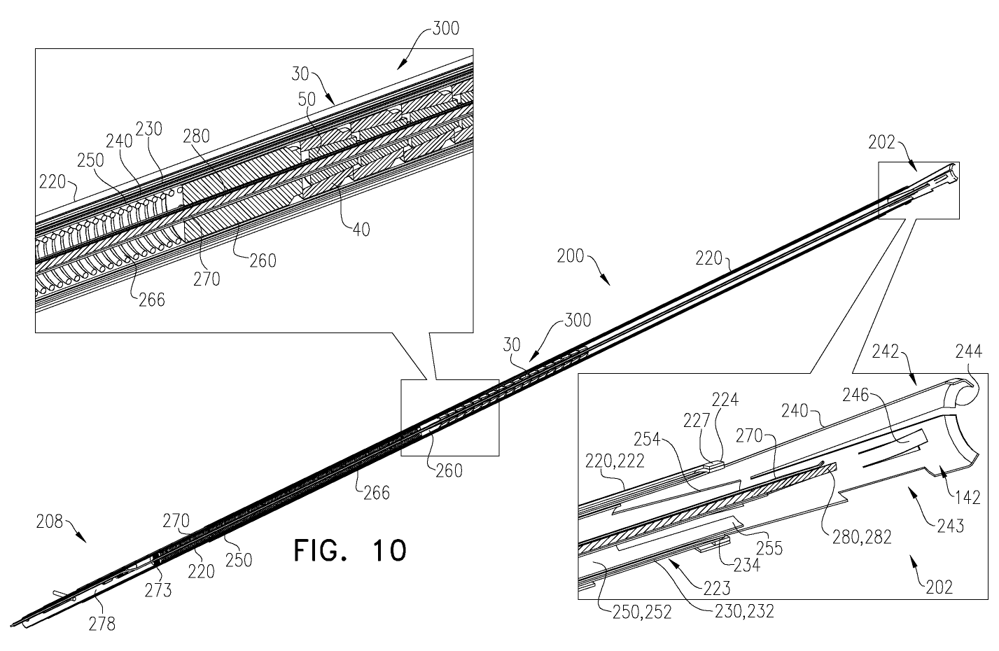

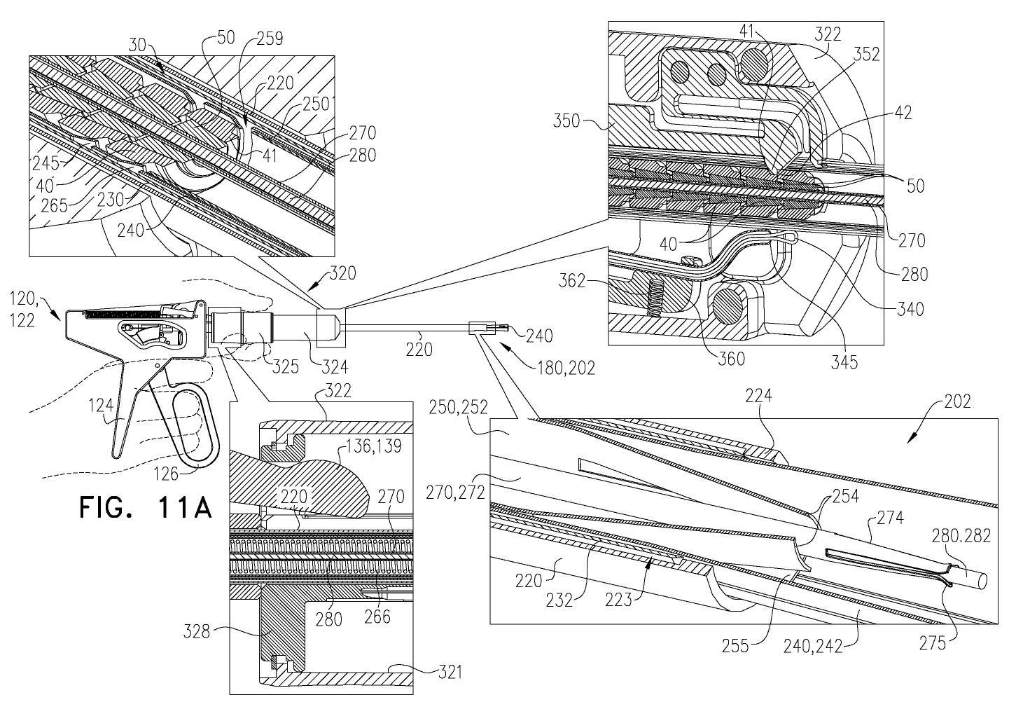

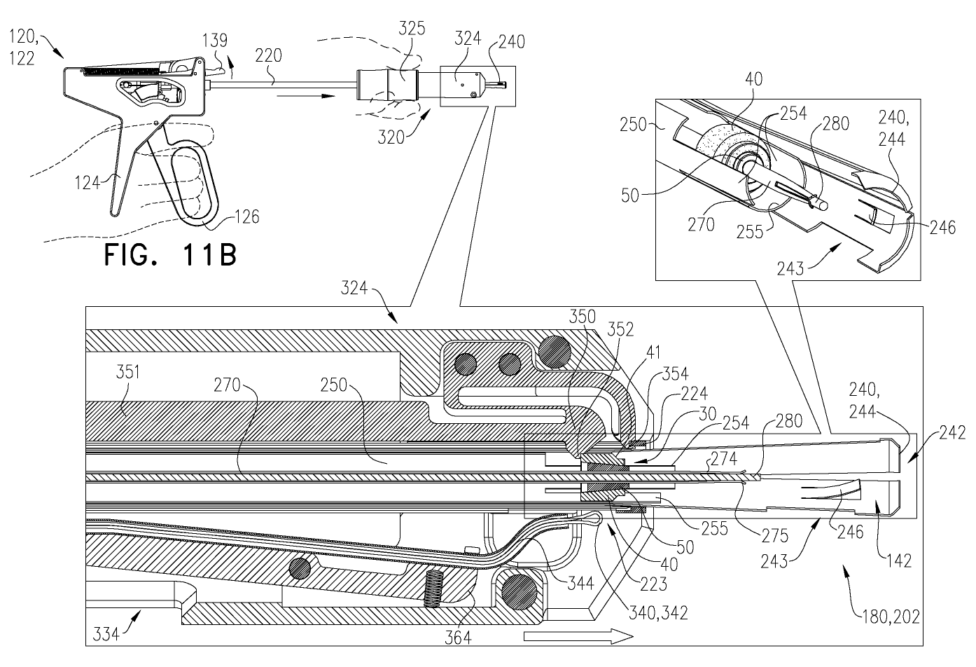

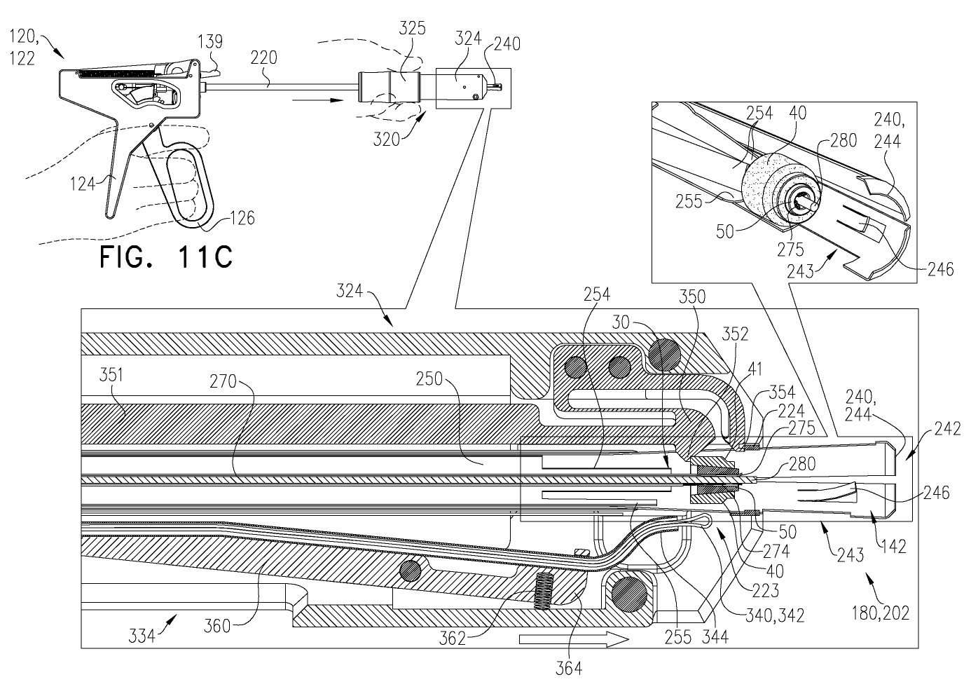

In some implementations, and as shown, multi-shaft system 200 comprises a lock shaft 240 having a distal portion 242 that extends distally from within an outer shaft 220. Multi-shaft system 200 is further described hereinbelow with reference to FIGS. 8A-B and FIGS. 9-10.

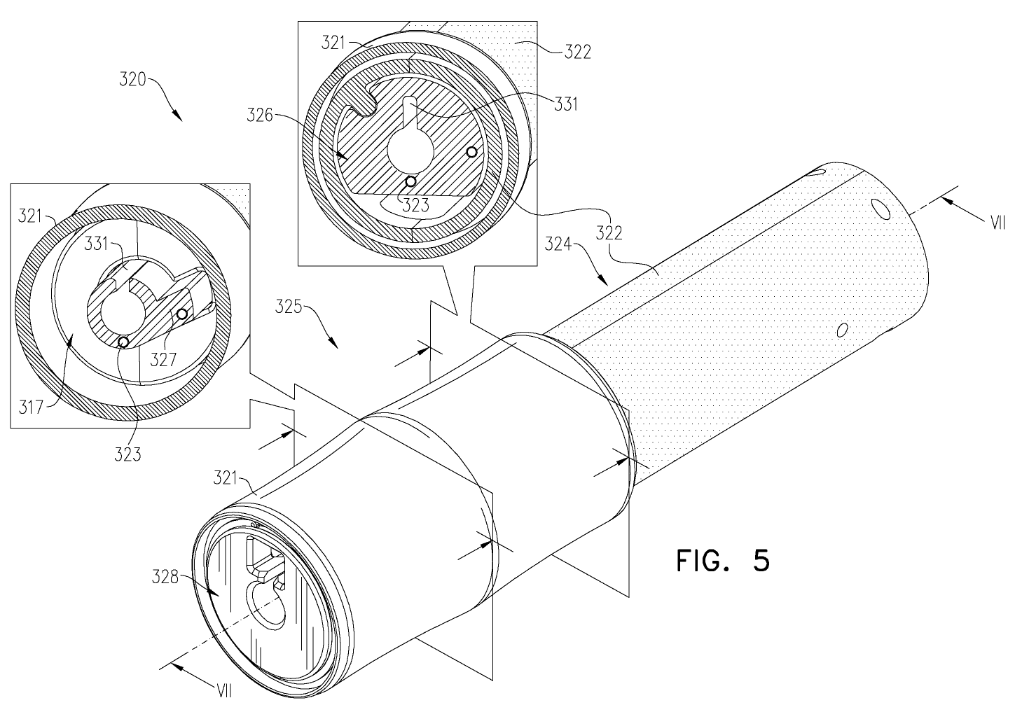

In some such implementations, and as described in greater detail hereinbelow with reference to FIGS. 5-7 and FIGS. 11E-H, shuttle 320 comprises a carriage 324 and a snare pump 325 that is slidable with respect to the carriage.

Reference is made to FIGS. 1B-F, which are schematic illustrations showing suture lock 30 comprising a tube 40 and a plug 50, in accordance with some implementations.

In some implementations, and as shown, tube 40 has an outer surface 47 and an inner surface 45 defining a tube-lumen 43 that is oriented along a tube axis a40. In some such implementations, and as shown, the tube’s inner surface 45 is nonparallel to tube axis a40.

In some implementations, and as shown, plug 50 has an outer surface 57, e.g., a frustoconically shaped outer surface that is nonparallel to plug axis a50. In some such implementations, and as shown, plug 50 defines an inner surface 55 defining a plug-lumen 53 that is oriented along a plug axis a50. For example, and as shown in FIG. 1E, the plug’s inner surface 55 can be parallel to plug axis a50.

In some implementations, and as shown, inner surface 45 of tube 40 and an outer surface 57 of plug 50 are dimensioned for the plug to fit snugly within the tube’s tube-lumen 43, e.g., such that the tube’s inner surface becomes parallel to the plug’s outer surface. In some such implementations, and as shown, inner surface 45 of tube 40 is frustoconically shaped to match the plug’s outer surface 57.

In some implementations, and as shown in FIGS. 1E, plug 50 is dimensioned to fit snugly within tube such that tube-lumen 43 and plug-lumen 53 together define the suture lock’s lock-lumen 33. In some such implementations, the luminal space common to tube-lumen 43 and plug-lumen 53 can define lock-lumen 33, e.g., excluding a portion of the tube-lumen that is occupied by the plug’s wall, e.g., between the plug’s inner surface 55 and outer surface 57. For example, and as shown, lock-lumen 33 can also exclude the portion of tube-lumen 43 defining proximal recess 36, and/or the portion of plug-lumen 53 defined by distal protrusion 38.

In some implementations, lock-lumen 33 is formed when tube-lumen 43 and plug-lumen 53 share a common luminal space, e.g., when plug 50 is fitted within tube-lumen 43. That is, lock-lumen 33 can be formed by locking suture lock 30 (FIG. 1E), subsequently split into tube-lumen 43 and plug-lumen 53 by separating plug 50 from tube 40 (FIG. 1B), after which the lock-lumen can be reformed by relocking the suture lock.

In some implementations, and as shown in FIG. 1F, tube-lumen 43 and plug-lumen 53 define the suture lock’s lock-lumen 33 when the suture lock is secured to suture 60. In some such implementations, and as shown, lock-lumen 33 excludes the portion of tube-lumen 43 defining distal recess 37, and/or the portion of plug-lumen 53 defined by proximal protrusion 35, while the suture lock is secured to suture 60. For example, suture 60 can be threaded through tube-lumen 43, but not necessarily through plug-lumen 53.

In some implementations, tube 40 and/or plug 50 comprise material that facilitates a friction fit of plug 50 within tube-lumen 43. In some such implementations, tube 40 and/or plug 50 comprise a thermoplastic polymer, such as polyether ether ketone (PEEK). In some such implementations, tube 40 and/or plug 50 further comprise a biocompatible radiopaque material, e.g., barium sulfate.

FIG. 1B is an exploded view of suture lock 30 in an unlocked state in which plug 50 is disposed outside of tube 40. FIGS. 1C–E are perspective views (FIGS. 1C–D) and a cross-sectional view (FIG. 1E) of suture lock 30 in an unloaded locked state in which tube 40 houses plug 50, in the absence of suture 60. FIG. 1F is a cross-sectional view of suture lock 30 in a loaded locked state in which tube 40 houses at least a portion of plug 50 and suture 60.

In some implementations, and as shown, tube 40 is narrower at the tube’s distal portion 44 than at the tube’s proximal portion 42. In some such implementations, and as shown, the tube’s outer surface 47 is shaped to define a distally-facing shoulder 41, e.g., between a wide portion 48 having an outer proximal diameter pd46 and a narrow portion 49 having an outer distal diameter dd48 (FIG. 1E). For example, and as shown, narrow portion 49 can extend axially from shoulder 41 to the plug’s distal face.

In some implementations, and as shown in FIGS. 1C–E, plug 50 is longitudinally offset from tube 40 when the plug is fit into tube-lumen 43. In some such implementations, in the absence of suture 60 (FIGS. 1C–E), plug 50 is distally advanceable within tube 40 such that a distal portion 54 of the plug protrudes from a distal portion 44 of the tube, defining a distal protrusion 38 of the suture lock. For example, and as shown, in the absence of suture 60, a distal portion 54 of plug 50 can define the suture lock’s distal portion 34, and a proximal portion 42 of tube 40 can define the suture lock’s proximal portion 32.

In some implementations, and as shown in FIGS. 1C and 1E, a portion of tube-lumen 43 defines a proximal recess 36 when plug 50 is fit into the tube-lumen. In some such implementations, proximal recess 36 is wider than distal protrusion 38, e.g., since an inner proximal diameter pd42 of the tube is greater than an outer distal diameter dd58 of the plug (FIG. 1E). In some such implementations, proximal recess 36 is greater in depth than a length of distal protrusion 38. As such, several suture locks 30 can be aligned in series (e.g., within a storage zone 300 of tool 100, as described hereinbelow with reference to FIGS. 10 and 11A), such that distal protrusion 38 of one suture lock fits within proximal recess 36 of another suture lock.

In some implementations, and as shown in FIG. 1F, suture 60 is secured by locking suture lock 30 onto the suture, by fitting plug 50 into the tube-lumen 43 while the suture is threaded through the tube-lumen, such that outer surface 57 of the plug grips inner surface 45 of the tube. That is, a friction fit of plug 50 and suture 60 within tube-lumen 43 can be sufficient to secure the suture.

In accordance with some implementations, FIG. 1F shows suture lock 30 in the loaded locked state in which tube 40 houses at least a portion of plug 50 and suture 60, such that a portion of the suture is sandwiched between an outer surface 57 of the plug and an inner surface 45 of the tube, e.g., laterally of plug-lumen 53. In some such implementations, and as shown in FIG. 1F’s cross-sectional view, suture 60 is sandwiched between the tube’s inner surface 45 and one lateral side of the plug’s outer surface 57, while the opposite lateral side of the plug’s outer surface grips the tube’s inner surface 45.

In some implementations, and as shown, tube axis a40 and plug axis a50 are coaxial, e.g., parallel, when suture 60 is sandwiched therebetween. Alternatively, tube axis a40 and plug axis a50 can intersect (not shown) while suture lock 30 secures the suture.

In some implementations, and as shown in FIGS. 1E–F, the longitudinal offset of plug 50 within tube 40 is altered when suture lock 30 is locked onto suture 60. In some such implementations, plug 50 advances distally within tube-lumen 43 until the plug’s outer surface 57 grips the tube’s inner surface 45, and the depth to which the plug advances within the tube depends on factors including characteristics (e.g., width and/or compressibility) of suture 60.

FIG. 1F therefore schematically represents suture lock 30 secured to a length of suture 60 that is not necessarily drawn to scale. In this way, suture lock 30 can be secured to suture 60 at a continuum of depths of plug 50 within tube-lumen 43. For example, suture lock 30 can secure a thinner and/or more compressible suture 60 by plug 50 advancing a greater distal distance withing tube-lumen 43.

In some implementations, and as shown, a distal portion of tube-lumen 43 defines a distal recess 37, and proximal portion 52 of plug 50 defines a proximal protrusion 35 when suture lock 30 is locked onto suture 60. In this way, while suture lock 30 is locked onto suture 60, a distal portion 44 of tube 40 defines the suture lock’s distal portion 34, and a proximal portion 52 of plug 50 defines the suture lock’s proximal portion 32.

Reference is made to FIGS. 2–3 and 4A–B, which are schematic illustrations showing handle 122 of tool 100, in accordance with some implementations.

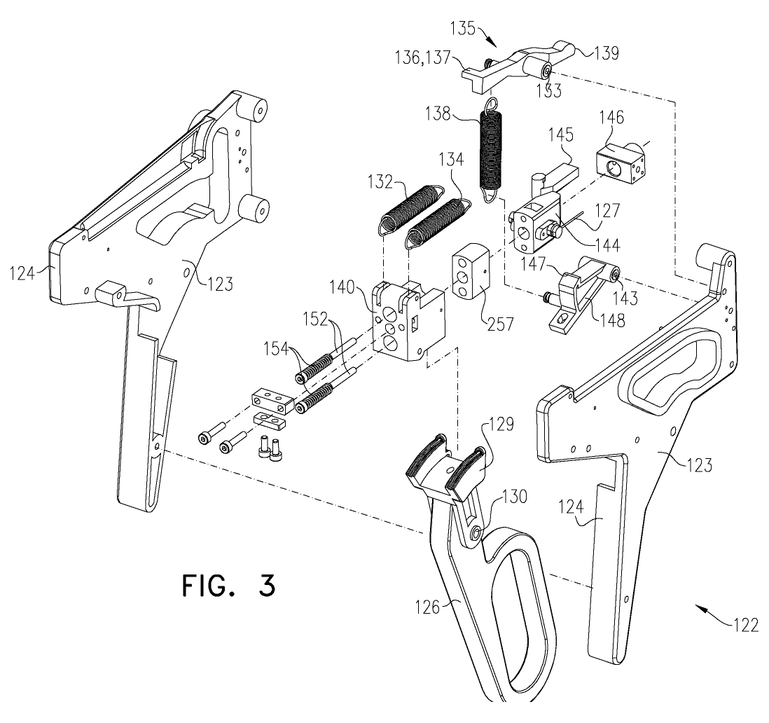

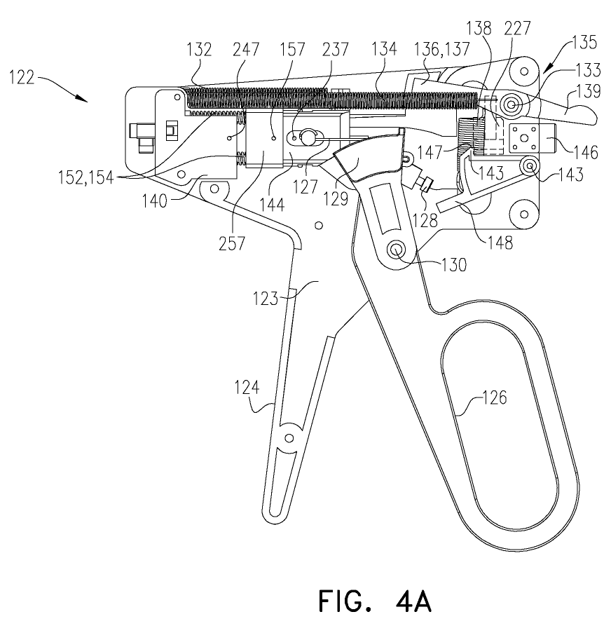

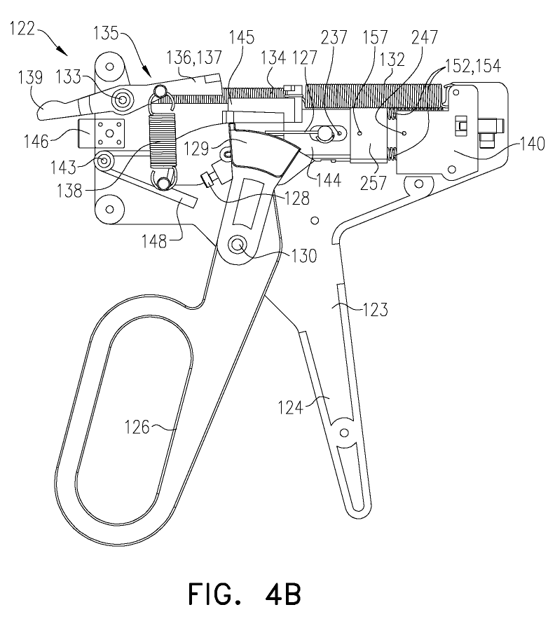

FIGS. 2–3 show perspective and exploded views of handle 122, and FIGS. 4A–B are cutaway views of the handle with alternate halves of the handle’s housing 123 removed.

In some implementations, trigger 126 is coupled to handle 122 at hinge 130 such that pivoting the trigger toward base 124 advances a pivotable portion 129 of the handle distally, e.g., toward a distal interface 146 of handle 122. In some implementations, and as shown in FIGS. 4A–B, pivotable portion 129 is coupled, e.g., via connector 127, to a trigger block 144 such that the trigger block advances distally in tandem with the pivotable portion as trigger 126 is pivoted toward the handle’s base 124.

In some implementations, and as shown in FIG. 4B, handle 122 comprises a trigger interlock 135 that prevents the user from inadvertently pivoting trigger 126 toward base 124. In some such implementations, an interlock spring 138 pulls an interlock lever 136, e.g., a proximal portion 137 thereof, into contact with a distal end 145 of trigger block 144, thereby inhibiting advancement of the trigger block unless interlock 135 has been unlocked (as shown in FIG. 4B). For example, and as described hereinbelow with reference to FIG. 11I, interlock 135 can be unlocked by retracting shuttle 320 such that the shuttle unlocks the interlock by pivoting interlock lever 136 with respect to interlock hinge 133.

In some such implementations, and as shown, a trigger spring 132 (e.g., a tension spring) couples trigger block 144 to a handle block 140 that is fixed to the handle’s housing 123. In this way, trigger spring 132 is tensioned as pivoting portion 129 pulls trigger block 144 distally, which resists the actuation force that is applied to trigger 126 and/or pulls the trigger block proximally upon release of the trigger.

In some implementations, and as shown, the handle’s pivotable portion 129 defines a hammer 128 that faces an anvil 148, such that distally advancing the pivotable portion swings the hammer toward the anvil, e.g., such that the hammer contacts the anvil. In some such implementations, and as shown in the lower inset of FIG. 2 and in FIG. 4A, an outer shaft block 227 is coupled to handle block 140 via an outer shaft spring 134. For example, and as shown, outer shaft spring 134 can be tensioned as outer shaft block 227 advances distally, e.g., holding the outer shaft block against the anvil’s lip 147.

In some implementations, and as shown in the insets of FIG. 2, handle 122 is engaged to proximal portion 208 of multi-shaft system 200 via a force regulator 150 that distributes force (e.g., from trigger 126) to the multi-shaft system. In some such implementations, force regulator 150 differentially limits the force that can be transferred from trigger 126 to members of system 200, as described hereinbelow with reference to FIGS. 12A–E.

In some implementations, and as shown in FIGS. 4A-B, pivotable portion 129 is coupled to trigger block 144 via connector 127. In some such implementations, a cutter pin 237 couples trigger block 144 to a cutter shaft 230 of system 200 described hereinbelow. In this way, pivoting trigger 126 toward base 124 distally advances trigger block 144, cutter pin 237 and cutter shaft 230.

In some such implementations, and described hereinbelow with reference to an exemplary implementation, a lock block 257 is coupled (e.g., adjustably coupled) to trigger block 144 via lock bolts 152. In some such implementations, when the actuation force applied to trigger 126 is below a force threshold, trigger block 144 and lock block 257 advance in tandem, and when the actuation force reaches the force threshold, the trigger block advances with respect to the lock block.

In some implementations, the force threshold is selected in order to allow force regulator 150 to transfer sufficient force from trigger 126 to system 200 in order to effectively lock suture lock 30, while avoiding transferring excessive force that could possibly damage the suture lock. For example, the force threshold can be between 1.5–2.5 kg of force, such as 1.75–2.25 kg of force, e.g., 2 kg of force.

In some implementations, and as shown, lock block 257 is coupled via plug pins 157 to a plug shaft 250 of system 200 described hereinbelow. In this way, when the actuation force applied to trigger 126 is below the force threshold, cutter shaft 230 and plug shaft 250 advance in tandem, and when the actuation force reaches the force threshold, the cutter shaft advances with respect to the plug shaft.

In some implementations, and as shown, a pair of lock nuts 153 are each seated against a respective distally-facing floor portion 149 of trigger block 144. In some such implementations, and as shown, lock springs 154 fit along lock bolts 152, such that the lock springs extend from heads 155 of the lock bolts to a respective proximally-facing floor portion 151 of the lock block.

In some implementations, compression of lock springs 154 facilitates distal advancement of trigger block 144 with respect to lock block 257. In some such implementations, a position of lock nuts 153 along lock bolts 152 can be altered in order to change a length of lock bolts 152 along which lock springs 154 extend, thereby adjusting the force threshold to either inhibit or facilitate distal motion of trigger block 144 with respect to lock block 257.

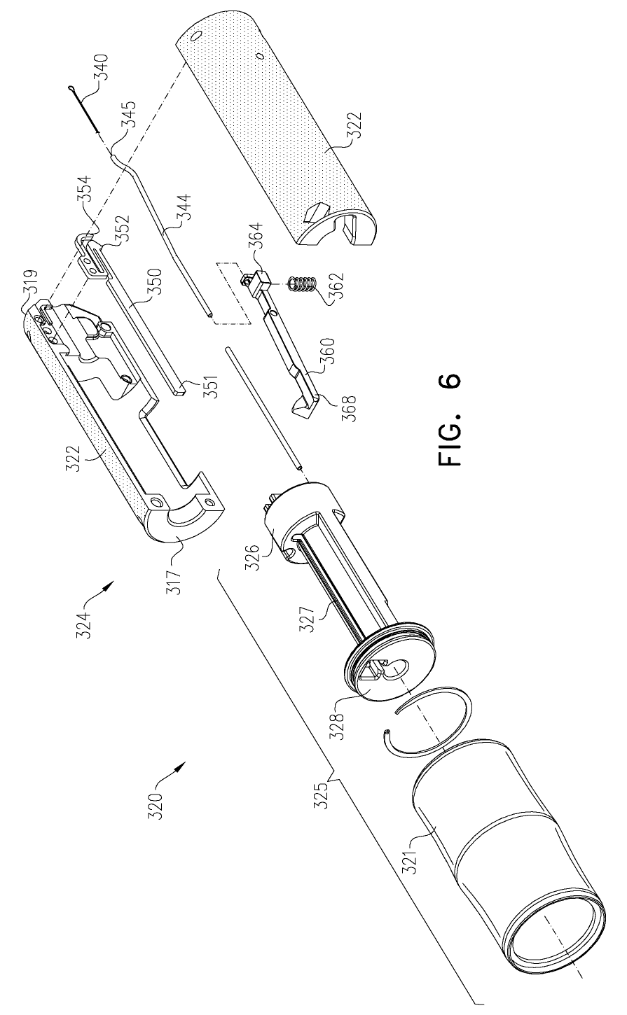

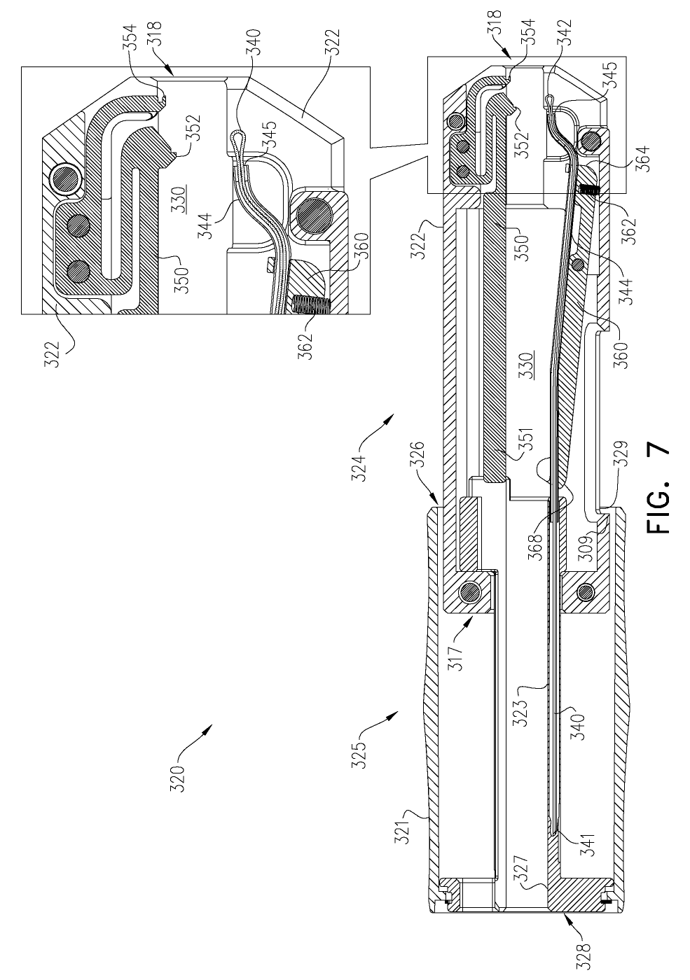

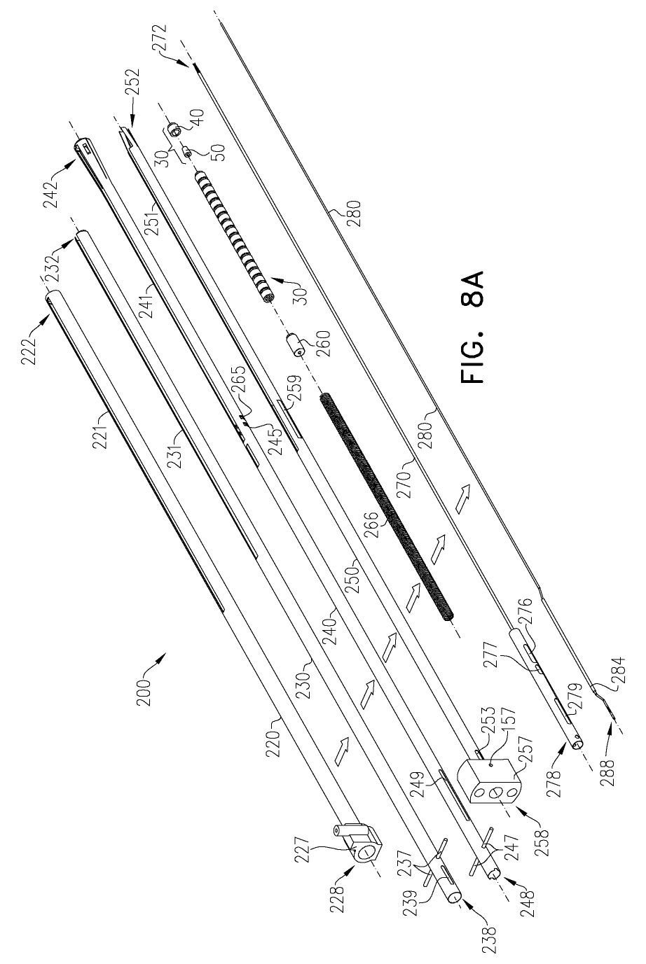

Reference is made to FIGS. 5–7, which are schematic illustrations showing a perspective view (FIG. 5), exploded view (FIG. 6) and a cross-sectional view (FIG. 7) of shuttle 320, in accordance with some implementations.

As shown, shuttle 320 comprises a carriage 324 and a snare pump 325 that is longitudinally slidable (e.g., bidirectionally slidable) with respect to the carriage. In some implementations, snare pump 325 is operated by sliding the snare pump with respect to carriage 324. In accordance with some such implementations, FIGS. 5 and 7 show shuttle 320 in an extended state in which a proximal portion 317 of carriage 324 contacts distal portion 326 of snare pump 325, e.g., a lip 329 of the snare pump’s housing 321 abuts a shoulder 309 of the carriage’s housing 322, thereby inhibiting further extension of shuttle 320.

In some implementations, sliding snare pump 325 in one direction with respect to carriage 324 releases snare 340 via the carriage’s distal aperture 318 (e.g., defined by the carriage’s distal portion 319), in order to snare a suture 60. In some such implementations, sliding snare pump 325 in an opposite direction with respect to carriage retracts snare 340 (e.g., and the suture) into the carriage, via aperture 318.

In some implementations, and as shown in FIGS. 6-7, carriage 324 comprises a housing 322 to which a pusher 350 is attached. In some such implementations, and as shown, the pusher’s proximal portion defines a rail 351, and the snare pump’s pump body 327 defines a longitudinal groove 331 that is aligned with the rail, e.g., while pusher 350 engages suture lock 30 (not shown). In this way, rail 351 can slide within groove 331 as the snare pump’s housing 321 is slid over the carriage’s housing 322, such that the user can actuate snare pump 325 while pusher 350 engages suture lock 30.

In some such implementations, the pusher’s distal portion defines a lip 352 that is dimensioned to interact with lock 30, e.g., with proximal portion 42 and/or narrow portion 49 of the suture lock’s tube 40. In this way, and as described hereinbelow with reference to FIGS. 11A-H, shuttle 320 can serve two functions: (i) sliding shuttle 320 distally along system 200, while pusher 350 engages suture lock 30, advances the suture lock to the system’s distal portion 202; and (ii) sliding snare pump 325 with respect to carriage 324 facilitates snaring suture 60 with snare 340 and drawing the suture into carriage 324.

In some implementations, and as shown in FIGS. 6-7, shuttle 320 comprises a snare tube 344 housing a snare 340, e.g., the snare is constrained within the snare tube. In some such implementations, and as shown, a snare sleeve 323 houses a proximal portion 341 of snare 340. For example, and as shown, proximal portion 341 can be fixedly coupled within snare sleeve 323.

Snare sleeve 323 may be a component of, or defined by, snare pump 325 (e.g., housing 321 thereof). Snare tube 344 may be a component of, or defined by, carriage 324 (e.g., housing 322 thereof).

Advancing snare pump 325 distally with respect to carriage 324 (e.g., such that proximal portion 328 of the snare pump approaches proximal portion 317 of carriage 324) pushes snare 340 (e.g., a distal portion 342 thereof) out from a distal opening 345 of snare tube 344, and from shuttle 320 as a whole.

In some implementations, and as shown in FIG. 7, a snare spring 362 (e.g., a compression spring) supports a snare interlock lever 360, e.g., a distal portion 364 thereof, which presses snare tube 344 towards a shuttle-lumen 330 of shuttle 320 in which system 200 (not shown in FIG. 7) is disposed. In this way, when a window of system 200 is disposed at distal opening 345 of snare tube 344, snare 340 can enter the system, e.g., in order to ensnare a suture 60, as described hereinbelow with reference to FIGS. 11E-F.

Reference is made to FIGS. 8A-B and FIGS. 9-10, which are schematic illustrations showing multi-shaft system 200 of tool 100, in accordance with some implementations.

In some implementations, and as shown, system 200 comprises a series of coaxial shafts which are used in conjunction with suture lock 30. FIGS. 8A-B are exploded views of system 200, which extends from a proximal portion 208 of the system to a distal portion 202 of the system. The shafts of system 200 are arranged in FIGS. 8A-B in order of descending diameter, with arrows pointing from each shaft to the next shaft of smaller diameter.

In some implementations, and as shown, the outermost shaft of system 200 is outer shaft 220, which extends distally from outer shaft block 227 (which is shown in FIG. 2 coupled to handle block 140 via an outer shaft spring 134) at a proximal portion 228 of the outer shaft, to a distal portion 222 of the outer shaft. In some such implementations, and as shown, outer shaft 220 defines a longitudinal slot 221 that is shaped to allow pusher 350 (e.g., lip 352 thereof) to slide suture lock 30 to distal portion 222 of the outer shaft (FIGS. 11A-B). In some implementations, the pusher’s outer shaft-engaging tooth 354 is shaped to engage a distal tooth 224 at the outer shaft’s distal portion 222, in order to advance outer shaft 220 with respect to other shafts of system 200 (FIGS. 11B-F).

In some implementations, and as shown, a cutter shaft 230 extends distally from a proximal portion 238 of the cutter shaft to a distal portion 232 of the cutter shaft. In some such implementations, cutter shaft 230 fits slidably within outer shaft 220 and defines a blade 234 at distal portion 232, such that the blade can be advanced With respect to other shafts of system 200, e.g., outer shaft 220.

In some implementations, and as shown in FIGS. 8A-B, the cutter shaft’s proximal portion 238 is fixedly coupled to cutter pin 237, which is itself fixed to trigger block 144 (FIGS. 2, 4A-B). In some such implementations, and as shown, cutter shaft 230 defines a longitudinal slot 231 that is shaped to allow pusher 350 (e.g., lip 352 thereof) to distally slide suture lock 30 to within distal portion 232 of the cutter shaft.

In some implementations, and as shown, a lock shaft 240 dimensioned to slidably fit within cutter shaft 230 extends distally from a proximal portion 248 of the lock shaft to a distal portion 242 of the lock shaft. In some such implementations, and as shown, the lock shaft’s proximal portion 248 defines a lock pin 247 that is fixedly coupled to handle block 140, as shown above in FIGS. 4A-B.

In some implementations, and as shown, the lock shaft’s proximal portion 248 is shaped to define a proximal slot 249 that facilitates sliding other shafts of system 200 with respect to the lock shaft, e.g., by allowing cutter pin 237 to slide through the proximal slot, as described hereinbelow with reference to FIGS. 12A-E.

In some such implementations, and as shown, distal portion 242 defines a longitudinal slot 241 that is shaped to allow pusher 350 (e.g., lip 352 thereof) to distally slide a suture lock 30 to within distal portion 242 of the lock shaft.

In some implementations, and as shown, distal end 244 of lock shaft 240 defines teeth that are biased (e.g., heat-set) to flare radially outwards in the absence of an external force (e.g., cutter shaft 230 and/or outer shaft 220 surrounding and compressing the lock shaft’s distal portion 242) that can compress distal end 244. In some such implementations, and as shown in FIGS. 8B and 10, the lock shaft’s distal portion 242 is shaped to define a holding zone 142 that is shaped to hold suture lock 30 in position at the distal portion when the teeth of distal end 244 are compressed around the suture lock.

Although lock shaft 240 is shown being used with suture lock 30, the lock shaft, and particularly holding zone 142 thereof, can be suitable for other suture locks. For example, lock shaft 240 can be used with suture locks (e.g., suture locks that do not comprise tubes and plugs) having different dimensions and/or comprising different materials, mutatis mutandis.

In some implementations, and as described in greater detail hereinbelow with reference to FIG. 11A, lock shaft 240 defines retention tabs (e.g., a proximal pair 245 and a distal pair 265 of retention tabs) that extend inward into the lock shaft’s lumen.

In some implementations, and as shown, a plug shaft 250, dimensioned to fit slidably within lock shaft 240, extends distally from a proximal portion 258 of the lock shaft to a distal portion 252 of the lock shaft that defines plug-pusher arms 254 at the plug shaft’s distal portion 252. In some such implementations, and as shown, plug-pusher arms 254 are biased (e.g., heat-set) radially inwards in the absence of an external force (e.g., presence of a suture lock 30 between the plug-pusher arms) that can push the plug-pusher arms apart. In some such implementations, distal portion 252 further defines a suture support 255, e.g., which, in contrast to plug-pusher arms 254, is not biased to extend radially inwards, as shown.

In some implementations, and as shown, proximal portion 258 of plug shaft 250 defines a longitudinal slot 251 that is shaped to allow pusher 350 (e.g., lip 352 thereof) to distally slide a suture lock 30 to within distal portion 252 of the plug shaft.

In some implementations, and as shown, plug shaft 250 defines a pair of lateral windows 259 that allow the lock shaft’s retention tabs 245, 265 to protrude into the plug shaft’s lumen (FIG. 11A). As shown, each pair of retention tabs 245, 265 are longitudinally spaced from each other at approximately the distance between respective shoulders 41 of consecutive suture locks 30. In this way, retention tabs 245, 265 can define a location within plug shaft 250 at which a suture lock 30 is positioned.

In some implementations, and as shown, the plug shaft’s proximal portion 258 is coupled by plug pins 157 to lock block 257 (FIGS. 4A-B). In some such implementations, and as shown, the plug shaft’s proximal portion 258 is shaped to define a proximal slot 253 that facilitate sliding cutter pin 237 with respect to plug shaft 250, as described hereinbelow with reference to FIGS. 12C.

In some implementations, and as shown in FIG. 10, system 200 (e.g., plug shaft 250 thereof) is dimensioned to slidably house the row of suture locks 30 in a storage zone 300 of the system. In some such implementations, and as shown in FIGS. 8A-B and FIG. 10, a plurality of suture locks 30 are arranged in a row, e.g., such that the suture locks share a common lock axis. A proximalmost suture lock 30 is shown in FIGS. 8A-B disassembled into tube 40 and plug 50, for illustrative purposes. The number of suture locks 30 shown is exemplary, and more or fewer suture locks can be loaded within storage zone 300, mutatis mutandis.

In some implementations, a loading spring 266 is also disposed within plug shaft 250, proximally of suture locks 30. In some such implementations, and as shown, a lock pusher 260 is disposed between the loading spring and the suture locks. Typically for such implementations, loading spring 266 is calibrated to apply a distal force to lock pusher 260 that is sufficient to push the distal-most suture lock 30 into the space between retention tabs 245, 265.

In some implementations, and as shown, system 200 comprises an inner shaft 270 dimensioned to fit slidably within plug shaft 250, and that extends distally from a proximal sleeve 278 of the inner shaft to a distal portion 272 of the inner shaft that defines branches 274. In some such implementations, branches 274 define a detent, e.g., tips 275 that flare radially outward.

In some implementations, and as shown, inner shaft’s proximal sleeve 278 is dimensioned to fit slidably within plug shaft 250. In some such implementations, proximal sleeve 278 defines a proximal slot 279, a middle slot 277 and a distal slot 276 that facilitate sliding inner shaft 270 with respect to pins 157, 237, 247 as described hereinbelow with reference to FIGS. 12A-E.

In some implementations, inner shaft’s proximal sleeve 278 defines a distal lock stopper 273. In some such implementations, lock stopper 273 applies a compression force to loading spring 266 by supporting the loading spring’s distal end, as described hereinbelow with reference to FIG. 10.

In some implementations, and as shown, distal portion 272 of inner shaft 270 is narrower than proximal sleeve 278, such that the inner shaft passes through an internal space defined by loading spring 266, and through lumens 43, 53 of the row of suture locks 30. In this way, distal portion 272 extends distally from within the suture locks such that the inner shaft’s branches 274 extend distally from within plug shaft 250 and into distal portion 242 of lock shaft 240.

In some implementations, and as shown, system 200 comprises an inner wire 280 that extends from a proximal portion 288 to a distal portion 282 that extends distally from within inner shaft 270. In some implementations, wire 280 is fixedly coupled to handle 122, such that inner shaft 270 is slidable along wire 280. In some such implementations, while the inner shaft’s distal portion 272 is disposed over and along the wire, the wire keeps branches 274 of the inner shaft’s distal portion distanced from each other, thereby maintaining a radial width of distal tips 275. In some such implementations, advancing the inner shaft’s distal portion 272 distally beyond wire 280 increases compressibility of the distal portion’s branches 274, thereby reducing the width of distal tips 275.

FIGS. 9-10 show perspective and cross-sectional views of system 200 assembled such that the system extends from proximal portion 288 of wire 280 to distal portion 242 of lock shaft 240.

The inset of FIG. 9 is a cross-sectional view of proximal portion 208 of multi-shaft system 200, showing (i) lock pin 247 disposed within inner shaft’s proximal slot 279, (ii) plug pin 157 disposed within both the inner shaft’s middle slot 277 and the cutter shaft’s proximal slot 239, and (iii) cutter pin 237 disposed within both the proximal sleeve’s distal slot 276 and plug shaft’s proximal slot 253. Proximal slot 249 of lock shaft 240 (through which cutter pin 237 is slidable) is shown in FIGS. 8A-B but is not readily visible in FIG. 9.

In some implementations, and as shown in the inset of FIG. 9, proximal portion 288 of wire 280 passes through a sleeve 284. In some such implementations, sleeve 284 is fixedly coupled to the interior of inner shaft’s proximal sleeve 278, which reduces interference from proximal portion 288 of inner wire 280 upon movement of other component of system 200, e.g., by reducing contact between the inner wire and pins 157, 237, 247.

FIG. 10 shows cross-sectional views of system 200. In some implementations, and as shown in the upper inset of FIG. 10, loading spring 266 and a row of suture locks 30 are disposed on respective sides of lock pusher 260, within the lumen of plug shaft 250. In some such implementations, and as shown, several suture locks 30 are aligned in series such that distal protrusion 38 of one suture lock fits within proximal recess 36 of another suture lock. In this way, respective tubes 40 of the suture locks contact each other, yet respective plugs 50 do not necessarily contact each other. Such an arrangement of suture locks 30 may be advantageous by allowing the compressed loading spring 266 to support and/or advance the row of suture locks by pushing on a proximal suture lock, without overtightening the suture locks’ plugs 50 within their respective tubes 40.

The lower inset of FIG. 10 shown distal portion 202 of the assembled system 200. In some implementations, and as shown, the respective distal portions of the shafts comprising system 200 are longitudinally offset from each other, in order to facilitate use of the system as described hereinbelow with reference to FIGS. 11A-Q.

In some such implementations, and as shown, distal portion 242 of lock shaft 240 extends distally from within distal portion 222 of outer shaft 220. In this way, lock shaft’s distal portion 242 is shown having radially expanded such that the lock shaft’s distal end 244 is wider than the lock shaft’s proximal portion 248 and/or wider than outer shaft 220.

In some implementations, and as shown, holding zone 142 is defined by holding arm 246 that is biased radially inward (e.g., to support the suture lock’s proximal portion 32) and a distal end 244 of lock shaft 240 that is angled radially inward, e.g., to support the tube’s distal portion 44.

Reference is made to FIGS. 11A-H, which are schematic illustrations showing use of tool 100 to advance suture lock 30 to distal portion 202 of system 200, and to thread a length of suture 60 through the suture lock, in accordance with some implementations.

In some implementations, and as described hereinbelow, shuttle 320 is slidable along system 200 between handle 122 (e.g., distal interface 146 thereof) and distal portion 202 of the system. In some such implementations, pusher 350 can be slid distally in order to advance suture lock 30 (e.g., tube 40 thereof) and/or outer shaft 220 with respect to the tool’s proximal portion 120 and/or elements of multi-shaft system 200, as described hereinbelow.

FIG. 11A shows tool 100 in a pre-loading state in which distal portion 202 of multi-shaft system 200, e.g., holding zone 142 (not shown) thereof, does not house suture lock 30. In some implementations, and as shown in the lower right inset of FIG. 11A, the lock shaft’s distal portion 242 defines a lumen within which distal portion 252 of plug shaft 250 extends distally. In some such implementations, and as shown, plug-pusher arms 254 are biased radially inwards toward distal portion 272 of inner shaft 270, which extends distally from the plug shaft’s distal portion 252, e.g., between the plug shaft’s plug-pusher arms.

In some implementations, and as shown, wire 280 extends distally from within the inner shaft’s distal portion’s branches 274. In some such implementations, branches 274 are biased radially inwards such that presence of the wire’s distal portion 282 maintains a distance between the branches.

In some implementations, and as shown in the upper left inset of FIG. 11A, lock shaft 240 defines retention tabs (e.g., a proximal pair 245 and a distal pair 265 of retention tabs) that extend inward, through the plug shaft’s lumen, in order to hold the distal-most suture lock 30 in place. In some such implementations, retention tabs 245, 265 provide resistance to a pushing force that loading spring 266 applies to the row of suture locks 30, in order to prevent unintentional release of suture locks. For example, retention tabs 245, 265 can be dimensioned to provide a friction fit of suture lock 30 therebetween, e.g., by abutting shoulder 41 of one or more suture locks.

In some implementations, and as shown in the upper right inset of FIG. 11A, lip 352 of pusher 350 engages a distal-most suture lock 30 of the row of suture locks, e.g., at the suture lock’s proximal portion 32. In some such implementations, and as shown, lip 352 engages the proximal portion 42 of the suture lock’s tube 40, e.g., without engaging the suture lock’s plug 50. For example, and as shown, the pusher’s lip 352 fits between one suture lock’s shoulder 41 and another suture lock’s proximal portion 32.

With the pusher’s lip 352 engaging the proximalmost suture lock 30, shuttle 320 is used to advance the suture lock to the tool’s distal portion 180, e.g., to distal portion 202 of system 200 (FIG. 11B). In this way, the pusher’s lip 352 slides through longitudinal slots 221, 231, 241, 251 as the pusher pushes the distal-most suture lock 30 distally, e.g., with sufficient force to overcome resistance caused by the friction fit of the suture lock between retention tabs 245, 265.

In some implementations, and as shown, sliding shuttle 320 also helps to prevent prematurely pivoting trigger 126 toward base 124. That is, whereas the lower left inset of FIG. 11A shows distal portion 139 of interlock lever 136 disposed within the carriage’s housing 322, FIG. 11B shows distal portion 139 of interlock lever 136 having been released from shuttle 320. In some such implementations, and as described hereinabove with reference to FIG. 4B, release of interlock lever 136 places proximal portion 137 of the interlock lever into contact with distal end 145 of trigger block 144, thereby inhibiting advancement of the trigger block until shuttle is again pulled proximally over the interlock lever’s distal portion 139 (FIG. 11I).

FIG. 11B shows the pusher’s lip 352 having pushed suture lock 30 along inner shaft 270 to the plug shaft’s distal portion 252, such that plug-pusher arms 254 flare radially outward in order to fit suture lock 30 therebetween (upper right inset). At this point, the pusher’s outer shaft-engaging tooth 354 has engaged the outer shaft’s distal tooth 224, such that further advancement of shuttle 320 will push both outer shaft 220 and suture lock 30 toward the lock shaft’s distal portion 242.

FIG. 11C shows pusher 350 having further advanced outer shaft 220 and suture lock 30 as shuttle 320 is slid further distally along system 200. In some implementations, and as shown, carriage 324 and outer shaft 220 are advanced (e.g., in tandem with each other) such that distal portion 342 of snare 340 is aligned longitudinally with the outer shaft’s window 223. However, in the stage of advancement shown in FIG. 11C, distal portion 342 of snare 340 and the outer shaft’s window 223 are not yet aligned with window 243 of lock shaft 240.

The upper inset of FIG. 11C shows suture lock 30 having been slid past distal portion 252 of plug shaft 250, such that plug-pusher arms 254 reassume their inward radial bias (FIG. 11A). In some implementations, and as shown in the inset of FIG. 11C, suture lock 30 is slid distally such that the suture lock (e.g., plug 50 thereof) reaches tips 275 of inner shaft 270. In some such implementations, and as shown, suture lock 30 is held snugly at this point between the inner shaft’s tips 275 and lip 352 of the carriage’s pusher 350.

FIG. 11D shows shuttle 320 having been slid further along system 200, such that lip 352 unlocks suture lock 30 by pushing tube 40 distally away from (e.g., off of) plug 50. In some implementations, and as shown in the insets of FIG. 11D, tips 275 of the inner shaft’s distal branches 274 extend radially outward, distally from the plug’s distal portion 54, thereby longitudinally restraining plug 50 at the inner shaft’s distal portion 272 as pusher 350 pushes tube 40 away from the plug. In some such implementations, the inner shaft’s distal portion 272 is disposed along wire 280, which keeps tips 275 distanced from each other, thereby preventing plug 50 from sliding distally past the tips.

In some implementations, and as shown, distal portion 342 of snare 340 and the outer shaft’s window 223 have become partially aligned with window 243 of lock shaft 240. In some such implementations, and as shown, contact between snare tube 344 and system 200 (e.g., lock shaft 240 thereof) keeps snare 340 from entering lock shaft’s window 243 at this stage of the carriage’s advancement.

FIG. 11E shows shuttle 320 having been slid still further along system 200, such that tube 40 is seated at the lock shaft’s holding zone 142. In some implementations, and as shown, the pusher’s lip 352 advanced tube 40 over holding arm 246 during the transition from FIG. 11D to FIG. 11E (e.g., temporarily depressing the holding arm), such that pusher 350 supports the tube while the tube is seated at the lock shaft’s holding zone 142.

In some implementations, and as shown in FIGS. 11B-F, pusher 350 assumes a tensioned conformation while the pusher engages tube 40 to push the tube distally. In some such implementations, and as shown, snare pump 325 (e.g., snare pump body 327 thereof) is slidable with respect to pusher 350 while the pusher is tensioned. For example, and as shown, the pusher’s rail 351 is aligned with the snare pump body’s groove 331 (FIG. 11E).

In some implementations, and as shown, the pusher’s outer shaft-engaging tooth 354 advances outer shaft 220 distally over distal portion 242 of lock shaft 240 during this same stage of advancement. Typically for such implementations, distal portion 222 of outer shaft 220 is sufficiently rigid to compress distal portion 242 of lock shaft, thereby compressing holding zone 142 around tube 40. In some implementations, and as shown in FIG. 11E, the lock shaft’s distal end 244 is compressed around shoulder 41 at the tube’s distal portion 44, while holding arm 246 supports the tube’s proximal portion 42, e.g., effectively immobilizing the tube within holding zone 142 while the snare’s distal portion 342 faces the tube-lumen 43.

In some implementations, and as shown in FIG. 11E, the snare’s distal portion 342 and the outer shaft’s window 223 have become more fully aligned with lock shaft’s window 243. In some such implementations, and as shown in FIG. 11E, a distal opening 345 of snare tube 344 has become aligned with windows 223, 243, such that the force that snare spring 362 applies to snare interlock lever 360 pushes the snare’s distal portion 342 through the windows and into lock shaft 240. For example, and as shown, the force that snare spring 362 applies to snare interlock lever 360 also pivots proximal portion 368 of snare interlock lever 360 into a carriage window 334 defined by the carriage’s housing 322, e.g., clicking into place to indicate to the user that the tool is ready for operation of snare pump 325 by advancing the snare pump over carriage 324. The configuration of tool 100 shown in FIG. 11E may therefore be considered the tool’s ready state, in which the tool holds plug 50 and tube 40 separately, within the tool’s distal portion 180.

FIG. 11F shows snare pump 325 having been advanced distally over carriage 324, such that the snare pump pushes the snare’s proximal portion 341 through snare sleeve 323, and the snare’s distal portion 342 extends distally outward from the shuttle’s distal aperture 318.

FIG. 11G shows suture 60 having been threaded through snare 340, after which snare pump 325 is pulled proximally (FIG. 11H), in accordance with some implementations. In some such implementations, and as shown, snare pump 325 in turn pulls carriage 324 proximally with respect to lock shaft 240, such that the tube’s distal portion 44 (e.g., the tube’s distal face) protrudes distally from tool 100. Tube 40 protruding distally from tool 100 can facilitate tensioning suture 60 and drawing the tensioned suture proximally through tube-lumen 43, after which the suture can be released (not shown) such that the suture remains threaded through tube 40 and windows 223, 243 (FIG. 11I).

In some implementations, and as shown in the lower inset of FIG. 11H, pulling pump 325 proximally also draws outer shaft 220 and carriage 324 proximally with respect to lock shaft 240, such that the snare tube’s distal opening 345 is pushed out of lock shaft and into the carriage. For example, and as shown, the snare interlock lever’s distal portion 364 is pushed toward the carriage’s housing 322, thereby compressing snare spring 362. In this way, and as shown in the upper inset of FIG. 11H, the snare interlock lever’s proximal portion 368 (e.g., defining a brake, as shown) pivots toward pump body 327, thereby inhibiting operation of snare pump 325 while snare 340 and distal opening 345 of snare tube 344 are outside of lock shaft 240.

In some implementations, drawing carriage 324 proximally with respect to lock shaft 240 disengages suture lock 30 from the pusher’s lip 352, such that the pusher is allowed to assume a relaxed conformation in which (i) the pusher’s distal portion pivots through longitudinal slot 241 and into the lock shaft’s lumen, and (ii) the pusher’s rail 351 pivots out of alignment with the pump body’s groove 331, e.g., such that the rail abuts the pump body’s distal portion 326, thereby inhibiting operation of the snare pump while tube 40 is disengaged from pusher 350.

Reference is made to FIGS. 11I-P, 12A-E and FIG. 13, which are schematic illustrations showing use of tool 100 to lock suture lock 30 onto a length of suture 60 at an implant 20, in accordance with some implementations.

FIGS. 11I-P show tool 100 as a whole, as well as cross-sectional views of the multi-shaft system’s distal portion 202 in greater detail as the tool transitions through stages A–E, in order to lock suture lock 30 onto suture 60. It is to be noted that stages A–E are for the purpose of description only, and do not necessarily correspond to discrete stages at which tool 100 remains for a given period of time, but rather some or all of stages A–E may be transitory in nature.

FIGS. 12A–E show cutaway and cross-sectional views of the tool’s force regulator 150 and of the multi-shaft system’s proximal portion 208 as the tool transitions through stages A–E. In some implementations, and as described hereinbelow, the actuation force is transferred from trigger 126 via force regulator 150 to system 200 in a manner that coordinates locking suture lock 30, cutting suture 60 and releasing the suture lock from the system’s distal portion 202.

Stages A–E will therefore be described in parallel reference to FIGS. 11I-P and FIGS. 12A-E, in order to highlight the functional relationship between force regulator 150 and multi-shaft system 200, whereby force is transferred from the force regulator to a force applicator in the system’s distal portion 202. In some implementations, the force applicator is not necessarily a discrete component of system 200. Instead, force regulator 150 transfers force to different components of system 200 as tool 100 transitions through stages A–E. In this way, the force applicator may be considered to be the component or components of system 200 that apply force to suture lock 30 during a given stage of locking the suture lock. Advantageously, force regulator 150 transfers force to different components of system 200 as the user provides the actuation force to handle 122, e.g., without the user needing to select a particular component of the system as the force applicator. Tool 100 is therefore simpler to use than other suture-locking devices, making the tool less prone to human error, and more efficient to use.

FIGS. 11I and 12A show tool 100 in state A, in which handle 122 has not yet been actuated, such that system 200 retains the same orientation as in FIG. 11H and suture lock 30 is held, within the system’s distal portion 202, in a loaded unlocked state in which plug 50 is disposed outside of tube-lumen 43 and a portion of suture 60 is threaded through the tube-lumen. As shown, suture 60 has been released from snare 340, which has since been retracted into snare pump 325 and withdrawn proximally with shuttle 320 along outer shaft 220 toward handle 122.

As shown in the upper left inset of FIG. 11I, trigger interlock 135 has been unlocked by sliding distal portion 139 of interlock lever 136 into shuttle 320, thereby depressing distal portion 139 into the shuttle, which overcomes the pulling force applied by interlock spring 138 and elevates proximal portion 137 of the interlock lever 136 out of engagement with trigger block 144. In this way, trigger block 144 can be advanced distally by pivoting trigger 126 towards base 124.

FIG. 12A shows force regulator 150 and proximal portion 208 of system 200 in stage A (e.g., in a resting state), prior to actuating the handle by pivoting trigger 126 toward base 124. In some implementations, and as shown, trigger block 144 and lock block 257 are disposed in a proximal position, due to a pulling force that trigger spring 132 exerts upon trigger block 144, which pulls the trigger block, and in turn the lock block, toward handle block 140.

The inset of FIG. 12A is a cross-sectional view of proximal portion 208 of multi-shaft system 200 during stage A. In some implementations, and as shown, lock pin 247 fixedly attaches lock shaft 240 to handle block 140, which is itself attached to handle 122. As such, handle block 140, lock shaft 240 and lock pin 247 serve as points of reference, relative to which other components of force regulator 150 and system 200 advance during stages B–E.

Pins 157, 237, 247 are slidably disposed within lateral slots (FIGS. 8A-B) defined by various components of system 200 in a manner that facilitates relative motion of the pins and components of the system, as tool 100 transitions through stages B–E. In some implementations, and as shown, lock pin 247 is disposed within proximal slot 279 of inner shaft 270, plug pin 157 is disposed within both inner shaft’s middle slot 277 and cutter shaft’s proximal slot 239, and cutter pin 237 is disposed within both proximal sleeve’s distal slot 276 and plug shaft’s proximal slot 253. In some such implementations, proximal slot 249 of lock shaft 240 is of sufficient length (FIGS. 8A-B) to facilitate sliding of plug pin 157 and cutter pin 237 therethrough.

FIGS. 11J–K show tool 100 having been placed into position for use, such that distal portion 202 of system 200 is pointed towards implant 20, e.g., such that suture lock 30 is placed against the implant while the user pulls suture 60 taught through respective windows 223, 243 of outer shaft 220 and lock shaft 240.

FIGS. 11L and 12B show the user having transitioned tool 100 to stage B by pivoting trigger towards base 124. As shown in FIG. 12B, connector 127 pulls trigger block 144 and lock block 257 distally. Since cutter pin 237 fixes trigger block 144 to cutter shaft 230, the cutter pin and the cutter shaft advance distally, relative to lock block 257 and lock shaft 240 (inset of FIG. 12B).

In some implementations, and as described hereinabove with reference to FIG. 2, lock block 257 is coupled to trigger block 144 via lock bolts 152. In some such implementations, as long as the actuation force applied to handle 122 is below the force threshold, trigger block 144 and lock block 257 advance in tandem (inset of FIG. 12B) as tool 100 transitions from stage A to stage B. As shown, a proximal face of the trigger block remains in contact with a distal face of the lock block, and cutter pin 237 does not change its position within the plug shaft’s proximal slot 253 as both cutter shaft 230 and plug shaft 250 advance within lock shaft 240. Plug pin 157 and cutter pin 237 therefore advance distally within lock block’s proximal slot 249.

The inset of FIG. 11L shows distal portion 252 of plug shaft 250 having advanced distally such that the plug shaft’s plug-pusher arms 254 contact distal portion 54 of plug 50 and push the plug into the tube’s tube-lumen 43, sandwiching a portion of suture 60 between the tube’s inner surface 45 and the plug’s outer surface 57. Distal portion 252 of plug shaft 250 may therefore be considered the force applicator during stage B.

In some implementations, and as shown, inner shaft 270 is pulled distally along with plug 50 during stage B, as plug-pusher arms 254 push the plug distally. In some such implementations, pulling inner shaft 270 maintains alignment of plug 50 with tube-lumen 43 as the plug is advanced into the tube-lumen.

In accordance with some implementations, distal advancement of inner shaft 270 during stage B is represented in the inset of FIG. 12B by the advancement of the inner shaft’s proximal slot 279 with respect to lock pin 247. In some implementations, inner shaft 270 is not only pulled distally at the inner shaft’s distal portion 272, but is also pushed distally at the inner shaft’s proximal sleeve 278, e.g., by plug pin 157 advancing to a distal end of the proximal sleeve’s middle slot 277 (inset of FIG. 12B), such that plug shaft 250 pulls the inner shaft distally.

As shown in the inset of FIG. 11L, distal portion 232 of cutter shaft 230 also advances distally, e.g., in tandem with distal portion 252 of plug shaft 250. In some such implementations, and as shown, the plug shaft’s suture support 255 guards suture 60 from contact with cutter shaft’s blade 234 during stage B, thereby preventing the blade from trimming the suture before suture lock 30 is locked.

FIGS. 11M and 12C show tool 100 having transitioned into stage C, in which trigger 126 continues to pivot forward (e.g., such that the trigger approaches anvil 148), pulling trigger block 144 and therefore cutter shaft 230 further distally.

FIG. 11M shows cutter shaft 230 having advanced distally such that blade 234 trims suture 60, thereby releasing free ends 60' of the suture. Distal portion 232 of cutter shaft 230 may therefore be considered the force applicator during stage C. In some implementations, and as shown in FIG. 11M, the plug shaft’s plug-pusher arms 254 remain stationary, holding plug 50 in place as cutter shaft 230 advances distally.

FIG. 12C shows changes in force regulator 150 that facilitate the distal advancement of cutter shaft 230 as tool 100 transitions from stage B to stage C. In some implementations, contact between the plug shaft’s plug-pusher arms 254 and plug 50 provides resistance to advancement of plug shaft 250. In some such implementations, the user applies a greater actuation force to handle 122 (e.g., reaching the force threshold), which compresses lock springs 154 between heads 155 of lock bolts 152 and floor portion 151 of lock block 257. Trigger block 144 and cutter shaft 230 therefore advance distally while lock block 257 and plug shaft 250 remain stationary (FIG. 12C).

As shown in the inset of FIG. 12C, trigger block 144 pulls cutter pin 237 distally within (i) proximal slot 249 of lock shaft 240, and (ii) proximal slot 253 of plug shaft 250, and (iii) distal slot 276 of the inner shaft’s proximal sleeve 278, thereby advancing cutter shaft 230 distally with respect to lock shaft 240, plug shaft 250 and inner shaft 270.

FIGS. 11N and 12D show handle 122 having been further actuated (e.g., such that trigger 126 is still further pivoted towards base 124), as tool 100 transitions into stage D. In some implementations, the actuation force must be continued to be provided above the force threshold in order to transition tool into stage D.

In some implementations, and as shown in FIG. 12D, trigger 126 pivots still further (e.g., until trigger 126 meets base 124) such that the trigger’s hammer 128 depresses the handle’s anvil 148. Whereas lip 147 held outer shaft block 227 in place during stages A–C (FIGS. 12A-C), FIG. 12D and the upper inset of FIG. 11N show anvil 148 having pivoted about an anvil hinge 143, thereby releasing outer shaft block 227 from the anvil’s lip 147. As shown, lip 147 no longer holds outer shaft spring 134 in tension, such that outer shaft spring 134 pulls outer shaft block 227, and therefore outer shaft 220, proximally.

FIG. 11N therefore shows distal portion 222 of outer shaft 220 having been retracted proximally, exposing distal portion 242 of lock shaft 240. As shown in the inset of FIG. 11N, cutter shaft 230 remains disposed over lock shaft 240, such that the lock shaft’s distal portion 242 remains contracted and suture lock 30 is still held inside of holding zone 142 during stage D.

In some implementations, after fully pivoting trigger 126 (FIG. 11N), the user releases the trigger (FIG. 11O), relaxing tension upon trigger spring 132, which pulls trigger block 144 and lock block 257, and therefore cutter shaft 230 and plug shaft 250, proximally as tool 100 transitions into stage E (FIG. 12E). As shown in FIG. 11O, withdrawing cutter shaft 230 over lock shaft 240 allows distal portion 242 of lock shaft 240 to reassume its radially biased configuration, such that holding zone 142 expands around suture lock 30.

In some implementations, and as shown in FIG. 12E, proximally retracting trigger block 144 and lock block 257 also retracts inner shaft 270 proximally with respect to lock shaft 240 and inner wire 280. For example, and as shown in the inset of FIG. 12E, plug pin 157 slides proximally within inner shaft’s middle slot 277 until the plug pin abuts the middle slot’s proximal end, such that retracting the plug pin further pulls inner shaft 270 proximally.

In some implementations, and as shown in FIG. 12E, retraction of inner shaft 270 with respect to inner wire 280 is facilitated by the inner shaft’s distal portion 272 extending distally from wire 280 during stage D (FIG. 11N). In some such implementations, advancing the inner shaft’s distal portion 272 distally beyond wire 280 makes the distal portion’s branches 274 radially compressible such that the branches’ tips 275 can be withdrawn proximally through the plug’s plug-lumen 53 as tool 100 transitions into stage E, thereby releasing plug 50 from the inner shaft 270.

FIG. 11P shows tool 100 having been retracted proximally away from suture lock 30, such that the suture lock exits holding zone 142 and remains secured to suture 60 at implant 20. In some implementations, radial expansion of lock shaft’s distal end 244 reduces resistance that suture lock 30 (e.g., shoulder 41 thereof) offers to the retracting lock shaft. In some such implementations, distal end 244 expands to define an internal diameter that is greater than a greatest outer diameter (e.g., outer proximal diameter pd46 of tube 40) of suture lock 30.

In some implementations, after releasing suture lock 30 such that the suture lock remains secured to suture 60 at implant, tool 100 is reset in order to prepare the tool for applying another suture lock to implant 20. As shown in FIG. 12E, in stage E, tool 100 is in a similar state to the resting state shown in FIG. 11A, with the exception that outer shaft block 227 is not held in tension by lip 147 of anvil 148.

In some implementations, it is desirable to prepare tool 100 for another cycle of use, in which another suture lock 30 will be unlocked, loaded with suture 60 and locked (e.g., relocked) onto the suture. In some such implementations, the pusher’s outer shaft-engaging tooth 354 reengages the outer shaft’s distal tooth 224 and shuttle 320 is advanced distally along outer shaft 220 to distal portion 202 of system 200, such that tool 100 reassumes the state shown in FIG. 11B. As shown in FIG. 13, pulling outer shaft 220 distally pulls outer shaft spring 134 into tension as the anvil’s lip 147 reengages outer shaft block 227. In some such implementations, the pusher’s lip 352 engages another suture lock 30 for delivery to the system’s distal portion 202, thereby preparing the tool for the next cycle of use.

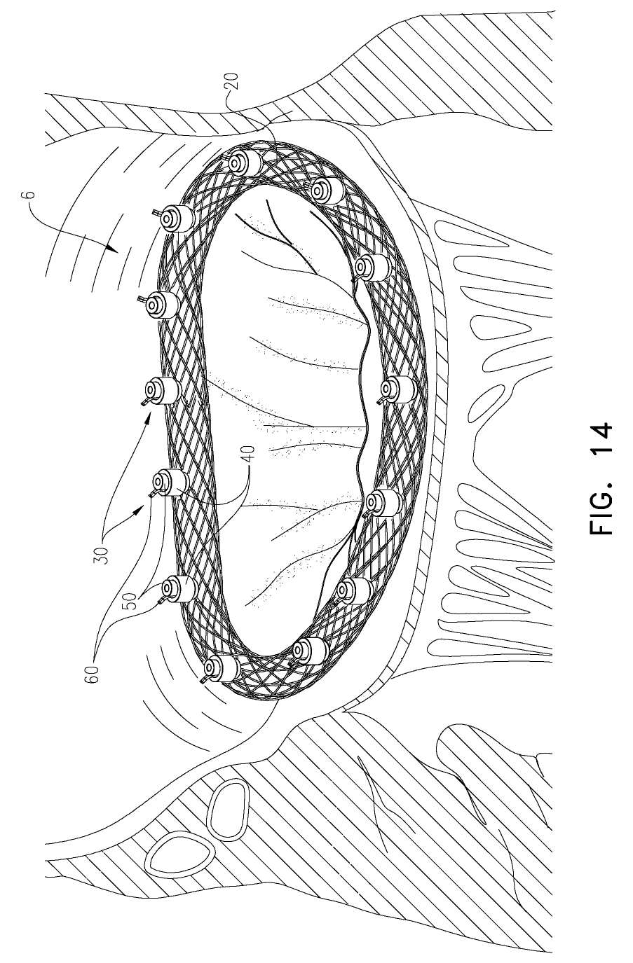

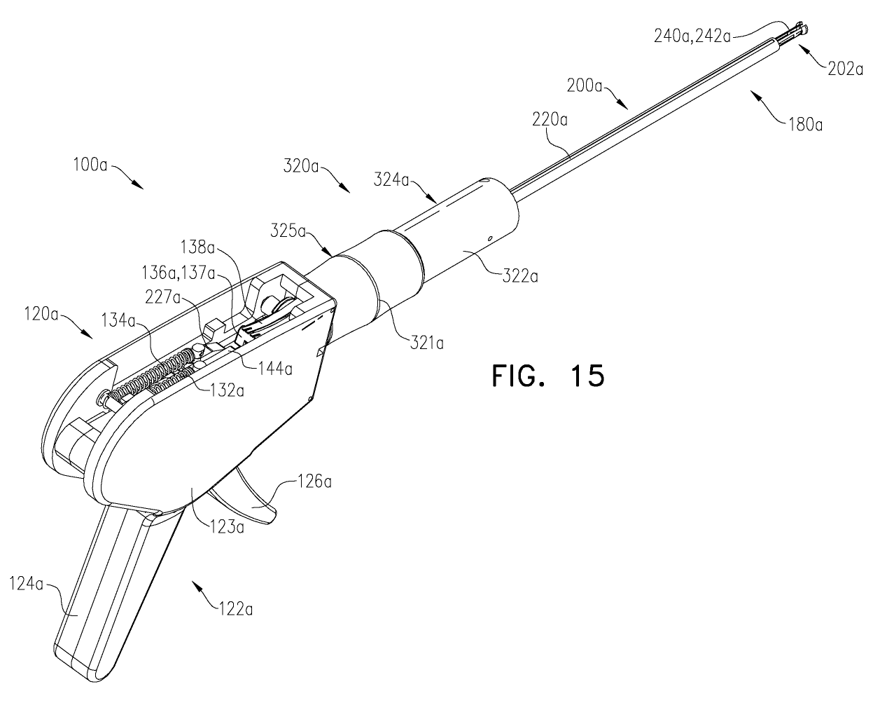

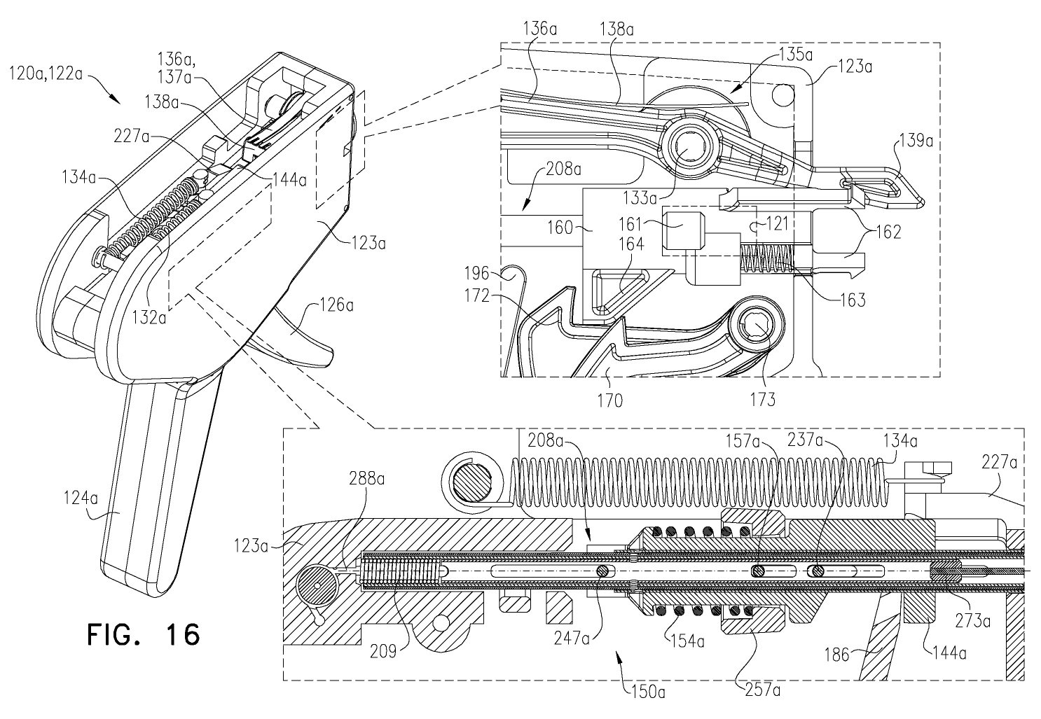

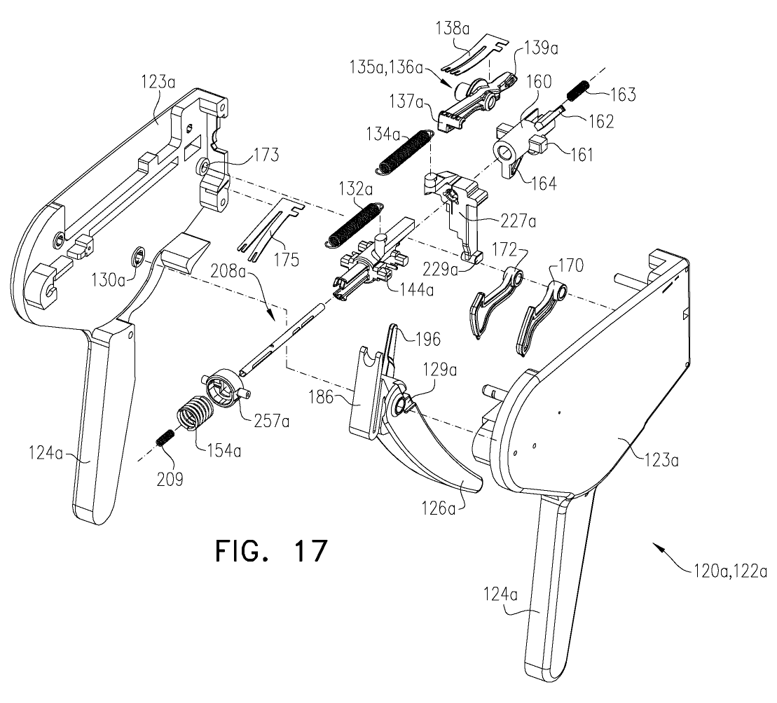

Reference is made to FIG. 14, which is a schematic illustration showing a plurality of suture locks 30 having been applied to respective sutures 60 at implant 20, in accordance with some implementations. In some implementations, the method described hereinabove with reference to FIGS. 11A-P, 12A-E and FIG. 13 is performed iteratively, in order to secure a plurality of suture locks 30 to lengths of suture 60 to implant 20.

Reference is made to FIG. 15, which is a schematic illustration showing a tool 100a (e.g., a delivery tool) for use with suture 60 and suture lock 30, and to FIGS. 16-17 and FIGS. 18A-B, which are schematic illustrations showing a handle 122a at the tool’s proximal portion 120a, in accordance with some implementations.

Tool 100a can be considered to be a variant of tool 100, and can be similar, at least in its general purpose, e.g., to secure multiple sutures 60 by applying a suture lock to each suture, to tool 100 disclosed hereinabove, mutatis mutandis. Components that are identically named between the tools 100, 100a typically share similar features and serve similar functions as each other. As such, the description below of tool 100a focuses upon features that are particular to tool 100a.

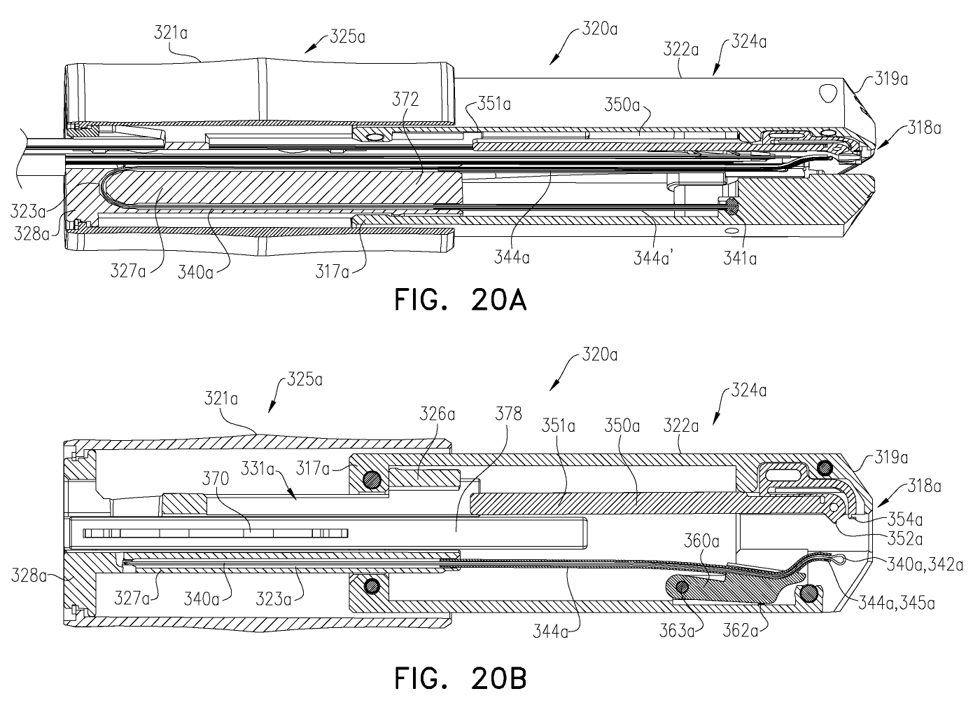

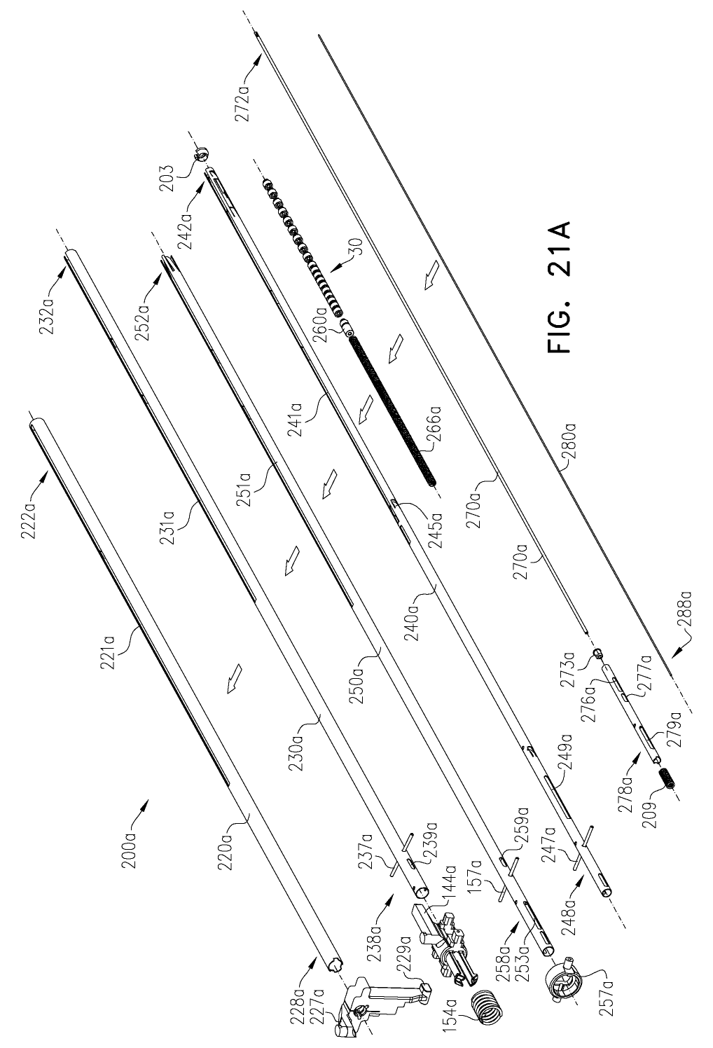

In some implementations, and as shown in FIG. 15, tool 100a has a multi-shaft system 200a that extends from handle 122a to the tool’s distal portion 180a, and a shuttle 320a that is slidable over and along the multi-shaft system, between the handle and a distal portion 202a of the multi-shaft system. In some such implementations, and as described in greater detail hereinbelow with reference to FIGS. 19 and 20A-B, shuttle 320a comprises a carriage 324a and a snare pump 325a that is slidable with respect to the carriage.

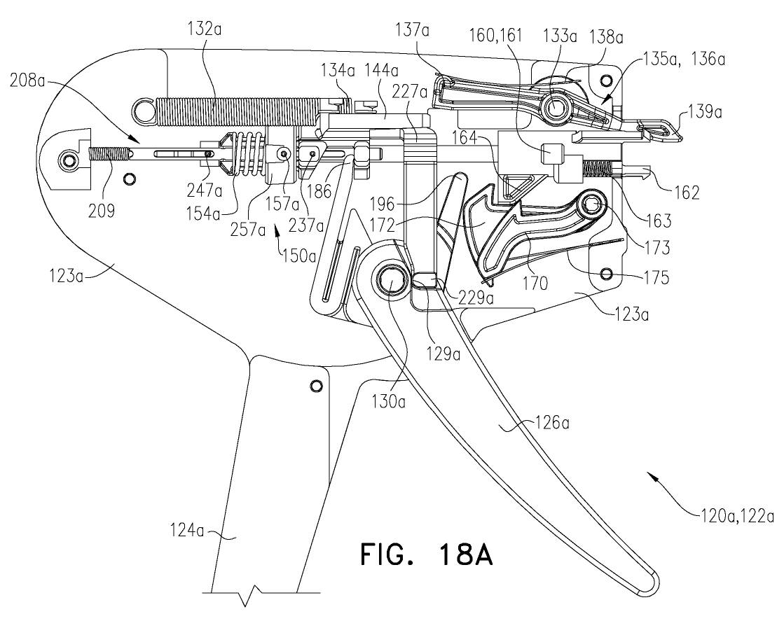

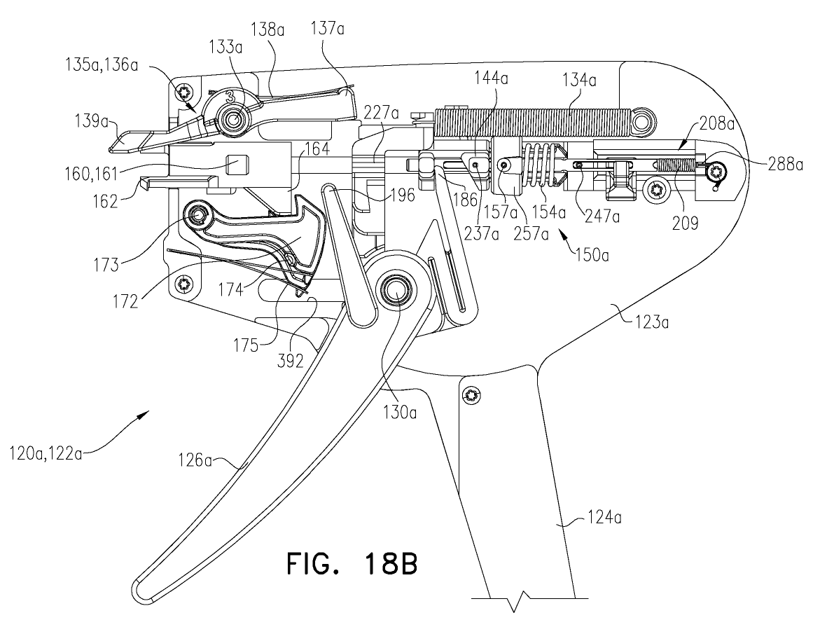

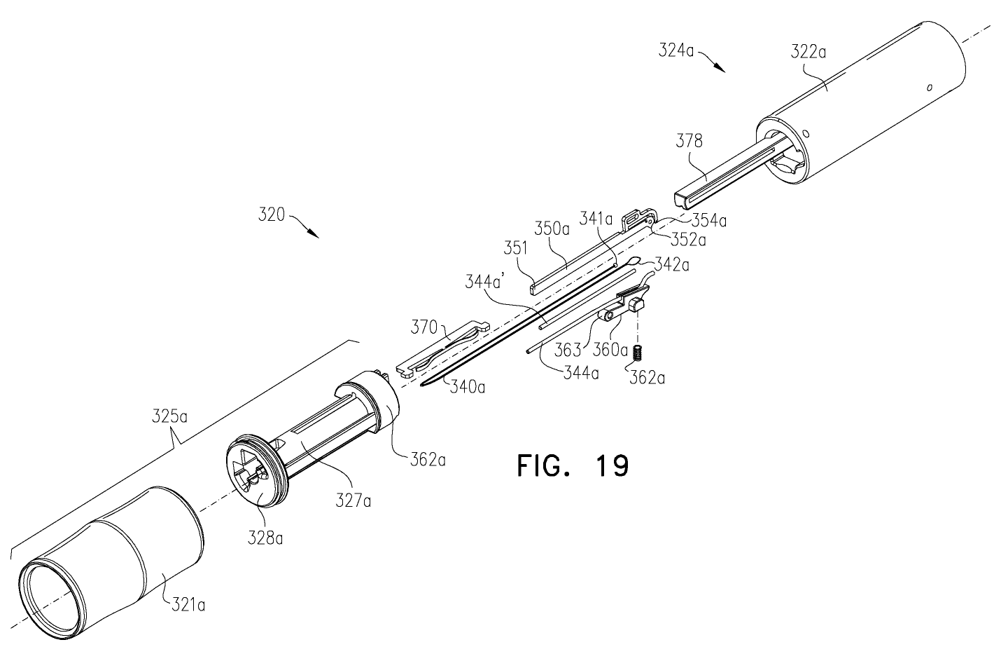

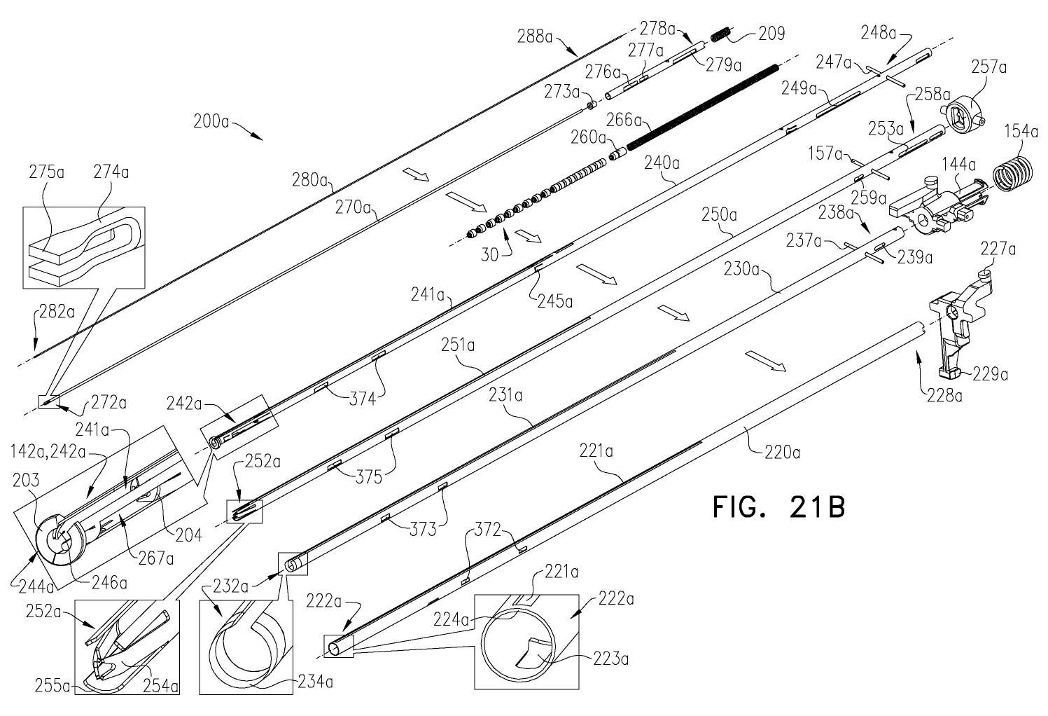

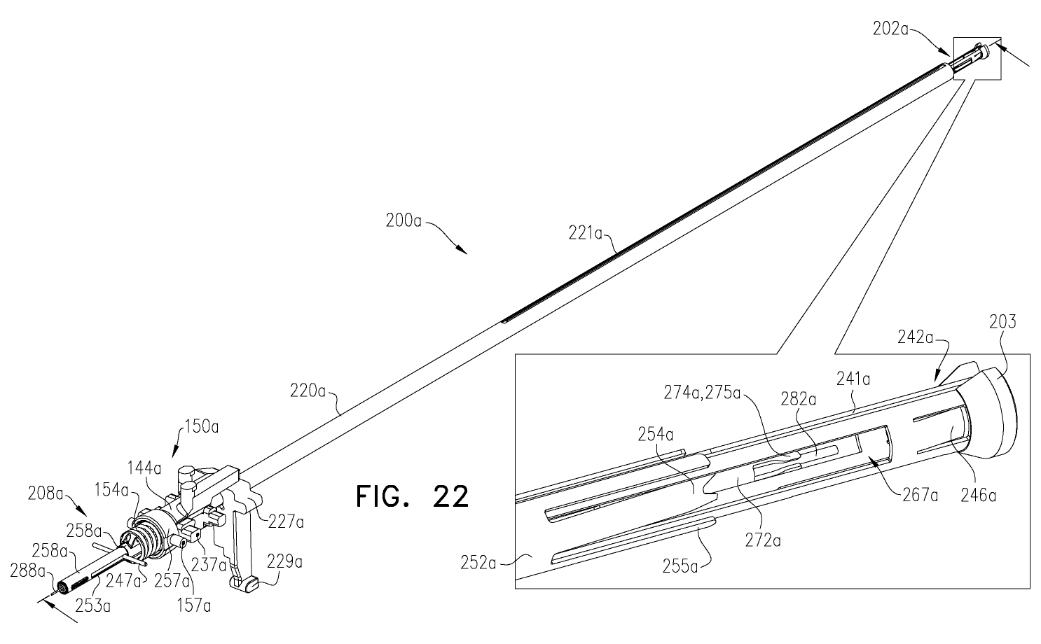

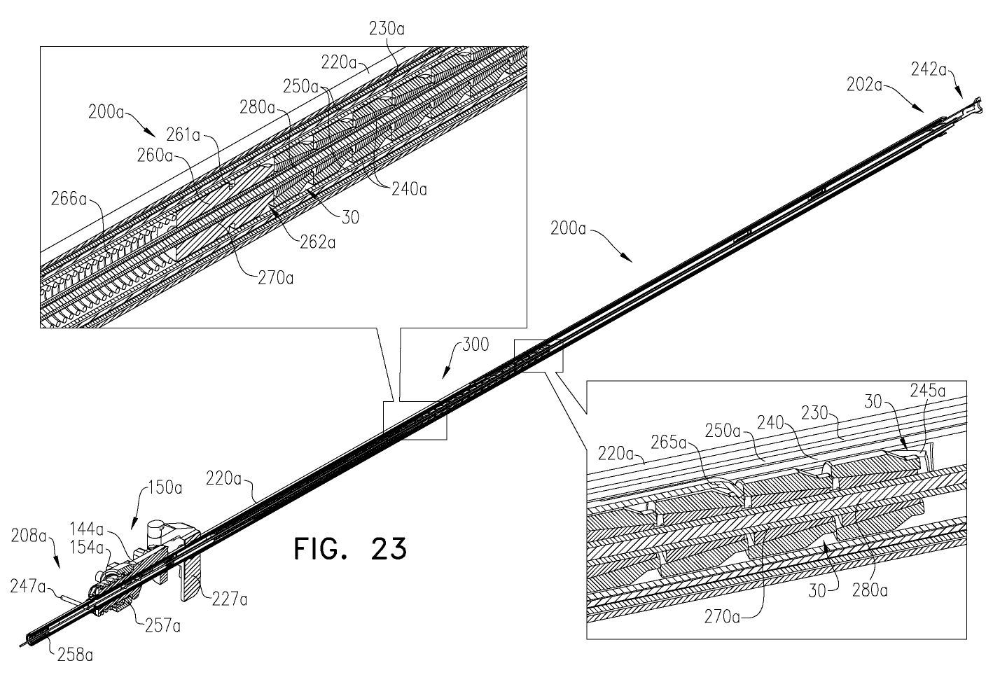

In some implementations, and as shown in FIG. 15, multi-shaft system 200a comprises a lock shaft 240a having a distal portion 242a that extends distally from within an outer shaft 220a. In accordance with some such implementations, FIGS. 18A-B show the multi-shaft system’s outer shaft block 227a, which is fixedly coupled to outer shaft 220a, such that the outer shaft and the outer shaft block slide together along the other shafts of system 200a. Additional features of multi-shaft system 200a are described hereinbelow with reference to FIGS. 21A-B and FIGS. 22-23.

In accordance with some implementations, FIG. 16 shows the tool’s proximal portion 120a. The main view of FIG. 16 is a perspective view of handle 122a, the upper inset is a partial cutaway view of the handle, and the lower inset is a cross-sectional view of the handle’s force regulator 150a. For the sake of clarity, the insets show the multi-shaft system’s proximal portion 208a within handle 122a (e.g., coupled to force regulator 150a). Multi-shaft system’s distal portion 202a is shown in FIG. 15 and in greater detail hereinbelow, e.g., in FIGS. 21A-B and FIGS. 22-23.

In some implementations, and as shown in the upper inset of FIG. 16, the tool’s proximal portion 120a comprises a pump adapter 160 by which shuttle 320a is reversibly coupled to handle 122a. For example, and as shown, pump adapter 160 defines catches 162 that reversibly engage shuttle 320a e.g., the shuttle’s pump body 327a.

In some implementations, longitudinal movement of pump adapter 160 is limited with relation to handle 122a. In some such implementations, and as shown in the upper inset of FIG. 16, pump adapter 160 defines a tenon 161 that slides within an adapter groove 121 defined by handle 122a. For example, and as shown, pump adapter 160 reaches its proximalmost position when tenon 161 abuts the adapter groove’s proximal edge.

In some implementations, and as shown, pump adapter 160 is biased to assume its proximalmost position, e.g., in absence of an external force that would pull the pump adapter distally. In some such implementations, and as shown, a pump adapter spring 163 is disposed within the handle’s housing 123a such that the pump adapter spring pushes pump adapter 160 proximally, e.g., such that the pump adapter assumes its proximalmost position.

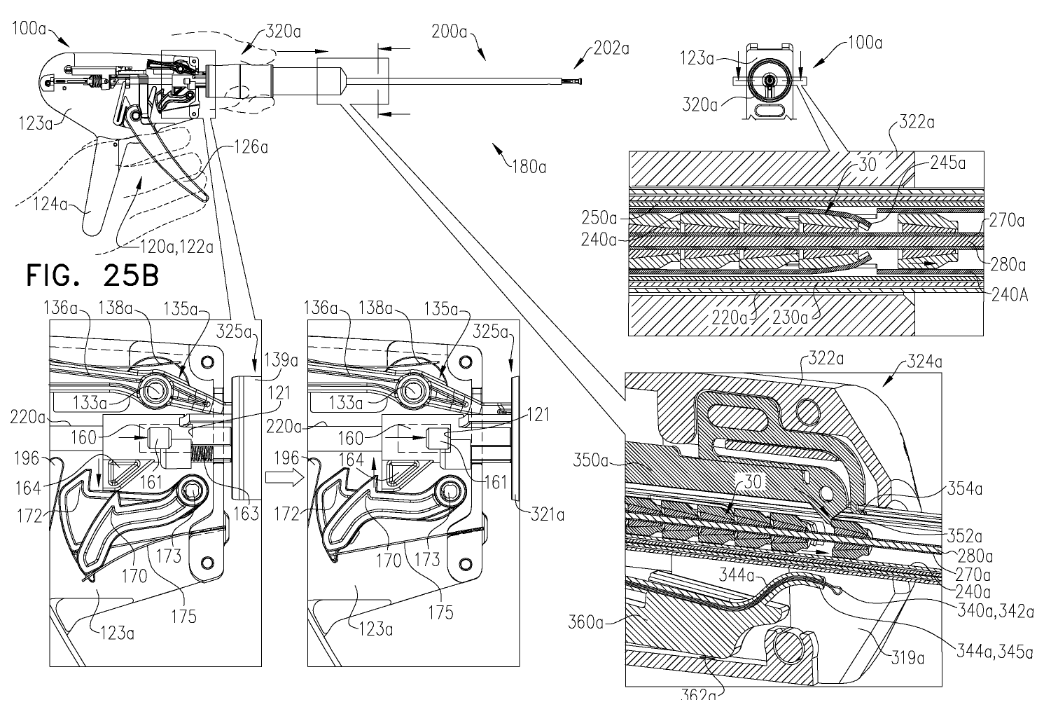

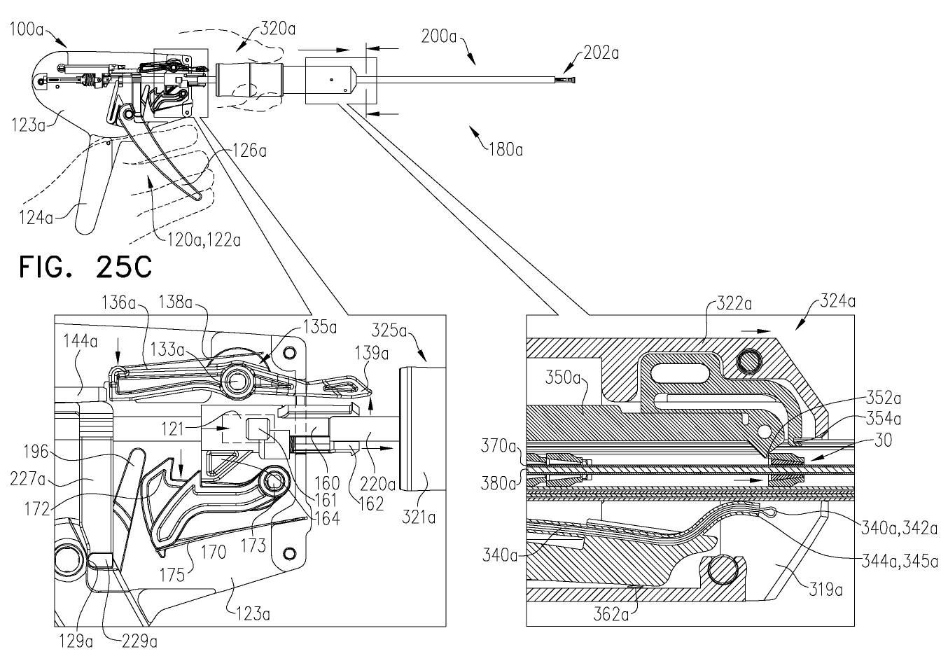

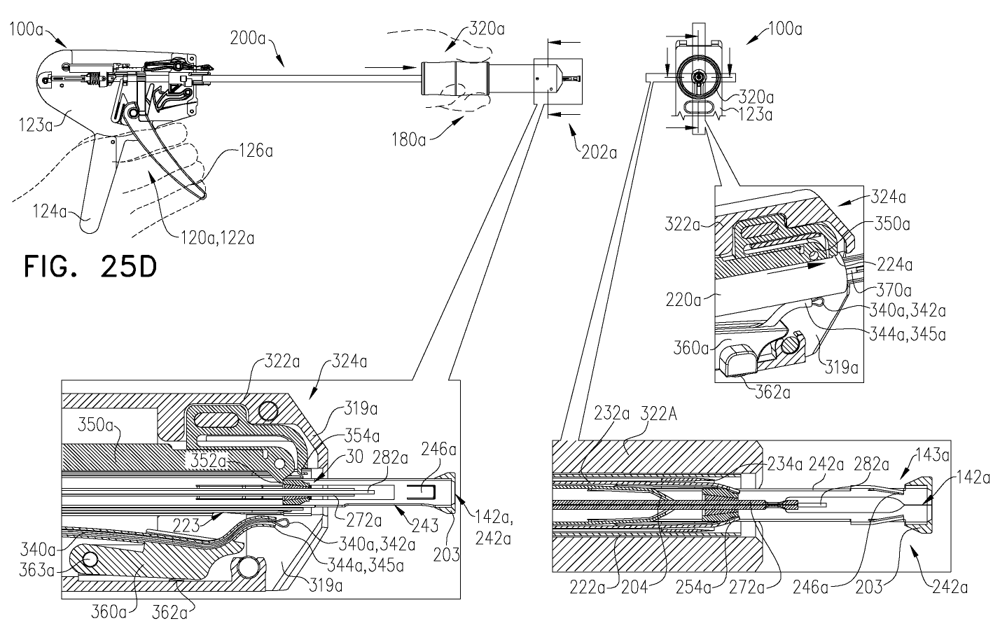

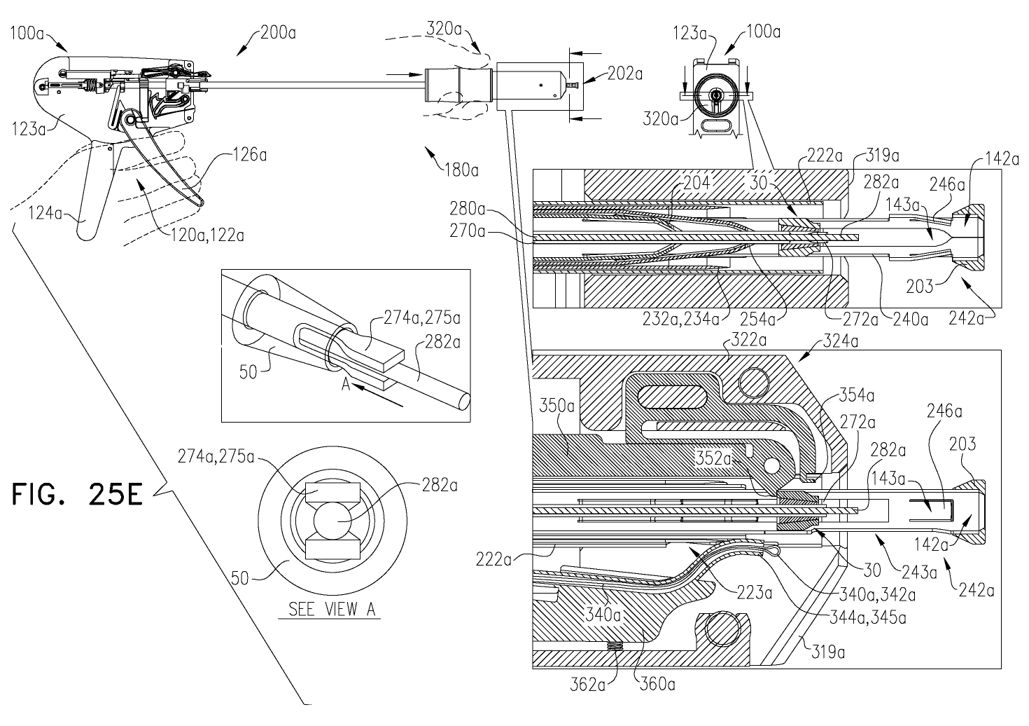

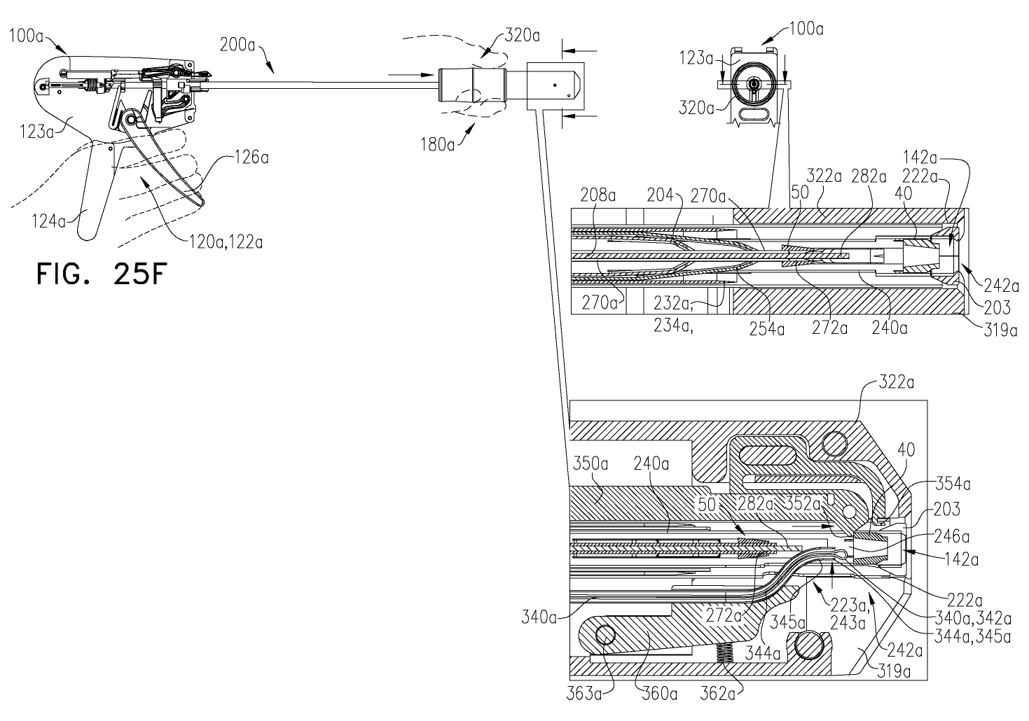

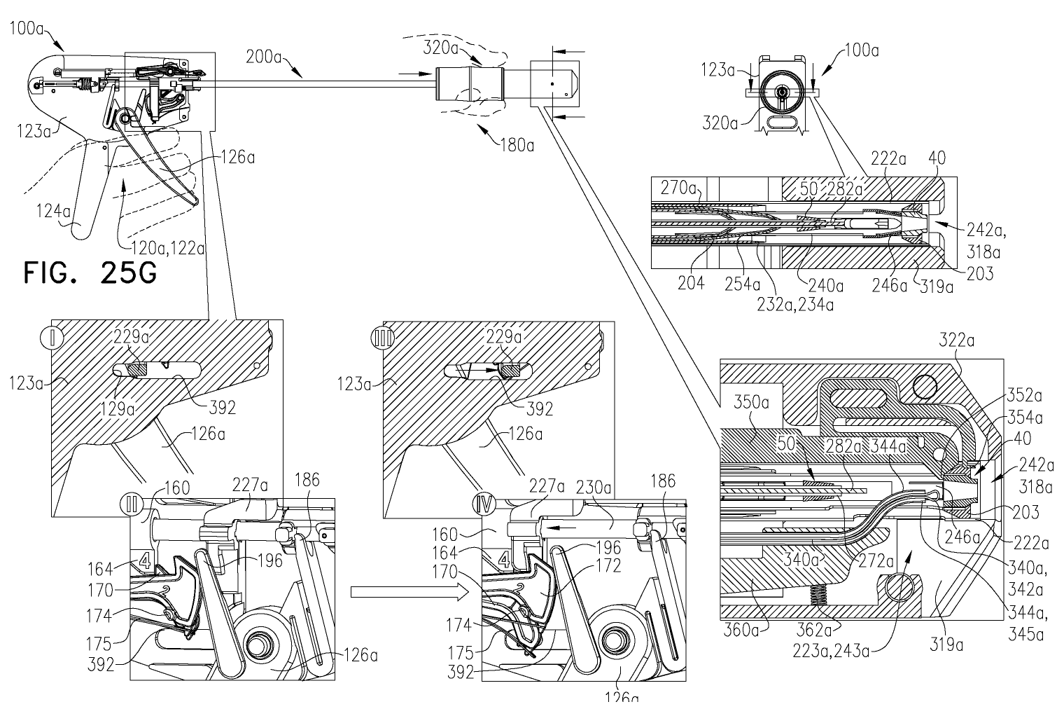

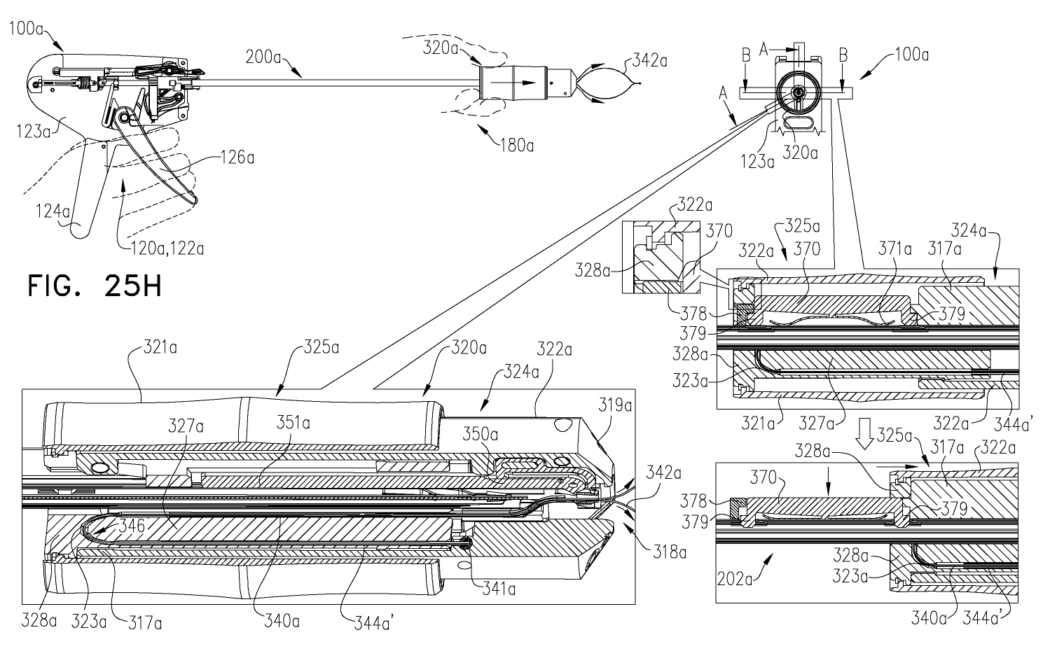

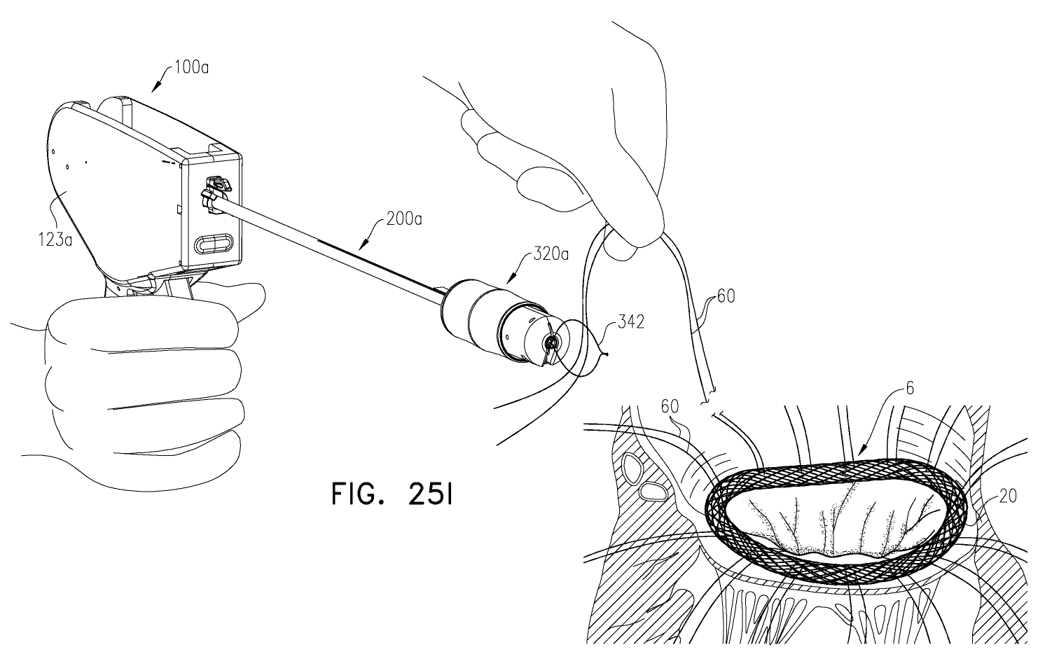

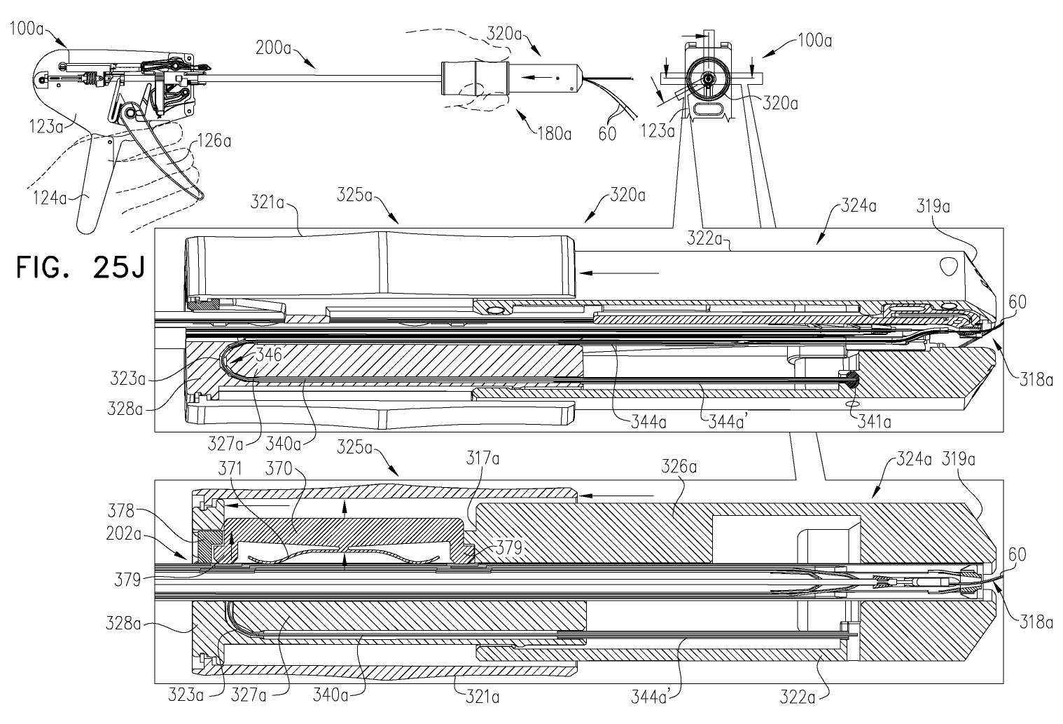

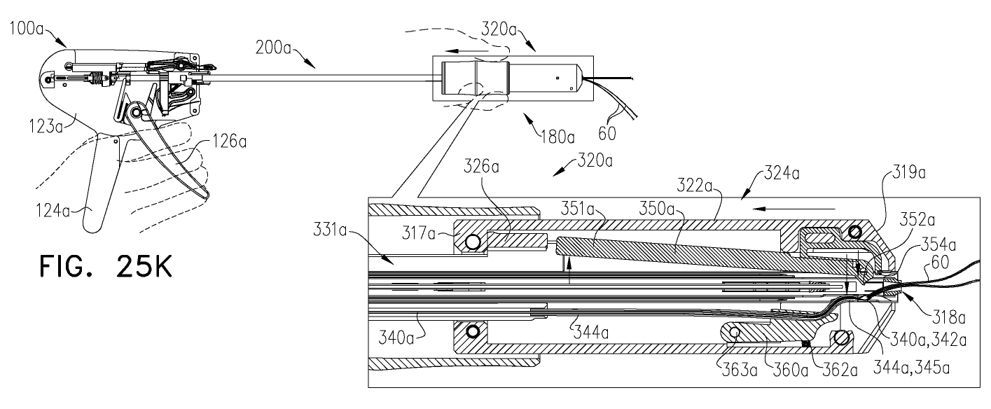

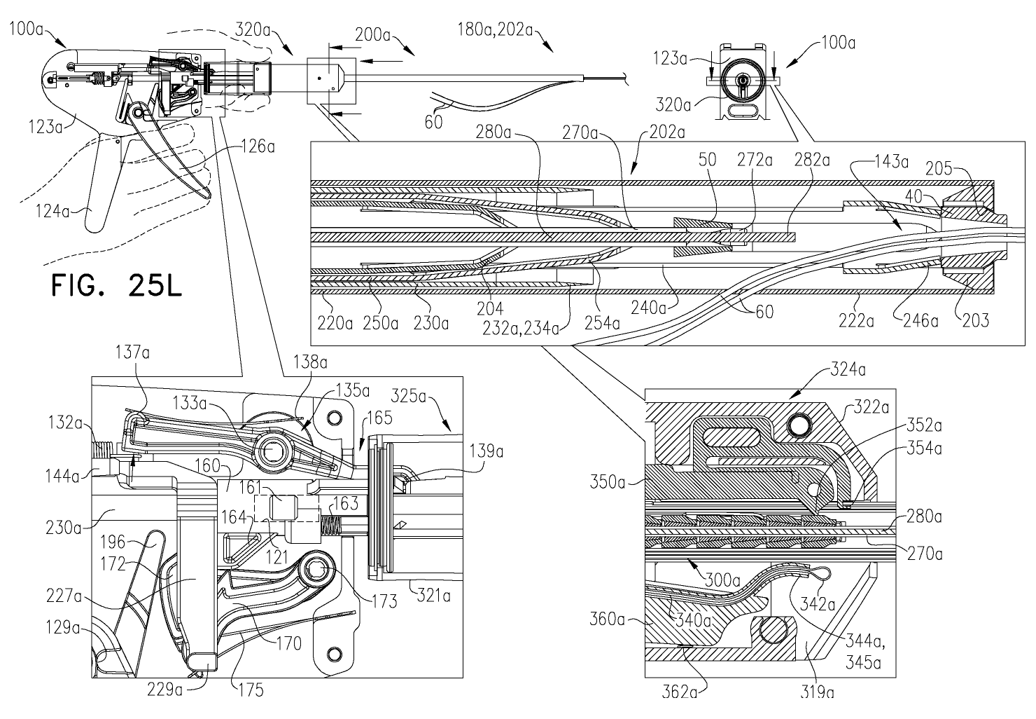

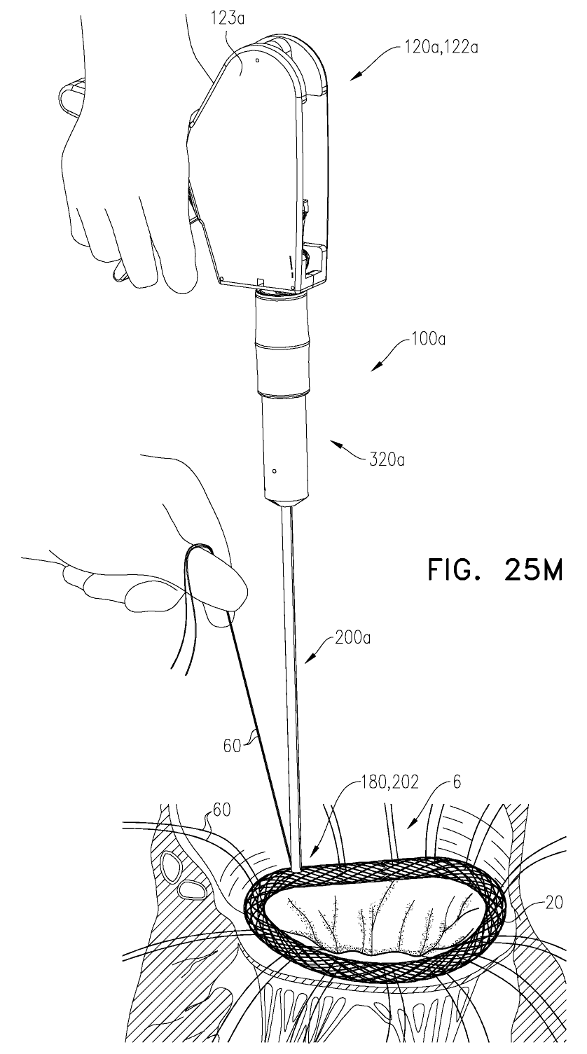

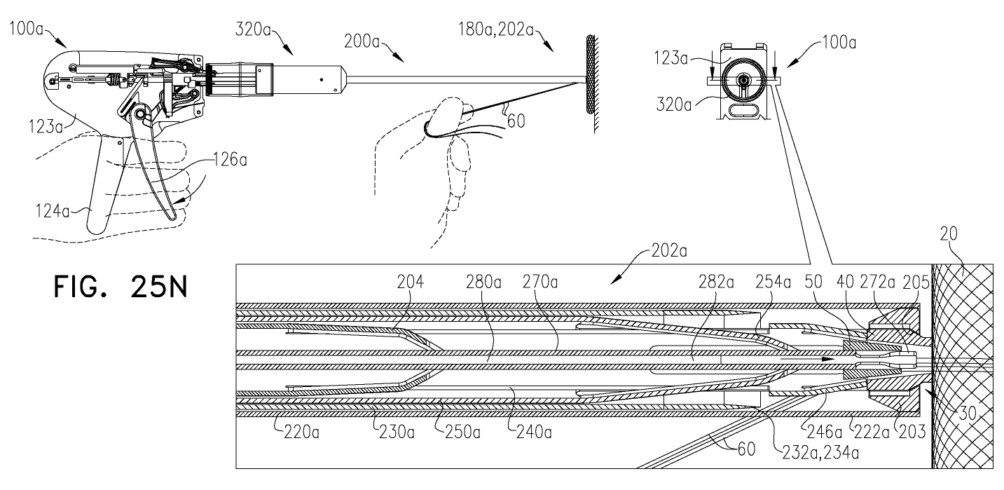

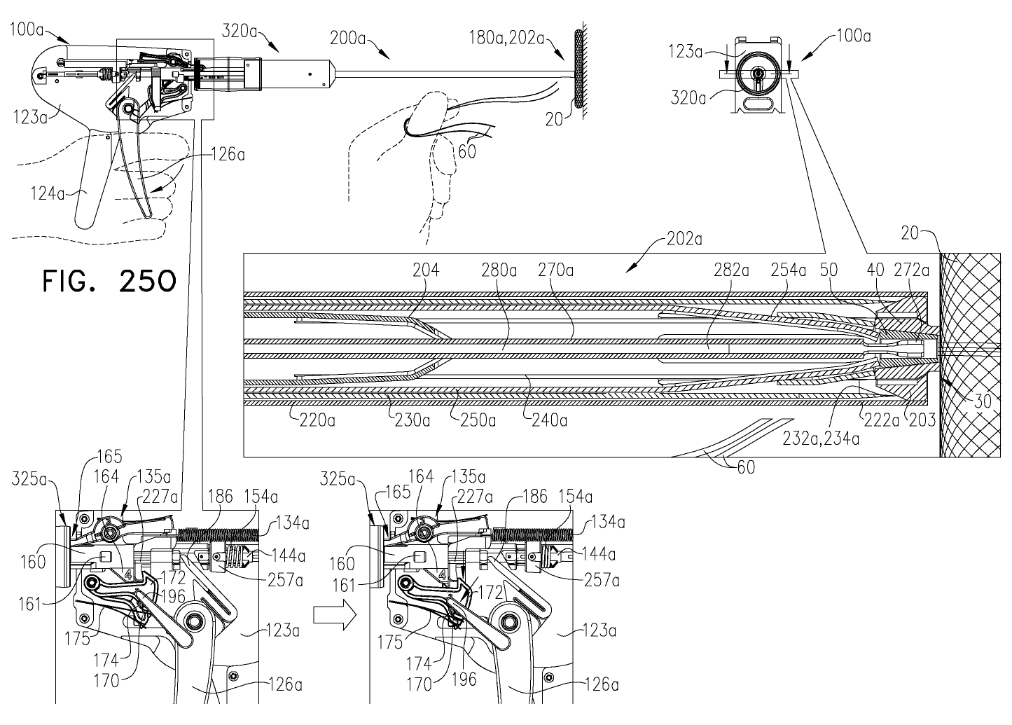

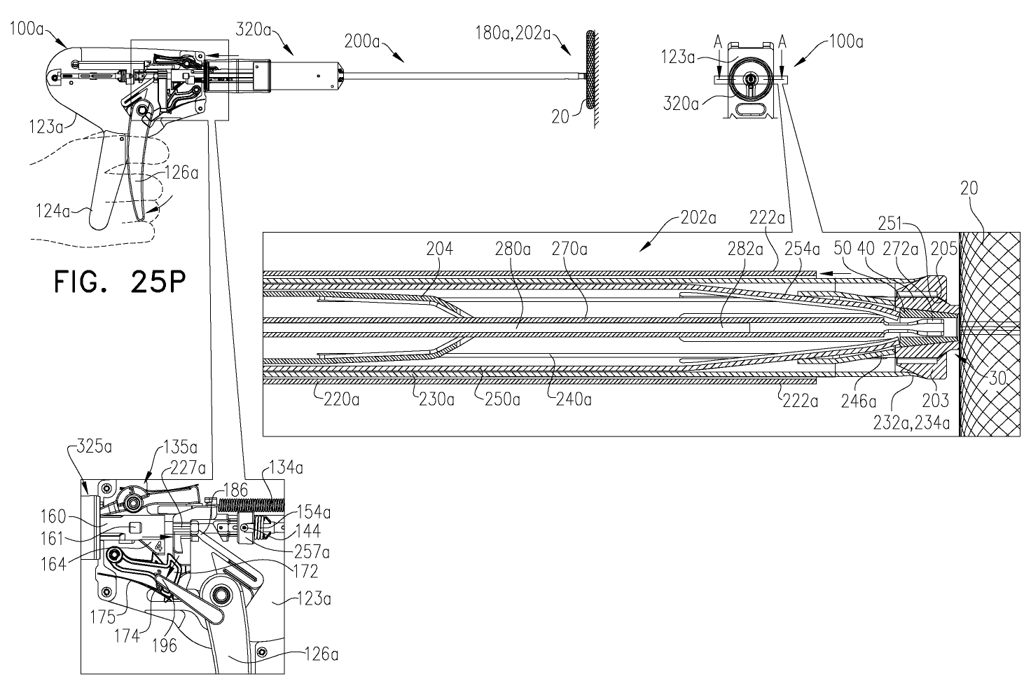

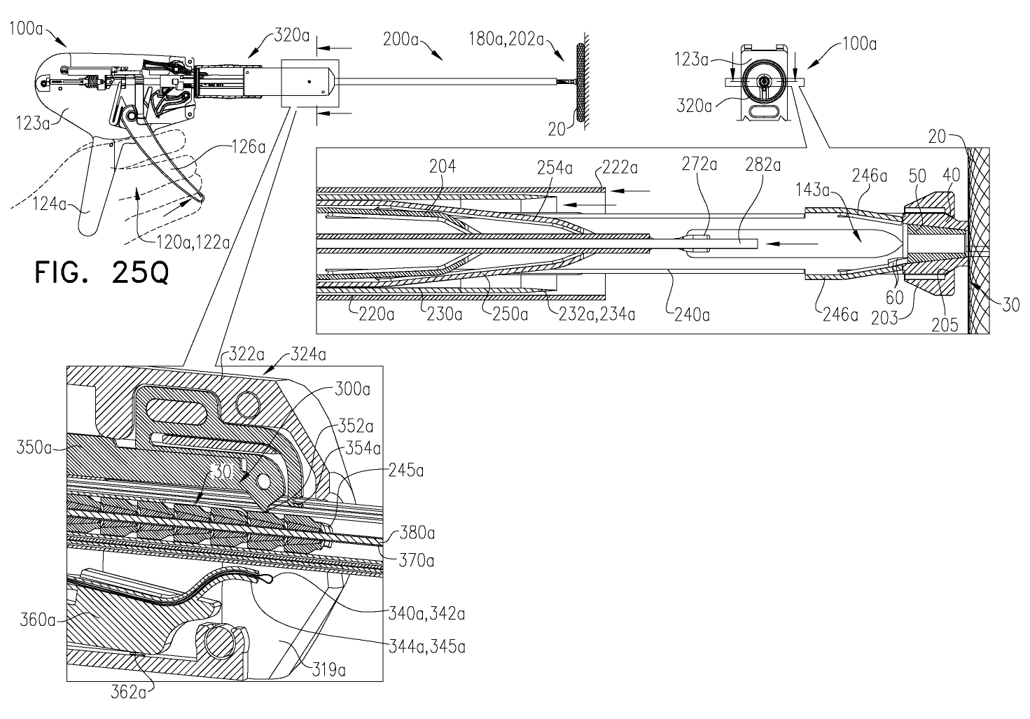

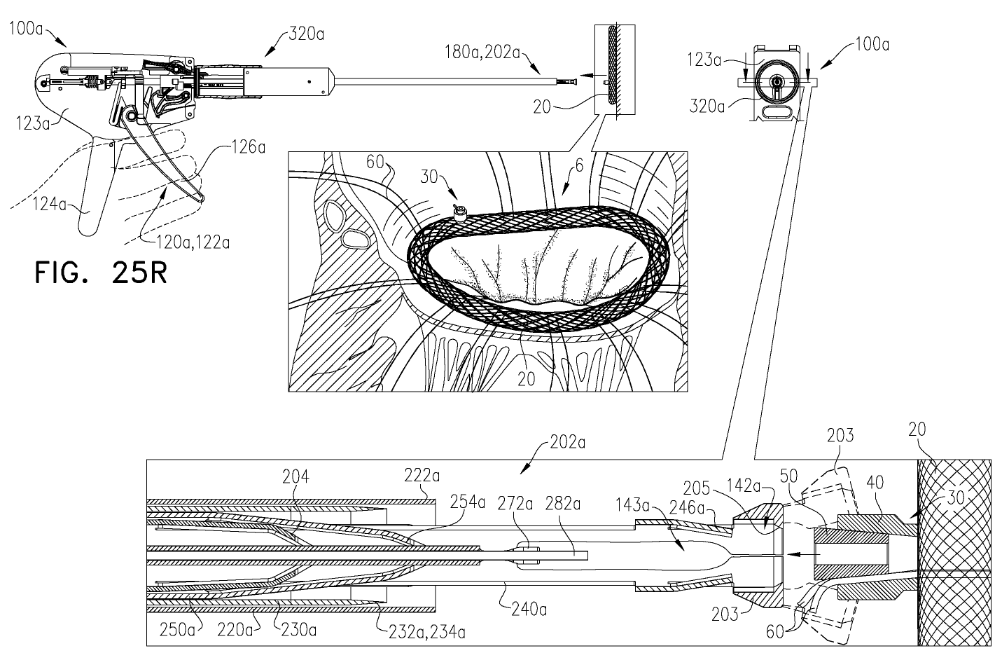

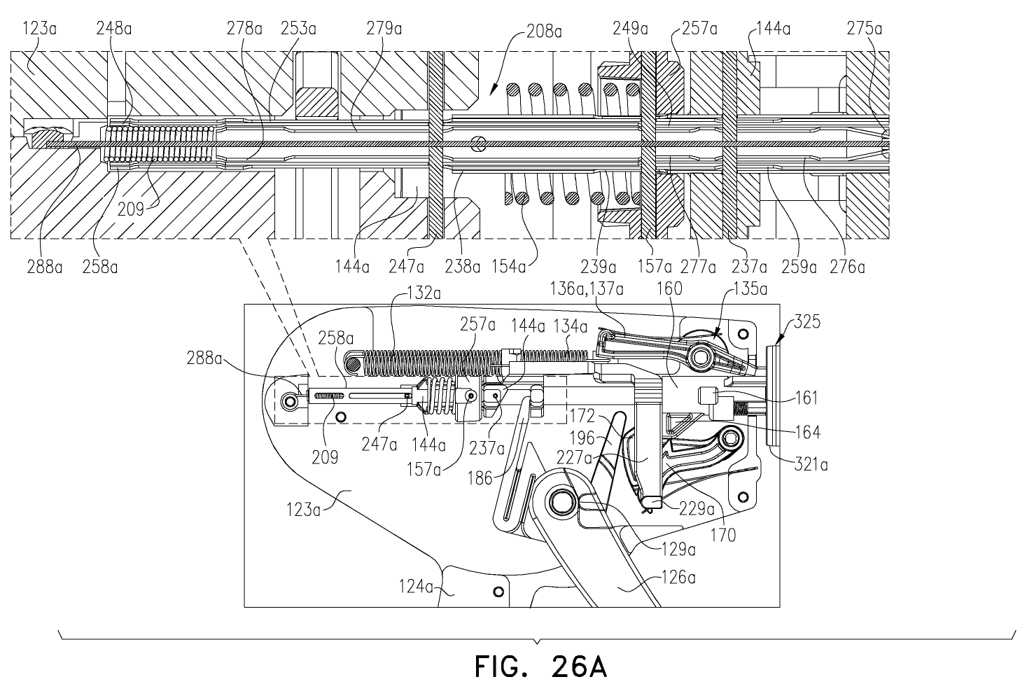

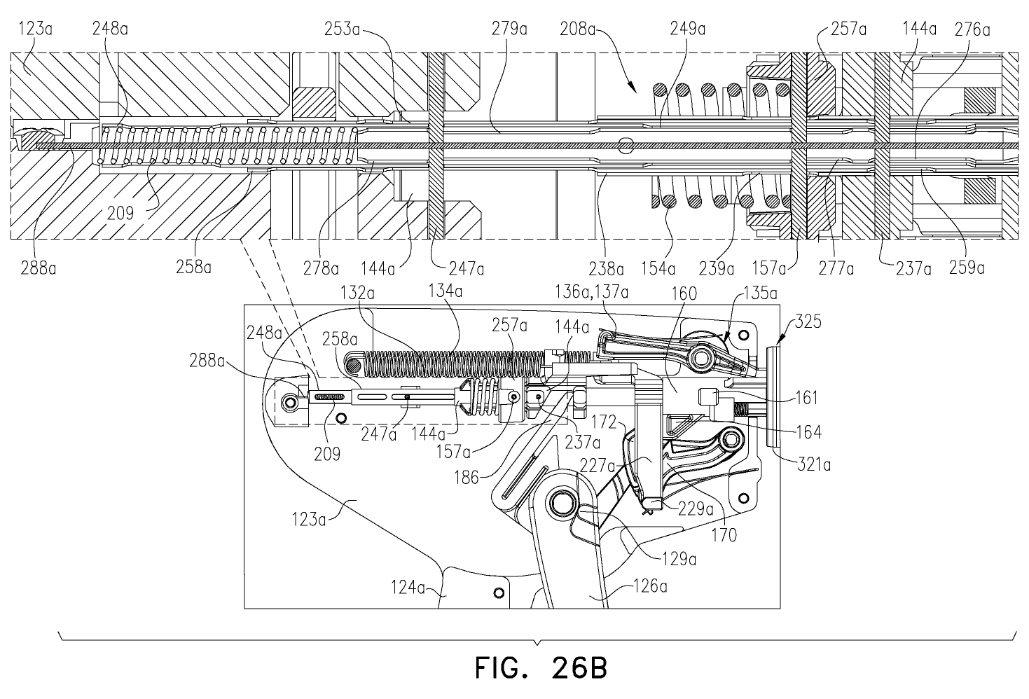

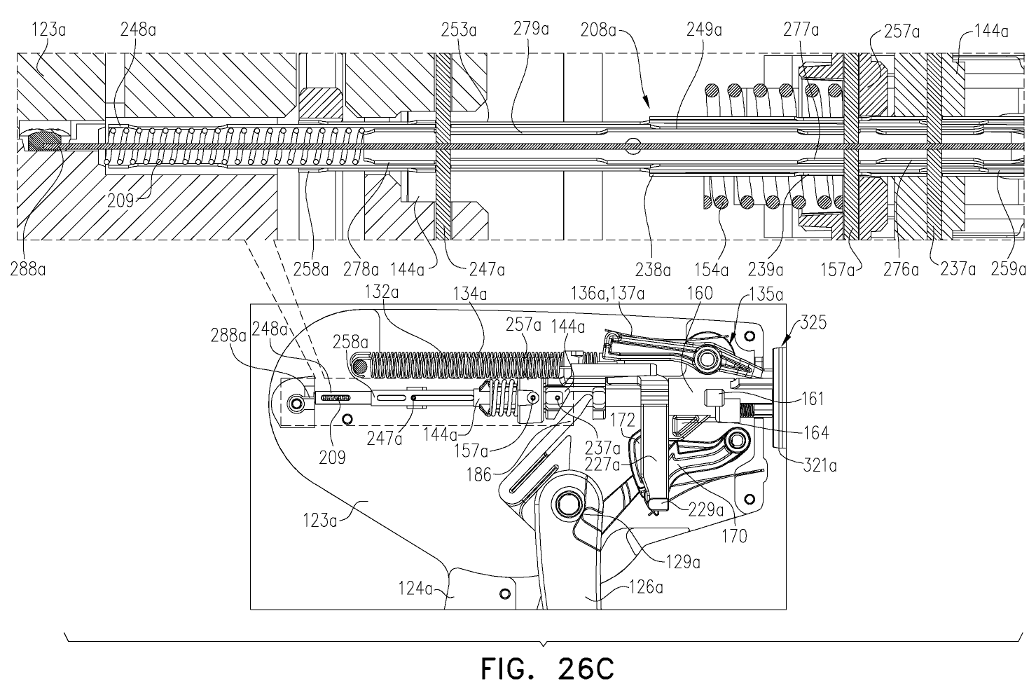

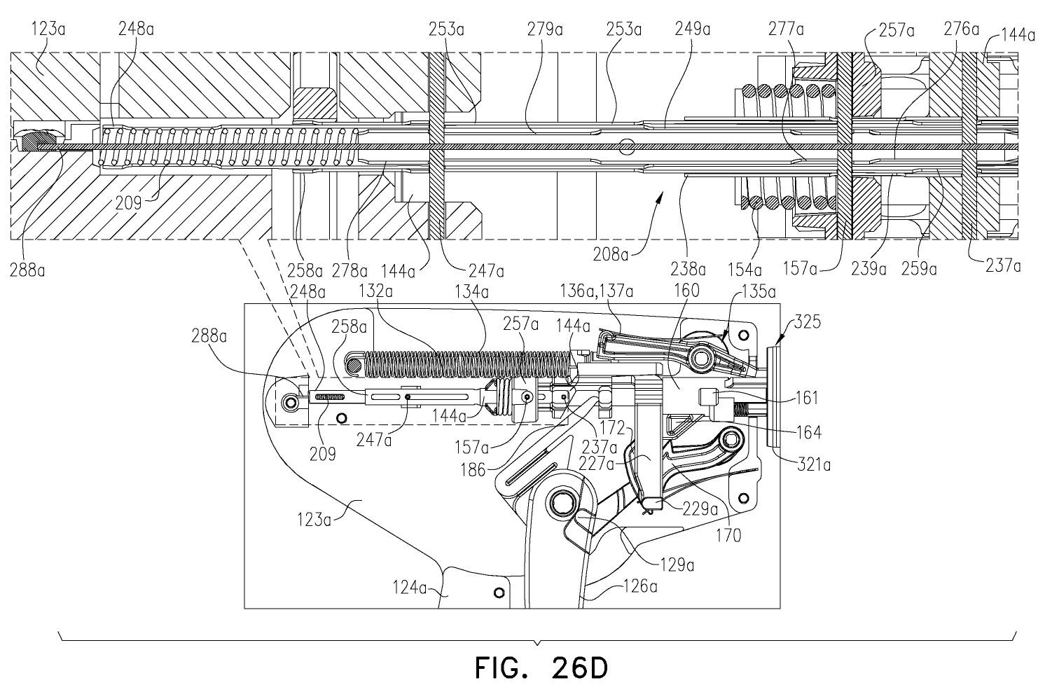

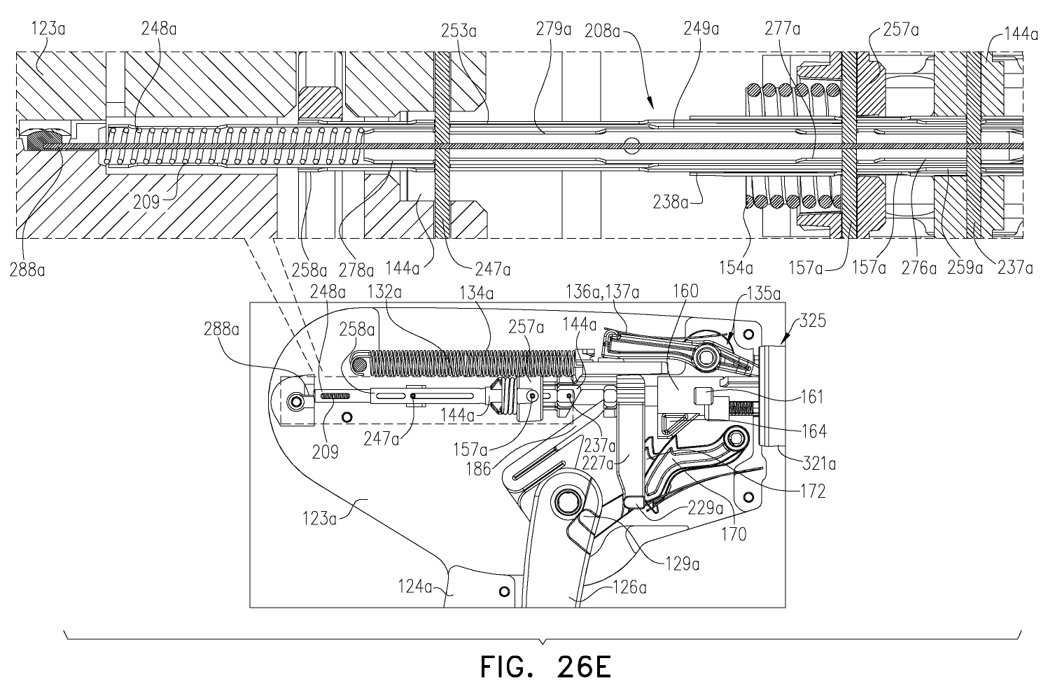

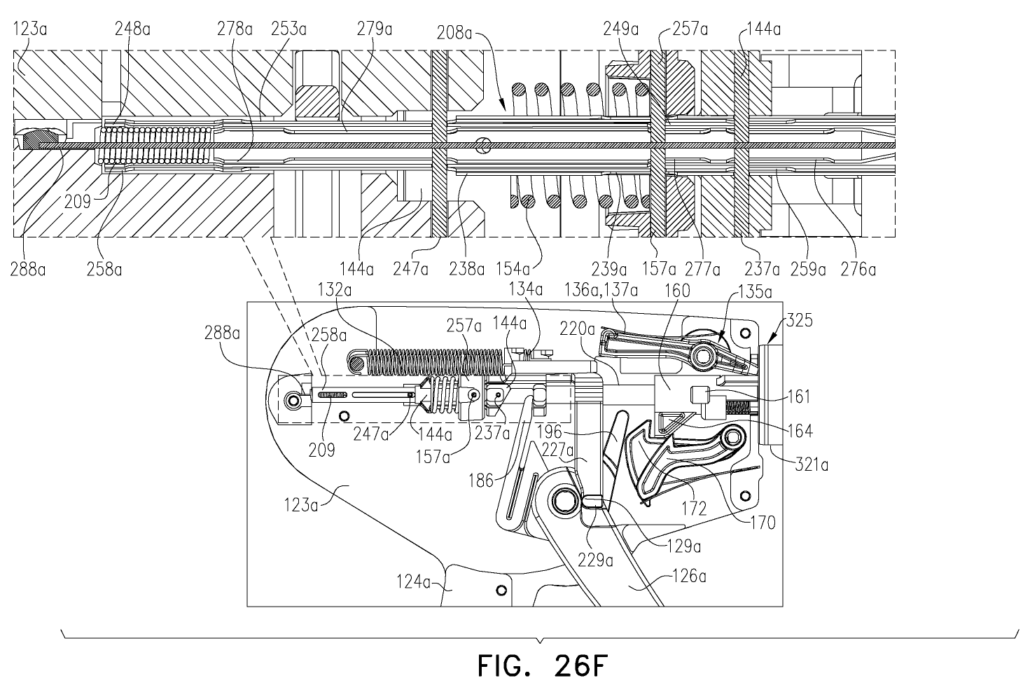

In some implementations, and as shown, pump adapter 160 defines a latch-interface 164 that is shaped to interact with handle 122a, e.g., with the handle’s pump adapter latch 170. In some such implementations, and as shown hereinbelow in FIG. 25B, advancing pump adapter 160 distally pushes pump adapter latch 170 away from latch-interface 164, thereby allowing the pump adapter to reach its distalmost position, e.g., wherein tenon 161 abuts the distal edge of adapter groove 121.

In some implementations, and as shown, pump adapter 160 is advanced or retracted in relation to handle 122a by manipulating pump adapter latch 170. In some such implementations, and as shown hereinbelow in FIG. 25A, pump adapter latch 170 and/or pump adapter spring 163 maintain pump adapter 160 in the pump adapter’s distalmost position.

In accordance with some implementations, FIG. 17 shows an exploded view of handle 122a, and FIGS. 18A–B are cutaway views of the handle with alternate halves of the handle’s housing 123a removed. That is, FIG. 18A, like most of the Figures described hereinbelow, show tool 100a from “starboard” (e.g., showing the tool’s distal portion on the right), such that the tool’s distal portion is on the right. However, FIG. 18B shows a “portside” view of tool 100a, in which the tool’s distal portion is on the left. As described hereinbelow, certain Figures show tool 100a from “portside”, e.g., in order to highlight features that are more readily visible in such views.

In some implementations, a latch spring 175 (FIGS. 18A–B) presses pump adapter latch 170 against the pump adapter’s latch-interface 164, e.g., until pump adapter 160 is pulled distally with sufficient force to compress pump adapter spring 163 and/or to deflect pump adapter latch 170 away from the pump adapter’s latch-interface 164. In some such implementations, and as shown in FIG. 17, latch spring 175 is bifurcated such that one tine of the latch spring presses pump adapter latch 170 against pump adapter 160, and the other tine of the latch spring presses against a trigger latch 172, e.g., as described hereinbelow with reference to FIG. 25G.

In some implementations, and as shown, trigger 126a defines block-interface 186 and a latch-interface 196. In some such implementations, block-interface 186 and latch-interface 196 respectively interact with trigger block 144a and trigger latch 172, e.g., a trigger-interface 174 that protrudes laterally from the trigger latch (FIG. 18B), as described hereinbelow with reference to FIGS. 25O–P. For example, and as shown, block-interface 186 can be shaped to flex, e.g., if trigger block 144a resists being pushed distally, e.g., in the case that trigger 126a is actuated while the trigger block is disposed at its distalmost position (FIG. 26E described hereinbelow).

In some implementations, and similarly to trigger interlock 135 of tool 100, handle 122a comprises a trigger interlock 135a (e.g., defining an interlock lever 136a, as shown) that prevents the user from inadvertently actuating trigger 126a. In some such implementations, an interlock spring 138a is coupled to handle 122a (e.g., to the handle’s housing 123a) such that the interlock spring pushes the interlock lever’s proximal portion 137a into contact with trigger block 144a (see left inset of FIG. 25C described hereinbelow), thereby inhibiting advancement of the trigger block unless interlock 135a has been unlocked. For example, and as described hereinbelow with reference to FIG. 25A, interlock 135a can be unlocked by proximally retracting shuttle 320a over system 200a such that the shuttle depresses the interlock lever’s distal portion 139a, thereby elevating the interlock lever’s proximal portion 137a away from trigger block 144a (e.g., as shown in FIGS. 18A–B).

In accordance with some implementations, and as shown in FIGS. 18A–B, the multi-shaft system’s proximal portion 208a is coupled to handle 122a (e.g., to force regulator 150a). In some such implementations, and as shown in FIG. 18B, a proximal portion 288a of the multi-shaft system’s inner wire 280a is anchored to housing 123a. In some such implementations, and as shown, the multi-shaft system’s outer shaft block 227a is coupled to housing 123a via an outer shaft spring 134a, such that sliding the outer shaft block distally along system 200a (e.g., from trigger block 144a towards pump adapter 160) tensions the outer shaft spring.

In some implementations, tool 100a is operated by applying an actuation force to handle 122a, e.g., by pivoting the handle’s trigger 126a toward the handle’s base 124a. In some such implementations, force regulator 150a (lower inset of FIG. 16) transfers the actuation force to a force applicator at the tool’s distal portion 180a, e.g., to a component of multi-shaft system 200a. In some such implementations, and as shown in the lower inset of FIG. 16, trigger 126a engages force regulator 150a in order to transfer the actuation force to system 200a.

In some implementations, and as shown in the lower inset of FIG. 16, the trigger’s block-interface 186 abuts trigger block 144a. In some such implementations, and as shown, a cutter pin 237a couples trigger block 144a to system 200a, e.g., a cutter shaft 230a thereof, as described hereinbelow. In this way, pivoting trigger 126a toward base 124a distally advances trigger block 144a, cutter pin 237a and cutter shaft 230a.

In some implementations, and as shown in the lower inset of FIG. 16, a lock block 257a is coupled via a plug pin 157a to a plug shaft 250a of system 200a described hereinbelow. In some such implementations, and as shown, a lock spring 154a is disposed between lock block 257a and the trigger block’s proximal portion. For example, lock spring 154a can be calibrated to compress between lock block 257a and the trigger block’s proximal portion when the trigger block applies sufficient force to the lock spring. In this way, and as described hereinbelow with reference to FIGS. 25N–P, (i) when the actuation force is applied to trigger 126a below a force threshold, cutter shaft 230a and plug shaft 250a advance in tandem, and (ii) when the actuation force reaches the force threshold, the actuation force compresses lock spring 154a, such that the trigger block and cutter shaft advance with respect to the lock block and plug shaft.

Reference is made to FIGS. 19 and 20A–B, which are schematic illustrations showing an exploded view (FIG. 19) and cross-sectional views (FIG. 20A–B) of shuttle 320a, in accordance with some implementations. Shuttle 320a can be considered to be a variant of shuttle 320, and components that are identically named between shuttles 320, 320a typically share similar features and serve similar functions as each other. As such, the description below of shuttle 320a focuses upon features that are particular to shuttle 320a.

As shown, shuttle 320a comprises a snare pump 325a that is longitudinally slidable (e.g., bidirectionally slidable) with respect to carriage 324a. In accordance with some implementations, FIGS. 20A–B show shuttle 320a in an extended state, e.g., in which a proximal portion 317a of carriage 324a contacts distal portion 326a of snare pump 325a.

In some implementations, and similarly to carriage 324 carriage 324a comprises a housing 322a to which a pusher 350a is attached. In some such implementations, and as shown in FIG. 20B, the pusher’s proximal portion defines a rail 351a, and the snare pump’s pump body 327a defines a longitudinal groove 331a that is aligned with the rail, e.g., while pusher 350a engages suture lock 30 (not shown). In this way, rail 351a can slide within groove 331a as the snare pump’s housing 321a is slid over the carriage’s housing 322a, such that the user can actuate snare pump 325a while pusher 350a engages suture lock 30.

In some implementations, sliding snare pump 325a with respect to carriage 324a releases and retracts snare 340a (e.g., through an aperture 318a in the carriage’s distal portion 319a), similarly to as described hereinabove with reference to shuttle 320. In some such implementations, and in contrast to the generally straight path that snare 340 follows through snare tube 344 and snare sleeve 323 of shuttle 320 (FIG. 6), snare 340a follows a looped path through a snare channel 346. In some such implementations, actuating snare pump 325a releases a greater length of snare 340a from the looped snare channel 346 than the length of snare 340 released by actuating snare pump 325. In some such implementations, snare 340a extends through snare channel 346 in a u-turn from the snare’s proximal portion 341a to its distal portion 342a, in order to increase the length of the snare that exits the snare tube’s distal opening 345a. For example, for each centimeter that snare pump 325a is slid along carriage 324a, the total length of snare channel 346 is reduced by two centimeters, thereby releasing two centimeters of snare 340a from the snare tube’s distal opening 345a.

FIG. 20A shows a compound cross-sectional view of shuttle 320a, in which the shuttle is cut (i) to show a side view of the shuttle, and (ii) along a plane, oblique to the side view, that shows the path that snare 340a extends from the snare’s proximal portion 341a through snare channel 346. In some implementations, and as shown, snare channel 346 comprises a two-portion snare tube 344a, 344a' (FIG. 19) and a snare sleeve 323a that is defined by pump body 327a (FIG. 20A). In some such implementations, snare sleeve 323a is slidable connected to snare tube 344a, 344', e.g., snare tubes 344a, 344a' fit inside snare sleeve 323a, such that sliding snare pump 325a over carriage 324a shortens the total length of snare channel 346 (described hereinbelow with reference to FIG. 25H).

In some implementations, and as shown in the side-view cross-section of shuttle 320a in FIG. 20B, the snare’s distal portion 342a extends distally from a distal opening 345a in snare tube 344a. Since the snare’s proximal portion 341a is fixedly coupled to carriage 324a (FIG. 20A), shortening the total length of snare channel 346 increases the length of the snare’s distal portion 342a extending from the distal snare tube’s distal opening 345a (shown below in FIG. 25H).

In some implementations, and as shown in FIGS. 19 and 20B, shuttle 320a comprises a snare spring 362a that is compressed while system 200a (not shown in FIGS. 19 and 20A–B) pushes the distal snare tube 344a away from the shuttle’s central axis. In some such implementations, and similarly to as described hereinabove with reference to system 200, shafts of system 200a define windows that, when aligned over distal snare tube 344a, allow snare spring 362a to pivot snare lever 360a about snare lever hinge 363a such that the distal snare tube 344a (e.g., distal opening 345a thereof) enters the shafts’ windows, and actuating snare pump 325a releases snare 340a through the shafts’ windows and exits tool 100a through the shuttle’s aperture 318a.

In some implementations, and as shown in FIGS. 19 and 20B, shuttle 320a comprises a pump lock 370 that is shaped to longitudinally lock the shafts of system 200a when snare 340a is extended from shuttle 320a, as described hereinbelow with reference to FIGS. 25H. In some such implementations, longitudinally locking the shafts keeps the shafts’ windows in place as the snare is extended from shuttle 320a, e.g., isolating the shafts from force applied by the user to operate snare pump 325a.

Reference is made to FIGS. 21A–B and 22–23, which are schematic illustrations showing multi-shaft system 200a of tool 100a, in accordance with some implementations. In some implementations, and similarly to system 200, system 200a comprises a series of coaxial shafts which are used in conjunction with suture lock 30. As such, system 200a can be considered to be a variant of system 200, and components that are identically named between the multi-shaft systems typically share similar features and serve similar functions as each other. As such, the description below of system 200a focuses upon features that are particular to system 200a.

FIGS. 21A–B are exploded views of multi-shaft system 200a, in which the system’s shafts are arranged in FIGS. 21A–B in order of ascending diameter, with arrows pointing from each shaft to the next shaft of greater diameter. FIGS. 22–23 show perspective and cross-sectional views of system 200a assembled such that the system extends from proximal portion 288a of wire 280a to distal portion 242a of lock shaft 240a. In some implementations, and as shown in FIG. 22, the shafts of system 200a are slidably coupled to each other via force regulator 150a that distributes force (e.g., from trigger 126a) to the system, as described hereinabove with reference to FIG. 16.

In some implementations, and as shown, system 200a comprises outer shaft 220a, cutter shaft 230a, lock shaft 240a, plug shaft 250a, an inner shaft 270a and an inner wire 280a. In some such implementations, and as shown, outer shaft 220a, cutter shaft 230a, lock shaft 240a and plug shaft 250a each define a longitudinal slot 221a, 231a, 241a, 251a that is shaped to allow pusher 350a (e.g., lip 352a thereof) to advance therethrough, in order to slide suture lock 30 to the system’s distal portion 202a. For In some such implementations, an outer shaft-engaging tooth 354a of pusher 350a is shaped to engage a distal tooth 224a at the outer shaft’s distal portion 222a, in order to advance outer shaft 220a, e.g., together with suture lock 30, with respect to other shafts of system 200a.

In some implementations, and as shown in FIG. 21B, shafts 220a, 230a, 240a, 250a each define lock slots 372, 373, 374, 375, e.g., each shaft defines a pair of lock slots. In some such implementations, each shaft’s lock slots are shaped to align with each other when tool 100a assumes a ready state (e.g., in which the tool is ready to snare a suture) described hereinbelow with reference to FIG. 25G.

In some implementations, and as shown, the outermost shaft of system 200a is outer shaft 220a, which extends distally from an outer shaft block 227a (shown coupled to the outer shaft’s proximal portion 228a in FIGS. 22–23), to the outer shaft’s distal portion 222a. In some implementations, and as shown in FIG. 21B, distal portion 222a defines a window 223a, e.g., through which distal snare tube 344a can enter the outer shaft’s lumen.

In some implementations, and as most readily visible in FIG. 22, outer shaft block 227a defines a double-sided tongue 229a that is shaped to engage (i) a groove 129a in the handle’s trigger 126a (FIGS. 17, 18A), as well as (ii) a groove 392 in the handle’s housing 123a (FIG. 18B). In some such implementations, and as shown, tongue 229a serves as a safety mechanism that inhibits actuation of trigger 126a while the outer shaft block’s tongue 229a is engaged with the trigger’s groove 129a, e.g., until the suture lock’s tube 40 is seated in the lock shaft’s holding zone 142a (FIG. 25G). Alternatively or in addition, the handle housing’s groove 392 guides longitudinal travel of the outer shaft block’s tongue 229a during operation of tool 100a.

In some implementations, and as shown, cutter shaft 230a extends distally from a proximal portion 238a to a distal portion 232a. In some such implementations, cutter shaft 230a fits slidably within outer shaft 220a and defines a blade 234a at distal portion 232a (FIG. 21B), such that the blade can be advanced with respect to other shafts of system 200a.

In some implementations, and as shown in FIGS. 21A–B, the cutter shaft’s proximal portion 238a is fixedly coupled to cutter pin 237a, which is itself fixed to trigger block 144a (also shown in lower inset of FIG. 16 and FIGS. 22–23). In some such implementations, and as shown, lock spring 154a is fitted onto the trigger block’s distal portion, facilitating the lock spring’s role in force regulator 150a, as described hereinabove with reference to FIG. 16.

In some implementations, and as shown, plug shaft 250a is dimensioned to fit slidably within cutter shaft 230a, and extends distally from a proximal portion 258a of the lock shaft to a distal portion 252a of the lock shaft. In some such implementations, and as shown in FIG. 21B, the lock shaft’s distal portion 252a defines (e.g., a pair of) plug-pusher arms 254a. In some such implementations, plug-pusher arms 254a are biased (e.g., heat-set) radially inwards in the absence of an external force (e.g., presence of suture lock 30 and/or lock shaft 240a between the plug-pusher arms) that would push the plug-pusher arms apart. In some such implementations, distal portion 252a further defines a suture support 255a, e.g., which, in contrast to plug-pusher arms 254a, is not biased to extend radially inwards, as shown in the inset of FIG. 21B showing the plug shaft’s distal portion 252a.

In some implementations, and as shown in the lower inset of FIG. 16, the plug shaft’s proximal portion 258a is coupled by plug pin 157a to lock block 257a. In some such implementations, and as shown in FIGS. 21A–B, the plug shaft’s proximal portion 258a is shaped to define a proximal slot 253a that facilitates sliding cutter pin 237a therethrough, thereby advancing cutter shaft 230a in order to trim suture 60 when the actuation force reaches the force threshold. For example, the force threshold can be between 2.5–3.5 kg of force, such as 2.75–3.25 kg of force, e.g., 3 kg of force.

In some implementations, and as shown in FIGS. 21A–B, a lock shaft 240a dimensioned to slidably fit within plug shaft 250a extends distally from a proximal portion 248a of the lock shaft to a distal portion 242a of the lock shaft. In some such implementations, and as shown, the lock shaft’s proximal portion 248a defines a lock pin 247a that is fixedly coupled to both the lock shaft and to the handle’s housing 123a.

In some implementations, and as shown, the lock shaft’s proximal portion 248a is shaped to define a proximal slot 249a that facilitates sliding other shafts of system 200a with respect to the lock shaft, e.g., by allowing plug pin 157a and/or cutter pin 237a to slide through the lock shaft’s proximal slot.

In some implementations, and similarly to lock shaft 240 of system 200, lock shaft 240a defines distal retention tabs 245a that protrude into the lock shaft’s lumen to define a location at which a distalmost suture lock 30 is positioned within storage zone 300a (FIG. 23). In contrast to system 200, lock shaft 240a of system 200a is disposed within plug shaft 250a, such that distal retention tabs 245a do not require lateral windows in the plug shaft in order to serve their function, e.g., retaining a distalmost suture lock 30.

In some implementations, and as shown in FIG. 21B, distal portion 242a of lock shaft 240a defines a holding zone 142a that is shaped to hold suture lock 30. In some such implementations, and as shown in FIG. 22, distal portion 242a defines a holding arm 246a that is shaped to retain suture lock 30 in holding zone 142a, similarly to holding arm 246 of lock shaft 240.

In some implementations, and in contrast to distal portion 242 of lock shaft 240, distal portion 242a may not necessarily be biased to flare radially outwards. In some such implementations, the lock shaft’s distal portion 242a does not require compression in order to retain its conformation in which holding zone 142a can stably hold suture lock 30.

In some implementations, distal portion 242a defines a pair of distal slots 267a, through which the plug shaft’s plug-pusher arms 254a can access the suture lock’s plug 50, e.g., as described hereinbelow with reference to FIG. 25O.

In some implementations, the lock shaft’s distal end 244a has an atraumatic crown 203 that can reduce a risk of damage to tissue that the lock shaft contacts.

In some implementations, and as shown, inner shaft 270a is dimensioned to fit slidably within lock shaft 240a and extends distally from a proximal sleeve 278a of the inner shaft to a distal portion 272a of the inner shaft that defines branches 274a. In some such implementations, branches 274a define a detent, e.g., tips 275a that are wider than the rest of the branches. In some such implementations, the inner shaft’s proximal sleeve 278a defines a proximal slot 279a, a middle slot 277a and a distal slot 276a that facilitate sliding inner shaft 270a with respect to pins 157a, 237a, 247a (FIG. 16), e.g., as described hereinbelow with reference to FIGS. 26A–F.

In some implementations, inner shaft’s proximal sleeve 278a defines a distal lock stopper 273a. In some such implementations, lock stopper 273a applies a compression force to loading spring 266a by supporting the loading spring’s distal end.

In some implementations, and as shown, distal portion 272a of inner shaft 270a is narrower than proximal sleeve 278a, such that the inner shaft passes through an internal space defined by loading spring 266a, and through lumens 43, 53 of the row of suture locks 30. In this way, distal portion 272a extends distally from within the suture locks and the inner shaft’s branches 274a extend distally from within lock shaft 240a and into distal portion 242a of lock shaft 240a.

In some implementations, and as shown, system 200a comprises an inner wire 280a that extends from a proximal portion 288a to a distal portion 282a that extends within inner shaft 270a. In some implementations, and as shown in the lower inset of FIG. 16, the inner wire’s proximal portion 288a is fixedly coupled to handle 122a, such that inner shaft 270a is slidable along wire 280a. In some such implementations, while the inner shaft’s distal portion 272a is disposed over and along the wire, the wire keeps branches 274a of the inner shaft’s distal portion distanced from each other, thereby maintaining a radial width of distal tips 275a. In some such implementations, advancing the inner shaft’s distal portion 272a distally beyond wire 280a increases compressibility of the distal portion’s branches 274a, thereby reducing the width of distal tips 275a.

In accordance with some implementations, the inset of FIG. 22 shows distal portion 202a of system 200a. In some implementations, and as shown, distal portion 242a of lock shaft 240a defines distal slots 267a through which the plug shaft’s plug-pusher arms 254a protrude into the lock shaft’s lumen. In some such implementations, and as shown, plug-pusher arms 254a support the inner shaft’s distal portion 272a, e.g., keeping the inner shaft centered within the lock shaft’s lumen. In some such implementations, inner shaft’s distal portion 272a protrudes distally from between plug shaft’s plug-pusher arms 254a, e.g., such that the inner wire’s distal portion 282a extends distally from between the inner shaft’s branches 274a.

In some implementations, and as shown in FIG. 23, system 200a (e.g., lock shaft 240a thereof) slidably houses suture locks 30 in storage zone 300a. In some such implementations, and as shown, a row of suture locks 30 are arranged such that the suture locks share a common lock axis. The number of suture locks 30 shown is exemplary, and more or fewer suture locks can be loaded within storage zone 300a, mutatis mutandis.

In some implementations, and as shown in the upper inset of FIG. 23, loading spring 266a and suture locks 30 are disposed on respective sides of lock pusher 260a, within the lumen of lock shaft 240a. In some such implementations, and similarly to system 200 described hereinabove, compressed loading spring 266a is aligned proximally of suture locks 30, in order to support and/or advance the row of suture locks by pushing on a proximal suture lock, yet without overtightening the suture locks’ plugs 50 within their respective tubes 40.

In accordance with some implementations, the lower inset of FIG. 23 shows a cross-section of the distalmost suture locks 30 that are held within storage zone 300a. In some such implementations, and as shown, the two distalmost suture locks 30 are held between a proximal retention tab 265a and the pair of distal retention tabs 245a. In some such implementations, and as shown, proximal retention tabs 265a can be radially offset from distal retention tabs 245a, e.g., the distal retention tabs can be located so as to not interfere with interaction between the shuttle’s pusher 350a (FIGS. 25A–B) and the distalmost suture lock 30.

In some implementations, loading spring 266a is calibrated to apply a distal force to lock pusher 260a that is sufficient to push the row of suture locks 30 distally such that the distal-most suture lock abuts distal retention tabs 245a. In this way, retention tabs 245a provide resistance to loading spring 266a, in order to prevent unintentional release of suture locks. For example, retention tabs 245a can be dimensioned to provide a friction fit of suture lock 30 therebetween, e.g., by abutting shoulder 41 of one or more suture locks.

In some implementations, and as shown in the upper inset of FIG. 23, distal portion 262a of lock pusher 260a is shaped similarly to suture lock 30, e.g., such that proximal retention tab 265a will restrain the lock pusher from advancing further distally after the lock pusher reaches its distalmost position by pushing the distalmost suture lock against distal retention tabs 245a. In some such implementations, lock pusher 260a defines a groove 261a that is shaped to engage with proximal retention tab 265a once the lock pusher reaches its distalmost position (not shown). In this way, proximal retention tab 265a can keep pusher 350a from inadvertently engaging lock pusher 260a.

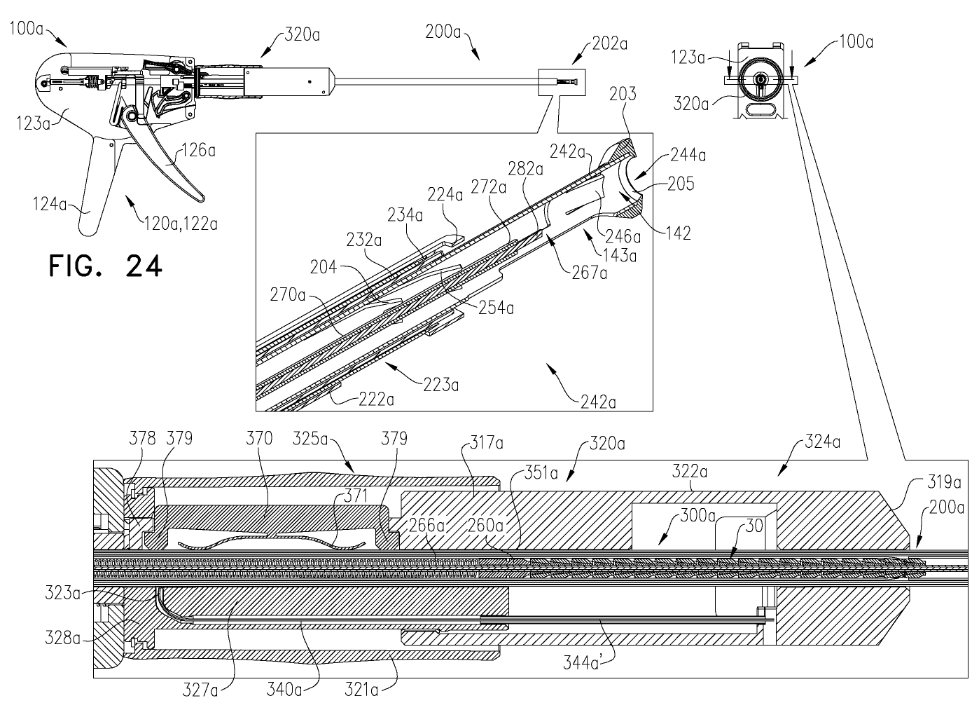

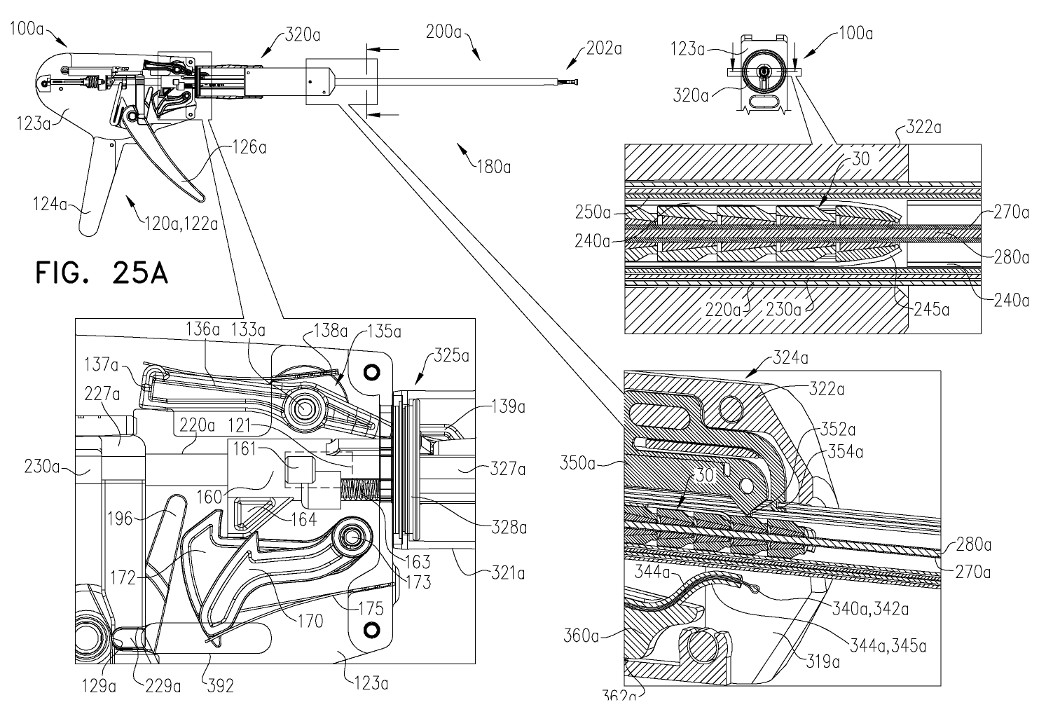

Reference is made to FIG. 24, which shows schematic illustration showing delivery tool 100a in a pre-loading state, in accordance with some implementations. In some implementations, while tool 100a is in the pre-loading state, distal portion 202a of multi-shaft system 200a, e.g., holding zone 142a thereof, does not house suture lock 30. The upper inset of FIG. 24 is a cross-sectional side-view of the multi-shaft system’s distal portion 202a showing, in accordance with some implementations, the plug shaft’s plug-pusher arms 254a supporting the inner shaft’s distal portion 272a. In accordance with some such implementations, FIG. 24 also shows the lock shaft’s support arms 204 supporting inner shaft 270a.

In some implementations, and as shown in the upper inset, holding zone 142a is dimensioned to support both the suture lock’s distal portion 34 and the suture lock’s proximal portion 32. In some such implementations, and as shown, the lock shaft’s distal portion’s holding arm 246a is biased radially inward to support a suture lock’s proximal portion 32 (not shown), and the lock shaft’s crown 203 defines a beveled internal edge 205 that is shaped support the tube’s distal portion 44. In this way, once seated in holding zone 142a, suture lock 30 will typically not fall out of the holding zone or otherwise be inadvertently released from tool 100a until an external force is applied to remove the suture lock from the tool, e.g., as described hereinbelow with reference to FIGS. 25Q–R.

The lower inset of FIG. 24 shows a top-view cross-section of shuttle 320a, disposed along storage zone 300a of system 200a, such that the shuttle abuts handle 122a. In accordance with some implementations, pump lock 370 is disposed within pump lock housing 378 that is defined by carriage 324a. In some such implementations, and as shown, pump lock 370 is held in place by the pump lock’s appendage 371 that pushes the pump lock away from outer shaft 220a, such that the pump lock’s detents 379 are held snugly within pump lock housing 378. For example, and as shown, the pump body’s proximal portion 328a and the carriage’s proximal portion 317a abut opposing portion of pump lock 370 while tool 100a is in the pre-loading state, such that the pump lock inhibits inadvertent actuation of snare pump 325a, e.g., as described hereinbelow with reference to FIG. 25H.

Reference is made to FIGS. 25A–L, which are schematic illustrations showing use of tool 100a to advance suture lock 30 to distal portion 202a of system 200a, and to thread a length of suture 60 through the suture lock, in accordance with some implementations.

FIG. 25A shows tool 100a in the pre-loading state. In some implementations, and as shown in the upper right inset, the lock shaft’s retention tabs 245a hold the distal-most suture lock 30 in place, as described hereinabove with reference to FIG. 23. In some such implementations, and as shown in the lower right inset, carriage’s pusher 350a is positioned proximal of the distalmost suture lock 30, such that the pusher’s lip 352a has not yet having engaged a suture lock.

In accordance with some implementations, the lower left inset of FIG. 25A is a cutaway view showing handle 122a interfacing with shuttle 320a (e.g., with pump 325a) via pump adapter 160. In some implementations, and as shown, pump adapter 160 is held in its proximalmost position, e.g., by pump adapter spring 163 that pushes the pump adapter proximally, holding the pump adapter’s tenon 161 against the proximal wall of the handle’s adapter groove 121. In some such implementations, pump adapter latch 170 abuts the pump adapter’s latch-interface 164, e.g., further inhibiting distal movement of pump adapter.

In some implementations, outer shaft 220a of system 200a is held in the outer shaft’s proximalmost position while tool 100a is in the pre-loading state. In some such implementations, and as shown, outer shaft block 227a (which is fixedly coupled to outer shaft 220a) is held in its proximalmost position by outer shaft spring 134a, such that the outer shaft block’s tongue 229a fits into the trigger’s groove 129a (bottom left inset of FIG. 25A). In this way, the outer shaft block’s tongue 229a can inhibit inadvertent actuation of trigger 126a, e.g., prior to using shuttle 320a to advance outer shaft 220a and a suture lock 30 to the tool’s distal portion 180a.

In some implementations, the outer shaft block’s tongue 229a is two-parted, e.g., while a first part fits into the trigger’s groove 129a, a second part fits into the outer shaft groove 392 defined by the handle’s housing 123a. In this way, outer shaft groove 392 guides the outer shaft block’s tongue 229a as the tool transitions from the pre-loaded state to a ready state (described hereinbelow with reference to FIG. 25G).

In accordance with some implementations, FIG. 25B shows shuttle 320a having been advanced distally, such that lip 352a of pusher 350a engages a distal-most suture lock 30 (lower right inset) and advances the suture lock distally within the lumen of lock shaft 240a, along inner shaft 270a toward the system’s distal portion 202a. In some such implementations, the pusher’s outer shaft-engaging tooth 354a advances distally within longitudinal slots 221a, 231a, 241a, 251a as shuttle 320a slides suture lock 30 distally toward the systems’ distal portion 202a. In some such implementations, and as shown in the upper right inset, the second-most distal suture lock 30 also advances distally within storage zone 300a, such that it abuts retention tabs 245a.

The lower left inset shows two cutaway views of handle 122a as shuttle 320a advances toward the system’s distal portion 202a. In some implementations, and as shown in the lower left inset, shuttle 320a pulls pump adapter 160 distally, e.g., such that the pump adapter’s latch-interface 164 engages pump adapter latch 170. For example, and as shown, latch-interface 164 temporally depresses pump adapter latch 170 (left frame) until latch-interface 164 distally clears latch 170, allows trigger latch spring 175 to push the latch back into place (right frame), thereby locking the pump adapter at its distalmost position. In this way, the pump adapter can act as a safety mechanism, e.g., while pump adapter 160 is locked its distalmost position, it prevents the shuttle from inadvertently regressing proximally and engaging a second suture lock 30 prior to trimming suture 60.

In accordance with some implementations, FIG. 25C shows shuttle 320a having been further advanced distally along system 200a, such that pusher 350a advances suture lock 30 further distally (lower right inset). In some such implementations, and as shown, pusher 350a slides suture lock 30 over and along inner shaft 270a while the inner shaft is disposed in the suture lock’s lock-lumen 33, e.g., as described hereinabove with reference to FIGS. 1E–F.

In some implementations, and as shown in the lower left inset of FIG. 25C, the shuttle’s pump 325a has been slid distally, overcoming the grasp of the pump adapter’s catches 162 to disengage from the pump adapter. In accordance with some such implementations, pump 325a has released interlock lever 136a (e.g., distal portion 139a thereof), such that interlock spring 138a pivots the interlock lever’s proximal portion 137a into position to block distal movement of trigger block 144a. In this way, interlock lever 136a inhibits inadvertent actuation of trigger 126a, e.g., prior to threading suture 60 through suture lock 30.

In accordance with some implementations, FIG. 25D shows shuttle 320a having advanced further toward the system’s distal portion 202a, e.g., such that shuttle 320a has advanced suture lock 30 to the system’s distal portion 202a. In accordance with some such implementations, and as shown in the upper right inset, the pusher’s outer shaft-engaging tooth 354a has engaged the outer shaft’s distal tooth 224a, such that further advancing shuttle 320a will slide both suture lock 30 and outer shaft 220a distally (e.g., in tandem with each other).

In accordance with some implementations, and as shown in the lower left inset, distal opening 345a of distal snare tube 344a is aligned longitudinally with the outer shaft’s window 223a, yet snare 340a and distal snare tube 344a remain outside of lock shaft 240a, since the distal snare tube and the outer shaft’s window 223a are not yet aligned with the lock shaft’s window 243a. In some such implementations, and as shown, contact between system 200a (e.g., lock shaft 240a thereof) and distal snare tube 344a compresses snare spring 362a, keeping snare lever pivoted away from the system.

In accordance with some implementations, the lower right inset of FIG. 25D shows suture lock 30 having slid past the lock shaft’s support arms 204, such that the suture lock abuts the plug shaft’s plug-pusher arms 254a. In some such implementations, support arms 204 keep inner shaft 270a and inner wire 280a centered within the lumen of lock shaft 240a as suture lock 30 advances toward holding zone 142a. For example, the lock shaft’s support arms 204 and the plug shaft’s plug-pusher arms 254a can flex (e.g., deflect radially, as shown regarding plug-pusher arms 254a) to maintain the shafts of system 200a coaxial with respect to each other as suture lock 30 advances toward holding zone 142a. For example, and as shown regarding support arms 204, plug-pusher arms 254a can also keep inner shaft 270a and inner wire 280a centered within the lumen of lock shaft 240a when suture lock 30 is not disposed adjacent to the plug-pusher arms.

In accordance with some implementations, FIG. 25E shows shuttle 320a having advanced further distally, in tandem with outer shaft 220a and suture lock 30. In accordance with some such implementations, suture lock 30 is shown having been advanced to a separation point that is the distalmost point to which the suture lock is advanced, prior to unlocking the suture lock, e.g., by sliding tube 40 distally off of plug 50. For example, and as shown below in FIGS. 25F–G, plug 50 is slid distally off of plug and into holding zone 142a while tube 40 remains at the separation point.

In some implementations, and as shown in the right insets of FIG. 25E, the carriage’s pusher 350a has advanced suture lock 30 to the separation point, at which the suture lock (e.g., plug 50 thereof) reaches the inner shaft’s tips 275a. In this way, the suture lock is held snugly between the pusher’s lip 352a on the suture lock’s proximal side, and the inner shaft’s tips 275a on the suture lock’s distal side. In some such implementations, and as shown in the bottom left insets, which show a perspective-view and a front-view of the interface between the inner shaft’s tips 275a and plug 50, the suture lock’s plug 50 defines a distal indentation shaped to mate with the inner shaft’s tips, which serve as a detent that restrain plug 50 longitudinally while tube 40 is slid of off the plug (FIG. 25F).

FIG. 25F shows shuttle 320a having been slid such that lip 352a unlocks suture lock 30 by pushing tube 40 distally away from (e.g., off of) plug 50. In some implementations, and as shown in the insets of FIG. 25F, tips 275a of the inner shaft’s distal branches 274a a longitudinally restrain plug 50 as pusher 350a pushes tube 40 away from the plug. In some such implementations, the inner shaft’s distal portion 272a is disposed along wire 280a, which keeps tips 275a distanced from each other, thereby preventing plug 50 from sliding distally past the tips.

In some such implementations, and as shown in the transition between FIGS. 25E–G, the lock shaft’s distal portion 242a defines a receiving zone 143a, proximally adjacent to holding zone 142a, that guides tube 40 as pusher 350a pushes it toward the holding zone. For example, and as shown, receiving zone 143a expands to hug the tube on its way to holding zone 142a, e.g., keeping the tube in a desired orientation as the tube slides through the receiving zone on its way to the holding zone.

In some implementations, and as shown in the insets of FIG. 25F, the suture lock’s tube 40 has begun to enter holding zone 142a. In some such implementations, tube 40 temporarily deflects the holding zone’s holding arm 246a as the tube enters the holding zone. In some such implementations, and as shown in the transition between the upper right inset of FIG. 25E and the upper inset of FIG. 25F, the lock shaft’s distal portion 242a (e.g., holding zone 142a thereof) expands radially to accommodate tube 40.

In accordance with some implementations, the lower inset of FIG. 25F shows the carriage’s pusher’s tooth 354a having advanced outer shaft 220a distally such that respective windows 223a, 243a of the outer shaft and lock shaft 240a are aligned with each other, which allows snare spring 362a to expand and push the distal snare tube’s distal opening 345a into the lock shaft’s lumen, facing tube 40. In some such implementations, windows 223a, 243a align with each other when outer shaft 220a is fully advanced distally.

In accordance with some implementations, FIG. 25G shows shuttle 320a having reached its distalmost position along system 200a, such that the shuttle has completed an outbound leg of its travel along the system. In accordance with some such implementations, the shuttle’s pusher 350a has seated tube 40 in the lock shaft’s holding zone 142a. This stage may be considered the tool’s ready state, in which the tool holds plug 50 and tube 40 separately, within the tool’s distal portion 180a. In some such implementations, and as shown, the pusher’s lip 352a has advanced tube 40 over holding arm 246a during the transition from FIG. 25F to FIG. 25G (e.g., temporarily depressing the holding arm), such that pusher 350a supports the tube while the tube is seated at the lock shaft’s holding zone 142a (lower right inset of FIG. 25G).

In some implementations, and as visualized in the transition between the respective upper right insets of FIG. 25F and FIG. 25G, the lock shaft’s distal portion 242a (e.g., the portion thereof defining holding zone 142a) contracts around the suture lock’s tube 40 once the tube is seated within the holding zone, e.g., such that the tube is friction-fit within the holding zone. In some such implementations, and as shown in the upper right inset of FIG. 25G, the suture lock’s tube 40 and the lock shaft’s distal portion 242a are shaped correspondingly such that lock shaft hugs the suture lock both laterally and proximally.

The lower left inset of FIG. 25G shows handle 122a as tool 100a transitions from state shown in FIG. 25E (frames I and II) to the ready state (frames III and IV). Frames I and III show “starboard” cross-sectional views of handle 122a in which (as per the rest of FIG. 25G) the tool’s distal portion is on the right, whereas frames II and IV are “portside” cutaway views of the handle in which the tool’s distal portion 180a is on the left (as per FIG. 18B).

In some implementations, and as shown in the transition between frames I and III, outer shaft 220a advances distally such that the outer shaft block’s tongue 229a advances distally within the handle housing’s outer shaft groove 392. In some such implementations, and as shown in frames II and IV, outer shaft block 227a passes distally over trigger latch 172 as outer shaft 220a advances distally, such that the trigger latch traps the outer shaft block against pump adapter 160, thereby locking the outer shaft in its distalmost position. For example, locking outer shaft 220a in its distalmost position can prevent the user from inadvertently retracting shuttle 320a to engage a second suture lock prior to trimming suture 60.

In accordance with some implementations, FIG. 25H shows snare pump 325a having been actuated such that tool 100a transitions into a snaring state by advancing distally over carriage 324a, pushing snare 340a through snare channel 346 such that the snare’s distal portion 342a extends distally outward from the shuttle’s distal aperture 318a.

In accordance with some implementations, the left inset of FIG. 25H shows the same compound cross-sectional view of shuttle 320a as in FIGS. 20A, in which the shuttle is cut (i) to show a side view of the shuttle, and (ii) along a plane oblique to the side view, that corresponds to the path that snare 340a extends through snare channel 346, tube 40 and out from the carriage via distal aperture 318a. As described hereinabove with reference to FIG. 20A, snare 340a extends through snare channel 346 in a u-turn, in order to increase the length of the snare that exits aperture 318a. For example, sliding snare pump 325a along a given length of the carriage can release twice that length of snare 340a from shuttle 320a.

In accordance with some implementations, the right inset of FIG. 25H shows the transition that pump lock 370 undergoes between the ready state shown in FIG. 25G (upper frame) and the snaring state (lower frame). In some such implementations, and as shown, actuating snare pump 325a not only extends the snare’s distal portion 342a from aperture 318a, but also locks the system’s shafts in relation to each other.

In some implementations, and as described hereinabove with reference to FIG. 21B, shafts 220a, 230a, 240a, 250a each define respective lock slots 372, 373, 374, 375. In some such implementations, each shaft’s lock slots align with each other when tool 100a assumes the ready state. In some such implementations, and as shown in the blow-up of the upper frame of FIG. 25H’s right inset, the snare pump’s proximal portion 328a abuts the pump lock’s proximal portion, such that advancing the pump’s housing 321a distally over carriage 324a pushes pump lock 370 (e.g., detents 379 thereof) into engagement with slots 372, 373, 374, 375, thereby longitudinally locking shafts 220a, 230a, 240a, 250a with respect to each other. In this way, pump lock 370 can assure that the respective shafts’ slots 372, 373, 374, 375 are aligned with each other in order to actuate snare pump 325a. That is, a misaligned shaft would block one or more of the pump lock’s detents 379 from entering the system’s lumen, such that pump lock 370 would remain in place between the carriage’s proximal portion 317a and the pump body’s proximal portion 328a, thereby preventing the user from actuating snare pump 325a. In this way, pump lock 370 can prevent inadvertent actuation of snare pump 325a before outer shaft 220a is pulled via shuttle 320a to its distalmost position, e.g., before tool 100a assumes the ready state. Alternatively or in addition, pump lock 370 can prevent undesirable movement of the shafts of system 200a with respect to each other, and/or with respect to tube 40, while using tool 100a to snare suture 60.

In accordance with some implementations, FIG. 25I shows the user threading suture 60 through the snare’s distal portion 342a, after which pump 325a is pulled proximally (FIG. 25J), which withdraws the snare’s distal portion 342a back into distal snare tube 344a.

In accordance with some implementations, the upper inset of FIG. 25J shows the same compound cross-sectional view of shuttle 320a as in FIGS. 20A and 25H, after retracting pump 325a has pulled the snare’s distal portion 342a back into snare channel 346, such that sutures 60 enter tool 100a via aperture 318a. In accordance with some implementations, the lower inset of FIG. 25J shows a top-view cross-section of shuttle 320a, showing that the pump’s proximal portion 328a has slid proximally of pump lock 370, thereby releasing the pump lock from the shafts’ slots 372, 373, 374, 375. In some such implementations, and as shown, the pump lock’s appendage 371 pushes against system 200a (e.g., against outer shaft 220a) to disengage the pump lock from system 200a.

In some implementations, and as shown in FIG. 25K, pulling snare pump 325a further proximally pulls carriage 324a (e.g., pulls shuttle 320a in its entirety) proximally along system 200a. In some such implementations, and as shown in the inset, retracting shuttle 320a along system 200a pushes the distal snare tube 344a out of lock shaft and back into carriage 324a.

In some implementations, and as shown in the bottom inset frame, retracting shuttle 320a over system 200a exposes suture lock 30 (e.g., the tube’s distal portion 44), which protrudes distally from tool 100a. Tube 40 protruding distally from tool 100a can facilitate tensioning suture 60 and drawing the tensioned suture proximally through tube-lumen 43, after which the suture can be released (not shown) such that the suture remains threaded through tube 40 and windows 223a, 243a at the system’s distal portion 202a, e.g., similarly to as shown regarding tool 100 in FIG. 11K.

In some implementations, and as also shown in the bottom inset frame of FIG. 25K, pulling shuttle 320a proximally disengages the pusher’s lip 352a from tube 40. In some such implementations, and as shown, disengaging pusher 350a from the tube releases tension upon the pusher, such that the pusher’s rail 351a shifts out of alignment with pump body’s longitudinal groove 331a (e.g., such that the rail faces the snare pump’s distal portion 326a), thereby inhibiting operation of snare pump 325a while tube 40 is disengaged from pusher 350a.

In accordance with some implementations, FIG. 25L shows shuttle 320a having been retracted proximally to complete a return leg of its travel along system 200a. In some such implementations, and as shown, tool 100a has adopted a resting state in which the unlocked and loaded suture lock 30 is disposed within the system’s distal portion 202a and trigger 126a has not yet been actuated. In accordance with some such implementations, the upper inset of FIG. 25L shows a top-view cross-section of system’s distal portion 202a, in which the threaded tube 40 is seated in the lock shaft’s holding zone 142a, in preparation for actuation of trigger 126a to secure suture 60 to suture lock 30.

In some implementations, and as shown in the bottom left inset, shuttle 320a has been pulled proximally such that the pump adapter 160 (e.g., the pump adapter’s catches 162) reengages the snare pump 325a. In some implementations, and as also shown, the interlock lever’s distal portion 139a is depressed into snare pump 325a, such that the interlock lever’s proximal portion 137a elevates out of alignment with trigger block 144a, thereby allowing the operator to actuate trigger 126a.

In accordance with some implementations, the bottom left inset of FIG. 25L shows shuttle 320a having engaged pump adapter 160, e.g., such that the pump adapter’s catches 162 engage shuttle’s carriage body 127a. In some implementations, engaging shuttle 320a with pump adapter 160 prevents the user from inadvertently engaging a second suture lock prior to trimming suture 60. For example, and as shown, a gap 165 remains between the shuttle and handle 122a, such that the pusher’s lip 352a is disposed distally of the proximal portion of the distalmost suture lock in the tool’s storage zone 300a (lower right inset of FIG. 25L).

In some implementations, and as described hereinabove with reference to frame IV of the bottom left inset of FIG. 25G, trigger latch 172 holds outer shaft block 227a in its distalmost position by holding the outer shaft block against pump adapter 160. In this way, and as shown in the bottom right inset of FIG. 25L, pump adapter 160 does not allow shuttle to engage another suture lock until tool 100a is used to trim suture 60. For example, and as shown, the pusher’s lip 352a is disposed distally of the distalmost suture lock’s proximal end, such that if the operator inadvertently advances shuttle 320a distally, the pusher will not engage a suture lock.

Reference is made to FIGS. 25M–R and 26A–F, which are schematic illustrations showing use of tool 100a to lock suture lock 30 onto a length of suture 60 at an implant 20, in accordance with some implementations.

FIGS. 25M–R show tool 100a as a whole, as well as cross-sectional views of shuttle 320a and the multi-shaft system’s distal portion 202a, as well as cutaway “portside” views of handle 122a, as the tool transitions through stages A–F in order to lock suture lock 30 onto suture 60. It is to be noted that stages A–F are for the purpose of description only, and do not necessarily correspond to discrete stages at which tool 100a remains for a given period of time, but rather some or all of stages A–F may be transitory in nature.

FIGS. 26A–F show cutaway “starboard” views of handle 122a and top-view cross-sections of the tool’s force regulator 150a and of the multi-shaft system’s proximal portion 208a as the tool transitions through stages A–F. In some implementations, and as described hereinbelow, actuation force is transferred from trigger 126a via force regulator 150a to system 200a in a manner that coordinates locking suture lock 30 and cutting suture 60. In some such implementations, and in contrast to force regulator 150 of tool 100, trigger 126a and force regulator 150a do not mediate release of suture lock 30 from tool 100a. For example, the locked suture lock can remain within the tool’s distal portion 180a (e.g., within holding zone 142a) until the suture lock is removed by applying an external force to tool 100a and/or to suture lock 30, e.g., the force can be considered to be “external” in that it is not mediated by trigger 126a or force regulator 150a.

Stages A–F will be described below in parallel reference to FIGS. 25M–R and FIGS. 26A–F, in order to highlight the functional relationship between force regulator 150a and multi-shaft system 200a, whereby force is transferred from the force regulator to a force applicator in the system’s distal portion 202a. In some implementations, the force applicator is not necessarily a discrete component of system 200a. Instead, force regulator 150a can transfer force to different components of system 200a as tool 100a transitions through stages A–F. In this way, the force applicator may be considered to be the component or components of system 200a that apply force to suture lock 30 during a given stage of locking the suture lock. Advantageously, force regulator 150a transfers force to different components of system 200a as the user provides the actuation force to handle 122a, e.g., without the user needing to select a particular component of the system as the force applicator. Tool 100a is therefore simpler to use than other suture-locking devices, making the tool less prone to human error, and more efficient to use.

FIGS. 25L and 26A show tool 100a in stage A (e.g., in the resting state), in which trigger 126a has not yet been actuated, and FIG. 25M show tool 100a having been placed into position for use, such that distal portion 202a of system 200a is pointed towards implant 20, e.g., such that suture lock 30 is placed against the implant while the user pulls suture 60 taught through respective windows 223a, 243a of outer shaft 220a and lock shaft 240a. In accordance with some implementations, FIG. 25L shows the outer shaft block 227a blocking the proximal travel of pump adapter 160, which maintains gap 165 between pump 325a and handle 122a (lower left inset), such that carriage 324a cannot withdraw sufficiently proximally for pusher 350a to engage the distalmost suture lock in storage zone 300a (lower right inset).

FIG. 26A shows force regulator 150a and proximal portion 208a of system 200a in stage A prior to actuating trigger 126a. In some implementations, and as shown, trigger block 144a and lock block 257a are disposed in a proximal position, due to a proximal pulling force that trigger spring 132a exerts upon the trigger block and the lock block.

The inset of FIG. 26A is a cross-sectional view of force regulator 150a and proximal portion 208a of multi-shaft system 200a during stage A. In some implementations, and as described hereinabove with reference to FIG. 16, pins 157a, 237a, 247a are slidably disposed within the lateral slots (FIGS. 21A–B) defined by various components of system 200a in a manner that facilitates relative motion of cutter pin 237a and plug pin 157a in relation to lock pin 247a (e.g., which is fixedly coupled to the handle’s housing 123a) and other components of tool 100a, as the tool transitions through stages B–F. In some such implementations, and as shown, (i) lock pin 247a is disposed within inner shaft’s proximal slot 279a and the plug shaft’s proximal slot 253a, (ii) plug pin 157a is disposed within the inner shaft’s middle slot 277a, the cutter shaft’s proximal slot 239a and the lock shaft’s proximal slot 249a, and (iii) cutter pin 237a is disposed within the inner shaft’s distal slot 276a, the plug shaft’s proximal slot 253a and the lock shaft’s proximal slot 249a. Proximal slot 249a of lock shaft 240a (through which plug pin 157a and cutter pin 237a are slidable) is shown in FIGS. 21A–B but is not readily visible in FIG. 26A–F.

In some implementations, and as shown in FIG. 26A, inner shaft spring 209 (e.g., a compression spring) is disposed between the handle’s housing 123a and the inner shaft’s proximal portion 178a such that the inner shaft spring applies a distal pushing force to the inner shaft’s proximal portion 178a. In some such implementations, force regulator 150a governs whether inner shaft 270a (and plug 50 that is disposed at the inner shaft’s distal portion 272a) slides distally in response to the inner shaft spring’s pushing force. For example, contact between plug pin 157a and the proximal end of the inner shaft’s middle slot 277a (FIGS. 21A–B) counteracts the inner shaft spring’s distal pushing force while system 200a is in the resting state.

In accordance with some implementations, FIGS. 25N and 26B show tool 100a having advanced to stage B, in which trigger 126a has been partially actuated, such that inner shaft 170a carries plug 50 into the tube’s tube-lumen 43 (inset of FIG. 25N), e.g., reengaging plug 50 and tube 40 such that the inner shaft’s distal portion 272a is disposed within the suture lock’s lock-lumen 33. In some implementations, and as shown in the transition between the insets of FIGS. 25L and 25N, the inner shaft’s distal portion 172a slides distally off of the inner wire’s distal portion 282a as the inner shaft carries plug 50 into the tube’s tube-lumen 43.

In some implementations, and as shown in FIG. 25N, plug 50, inner shaft 270a, plug shaft 250a and cutter shaft 230a advance distally (e.g., in tandem) during stage B, in relation to outer shaft 220a and lock shaft 240a. In some such implementations, and as shown in FIG. 26B, trigger’s block-interface 186 pushes trigger block 144a distally, overcoming the trigger spring’s proximal pulling force. In some such implementations, and as shown, the trigger block’s proximal portion pushes distally against lock spring 154a, which in turn pushes lock block 257a against the trigger block’s distal portion, such that both trigger block 144a and lock block 257a advance distally. For example, and as shown in the inset of FIG. 26B, both cutter pin 237a and plug pin 157a advance distally relative to lock shaft 240a, which is held in place by lock pin 247a. In this way, advancement of cutter pin 237a and plug pin 157a allows inner shaft spring 209 to slide the inner shaft distally, thereby pushing plug 50 from the separation point and towards tube 40.

In some such implementations, and as shown in the inset of FIG. 25N, plug-pusher arms 254a help keep inner shaft 270a centered within lock shaft 240a as the inner shaft slides distally between the plug-pusher arms. In this way, the plug shaft’s plug-pusher arms 254a and/or the lock shaft’s support arms 204 can help ensure that inner shaft 270a accurately places plug 50 within the suture lock’s tube 40, e.g., while the inner shaft is disposed in the suture lock’s lock-lumen 33.

In accordance with some implementations, the inset of FIG. 26B shows inner shaft spring 209 having expanded, which pushes inner shaft 270a distally as cutter pin 237a and plug pin 157a remain in contact with the proximal end of respective slots of the inner shaft. In this way, distal advancement of trigger block 144a and lock block 257a determine the degree to which inner shaft 270a advances distally.

In accordance with some implementations, FIGS. 25O and 26C show tool 100a having advanced to stage C, as the operator continues to apply actuation force to trigger 126a. In some implementations, applying actuation force below the force threshold to trigger 126a secures suture lock 30 to suture 60 by engaging plug 50 with the tube 40. In accordance with some such implementations, the upper inset of FIG. 25O shows that actuating trigger 126a advances plug shaft 250a further distally, such that the plug shaft’s plug-pusher arms 254a apply a distal locking force that pushes plug 50 further into the tube’s lumen 43, thereby locking suture lock 30, e.g., by friction-fitting plug 50 and suture 60 within tube 40.

In some implementations, and as shown in FIG. 25N, plug 50 is first placed within tube 40 using a lesser force (e.g., exerted by inner shaft spring 209 upon inner shaft 270a), before plug shaft 250a (e.g., plug-pusher arms 254a thereof) applies a greater force to lock the suture lock by pushing the plug more firmly into the tube (FIG. 25O). In some such implementations, and as shown in FIG. 25O, suture lock 30 is locked while the inner shaft’s distal portion 272a is already disposed within plug-lumen 53 and/or lock-lumen 33 (FIG. 1F). For example, centering plug 50 within lock-lumen 33 prior to locking suture lock 30 and/or trimming suture 60 can help assure foolproof operation of tool 100a. In some such implementations, threading suture through tube-lumen 43 but not through plug-lumen 53 can help avoid interference between the suture and inner shaft 170a as the inner shaft is used to guide plug 50 into tube 40.

In some implementations, and as shown in FIG. 26C, further actuating trigger 126a pushes trigger block 144a (via the trigger’s block-interface 186) distally, such that the trigger block’s proximal portion pushes distally against lock spring 154a, which in turn pushes lock block 257a against the trigger block’s distal portion, such that both trigger block 144a and lock block 257a advance distally. In some such implementations, and as shown, trigger block 144a and lock block 257a (and therefore cutter shaft 230a and plug shaft 250a) advance distally (e.g., in tandem) with respect to lock shaft 240a during stage C.

In some implementations, advancement of cutter pin 237a and plug pin 157a within respective slots of inner shaft 270a allow inner shaft spring 209 to advance the inner shaft further distally. In some such implementations, and as shown in FIG. 26C, the inner shaft’s distal movement is halted when the proximal edge of the inner shaft’s proximal slot 279a reaches lock pin 247a, e.g., the inner shaft’s proximal slot 279a can be dimensioned such that lock pin 247a arrests the inner shaft such that the inner shaft’s distal portion 272a cannot protrude distally from tool 100a and/or suture lock 30 (upper inset of FIG. 25O) during normal use of tool 100a.

In accordance with some implementations, FIG. 26D shows tool 100a having advanced from stage C to stage D, in which trigger block 144a and cutter shaft 230a advance distally, relative to lock block 257a and plug shaft 250a. In some such implementations, and as shown, further actuating trigger 126a by applying actuation force in excess of the force threshold cuts suture 60 (FIG. 25O) after plug 50 has been fully seated within tube 40.

In some implementations, and as shown in FIG. 26D, trigger block 144a and cutter shaft 230a advance the cutter shaft’s blade 234a while lock pin 247a halts the inner shaft’s distal movement. In some such implementations, trigger block 144a advances cutter shaft 230a with relation to plug shaft 250a, e.g., after lock pin 247a halts the plug shaft’s distal travel by arresting the proximal portion of the plug shaft’s proximal slot 253a and/or after the plug shaft’s plug-pusher arms abut plug 50. For example, and as shown in FIG. 26D, actuating trigger 126a while plug shaft 250a has reached its distal travel limit compresses lock spring 154a (e.g., between the trigger block’s proximal portion and the lock shaft), which resists further actuation of the trigger. In order to overcome the resistance, the user applies force exceeding the force threshold, which compresses lock spring 154a such that trigger block 144a and cutter shaft 230a advance with respect to lock block 257a and plug shaft 250a, thereby trimming suture 60. In this way, suture 60 can be cut both (i) after securing suture 60 by locking suture lock 30, and (ii) without applying excessive force to plug 50 and/or plug shaft 250a.

In some implementations, suture 60 is cut while outer shaft 220a remains in its distalmost position. In some such implementations, and as shown in the transition between the lower (“portside”) insets of FIG. 25O, as tool 100a transitions from stage C to stage D, trigger latch 172 retains outer shaft block 227a in its distalmost position. In some such implementations, while in its distalmost position, outer shaft block 227a blocks pump adapter 160 from reaching its proximalmost position, such that carriage 324a can still not withdraw sufficiently proximally for pusher 350a to engage the distalmost suture lock in storage zone 300a (lower right inset of FIG. 25L).

In some implementations, actuating trigger 126a during the tool’s transition from stage C to stage D reduces a distance between the trigger’s latch-interface 196 and the trigger latch’s trigger-interface 174. For example, and as shown in the right lower inset of FIG. 25O, the trigger’s latch-interface 196 contacts (e.g., partially depresses) the trigger latch’s trigger-interface 174 during stage D.

In some implementations, and as shown in FIGS. 25P and 26E, further actuating trigger 126a transitions tool 100a into stage E, in which outer shaft 220a begins to retract proximally away from the locked suture lock 30. In some such implementations, and as shown in FIG. 25P’s “portside” lower inset, trigger’s latch-interface 196 depresses the trigger latch’s trigger-interface 174, such that outer shaft block 227a clears trigger latch 172, allowing outer shaft spring 134a to pull outer shaft block 227a and outer shaft 220a proximally. In some implementations, pulling outer shaft block 227a and outer shaft 220a proximally reduces resistance that the outer shaft block applies to pump adapter spring 163, such that the pump adapter spring pushes pump adapter 160 such that the pump adapter’s tenon slides to its proximalmost position within the handle’s adapter groove 121 (FIG. 25A), closing gap 165 (FIG. 25O) between pump adapter 160 and handle 122a, as the pump adapter abuts the handle.

In some implementations, tool 100a can reset itself, e.g., securing suture lock 30 to suture 60 automatically returns the tool to the ready state in which the tool (e.g., shuttle 320a thereof) is ready to engage the next suture lock, e.g., the suture lock that has assumed the distalmost position in the tool’s storage zone 300a. In some such implementations, closing gap 165 between pump adapter 160 and handle 122a allows shuttle 320a to engage the next suture lock. For example, and as shown in the lower inset of FIG. 25Q, closing the gap between the pump adapter and the handle has positioned lip 352a of the carriage’s pusher 350a distally of the next suture lock’s proximal portion, such that the next outgoing leg of the shuttle will engage the next suture lock and advance it to the tool’s distal portion 180a.

In some implementations, and as shown in FIG. 26E, trigger block 144a and cutter shaft 230a advance still further distally during stage E, e.g., the trigger’s block-interface 186 pushes the trigger block, lock spring 154a and lock block 257a distally in tandem. In some such implementations, and as shown, the cutter shaft’s distal portion 232a reaches its distal travel limit (e.g., abutting the lock shaft’s crown 203, as shown in FIG. 25P), as cutter pin 237a approaches the distal end of the plug shaft’s proximal slot 253a (FIG. 26E).

In accordance with some implementations, FIGS. 25Q and 26F show trigger 126a having been released, such that tool 100a has transitioned to stage F. In some such implementations, and as shown, tool 100a has readopted aspects of the pre-loading state described hereinabove with reference to FIGS. 24 and 25A. In accordance with some implementations, the bottom inset of FIG. 25Q shows that the shuttle’s pusher 350a is positioned to engage the suture lock that now occupies the distalmost position within storage zone 300a, similarly to as shown in the bottom right inset of FIG. 25A. In accordance with some implementations, the upper inset of FIG. 25Q shows the system’s distal portion 202a having reassumed the orientation shown in the upper inset of FIG. 24. In some such implementations, and in contrast to distal portion 242 of lock shaft 240 (shown above in FIG. 11O as being biased to expand radially when not compressed by cutter shaft 230 and/or outer shaft 220), distal portion 242a of lock shaft 240a retains its conformation after cutter shaft 230a and outer shaft 220a retract proximally, such that holding zone 142a remains closed around suture lock 30.

In some implementations, and as shown in FIG. 26F, release of trigger 126a allows trigger spring 132a to pull trigger block 144a and lock block 257a, such that cutter shaft 230a and plug shaft 250a slide proximally. In some such implementations, and as shown in the inset of FIG. 26F, cutter pin 237a slides proximally within the inner shaft’s distal slot 276a, and plug pin 157a slides proximally within the inner shaft’s middle slot 277a. For example, cutter pin 237a and/or plug pin 157a can pull inner shaft 270a proximally by the proximal edge of its respective slots, thereby recompressing inner shaft spring 209 and/or restraining the inner shaft at its proximalmost position.

In some implementations, and as shown in FIG. 26F, outer shaft spring 134a has pulled outer shaft block 227a and outer shaft 220a proximally, e.g., such that the outer shaft block’s tongue 229a reengages the trigger’s groove 129a, thereby inhibiting inadvertent actuation of trigger 126a. In this way, handle 122a inhibits actuating trigger a second time while the same suture lock 30 is disposed at the tool’s distal portion 180a.

In accordance with some implementations, and as shown in the lower inset of FIG. 25Q, tool 100a has completed a complete cycle of operation, such that tool 100a is reset (e.g., the shuttle’s pusher 350a has returned to the storage zone 300a, proximally of the next suture lock 30) in order to prepare the tool for applying another suture lock 30 to implant 20. In this way, tool 100a can be used iteratively, in order to secure a plurality of suture locks 30 to lengths of suture 60 to implant 20, e.g., as shown hereinabove in FIG. 14. In some such implementations, tool 100a is reset such that system 200a inhibits operation of trigger 126a until shuttle 320a (e.g., the carriage’s pusher 350a) slides the next suture lock distally from storage zone 300a to the tool’s distal portion 180a. For example, and as shown in FIG. 26F, outer shaft block 227a has fully retracted, such that the outer shaft block’s tongue 229a engages the trigger’s groove 129a, thereby inhibiting operation of the trigger until shuttle 320a pulls outer shaft 220a, and therefore the outer shaft block to its distalmost position (FIG. 25G).

In some implementations, and as shown in FIG. 25R, locked suture lock 30 is then removed from tool 100a, such that the suture lock secures implant 20 to heart 6 (upper inset). In some such implementations, the suture lock’s removal is not mediated by trigger 126a or force regulator 150a but rather is performed by applying an external force (e.g., 50–100 g of force) to tool 100a, e.g., by retracting the tool in its entirety from the locked suture lock. In this way, suture lock 30 generally remains within tool 100a (e.g., within holding zone 142a) until the operator removes the suture lock from the tool. For example, and as shown in phantom in the bottom inset, the lock shaft’s distal portion 242a can temporarily expand (e.g., portions of the lock shaft’s distal portion on either side of longitudinal slot 241a can deflect radially away from the suture lock), in order to extricate suture lock 30 from holding zone 142a as tool 100a is withdrawn.

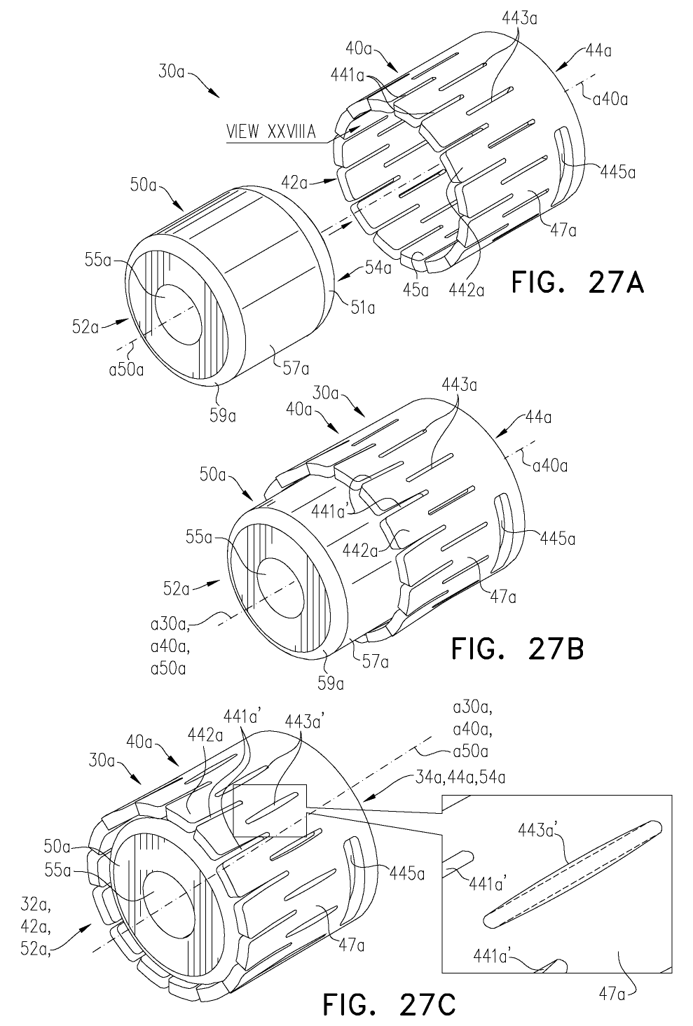

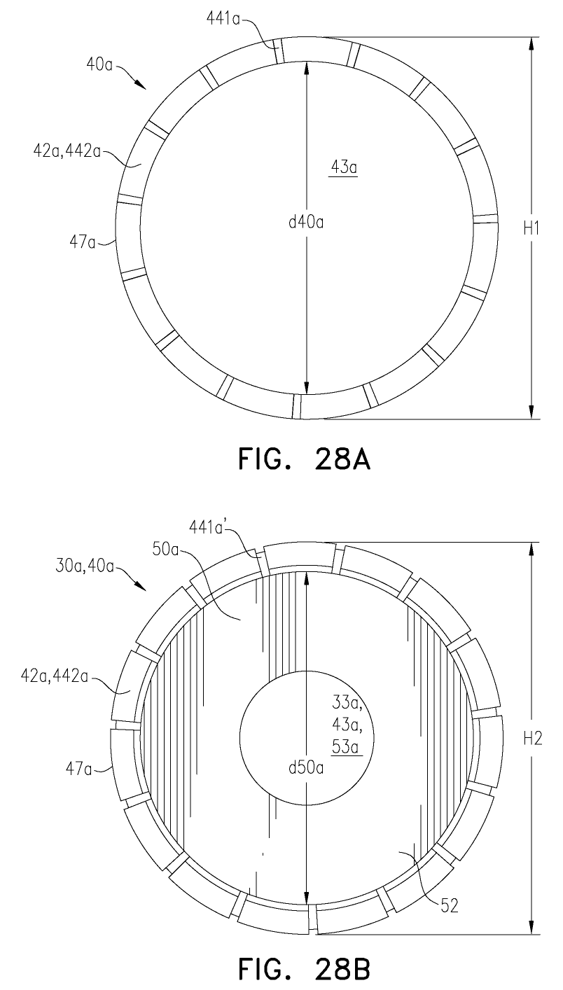

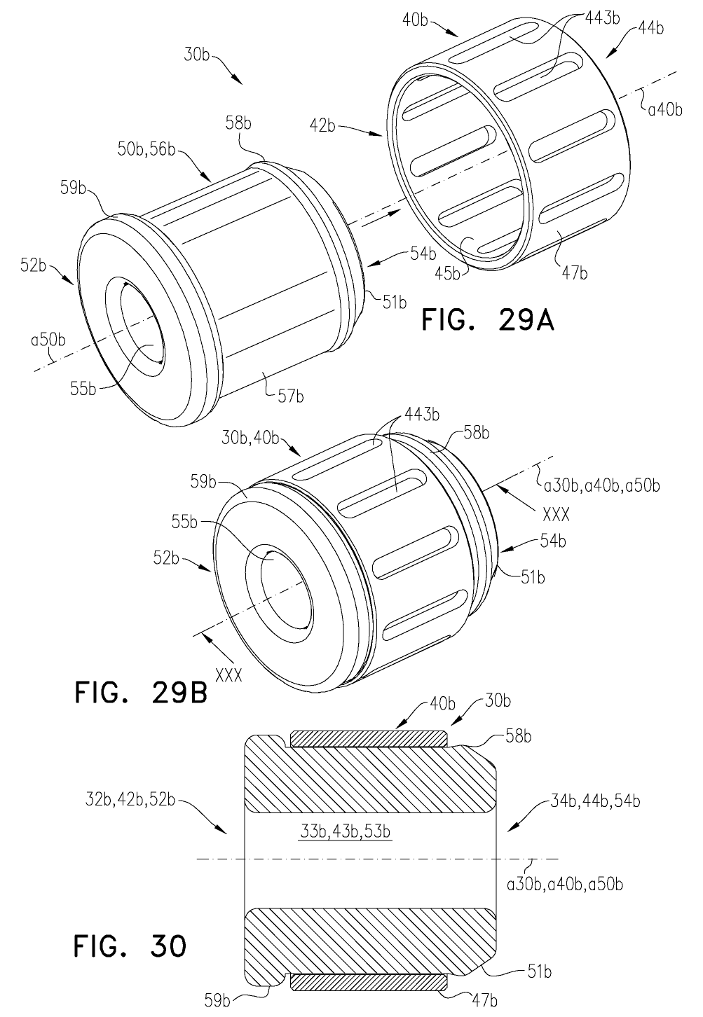

Reference is made to FIGS. 27A-C, 28A-B, 29A-B and 30 which are schematic illustrations showing suture locks 30a, 30b, in accordance with some implementations. In some implementations, and as shown, suture locks 30a, 30b each comprise a tube 40a, 40b and a plug 50a, 50b that is dimensioned to fit within the tube. Similarly to suture lock 30, inner surface 45a, 45b of tube 40a, 40b defines a lumen 43a, 43b into which plug 50a, 50b is fitted in order to lock the suture lock 30a, 30b (FIGS. 27C, 28B, 29B, 30).

In some implementations, and further similarly to suture lock 30, suture locks 30a, 30b can be used with any of the delivery tools 100, 100a, 100b, 100c described herein, mutatis mutandis. In some such implementations, and as shown, the plug’s inner surface 55a, 55b defines a plug-lumen 53a, 53b that is available to the delivery tool for retaining the plug while suture 60 is threaded into the tube’s tube-lumen 43a, 43b, e.g., as described hereinabove with reference to suture lock 30.

As such, suture locks 30a, 30b can be considered to be variants of suture lock 30, and portions of the suture locks that are identically named typically share similar features and serve similar functions as each other. As such, the description below of suture locks 30a, 30b focuses upon features that are particular to suture locks 30a, 30b.

In some implementations, suture locks 30a, 30b are configured to help ensure that the suture locks are accurately tightened. In some such implementations, suture locks 30a, 30b are configured to retain the locked state after being locked, e.g., to resist being unintentionally unlocked. In some such implementations, tube 40a, 40b is compliant to plug 50a, 50b in a manner that facilitates insertion of the plug into the tube, as well as retention of the plug by the tube. For example, the tube’s compliance can facilitate an interference fit of the plug within the tube. Alternatively or in addition, the plug can be shaped to resist release from the tube once the plug is fitted within the tube-lumen.

In some such implementations, suture lock 30 is configured to resist being overtightened. For example, suture locks 30a, 30b can prevent plug 50a, 50b from exiting tube 40a, 40b, e.g., by advancing distally beyond tube 40a, 40b. Alternatively or in addition, suture locks 30a, 30b are configured to provide the user with tactile feedback (e.g., a click) that indicates that the suture lock has been locked.

In some implementations, plug 50a, 50b and tube 40a, 40b are manufactured to facilitate locking suture lock 30a, 30b. In some such implementations, tube 40a, 40b is manufactured to have greater compliance than plug 50a, 50b. For example, plug 50a, 50b can comprise a hardened alloy of cobalt-chrome, and tube 40a, 40b can comprise a relatively elastic material, such as nitinol. In some such implementations, the tube’s proximal portion 42a, 42b has greater compliance than the tube’s distal portion 44a, 44b.

In some implementations, and as described hereinbelow, tube 40a, 40b has axial slits 441a, 443a, 445a, 443b that provide the tube’s wall with compliance that facilitates locking suture lock 30a, 30b. In some such implementations, tube 40a, 40b can elastically deform as plug 50a, 50b is advanced into the tube, after which the can tube recover its initial shape, thereby retaining the plug within the tube, e.g., by interference fit. In this way, plug 50a, 50b can be advanced into tube 40a, 40b with a lesser locking force than would be required in absence of slits 441a, 443a, 445a, 443b.

In some implementations, and as shown, plug 50a defines a rounded distal shoulder 51a that helps to guide the plug into tube 40a, e.g., in the case that plug axis a50a is imperfectly aligned with tube axis a40a. Alternatively or in addition, the plug’s proximal portion 52a can define a rounded proximal edge 59a, as shown. In some such implementations, the rounded shape of shoulder 51a and proximal edge 59a can reduce a risk of damaging tissue with which shoulder 51a or proximal edge 59a may come into contact while the suture lock is locked, e.g., in case that the plug’s distal portion 54a (e.g., shoulder 51a thereof) protrudes distally from the tube’s distal portion 44a, or in the case that the plug’s proximal portion 52a protrudes proximally from the tube’s proximal portion 42a.

FIGS. 27A-C are perspective views of suture lock 30a in an unlocked state (FIG. 27A) and in transition (FIG. 27B) into a locked state (FIG. 27C). FIG. 27A shows tube 40a and plug 50a oriented with respect to each other such that tube axis a40a is coaxial with plug axis a50a, and the plug’s distal portion 54a is facing the tube’s proximal portion 42a.

In some implementations, and as shown in FIG. 27A, tube 40a has a wall (e.g., between the tube’s inner surface 45a and outer surface 47a) that defines slits therein, such as proximal slits 441a, middle slits 443a and distal slits 445a (e.g., two distal slits, one of which is visible in FIGS. 27A-C. In some such implementations, and as shown, slits 441a, 443a are parallel to each other, e.g., thereby helping to steer plug 50a along the tube-axis as the plug is pushed into the tube. In some such implementations, and as shown, proximal slits 441 define proximal tabs 442a therebetween.

In some implementations, suture lock 30a can be modified to define only one or two of the three types of slits shown. Similarly, suture lock 30a can be modified to alter the number of each type of slits 441a, 443a, 445a from the number shown, which is for illustrative purposes only.

In some implementations, and as shown, at least some of the slits change shape (e.g., distort and/or expand) as suture lock 30a transitions from the unlocked to locked states. In some slits implementations, slits 441a, 443a, 445a facilitate locking suture lock 30a by providing tactile feedback to the user, e.g., the different types of slits 441a, 443a, 445a each provide different amounts of resistance to locking force that the user provides in order to advance plug 50a into tube 40a. In some such implementations, proximal slits 441a can provide minimal resistance to the locking force as plug 50a is advanced into tube 40a, e.g., proximal tabs 442a flare out radially and proximal slits 441a' widen as plug 50a partially enters tube 40a (FIG. 27B).

In some implementations, middle slits 443a provide greater resistance than proximal slits 441a to the locking force as plug 50a is further advanced into tube 40a. That is, greater force may be required to expand middle slits (443a' in FIG. 27C) than is required to expand proximal slits 441a (FIG. 27B). In this way, the resistance provided by middle slits 443a can indicate to the operator that plug 50a is nearly completely advanced into tube 40a.

In some implementations, distal slits 445a can act as a brake that resists advancing the plug 50a (e.g., when the plug’s shoulder 51a) distally past the distal slits. For example, distal slits 4454a are perpendicular to tube axis a40a, which can increase the degree to which the slits resist to the plug’s outer surface 57a sliding therethrough. In some such implementations, distal slits 445a provide resistance that signals to the user that suture lock 30a is fully locked. Alternatively, or in addition, such resistance prevents the user from overtightening the suture lock, e.g., preventing the user from advancing plug 50a past the tube’s distal portion 44a.

FIGS. 28A-B show proximal portion 42a of tube 40a (FIG. 28A) and proximal portion 32a of the locked suture lock 30a (FIG. 28B). In some implementations, tube 40a must change its dimensions (e.g., expand radially) in order to accommodate plug 50a within tube-lumen 43a. For example, the plug’s external diameter d50a (FIG. 28B) can be greater than the tube’s internal diameter d40a while the suture lock is unlocked (FIG. 28A). In some such implementations, and as shown in FIG. 28B, slits 441a' widen and tabs 442a flare radially outward to accommodate plug 50a, e.g., such that the tube’s diameter is greater while the suture lock is locked (H2 in FIG. 28B) than when the tube’s lumen 43a is empty (H1 in FIG. 28A).

FIGS. 29A is a perspective view of unlocked suture lock 30b in its unlocked state, with tube 40a and plug 50b oriented with respect to each other such that tube axis a40b is coaxial with plug axis a50b, and the plug’s distal portion 54b is facing the tube’s proximal portion 42b. FIGS. 29B and 30 respectively show perspective and cross-sectional views of locked suture lock 30b.

In some implementations, and as shown, tube 40b is shaped (e.g., laser-cut from a portion of hypotube) to define slits 443b that facilitate locking suture lock 30b by increasing the tube’s compliance to forces applied to the tube as plug 50b advances into the tube. In some such implementations, slits 443b allow tube 40b to elastically deform as plug 50b advances into the tube. Alternatively or in addition, slits 443b can be oriented in parallel along tube-axis a40b in a manner that helps to steer plug 50b along the tube-axis as the plug is pushed into the tube, and/or the slits can reduce contact (and therefore reduce friction) between plug 50b and tube 40b as suture lock 30b is locked.

In some implementations, and as shown, the plug’s outer surface 57b is shaped to define a recessed portion 56b into which tube 40b is shaped to fit while suture lock 30b is locked (FIGS. 29B, 30). In some such implementations, and as shown, recessed portion 56b is disposed longitudinally between a pair of ridges, e.g., between a proximal ridge 59b and a distal ridge 58b that are dimensioned such that tube 40b snaps into the recessed portion as suture lock 30b is locked, e.g., providing feedback to the user indicating that the suture lock is now locked. For example, tube 40b can elastically expand as distal ridge 58b advances therethrough, and the tube recovers its initial shape when it snaps into the recessed portion, thereby retaining plug 50b within the tube.

In some implementations, and as shown in FIG. 30, ridges 58b, 59b can be of different heights relative to recessed portion 56b. In some such implementations, and as shown, distal ridge 58b protrudes less than proximal ridge 59b. For example, distal ridge 58b (e.g., a distal shoulder 51b thereof) can offer limited resistance as plug 50b is initially advanced into tube 40b, whereas proximal ridge 59b can offer greater resistance when the tube’s proximal portion 42b reaches the proximal ridge. In this way, suture lock 30b can help the user to avoid overlocking the suture lock.

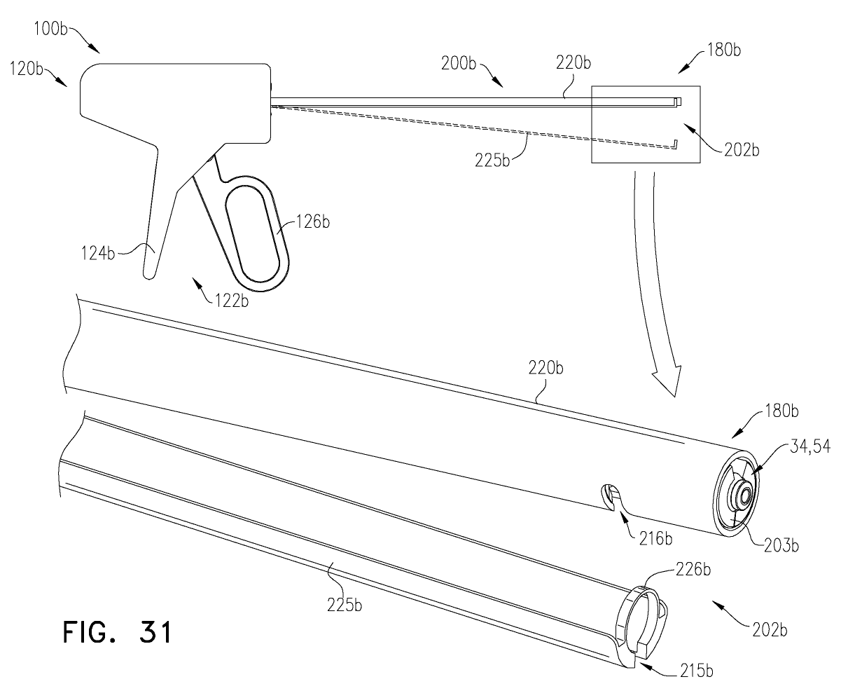

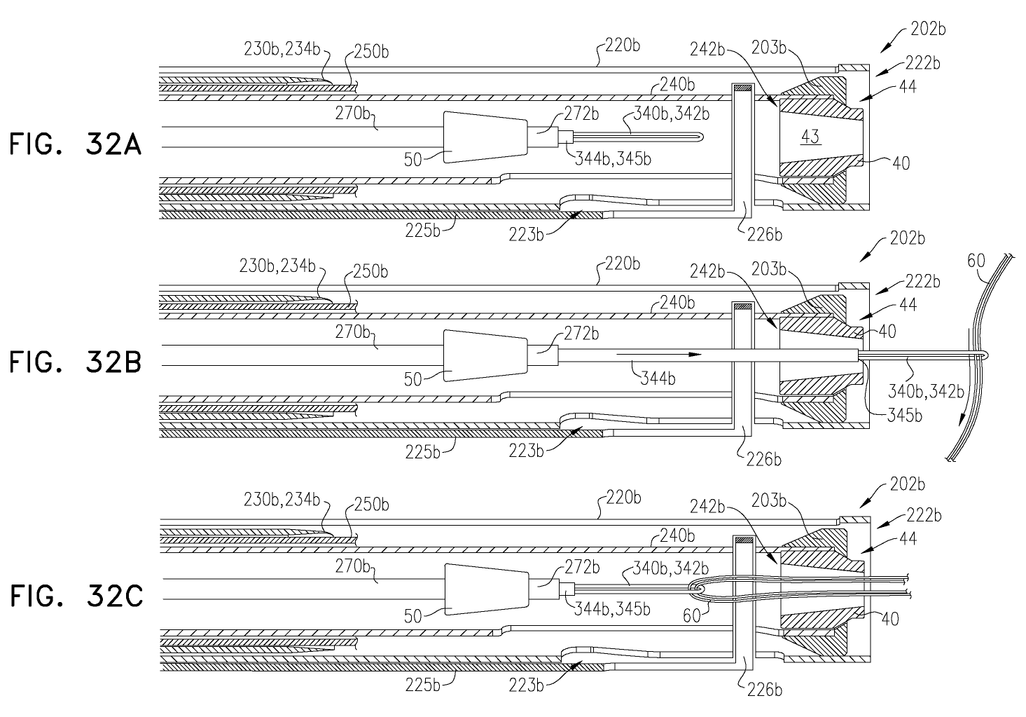

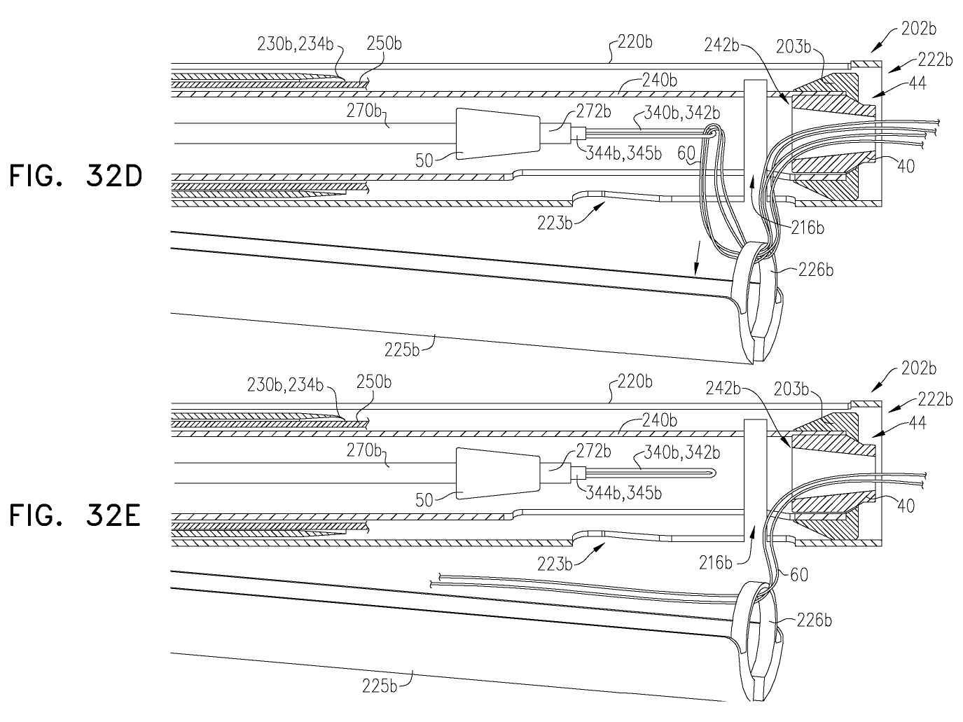

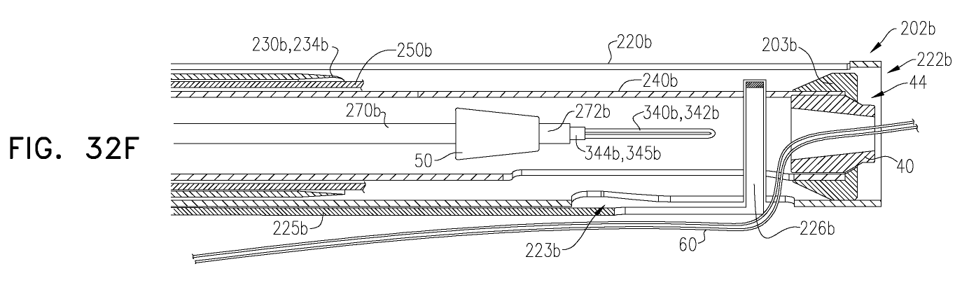

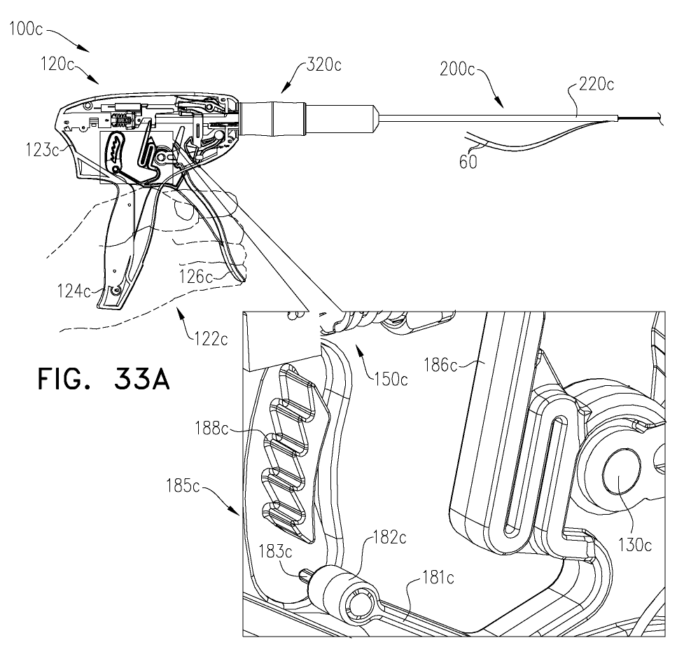

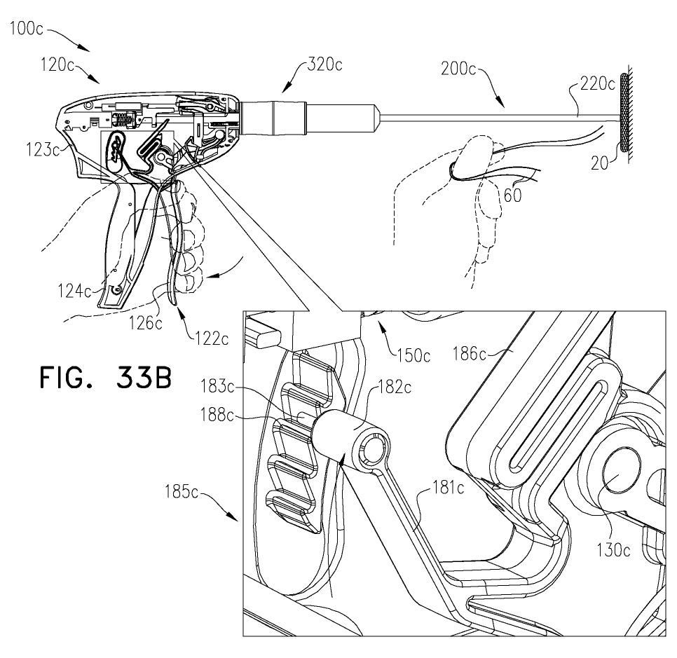

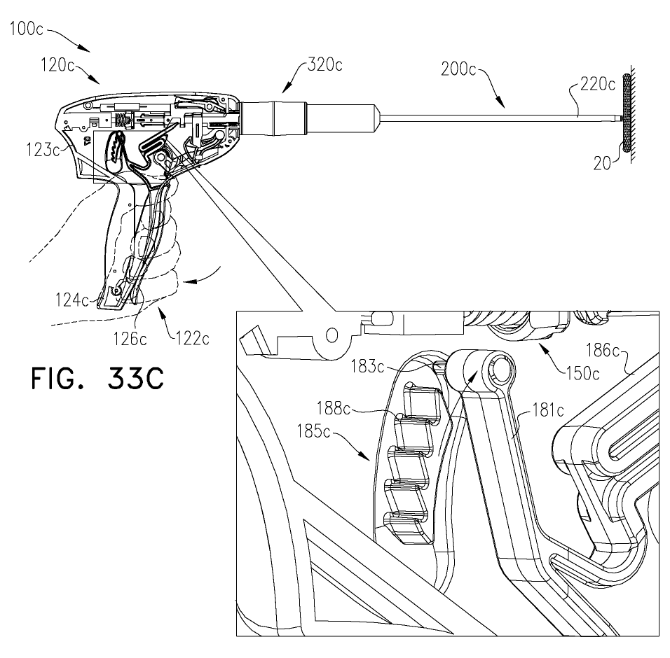

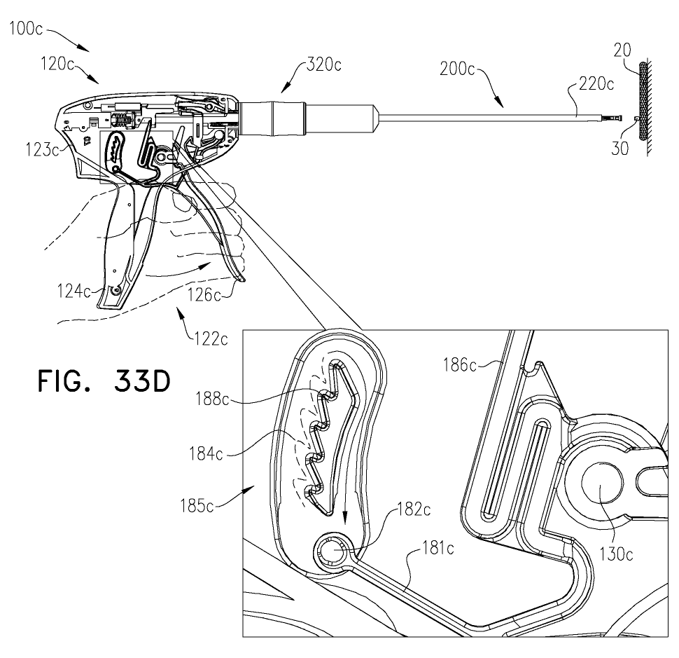

Reference is made to FIGS. 31, 32A-F and 33A-D which are schematic illustrations showing delivery tools 100b, 100c in accordance with some implementations. Delivery tools 100b, 100c can be considered to be variants of tools 100, 100a described hereinabove, and components that are identically named between the delivery tools typically share similar features and serve similar functions as each other. For example, and as shown, delivery tools 100b, 100c each have a handle 122b, 122c at the tool’s proximal portion 120b, 120c, from which a multi-shaft system 200b, 200c extends to the tool’s distal portion. In some such implementations, and similarly to tools, 100, 100a, the tool can be actuated by actuating handle 122b, 122c, e.g., by pivoting trigger 126b, 126c toward the handle’s base 124b, 124c. The description below of delivery tools 100b, 100c focuses upon features that are particular to these tools.

FIGS. 31 and 32A-F show delivery tool 100b and use thereof to thread suture 60 through suture lock 30 (e.g., through tube 40 thereof) . FIG. 31 shows the tool’s multi-shaft system 200b that shares certain features with system 200a described hereinabove. In contrast to system 200a, system 200b (e.g., outer shaft 220b thereof) includes a suture-exposing mechanism that aids the user in unthreading suture 60 from the snare after threading the suture through the tube 40. For example, the suture-exposing mechanism can remove a portion of suture 60 from within system 200b.

In accordance with some implementations, FIG. 31 shows tool 100b not comprising a shuttle, such as shuttle 320, 320a of tools 100, 100a. In some such implementations, at least some functions of shuttle 320, 320a are performed by other components (e.g., handle 122b and/or system 200b) of tool 100b. For example, actuating trigger 126b can in turn actuate system 200b, thereby (i) advancing suture lock 30 from the tool’s proximal portion 120b to the tool’s distal portion 180b, and/or (ii) advancing and retracting snare 340b through the system’s distal portion 202b in order to thread suture through the suture lock’s tube 40 (FIGS. 32A-C).

In some implementations, and as shown, the suture-exposing mechanism comprises an adjustable cantilever 225b, e.g., defining a ring 226b. FIG. 31 shows cantilever 225b pivoted away from the multi-shaft system’s longitudinal axis a200b, e.g., such that the cantilever’s ring 226b has vacated a slot 216b in outer shaft 220b. In some such implementations, the user controls cantilever 225b by pivoting the cantilever about a hinge (not shown).

In accordance with some implementations, FIGS. 32A-F are a series of cross-sectional views of the multi-shaft system’s distal portion 202b being used to thread suture 60 through the suture lock’s tube 40. In some such implementations, and as shown, suture 60 is threaded through tube 40 while plug 50 is retained on inner shaft 270b, proximally of the tube. System 200b can include certain aspects of system 200a (e.g., the inner shaft’s distal portion 272b can define branches such as those at distal portion 272a of inner shaft 270a, and/or plug shaft 250 can define pusher arms such as those of plug shaft 250a), which are not shown in FIGS. 32A-F.

FIG. 32A shows the suture lock’s tube 40 disposed in distal portion 242b of lock shaft 240b, and cantilever 225b aligned along system 200b. In some implementations, and as shown, snare tube 344b extends distally from within distal portion 272 of inner shaft 270, such that the snare’s distal portion 342b extends distally from the snare tube’s distal opening 345a, toward the tube.

In accordance with some implementations, and as shown in FIG. 32B, snare tube 344b and/or snare 340b are advanced distally through tube 40 and out from the outer tube’s distal portion 222b, such that suture 60 can be threaded through the snare. FIG. 32C shows snare 340b having been retracted proximally, e.g., together with snare tube 344b, into inner shaft 270b. Retracting snare 340b in this way pulls suture 60 proximally through the tube’s lumen 43. In some such implementations, and as shown, retracting snare 340b also draws suture 60 through the inner circumference of ring 226b.

In some implementations, and as shown in FIG. 32D, cantilever 225b is then pivoted to pull a length of suture 60 outside of system 200b, e.g., through the outer shaft’s window 223b. The user can then unthread suture 60 from snare 340b (not shown), such that the suture remains threaded through the suture lock’s tube (FIG. 32E), after which cantilever 225b can be pivoted back into alignment with system 200b, such that ring 226b reenters the outer shaft’s slot 216b (FIG. 32F). At this stage, tool 100b can be placed into position and suture lock 30 can be locked, e.g., as shown hereinabove with reference to tool 100a in FIGS. 25M-R.

FIGS. 33A-D show tool 100c during different stages of locking suture lock 30 (e.g., corresponding to certain stages among those described hereinabove with reference to tool 100 in Figs. FIGS. 11I-P and FIGS. 12A-E, or with reference to tool 100a in FIGS. 25M-R and FIGS. 26A-F) by actuating the tool’s handle 122c.

In some implementations, the stages by which suture lock 30 is locked are meant to advance in sequence (e.g., irreversibly), from one stage to the next. In some such implementations, it is therefore desirable to inhibit the delivery tool from reverting from one stage to a previous stage. Tool 100c therefore comprises a safety mechanism that prevents the tool from reverting to an earlier stage, e.g., in the case that the user releases trigger 126c before fully actuating the trigger.

In some implementations, and as shown, housing 123c of the tool’s handle 122c is shaped to define the safety mechanism that prevents trigger 126c, and therefore block-interface 186c, from reverting to an earlier stage of actuation, e.g., in the case that the user inadvertently releases the trigger. In some such implementations, and as shown, the safety mechanism is defined by a ratchet 185c that facilitates one-way advancement of trigger 126c. For example, and as shown, trigger 126c can comprise a ratchet-interface 181c, (e.g., defining a pin 183c at the ratchet-interface’s distal portion 182c), that interacts with a series of teeth 188c defined by housing 123c. In this way, pin 183c advances from one tooth 188c to the next tooth as trigger 126c pivots about hinge 130c, and if the user releases trigger 126c before fully locking the suture lock, one of the teeth 188c will engage pin 183c. Thus, if the user releases trigger 126c prematurely, ratchet 185c will prevent the tool from returning to the ready state (e.g., even though, as described hereinabove with reference to tool 100a, trigger spring 132a pulls trigger block 144a and therefore block-interface 186a proximally), thereby keeping tool 100c at the stage to which it had advanced, such that the user can then resume locking the suture lock by again actuating trigger 126.

Although ratchet 185c is discussed herein with reference to its interaction with tool 100a, incorporation of safety mechanisms such as ratchet 185c into other delivery tools, e.g., tool 100 or 100b, is contemplated.

FIG. 33A shows tool 100c in a resting state corresponding to stage A of tool 100a described hereinabove with reference to FIGS. 25L and 26A. In some implementations, and as shown in the inset of FIG. 33A, the ratchet-interface 181 is disengaged from ratchet 185c while tool 100c is in the resting state.

In accordance with some implementations, FIG. 33B shows tool 100c having advanced to a stage corresponding to stage C described hereinabove with reference to FIGS. 25O and 26C. In accordance with some such implementations, the user has actuated handle 122c by pivoting trigger 126c toward base 124c, and as shown in the inset of FIG. 33A, both the trigger’s block-interface 186c and ratchet-interface 181c have advanced (e.g., pivoted about hinge 130c), such that the ratchet-interface’s pin 183c has engaged one of the ratchet’s teeth 188c. At this point, were the user to release trigger 126c, tooth 188c and pin 183c would together serve as a brake, e.g., countering the pulling force applied by trigger spring 132a upon trigger block 144a of tool 100a. In this way, the plug shaft’s plug-pusher arms 254a would remain engaged with the suture lock’s plug even though the user releases the trigger, reducing a risk of undesirably unlocking the suture lock.

In accordance with some implementations, FIG. 33C shows tool 100c having advanced to a stage corresponding to stage E described hereinabove with reference to FIGS. 25P and 26E. In accordance with some such implementations, ratchet-interface 181c has disengaged from ratchet 185c, e.g., as cutter shaft 230a of tool 100a reaches its distal travel limit, as described hereinabove with reference to FIG. 26E.

In this way, ratchet 185c is ready to allow tool 100c to reset as the user releases trigger 126c, e.g., by transitioning into a state corresponding to stage F, similarly to as described hereinabove with reference to FIGS. 25Q and 26F. FIG. 33D shows tool 100c having completed a complete cycle of operation. The inset of FIG. 33D shows the path 184c traveled by the ratchet-interface’s pin 183c during a complete cycle of operation, such that tool is reset and ready to engage another suture lock 30 and advance it to the tool’s distal portion.

The described systems, apparatuses, devices, methods, etc. should not be construed as limiting in any way. Instead, the present disclosure is directed toward all novel and nonobvious features and aspects of the various disclosed implementations and implementations, alone and in various combinations and sub-combinations with one another. The disclosed systems, apparatuses, devices, methods, etc. are not limited to any specific aspect, feature, or combination thereof, nor do the disclosed systems, apparatuses, devices, methods, etc. require that any one or more specific advantages be present or problems be solved.

The various systems, devices, apparatuses, etc. in this disclosure can be sterilized (e.g., with heat, radiation, ethylene oxide, hydrogen peroxide, etc.) to ensure they are safe for use with patients, and the methods herein can comprise such sterilization of the associated system, device, apparatus, etc. Furthermore, the scope of the present disclosure includes, for some implementations, sterilizing one or more of any of the various systems, devices, apparatuses, etc. in this disclosure.

Any of the above method(s) can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can comprise, for example, computerized and/or physical representations.

Various implementations of systems, devices, methods, etc. are disclosed herein, and any combination of their features, components, and options can be made unless specifically excluded. In short, individual components of the disclosed systems can be combined unless mutually exclusive or physically impossible.

Although the operations of some of the disclosed methods are described in a particular, sequential order for convenient presentation, it should be understood that this manner of description encompasses rearrangement, unless a particular ordering is required by specific language set forth herein. For example, operations described sequentially can in some cases be rearranged or performed concurrently. Moreover, for the sake of simplicity, the attached figures may not show the various ways in which the disclosed systems, apparatuses, devices, methods, etc. can be used in conjunction with other systems, apparatuses, devices, methods, etc.

The present disclosure is not limited to the examples that have been particularly shown and described hereinabove. Rather, the scope includes both combinations and subcombinations of the various features described hereinabove, as well as variations and modifications thereof that are not in the prior art, which would occur to persons skilled in the art upon reading the foregoing description.

Example Applications (some non-limiting examples of the concepts herein are recited below):

Example 1. A system for use with a suture, the system comprising: multiple suture locks, each suture lock comprising: a first lock piece, and/or a second lock piece; and/or a tool: comprising: at a proximal portion of the tool, a storage zone in which the multiple suture locks are disposed, and/or a multi-shaft system, extending from the proximal portion of the tool to a distal portion of the tool, and/or operable to, for each of the multiple suture locks, sequentially: advance the first lock piece and the second lock piece distally from the storage zone to the distal portion, and/or within the distal portion, secure the suture lock to the suture by engaging the first lock piece with the second lock piece.

Example 2. The system according to example 1, wherein at least one of the tool and the suture locks is sterile.

Example 3. The system according to any one of examples 1–2, wherein the tool is operable to, for each of the multiple suture locks, secure the suture lock to the suture by sandwiching the suture between first lock piece and the second lock piece.

Example 4. The system according to any one of examples 1–3, wherein the tool is operable to, for each of the multiple suture locks, sequentially, within the distal portion: thread the suture through the first lock piece, and/or secure the suture lock to the suture by engaging the first lock piece and the suture with the second lock piece.

Example 5. The system according to example 4, wherein the tool further comprises a trigger, the trigger operable to, upon application of an actuation force to the trigger, actuate the multi-shaft system to, while a length of suture is threaded through the first lock piece: secure the suture lock to the suture by engaging the first lock piece with the second lock piece, and/or trim the suture.

Example 6. The system according to example 5, wherein the trigger is configured to, upon application of the actuation force to the trigger, actuate the multi-shaft system to trim the suture while engaging the first lock piece with the second lock piece.

Example 7. The system according to any one of examples 5–6, wherein the trigger is operable such that: upon ceasing application of the actuation force to the trigger, subsequently to securing the suture lock to the suture by engaging the first lock piece with the second lock piece, and/or prior to actuation of the multi-shaft system to trim the suture; the multi-shaft system continues to engage the first lock piece with the second lock piece.

Example 8. The system according to example 7, wherein: the suture lock is a first suture lock of the multiple suture locks, the trigger is operable to, upon application of the actuation force to the trigger, cyclically actuate the multi-shaft system such that trimming the suture advances the tool toward a ready state in which the tool is actuatable to: engage a second suture lock of the multiple suture locks, and/or advance the second suture lock to the distal portion of the tool.

Example 9. The system according to example 4, wherein the tool comprises: a snare, operable to thread a length of the suture through the first lock piece and into the multi-shaft system, and/or a suture-exposing mechanism, operable to remove the length of suture from the multi-shaft system.

Example 10. The system according to example 9, wherein the suture-exposing mechanism is operable to remove the length of suture from the multi-shaft system while the length of suture remains threaded through the first lock piece.

Example 11. The system according to any one of examples 9–10, wherein the snare is operable to: extend distally through the first lock piece, and/or retract distally, drawing the length of suture proximally through the first lock piece.

Example 12. The system according to any one of examples 9–11, wherein the suture-exposing mechanism comprises a cantilever that is pivotable between: an aligned state in which the cantilever is aligned along the multi-shaft system, and/or an oblique state in which the cantilever is angled away from the multi-shaft system.

Example 13. The system according to example 12, wherein: the snare is operable to thread the suture through the first lock piece and through a portion of the cantilever, while the cantilever is in the aligned state, and/or pivoting the cantilever away from the system, while the length of suture is threaded through the portion, exposes the suture from the system.

Example 14. The system according to any one of examples 1–4, wherein: the first lock piece comprises a tube, and/or the second lock piece comprises a plug.

Example 15. The system according to example 14, wherein: the tube has: a distal face, and/or an outer surface that defines: at a first portion of the tube, a first width of the tube, at a second portion of the tube, a second width of the tube, the second width being smaller than the first width, and/or a shoulder between the first portion and the second portion, the second portion extending axially between the distal face and the shoulder; and/or wherein the tool is configured to hold the tube in the distal portion of the tool by engaging the shoulder, such that the distal face of the tube protrudes distally from a distal aperture of the tool.

Example 16. The system according to example 15, wherein: the first portion of the tube is a proximal portion of the tube, and/or the second portion of the tube is a distal portion of the tube having a distal outer diameter.

Example 17. The system according to example 16, wherein: the shoulder of the tube is wider than a distal surface of the tube, and/or the tool is configured to hold the tube in the distal portion of the tool by the shoulder of the tube, such that the distal surface of the tube protrudes medially and distally from the tool.

Example 18. The system according to any one of examples 1–17, wherein the tool is the tool is operable such that, for each of the multiple suture locks, following the securing of the suture lock to the suture, the suture lock remains within the distal portion of the tool until an external force is applied to remove the suture lock from the tool.

Example 19. The system according to example 18, wherein the tool is operable such that, for each of the multiple suture locks, following the securing of the suture lock to the suture, the suture lock remains within the distal portion of the tool, until an external force between 50–100 g is applied to remove the suture lock from the tool.

Example 20. The system according to any one of examples 1–19, wherein the tool further comprises a shuttle: mounted to be repeatedly shuttled, each shuttling including (i) an outbound leg in which the shuttle is slid distally over and along the multi-shaft system from the proximal portion of the tool to the distal portion of the tool, and (ii) a return leg in which the shuttle is subsequently slid proximally over and along the multi-shaft system to return from the distal portion of the tool to the proximal portion of the tool; and/or operable such that, during each shuttling: the outbound leg slides one of the suture locks distally from the storage zone to the distal portion, and/or the return leg leaves the suture lock at the distal portion.

Example 21. The system according to example 20, wherein the tool is configured such that, upon completion of an outbound leg of the shuttling, the shuttle is inhibited from being shuttled on a second outbound leg of the shuttling until the tool has been actuated to secure the suture lock to the suture within the distal portion of the tool.

Example 22. The system according to example 21, wherein: the shuttle is configured to perform a subsequent outbound leg of the shuttling after completion of a return leg of the shuttling, and/or the tool is configured to inhibit completion of the return leg of the shuttling until the tool has been actuated to secure the suture lock to the suture within the distal portion of the tool.

Example 23. The system according to any one of examples 20–22, wherein the shuttle is operable such that during each shuttling, the respective outbound leg unlocks the suture lock by separating the first lock piece from the second lock piece, such that the tool assumes a ready state in which the tool holds the first lock piece and the second lock piece, separated from each other, within the distal portion of the tool.

Example 24. The system according to example 23, wherein: the tool further comprises a trigger, operable to secure the suture lock to the suture within the distal portion of the tool by actuating the multi-shaft system to engage the first lock piece with the second lock piece, and/or the tool is configured such that the respective return leg of each shuttling facilitates operation of the trigger.

Example 25. The system according to example 24, wherein: the proximal portion of the tool comprises an interlock, the interlock: lockable such that the interlock inhibits operation of the trigger, and/or unlockable such that the interlock facilitates operation of the trigger; and/or the respective return leg of each shuttling facilitates operation of the trigger by unlocking the interlock.

Example 26. The system according to any one of examples 24–25, wherein: the suture lock is a first suture lock, and/or the tool is configured such that securing the first suture lock to the suture by operating the trigger resets the tool, thereby facilitating the shuttle, during a subsequent outbound leg of the shuttling, sliding a second suture lock distally from the storage zone to the distal portion.

Example 27. The system according to example 26, wherein the tool is configured such that securing the first suture lock to the suture by operating the trigger resets the tool such that the multi-shaft system inhibits operation of the trigger until the shuttle, during the subsequent outbound leg of the shuttling, slides the second suture lock distally from the storage zone to the distal portion.

Example 28. The system according to any one of examples 26–27, wherein the proximal portion of the tool comprises a shuttle adapter, the shuttle adapter configured to: engage the shuttle upon the shuttle completing a return leg of the shuttling in a manner that inhibits the shuttle from engaging the second suture lock, and/or upon the tool securing the first suture lock to the suture by operating the trigger, facilitate the shuttle, during the subsequent outbound leg of the shuttling, sliding the second suture lock distally from the storage zone to the distal portion.

Example 29. The system according to any one of examples 23–28, wherein: the tool further comprises a snare, housed by the shuttle; and/or the shuttle is operable to, while the tool is in the ready state: release the snare from the shuttle distally through the first lock piece, such that the snare snares a length of suture, and/or draw the snared suture proximally back through the first lock piece by proximally retracting the snare.

Example 30. The system according to example 29, wherein the tool is configured such that: distally releasing the snare inhibits operation of the tool to secure the suture lock to the suture, and/or drawing the snare proximally facilitates operation of the tool to secure the suture lock to the suture.

Example 31. The system according to example 30, wherein: the multi-shaft system comprises a plurality of coaxial shafts; and/or the tool is configured to, for each of the multiple suture locks: upon the tool distally releasing the snare, inhibit operation of the tool to secure the suture lock to the suture by locking the shafts of the multi-shaft system with respect to each other, and/or upon the tool drawing the snare proximally, facilitate operation of the tool to secure the suture lock to the suture by unlocking the shafts of the multi-shaft system with respect to each other.

Example 32. The system according to any one of examples 29–31, wherein the shuttle comprises a carriage and a snare pump, and the snare pump is operable such that, while the tool is in the ready state, sliding the snare pump with respect to the carriage releases the snare from the shuttle distally through the first lock piece, such that the snare snares a length of suture.

Example 33. The system according to example 32, wherein the shuttle is operable such that sliding the snare pump along a given length of the carriage, releases twice the given length of snare from the shuttle, distally through the first lock piece.

Example 34. The system according to any one of examples 32–33, wherein the tool is configured to inhibit operation of the snare pump until the tool assumes the ready state.

Example 35. The system according to example 34, wherein the tool comprises a pump lock, configured to obstruct sliding of the snare pump with respect to the carriage, until the tool assumes the ready state.

Example 36. The system according to any one of examples 34–35, wherein the shuttle comprises a pusher, reversibly engageable to the suture lock and configured to: while supporting the suture lock within the distal portion of the tool, facilitate sliding of the snare pump with respect to the carriage, and/or while disengaged from the suture lock, obstruct sliding of the snare pump with respect to the carriage.

Example 37. The system according to any one of examples 23–36, wherein: the multi-shaft system comprises a lock shaft, a distal portion of the lock shaft defining a holding zone; and/or the tool is operable to, during each outbound leg of the shuttling: transport the suture lock from the storage zone toward the holding zone, and/or unlock the suture lock by separating the first lock piece from the second lock piece, such that the tool assumes the ready state.

Example 38. The system according to example 37, wherein the tool is operable to, during each outbound leg of the shuttling, unlock the suture lock by distally advancing the first lock piece away from the second lock piece and into the holding zone.

Example 39. The system according to example 38, wherein the tool is operable to, during each outbound leg of the shuttling, unlock the suture lock by distally advancing the first lock piece away from the second lock piece such that the first lock piece is friction-fit within the holding zone.

Example 40. The system according to any one of examples 38–39, wherein the tool is configured such that, during each outbound leg of the shuttling, the shuttle unlocks the suture lock such that the first lock piece protrudes distally from the tool.

Example 41. The system according to any one of examples 38–40, wherein the tool is operable to, during each outbound leg of the shuttling, unlock the suture lock by distally advancing the first lock piece away from the second lock piece such that the lock shaft hugs the first lock piece laterally and proximally.

Example 42. The system according to any one of examples 38–41, wherein the tool is operable to, during each outbound leg of the shuttling, unlock the suture lock such that distally advancing the first lock piece away from the second lock piece radially expands the holding zone.

Example 43. The system according to example 42, wherein: the distal portion of the lock shaft defines a receiving zone, proximally adjacent to the holding zone, and/or the shuttle is operable to, during each outbound leg of the shuttling, unlock the suture lock such that the receiving zone guides the first lock piece into the holding zone.

Example 44. The system according to any one of examples 24–28, wherein the tool further comprises: a force applicator at the distal portion of the tool; and/or a force regulator, proximal from the force applicator, the force regulator defining a force threshold, and being configured to, upon application of an actuation force to the trigger, transfer the actuation force to the force applicator in a manner that: while the actuation force remains below the force threshold, secures the suture lock to the suture by engaging the first lock piece with the second lock piece, and/or upon the actuation force reaching the force threshold, trims the suture.

Example 45. The system according to example 44, wherein the force threshold is between 2.5–3.5 kg of force.

Example 46. The system according to example 45, wherein the force threshold is between 2.75–3.25 kg of force.

Example 47. The system according to example 46, wherein the force threshold is 3 kg of force.

Example 48. The system according to any one of examples 44–47, wherein: the multi-shaft system comprises: a first shaft defining a lumen, a distal portion of the first shaft defining a holding zone, a second shaft, configured to, by sliding distally, secure the suture lock to the suture by engaging the first lock piece with the second lock piece at the holding zone, and/or a third shaft defining a blade at a distal portion of the third shaft, the third shaft configured to trim the suture by sliding distally; and/or wherein: while the actuation force remains below the force threshold, the second shaft defines the force applicator, and/or upon the actuation force reaching the force threshold, the third shaft defines the force applicator.

Example 49. The system according to example 48, wherein the tool is configured such that: application of the actuation force, below the force threshold, to the trigger advances the second shaft with respect to the first shaft, thereby securing the suture lock to the suture by engaging the first lock piece with the second lock piece at the holding zone, and/or application of the actuation force beyond the force threshold to the trigger advances the third shaft with respect to the first shaft, thereby trimming the suture.

Example 50. The system according to any one of examples 48–49, wherein the shuttle is operable such that, during each shuttling: the outbound leg slides one of the suture locks distally through the lumen of the first shaft, toward the holding zone, and/or the force regulator is configured to, upon application of the actuation force below the force threshold to the trigger, transfer the actuation force to the second shaft such that the second shaft secures the suture lock to the suture.

Example 51. The system according to example 50, wherein the tool is operable to, upon application of the actuation force below the force threshold to the trigger, secure the suture lock to the suture by pushing distally upon the second lock piece.

Example 52. The system according to any one of examples 50–51, wherein the tool is operable to, upon application of the actuation force below the force threshold to the trigger, secure the suture lock to the suture by engaging the first lock piece with the second lock piece.

Example 53. The system according to any one of examples 50–52, wherein: the multi-shaft system comprises an inner shaft, the suture lock defines a suture lock-lumen, and/or the shuttle is operable such that, during each shuttling, the outbound leg slides one of the suture locks distally from the storage zone toward the holding zone by sliding the suture lock over and along the inner shaft while the inner shaft is disposed in the suture lock-lumen.

Example 54. The system according to example 53, wherein the shuttle is operable such that, during each shuttling, the outbound leg slides one of the suture locks distally from the storage zone toward the holding zone by sliding the suture lock over and along the inner shaft while the suture lock is disposed within the lumen of the first shaft.

Example 55. The system according to example 54, wherein the first shaft defines support arms that are configured to, while the shuttle slides the suture lock distally from the storage zone toward the holding zone: maintain a radial position of the inner shaft within the lumen of the first shaft, and/or deflect radially to facilitate distal movement of the suture lock between the support arms.

Example 56. The system according to any one of examples 53–55, wherein the second shaft defines pusher arms that are configured to, while the shuttle slides the suture lock distally from the storage zone toward the holding zone: maintain a radial position of the inner shaft within the lumen of the first shaft, and/or deflect radially to facilitate distal movement of the suture lock between the pusher arms.

Example 57. The system according to any one of examples 53–56, wherein the tool is configured to, upon application of the actuation force below the force threshold to the trigger, secure the suture lock to the suture by engaging the first lock piece with the second lock piece while the inner shaft is disposed in the suture lock-lumen.

Example 58. The system according to any one of examples 53–57, wherein the tool is configured to, upon application of the actuation force beyond the force threshold to the trigger, trim the suture while the inner shaft is disposed in the suture lock-lumen.

Example 59. The system according to any one of examples 53–58, wherein the shuttle is operable such that, during each shuttling, the outbound leg slides one of the suture locks distally from the storage zone toward the holding zone, up to a separation point at which sliding the shuttle further distally: separates the first lock piece from the second lock piece, and/or seats the first lock piece in the holding zone.

Example 60. The system according to example 59, wherein the tool is operable such that: sliding the shuttle distally to the separation point pushes the suture lock over and along the inner shaft, while the inner shaft is disposed within the suture lock-lumen, and/or sliding the shuttle further distally from the separation point pushes the first lock piece distally off of the inner shaft and into the holding zone.

Example 61. The system according to any one of examples 59–60, wherein the tool is operable such that sliding the shuttle further distally from the separation point separates the first lock piece from the second lock piece such that the first lock piece defines a first lock-piece lumen and the second lock piece defines a second lock-piece lumen.

Example 62. The system according to example 61, wherein the tool is operable to, for each of the multiple suture locks, thread the suture through the first lock-piece lumen while: the first lock piece is seated in the holding zone, and/or the inner shaft is disposed within the second lock-piece lumen.

Example 63. The system according to example 62, wherein the tool is operable to, for each of the multiple suture locks, thread the suture through the first lock-piece lumen, without threading the suture through the second lock-piece.

Example 64. The system according to any one of examples 62–63, wherein the tool is operable to, for each of the multiple suture locks, thread the suture through the first lock-piece lumen while the inner shaft is not disposed within the first lock-piece lumen.

Example 65. The system according to any one of examples 62–64, wherein the tool is operable to, for each of the multiple suture locks, secure the suture lock to the suture by engaging the first lock-piece with the second lock-piece while the suture is disposed within the first lock-piece lumen.

Example 66. The system according to example 65, wherein the tool is operable to, for each of the multiple suture locks, secure the suture lock to the suture by engaging the first lock-piece with the second lock-piece while the suture is disposed outside of the second lock-piece lumen.

Example 67. The system according to any one of examples 65–66, wherein the tool is operable to, for each of the multiple suture locks, trim the suture while: the suture is disposed within the first lock-piece lumen, and/or the inner shaft is disposed within the suture-lock lumen.

Example 68. The system according to any one of examples 65–67, wherein the tool is operable to, for each of the multiple suture locks, trim the suture while the suture is disposed outside of the second lock-piece lumen.

Example 69. The system according to any one of examples 59–68, wherein, upon application of the actuation force below the force threshold to the trigger, the actuation force pushes the second lock piece distally such that the first lock piece engages with the second lock piece in the holding zone.

Example 70. The system according to any one of examples 59–69, wherein the inner shaft is configured to longitudinally restrain the second lock piece while the shuttle separates the first lock piece from the second lock piece.

Example 71. The system according to example 70, wherein a distal portion of the inner shaft defines a detent, the detent configured to inhibit distal sliding of the second lock piece past the detent while the shuttle separates the first lock piece from the second lock piece.

Example 72. The system according to any one of examples 70–71, wherein the tool is configured such that application of the actuation force below the force threshold to the trigger engages the first lock piece with the second lock piece in the holding zone by pushing the second lock piece from the separation point toward the first lock piece.

Example 73. The system according to example 72, wherein the tool comprises an inner shaft spring, the inner shaft spring configured to, upon application of the actuation force below the force threshold to the trigger, engage the first lock piece with the second lock piece by pushing the inner shaft and the second lock piece from the separation point toward the first lock piece.

Example 74. The system according to example 73, wherein the tool is configured such that: while the actuation force is not applied to the trigger, the second shaft inhibits the inner shaft spring’s pushing the inner shaft and the second lock piece from the separation point toward the first lock piece, and/or upon application of the actuation force below the force threshold to the trigger, the second shaft facilitates the inner shaft spring’s pushing the inner shaft and the second lock piece from the separation point toward the first lock piece.

Example 75. A system for use with a suture, the system comprising: multiple suture locks, each suture lock comprising a plug and a tube; and/or a tool: comprising: at a proximal portion of the tool, a storage zone in which the multiple suture locks are disposed, and/or a multi-shaft system, extending from the proximal portion of the tool to a distal portion of the tool, and/or operable to, for each of the multiple suture locks, sequentially: advance the suture lock distally from the storage zone to the distal portion, and/or within the distal portion, secure the suture lock to the suture by pushing the plug into the tube.

Example 76. The system according to example 75, wherein at least one of the tool and the suture locks is sterile.

Example 77. The system according to any one of examples 75–76, wherein, for each of the suture locks disposed in the storage zone, the plug is disposed in the tube.

Example 78. The system according to any one of examples 75–77, wherein the tool is configured to advance each of the suture locks distally from the storage zone while the plug is disposed at least partly within the tube.

Example 79. The system according to any one of examples 75–78, wherein the tool is configured to separate the tube from the plug prior to pushing the plug into the tube.

Example 80. The system according to example 79, wherein the tool is configured to: advance each of the suture locks from the storage zone to the distal portion of the tool while the plug is at least partly disposed within the tube, and/or within the distal portion, separate the tube from the plug.

Example 81. The system according to example 80, wherein the tool is configured to separate the tube from the plug by advancing the tube, within the distal portion of the tool, distally from the plug.

Example 82. The system according to any one of examples 75–81, wherein the tool is configured such that, following the securing of the suture lock to the suture, the suture lock remains within the distal portion of the tool, until an external force is applied to remove the suture lock from the tool.

Example 83. The system according to example 82, wherein the tool is configured such that, following the securing of the suture lock to the suture, the suture lock remains within the distal portion of the tool, until an external force of 50–100 g is applied to remove the suture lock from the tool.

Example 84. The system according to any one of examples 75–83, wherein: the multi-shaft system defines a holding zone at the distal portion of the tool; and/or the tool is configured to, while holding the tube in the holding zone: thread the suture through the tube, secure the suture lock to the suture by pushing the plug into the tube such that a portion of the suture becomes sandwiched between an outer surface of the plug and an inner surface of the tube, and/or trim the suture.

Example 85. The system according to example 84, wherein: the distal portion of the multi-shaft system defines a holding arm bordering the holding zone, and/or the tool is configured to fit the tube into the holding zone by changing an orientation of the holding arm.

Example 86. The system according to example 85, wherein the tool is configured such that, after the tube fits into the holding zone, the holding arm supports the tube in the holding zone.

Example 87. The system according to any one of examples 84–86, wherein: the tube has: a distal face, and/or an outer surface that defines: at a first portion of the tube, a first width of the tube, at a second portion of the tube, a second width of the tube, the second width being smaller than the first width, and/or a shoulder between the first portion and the second portion, the second portion extending axially between the distal face and the shoulder; and/or wherein the tool is configured to hold the tube in the holding zone by engaging the shoulder, such that the distal face of the tube protrudes distally from a distal aperture of the tool.

Example 88. The system according to example 87, wherein: the first portion of the tube is a proximal portion of the tube, and/or the second portion of the tube is a distal portion of the tube having a distal outer diameter.

Example 89. The system according to any one of examples 87–88, wherein: the shoulder of the tube is wider than a distal surface of the tube, and/or the tool is configured to hold the tube in the holding zone by the shoulder of the tube, such that the distal surface of the tube protrudes medially and distally from the tool.

Example 90. The system according to any one of examples 75–89, wherein the tool further comprises a shuttle: mounted to be repeatedly shuttled, each shuttling including (i) an outbound leg in which the shuttle is slid distally over and along the multi-shaft system from the proximal portion of the tool to the distal portion of the tool, and (ii) a return leg in which the shuttle is subsequently slid proximally over and along the multi-shaft system to return from the distal portion of the tool to the proximal portion of the tool; and/or configured such that, during each shuttling: the outbound leg slides one of the suture locks distally from the storage zone to the distal portion, and/or the return leg leaves the suture lock at the distal portion.

Example 91. The system according to example 90, wherein the shuttle is configured such that the respective outbound leg of each shuttling: transports the suture lock from the storage zone to the distal portion of the tool, and/or unlocks the suture lock by separating the plug from the tube, such that the tool assumes a ready state in which the tool holds the plug and the tube, separated from each other, within the distal portion of the tool.

Example 92. The system according to example 91, wherein: the tool further comprises a snare, housed by the shuttle; and/or the shuttle is configured to, while the tool is in the ready state: release the snare from the shuttle distally through the tube, such that the snare snares a length of suture, and/or draw the snared suture proximally back through the tube by proximally retracting the snare.

Example 93. The system according to any one of examples 91–92, wherein: the multi-shaft system comprises a lock shaft, a distal portion of the lock shaft defining a distal holding zone; and/or the shuttle is configured to, by sliding distally along the lock shaft while the shuttle is engaged with the tube: transport the suture lock from the storage zone toward the holding zone, and/or unlock the suture lock by separating the plug from the tube, such that the tool assumes the ready state.

Example 94. The system according to example 93, wherein the shuttle is configured to, by sliding distally along the lock shaft while the shuttle is engaged with the tube: transport the suture lock from the storage zone toward the holding zone, and/or unlock the suture lock by advancing the tube away from the plug and into the holding zone, such that the tool assumes the ready state.

Example 95. The system according to example 94, wherein the shuttle is configured to, by sliding distally along the lock shaft while the shuttle is engaged with the tube: transport the suture lock from the storage zone toward the holding zone, and/or unlock the suture lock by advancing the tube away from the plug and into the holding zone, such that the tube protrudes distally from the tool and the tool assumes the ready state.

Example 96. The system according to any one of examples 94–95, wherein the shuttle is configured to, by sliding distally along the lock shaft while the shuttle is engaged with the tube and the tool is in the ready state, compress the holding zone around the tube.

Example 97. The system according to example 96, wherein: the tool comprises an outer shaft slidably housing the lock shaft, and/or the shuttle is configured to, by sliding distally along the lock shaft while the shuttle is engaged with the tube and the tool is in the ready state, compress the holding zone around the tube by passing the outer shaft over and along the holding zone.

Example 98. The system according to example 97, wherein: the distal portion of the lock shaft is biased to expand radially outward; and/or the shuttle is configured to, by sliding proximally along the lock shaft while the shuttle is: engaged to the outer shaft, and/or disengaged from the tube; allow the distal portion of the lock shaft to expand radially outward, thereby releasing the suture lock from the holding zone.

Example 99. The system according to any one of examples 93–98, wherein: the multi-shaft system comprises an inner shaft, the plug defines a plug-lumen, and/or the shuttle is configured to, by sliding distally along the lock shaft while the shuttle is engaged with the tube, transport the suture lock from the storage zone toward the holding zone by sliding the plug-lumen over and over and along the inner shaft while the inner shaft is disposed in the plug-lumen.

Example 100. The system according to example 99, wherein the inner shaft is configured to longitudinally restrain the plug while the shuttle unlocks the suture lock.

Example 101. The system according to example 100, wherein a distal portion of the inner shaft defines a detent, the detent configured to inhibit distal sliding of the plug past the detent while the shuttle unlocks the suture lock.

Example 102. The system according to any one of examples 99–101, wherein: the tube defines a tube-lumen, and/or the multi-shaft system comprises a plug shaft, and is actuatable to lock the suture lock by pushing the plug shaft to push the plug distally into the tube-lumen.

Example 103. The system according to example 102, wherein the multi-shaft system is actuatable to lock the suture lock by pushing the plug shaft to push the plug distally into the tube-lumen such that a portion of the suture becomes sandwiched between an outer surface of the plug and an inner surface of the tube, laterally from the plug-lumen.

Example 104. The system according to any one of examples 102–103, wherein the inner shaft is configured to be pulled distally by the plug as the plug is pushed distally into the tube-lumen, such that the inner shaft accompanies the plug in a manner that maintains the plug aligned with the tube-lumen.

Example 105. The system according to example 104, wherein: the multi-shaft system comprises an inner wire along which the inner shaft is slidably disposed; a distal portion of the inner shaft defines a compressible detent; and/or the multi-shaft system is configured such that: while the distal portion of the inner shaft is disposed over and along the inner wire, the inner wire inhibits radial compression of the detent, thereby inhibiting distal sliding of the plug past the detent while the shuttle unlocks the suture lock, and/or advancing the distal portion of the inner shaft distally beyond the inner wire increases compressibility of the detent, thereby facilitating withdrawal of the inner shaft from within the plug, while the plug is disposed withing the tube-lumen.

Example 106. The system according to any one of examples 90–105, wherein: tool further comprises a trigger, operable to secure the suture lock to the suture within the distal portion by actuating the multi-shaft system to push the plug into the tube, and/or the shuttle is configured such that the respective return leg of each shuttling facilitates operation of the trigger.

Example 107. The system according to example 106, wherein: the proximal portion of the tool comprises an interlock, the interlock: lockable such that the interlock inhibits operation of the trigger, and/or unlockable such that the interlock facilitates operation of the trigger; and/or the respective return leg of each shuttling facilitates operation of the trigger by unlocking the interlock.

Example 108. The system according to any one of examples 106–107, wherein the tool further comprises: a force applicator at the distal portion of the tool; and/or a force regulator, proximal from the force applicator, the force regulator defining a force threshold, and being configured to, upon application of an actuation force to the trigger, transfer the actuation force to the force applicator in a manner that: secures the suture lock to the suture by pushing the plug into the tube while the actuation force remains below the force threshold, and/or upon the actuation force reaching the force threshold, causes the tool to trim the suture.

Example 109. The system according to example 108, wherein: the multi-shaft system comprises: a plug shaft, configured to push the plug distally into the tube by the plug shaft sliding distally, and/or a cutter shaft defining a blade at a distal portion of the cutter shaft, the cutter shaft configured to trim the suture by sliding distally; and/or wherein: while the actuation force remains below the force threshold, the plug shaft defines the force applicator, and/or upon the actuation force reaching the force threshold, the cutter shaft defines the force applicator.

Example 110. The system according to any one of examples 108–109, wherein the force regulator is configured to, upon continued application of the actuation force to the trigger after the tool having trimmed the suture, transfer the actuation force to the force applicator in a manner that causes the tool to release the suture lock.

Example 111. The system according to any one of examples 108–110, wherein the force threshold is between 1.5–2.5 kg of force.

Example 112. The system according to example 111, wherein the force threshold is between 1.75–2.25 kg of force.

Example 113. The system according to example 112, wherein the force threshold is 2 kg of force.

Example 114. A system for locking a suture, the system comprising: a suture lock comprising: a plug, and/or a tube defining a tube-lumen; and/or a tool: having a ready state in which the tool holds the plug and the tube, separated from each other, within a distal portion of the tool, and/or comprising: a snare at the distal portion of the tool, the snare configured to draw the suture through the tube-lumen while the tool is in the ready state, a force applicator at the distal portion of the tool, and/or a force regulator, proximal from the force applicator, the force regulator defining a force threshold, and being configured to, upon application of an actuation force to a proximal portion of the tool while the tool is in the ready state and the suture is disposed through the tube, transfer the actuation force to the force applicator in a manner that: secures the suture lock to the suture by inserting the plug into the tube-lumen while the actuation force remains below the force threshold, and/or upon the actuation force reaching the force threshold, causes the tool to trim the suture.

Example 115. The system according to example 114, wherein at least one of the plug, the tube, and the tool is sterile.

Example 116. The system according to any one of examples 114–115, wherein the force regulator is configured to, upon continued application of the actuation force to the proximal portion of the tool after the tool having trimmed the suture, transfer the actuation force to the force applicator in a manner that causes the tool to release the suture lock.

Example 117. The system according to any one of examples 114–116, wherein the force threshold is between 1.5–2.5 kg of force.

Example 118. The system according to example 117, wherein the force threshold is between 1.75–2.25 kg of force.

Example 119. The system according to example 118, wherein the force threshold is 2 kg of force.

Example 120. The system according to any one of examples 114–119, wherein the tool comprises a multi-shaft system connecting the force regulator to the distal portion of the tool, a distal portion of the multi-shaft system defining the force applicator.

Example 121. The system according to example 120, wherein: while the actuation force remains below the force threshold, a first member of the multi-shaft system defines the force applicator, and/or upon the actuation force reaching the force threshold, a second member of the multi-shaft system defines the force applicator.

Example 122. The system according to any one of examples 114–121, wherein the tool further comprises a shuttle configured to: transport the suture lock from a proximal portion of the tool to the distal portion of the tool, and/or unlock the suture lock by separating the plug from the tube, such that the tool assumes the ready state.

Example 123. The system according to example 122, wherein: the shuttle houses the snare; and/or the shuttle is configured to, while the tool is in the ready state: release the snare from the shuttle and distally through the tube-lumen, such that the snare snares a length of suture, and/or draw the snared suture proximally back through the tube-lumen by proximally retracting the snare.

Example 124. The system according to any one of examples 122–123, wherein: the tool further comprises a multi-shaft system comprising a lock shaft that defines a proximal storage zone and a distal holding zone; and/or the shuttle is configured to, by sliding distally along the lock shaft while the shuttle is engaged with the tube: transport the suture lock from the storage zone toward the holding zone, and/or unlock the suture lock by separating the plug from the tube, such that the tool assumes the ready state.

Example 125. The system according to example 124, wherein the shuttle is configured to, by sliding distally along the lock shaft while the shuttle is engaged with the tube: transport the suture lock from the storage zone toward the holding zone, and/or unlock the suture lock by advancing the tube away from the plug and into the holding zone, such that the tool assumes the ready state.

Example 126. The system according to example 125, wherein the multi-shaft system comprises an inner shaft, configured to longitudinally restrain the plug while the shuttle unlocks the suture lock.

Example 127. The system according to any one of examples 125–126, wherein the multi-shaft system comprises a plug shaft that slidably fits within the lock shaft, the plug shaft configured to lock the suture lock by pushing the plug distally into the tube-lumen at the holding zone.

Example 128. The system according to example 127, wherein the tool is configured such that: the lock shaft holds the tube in the holding zone such that the tube protrudes distally from the tool, and/or while the plug locks the suture lock by pushing the plug distally into the tube at the holding zone.

Example 129. The system according to any one of examples 127–128, wherein: the multi-shaft system comprises an inner shaft, the plug is shaped to define a plug-lumen, the shuttle is configured to transport the suture lock from the storage zone toward the holding zone by sliding the suture lock over and along the inner shaft, while the inner shaft is disposed in the plug-lumen, and/or the plug shaft is configured to lock the suture lock by pushing the plug distally into the tube at the holding zone, such that the inner shaft is pulled distally along with the plug.

Example 130. The system according to any one of examples 125–129, wherein the shuttle is configured to compress the holding zone around the tube by sliding distally over and along the lock shaft while the tool is in the ready state.

Example 131. The system according to example 130, wherein: the tool comprises an outer shaft slidably housing the lock shaft; and/or the tool is configured such that, while: the tool is in the ready state, and/or the shuttle is engaged with the outer shaft, sliding the shuttle distally over and along the lock shaft compresses the holding zone around the tube by passing the outer shaft over and along the holding zone.

Example 132. A method, comprising: within a distal portion of a tool: unlocking a suture lock, while the suture lock remains unlocked, threading a suture through the suture lock, and/or while the suture remains threaded through the suture lock, securing the suture lock to the suture by locking the suture lock; and/or releasing, from the tool, the suture lock secured to the suture.

Example 133. The method according to example 132, further comprising sterilizing the suture lock.

Example 134. The method according to any one of examples 132–133, wherein: the suture lock includes a tube and a plug, the step of unlocking comprises pushing the tube distally away from the plug, and/or the step of securing comprises locking the suture lock by pushing the plug distally into the tube.

Example 135. The method according to example 134, wherein: the step of unlocking comprises pushing the tube distally away from the plug, such that the tube protrudes distally from the tool, and/or the step of locking comprises pushing the plug distally into the tube, while the tube protrudes distally from the tool.

Example 136. The method according to any one of examples 134–135, wherein: the tool includes a shuttle and a multi-shaft system extending from a proximal portion of the tool to a distal portion of the tool, the multi-shaft system defining a proximal storage zone and a distal holding zone; and/or the method further comprises, prior to the step of unlocking, distally advancing the suture lock from the storage zone toward the holding zone by: engaging the suture lock with the shuttle, and/or sliding the shuttle distally over and along the multi-shaft system.

Example 137. The method according to example 136, wherein the step of locking does not require sliding the shuttle over and along the multi-shaft system.

Example 138. The method according to any one of examples 136–137, wherein: the multi-shaft system comprises an inner shaft, and/or the step of distally advancing the suture lock comprises sliding the suture lock from the storage zone toward the holding zone by sliding the suture lock over and along the inner shaft.

Example 139. The method according to example 138, wherein: the step of engaging comprises engaging the tube of the suture lock with the shuttle, and/or the step of unlocking comprises, while the tube is engaged with the shuttle, pushing the tube distally away from the plug and into the holding zone by sliding the shuttle distally over and along the multi-shaft system.

Example 140. The method according to example 139, wherein the step of unlocking comprises, while the suture lock is engaged with the shuttle, pushing the tube distally away from the plug and the inner shaft, while the plug remains disposed over and along the inner shaft.

Example 141. The method according to example 140, wherein: the inner shaft defines a detent at a distal portion of the inner shaft, and/or the step of unlocking comprises, using the detent, longitudinally restraining the plug from sliding distally past the detent.

Example 142. The method according to any one of examples 139–141, wherein: the tube defines a tube-lumen, the multi-shaft system comprises a plug shaft that slidably houses the inner shaft, and/or the step of locking comprises, using the plug shaft, pushing the plug distally into the tube-lumen at the holding zone.

Example 143. The method according to example 142, wherein: the plug is shaped to define a plug-lumen; the inner shaft defines a detent at a distal portion of the inner shaft; and/or the step of locking comprises, while the inner shaft is disposed in the plug-lumen, using the plug shaft to: distally slide the plug over and along the inner shaft, such that the plug abuts the detent, and/or slide the inner shaft distally, along with the plug, such that the inner shaft aligns the plug with the tube-lumen.

Example 144. The method according to example 143, wherein: the multi-shaft system comprises an inner wire along which the inner shaft is slidably disposed, the step of locking comprises, using the plug shaft, sliding the distal portion of the inner shaft distally beyond the inner wire, and/or the step of releasing comprises sliding the inner shaft proximally, thereby radially compressing the detent and withdrawing the inner shaft proximally from the suture lock, through the plug-lumen.

Example 145. The method according to any one of examples 136–144, wherein: the tool further comprises a snare, housed within the shuttle; and/or the step of threading comprises threading the suture through the tube by: releasing the snare from the shuttle, distally through the tube, threading the suture through the snare, and/or retracting the suture and the snare proximally through the tube.

Example 146. The method according to example 145, wherein: the suture lock is a first suture lock; the step of threading comprises threading the suture through the tube of the first suture lock by sliding the shuttle proximally over and along the multi-shaft system; and/or the method further comprises advancing a second suture lock from the storage zone to the distal portion of the multi-shaft system by sliding the shuttle distally over and along the multi-shaft system.

Example 147. The method according to any one of examples 136–146, wherein: the step of unlocking comprises pushing the tube distally away from the plug and into the holding zone by: engaging the tube with the shuttle, and/or sliding the shuttle distally over and along the multi-shaft system; and/or the method further comprises compressing the holding zone around the tube such that the tube is seated at the holding zone.

Example 148. The method according to example 147, wherein: the multi-shaft system includes: a lock shaft, a distal portion of the lock shaft defining the holding zone, and/or an outer shaft slidably housing the lock shaft; and/or the step of compressing comprises: engaging the outer shaft with the shuttle, and/or compressing the holding zone around the tube by passing the outer shaft over and along the lock shaft.

Example 149. The method according to any one of examples 132–148, wherein: the tool includes: a force applicator at the distal portion of the tool, and/or a force regulator, proximal from the force applicator, the force regulator defining a force threshold; and/or the step of locking comprises locking the suture lock by applying an actuation force to a proximal portion of the tool while the suture remains threaded through the suture lock, such that the force regulator transfers the actuation force to the force applicator in a manner that: locks the suture lock while the actuation force remains below the force threshold, and/or trims the suture upon the actuation force reaching the force threshold.

Example 150. The method according to example 149, further comprising, subsequently to the step of locking, releasing the suture lock from the tool by continuing to apply the actuation force, exceeding the force threshold, to the proximal portion of the tool.

Example 151. Apparatus for use with a suture, the apparatus comprising a suture lock that comprises: a tube having an inner surface defining a tube-lumen, the tube having a tube axis along which the tube-lumen is oriented; and/or a plug having a plug axis along which the plug is oriented; wherein: the suture lock is configured to be secured to the suture by sandwiching a portion of the suture between an outer surface of the plug and the inner surface of the tube, and/or the suture lock is transitionable between: an unlocked state in which the plug is disposed outside of the tube-lumen, and/or a locked state in which the plug is partially disposed within the tube-lumen, such that: the plug protrudes distally from the tube, thereby defining a distal protrusion of the suture lock, and/or the plug is recessed within a proximal portion of the tube, thereby defining a proximal recess of the suture lock.

Example 152. The apparatus according to example 151, wherein at least one of the plug and the tube is sterile.

Example 153. The apparatus according to any one of examples 151–152, wherein an axial depth of the proximal recess is greater than an axial length of the distal protrusion.

Example 154. The apparatus according to any one of examples 151–153, wherein: the locked state is an unloaded locked state in which the tube houses the plug, in absence of the suture; and/or while the portion of the suture is threaded through the tube-lumen, the suture lock is transitionable between: a loaded unlocked state in which the plug is disposed outside of the tube-lumen, and/or a loaded locked state in which the plug is partially disposed within the tube-lumen, such that: the portion of the suture is between the outer surface of the plug and the inner surface of the tube, the plug protrudes proximally from the tube, thereby defining a proximal protrusion of the suture lock, and/or the plug is recessed within a distal portion of the tube, thereby defining a distal recess of the suture lock.

Example 155. The apparatus according to example 154, wherein, while the suture lock is in the unloaded locked state, the plug axis and the tube axis are colinear, thereby defining a lock axis.

Example 156. The apparatus according to any one of examples 154–155, wherein, while the suture lock is in the loaded locked state, the plug axis and the tube axis are coaxial.

Example 157. The apparatus according to any one of examples 154–155, wherein, while the suture lock is in the loaded locked state, the plug axis and the tube axis intersect.

Example 158. The apparatus according to any one of examples 154–157, wherein, while the suture lock is in the unloaded locked state, the inner surface of the tube is parallel to the outer surface of the plug.

Example 159. The apparatus according to example 158, wherein the inner surface of the tube is nonparallel to the tube axis.

Example 160. The apparatus according to any one of examples 158–159, wherein the outer surface of the plug is nonparallel to the plug axis.

Example 161. The apparatus according to any one of examples 151–160, wherein: the plug has an inner surface, defining a plug-lumen, oriented along the plug axis; and/or the suture lock is configured to be secured to the suture by insertion of the plug into the tube-lumen such that: the plug axis becomes coaxial with the tube axis, and/or the portion of the suture becomes sandwiched between the outer surface of the plug and the inner surface of the tube, laterally from the plug-lumen.

Example 162. The apparatus according to example 161, wherein: the tube axis is a central axis of the tube, and/or the plug axis is a central axis of the plug.

Example 163. The apparatus according to any one of examples 161–162, wherein the inner surface of the plug is parallel to the plug axis.

Example 164. The apparatus according to any one of examples 151–163, wherein the distal portion of the tube is narrower than the proximal portion of the tube.

Example 165. The apparatus according to example 164, wherein the outer surface of the tube defines a distally-facing shoulder between a wide portion of the tube and a narrow portion of the tube.

Example 166. The apparatus according to example 165, wherein the narrow portion of the tube extends axially from the shoulder to a distal face of the plug.

Example 167. The apparatus according to any one of examples 151–166, further comprising a tool: extending from a proximal portion of the tool to a distal portion of the tool, and/or defining a holding zone at the distal portion of the tool; wherein the tool is configured to thread the suture through the suture lock by pulling the suture proximally through the tube-lumen while holding the tube at the holding zone.

Example 168. The apparatus according to example 167, wherein the tool further comprises a shuttle, the shuttle configured to: slide the suture lock from the proximal portion of the tool to the distal portion of the tool, and/or unlock the suture lock by sliding the tube away from the plug and into the holding zone.

Example 169. The apparatus according to example 168, wherein the tool comprises an inner shaft, configured to longitudinally restrain the plug while the shuttle unlocks the suture lock.

Example 170. The apparatus according to example 169, wherein the shuttle is configured to slide the suture lock from the proximal portion of the tool to the distal portion of the tool by sliding the suture lock over and along the inner shaft, while the inner shaft is disposed in the plug-lumen.

Example 171. The apparatus according to example 170, wherein the tool comprises a plug shaft, configured to secure the suture lock to the suture by: pushing the plug distally into the tube-lumen, and/or pulling the inner shaft distally.

Example 172. Apparatus for use with a suture, the apparatus comprising a suture lock that comprises: a tube having a frustoconical inner surface defining a tube-lumen, the tube having a tube axis along which the tube-lumen is oriented; and/or a plug having: an inner surface defining a plug-lumen, the plug having a plug axis along which the plug-lumen is oriented, and/or a frustoconical outer surface; wherein the suture lock is securable to the suture by insertion of the plug into the tube-lumen such that: a portion of the suture becomes sandwiched between the outer surface of the plug and the inner surface of the tube, laterally from the plug-lumen, and/or the outer surface of the plug grips the inner surface of the tube.

Example 173. The apparatus according to example 172, wherein at least one of the plug and the tube is sterile.

Example 174. The apparatus according to any one of examples 172–173, wherein the suture lock is securable to the suture at a continuum of depths of the plug within the tube-lumen, dependent on characteristics of the suture.

Example 175. The apparatus according to any one of examples 172–174, wherein the suture lock is securable to the suture by insertion of the plug into the tube-lumen such that: the portion of the suture becomes sandwiched between the outer surface of the plug and the inner surface of the tube at a first lateral side of the plug, and/or the outer surface of the plug grips the inner surface of the tube at a second lateral side of the plug, opposite the first lateral side of the plug.

Example 176. The apparatus according to any one of examples 172–175, wherein: the tube axis is a central axis of the tube, and/or the plug axis is a central axis of the plug.

Example 177. The apparatus according to any one of examples 172–176, wherein the suture lock is configured such that, in the absence of the suture, insertion of the plug into the tube-lumen positions the plug axis to be colinear with the tube axis.

Example 178. The apparatus according to any one of examples 172–177, wherein the plug is configured to be inserted into the tube, in absence of the suture, such that the inner surface of the tube is parallel to the outer surface of the plug.

Example 179. The apparatus according to example 178, wherein the inner surface of the tube is nonparallel to the tube axis.

Example 180. The apparatus according to any one of examples 178–179, wherein the outer surface of the plug is nonparallel to the plug axis.

Example 181. The apparatus according to any one of examples 172–180, further comprising a tool, the tool: having a proximal portion and a distal portion, defining a holding zone at the distal portion of the tool, and/or configured to thread the suture through the suture lock by pulling the suture proximally through the tube-lumen while holding the tube at the holding zone.

Example 182. The apparatus according to example 181, wherein the tool comprises: a multi-shaft system that extends from the proximal portion to the distal portion; and/or a shuttle, slidable distally over and along the multi-shaft system in a manner that slides the suture lock distally toward the distal portion of the tool.

Example 183. The apparatus according to example 182, wherein the tool is configured such that, at the distal portion of the tool, the sliding of the shuttle distally slides the tube away from the plug and into the holding zone.

Example 184. The apparatus according to example 183, wherein the tool comprises an inner shaft, configured to restrain the plug while the shuttle slides the tube away from the plug and into the holding zone.

Example 185. The apparatus according to example 184, wherein the tool is configured such that the sliding of the shuttle distally slides the suture lock from the proximal portion of the tool to the distal portion of the tool by sliding the plug-lumen over and over and along the inner shaft.

Example 186. The apparatus according to example 185, wherein a distal portion of the inner shaft defines a detent, the detent configured to inhibit distal sliding of the plug past the detent.

Example 187. The apparatus according to any one of examples 185–186, wherein the tool comprises a plug shaft, and is actuatable to secure the suture lock to the suture by pushing the plug shaft to push the plug distally into the tube-lumen.

Example 188. The apparatus according to example 187, wherein the inner shaft is configured to be pulled distally by the plug as the plug is pushed distally into the tube-lumen, such that the inner shaft accompanies the plug in a manner that maintains the plug aligned with the tube-lumen.

Example 189. The apparatus according to example 188, wherein: the multi-shaft system comprises an inner wire along which the inner shaft is slidably disposed, a distal portion of the inner shaft defines a compressible detent, and/or the multi-shaft system is configured such that: while the distal portion of the inner shaft is disposed over and along the inner wire, the inner wire inhibits radial compression of the detent, thereby inhibiting distal sliding of the plug past the detent while the shuttle slides the tube away from the plug and into the holding zone, and/or advancing the distal portion of the inner shaft distally beyond the inner wire increases compressibility of the detent, thereby facilitating withdrawal of the inner shaft from within the plug, while the plug is disposed withing the tube-lumen.

Example 190. A system for use with a suture, the system comprising: a suture lock that comprises: a tube: defining a tube-lumen that defines a proximal-distal axis of the suture lock, having a distal face, and/or having an outer surface that defines: at a first portion of the tube, a first width of the tube, at a second portion of the tube, a second width of the tube, the second width being smaller than the first width, and/or a shoulder between the first portion and the second portion, the second portion extending axially between the distal face and the shoulder, and/or a plug; and/or a tool configured to: hold the tube in a distal holding zone by engaging the shoulder, such that the distal face of the tube protrudes distally from a distal aperture of the tool, while continuing to hold the suture lock in the holding zone, lock the suture lock by inserting the plug into the tube-lumen, and/or subsequently release the suture lock out of the distal aperture.

Example 191. The system according to example 190, wherein at least one of the plug, the tube, and the tool is sterile.

Example 192. The system according to any one of examples 190–191, wherein the tool is configured to, while holding the tube in the holding zone: thread the suture through the tube, secure the suture lock to the suture by inserting the plug into the tube-lumen such that a portion of the suture becomes sandwiched between an outer surface of the plug and an inner surface of the tube, and/or trim the suture.

Example 193. The system according to any one of examples 190–192, wherein: the first portion of the tube is a proximal portion of the tube, and/or the second portion of the tube is a distal portion of the tube having a distal outer diameter.

Example 194. The system according to any one of examples 190–193, wherein: the shoulder of the tube is wider than a distal surface of the tube, and/or the tool is configured to hold the tube in the holding zone by the shoulder of the tube, such that the distal surface of the tube protrudes medially and distally from the tool.

Example 195. The system according to any one of examples 190–194, wherein: the plug is shaped to define a plug-lumen, the tool comprises an inner shaft, and/or the tool is configured to advance the suture lock from a proximal portion of the tool to a distal portion of the tool by sliding the suture lock over and along the inner shaft, while the inner shaft is disposed in the plug-lumen.

Example 196. The system according to example 195, wherein the tool is configured to lock the suture lock by inserting the plug into the tube-lumen such that a portion of the suture becomes sandwiched between an outer surface of the plug and an inner surface of the tube, laterally from the plug-lumen.

Example 197. The system according to any one of examples 195–196, wherein the tool further comprises a plug shaft, and the tool is configured to secure the suture lock to the suture lock by advancing the plug shaft distally in a manner that: pushes the plug distally into the tube-lumen, and/or pulls the inner shaft distally.

Example 198. The system according to any one of examples 190–197, wherein: the tool has a ready state in which the tool holds: the tube in the holding zone, such that the distal face of the tube protrudes distally from the distal aperture of the tool, and/or the plug, separated from the tube, within a distal portion of the tool; and/or the tool further comprises: a snare at the distal portion of the tool, the snare configured to draw the suture through the tube-lumen while the tool is in the ready state, a force applicator at the distal portion of the tool, and/or a force regulator, proximal from the force applicator, the force regulator defining a force threshold, and being configured to, upon application of an actuation force to a proximal portion of the tool while the tool is in the ready state and the suture is disposed through the tube, transfer the actuation force to the force applicator in a manner that: locks the suture lock to the suture by inserting the plug into the tube-lumen while the actuation force remains below the force threshold, and/or upon the actuation force reaching the force threshold, causes the tool to trim the suture.

Example 199. The system according to example 198, wherein the force regulator is configured to, upon continued application of the actuation force to the proximal portion of the tool after the tool having trimmed the suture, transfer the actuation force to the force applicator in a manner that causes the tool to release the suture lock.

Example 200. The system according to any one of examples 198–199, wherein the force threshold is between 1.5–2.5 kg of force.

Example 201. The system according to example 200, wherein the force threshold is between 1.75–2.25 kg of force.

Example 202. The system according to example 201, wherein the force threshold is 2 kg of force.

Example 203. The system according to any one of examples 198–202, wherein the tool comprises a multi-shaft system connecting the force regulator to the distal portion of the tool, a distal portion of the multi-shaft system defining the force applicator.

Example 204. The system according to example 203, wherein: while the actuation force remains below the force threshold, a first member of the multi-shaft system defines the force applicator, and/or upon the actuation force reaching the force threshold, a second member of the multi-shaft system defines the force applicator.

Example 205. The system according to any one of examples 198–204, wherein: the tool further comprises: a lock shaft that defines a proximal storage zone and the distal holding zone, and/or a shuttle, slidable distally over and along the lock shaft in a manner that slides the suture lock distally toward the distal portion of the tool; and/or wherein the shuttle is configured to, by sliding distally over and along the lock shaft while the shuttle is engaged with the tube: transport the suture lock from the storage zone toward the holding zone, and/or unlock the suture lock by separating the plug from the tube, such that the tool assumes the ready state.

Example 206. The system according to example 205, wherein: the shuttle houses the snare; and/or the shuttle is configured to, while the tool is in the ready state: release the snare from the shuttle and distally through the tube-lumen, such that the snare snares a length of suture, and/or draw the snared suture proximally back through the tube-lumen by proximally retracting the snare.

Example 207. The system according to any one of examples 205–206, wherein: the distal portion of the lock shaft defines a holding arm bordering the holding zone, and/or the shuttle is configured to fit the tube into the holding zone by changing an orientation of the holding arm.

Example 208. The system according to example 207, wherein the distal portion of the lock shaft is configured such that, after the tube fits into the holding zone, the holding arm supports the tube in the holding zone.

Example 209. The system according to any one of examples 205–208, wherein the shuttle is configured to, by sliding distally over and along the lock shaft while the shuttle is engaged with the tube: transport the suture lock from the storage zone toward the holding zone, and/or unlock the suture lock by advancing the tube away from the plug and into the holding zone, such that the tool assumes the ready state.

Example 210. The system according to example 209, wherein the tool comprises an inner shaft, configured to longitudinally restrain the plug while the shuttle unlocks the suture lock.

Example 211. The system according to example 210, wherein: the tool further comprises a plug shaft that slidably fits within the lock shaft, and/or the plug shaft is configured to lock the suture lock by pushing the plug distally into the tube at the holding zone.

Example 212. The system according to example 211, wherein: the plug is shaped to define a plug-lumen, the shuttle is configured to transport the suture lock from the storage zone toward the holding zone by sliding the suture lock over and along the inner shaft, while the inner shaft is disposed in the plug-lumen, and/or the plug shaft is configured to lock the suture lock by pushing the plug distally into the tube at the holding zone, such that the inner shaft is pulled distally along with the plug.

Example 213. The system according to any one of examples 209–212, wherein the shuttle is configured to compress the holding zone around the tube by sliding distally over and along the lock shaft while the tool is in the ready state.

Example 214. The system according to example 213, wherein: the tool further comprises an outer shaft slidably housing the lock shaft; and/or the tool is configured such that, while: the tool is in the ready state, and/or the shuttle is engaged with the outer shaft, sliding the shuttle distally over and along the lock shaft compresses the holding zone around the tube by passing the outer shaft over and along the holding zone.

Example 215. A system for use with a suture, the system comprising: a suture lock that has an outer surface that defines a shoulder; and/or a tool configured to: advance the suture lock from a proximal portion of the tool to a holding zone at a distal portion of the tool, hold at least a part of the suture lock in the holding zone by engaging the shoulder, such that a distal face of the suture lock protrudes distally from a distal aperture of the tool, secure the suture lock to the suture while continuing to hold the suture lock in the holding zone, and/or subsequently release the suture lock out of the distal aperture.

Example 216. The system according to example 215, wherein at least one of the suture lock and the tool is sterile.

Example 217. The system according to any one of examples 215–216, wherein: the shoulder of the suture lock is wider than a distal surface of the suture lock, and/or the tool is configured to hold at least the part of the suture lock in the holding zone by the shoulder of the suture lock, such that the distal surface of the suture lock protrudes medially and distally from the tool.

Example 218. The system according to any one of examples 215–217, wherein: the tool further comprises: a snare at the distal portion of the tool, the snare configured to draw the suture through at least the part of the suture lock; a force applicator at the distal portion of the tool; and/or a force regulator, proximal from the force applicator, the force regulator defining a force threshold, and being configured to, upon application of an actuation force to a proximal portion of the tool while the suture is disposed through at least the part of the suture lock, transfer the actuation force to the force applicator in a manner that: secures the suture lock to the suture while the actuation force remains below the force threshold, and/or upon the actuation force reaching the force threshold, causes the tool to trim the suture.

Example 219. The system according to example 218, wherein the force regulator is configured to, upon continued application of the actuation force to the proximal portion of the tool after the tool having trimmed the suture, transfer the actuation force to the force applicator in a manner that causes the tool to release the suture lock.

Example 220. The system according to any one of examples 218–219, wherein the force threshold is between 1.5–2.5 kg of force.

Example 221. The system according to example 220, wherein the force threshold is between 1.75–2.25 kg of force.

Example 222. The system according to example 221, wherein the force threshold is 2 kg of force.

Example 223. The system according to any one of examples 218–222, wherein the tool comprises a multi-shaft system connecting the force regulator to the distal portion of the tool, a distal portion of the multi-shaft system defining the force applicator.

Example 224. The system according to example 223, wherein: while the actuation force remains below the force threshold, a first member of the multi-shaft system defines the force applicator, and/or upon the actuation force reaching the force threshold, a second member of the multi-shaft system defines the force applicator.

Example 225. The system according to any one of examples 223–224, wherein the tool further comprises a shuttle configured to, by sliding distally over and along the multi-shaft system: transport the suture lock from the proximal portion of the tool to the distal portion of the tool, and/or unlock the suture lock, such that the tool assumes a ready state in which at least the part of the unlocked suture lock is disposed in the holding zone.

Example 226. The system according to example 225, wherein: the shuttle houses the snare; and/or the shuttle is configured to, while the tool is in the ready state: release the snare from the shuttle and distally through at least the part of the suture lock, such that the snare snares a length of suture, and/or draw the snared suture proximally back through at least the part of the suture lock by proximally retracting the snare.

Example 227. The system according to any one of examples 225–226, wherein the multi-shaft system comprises an inner shaft, configured to longitudinally restrain the suture lock while the shuttle unlocks the suture lock.

Example 228. The system according to any one of examples 225–227, wherein: the distal portion of the tool defines a holding arm bordering the holding zone, and/or the shuttle is configured to fit at least the part of the suture lock into the holding zone by changing an orientation of the holding arm.

Example 229. The system according to example 228, wherein the distal portion of the tool is configured such that, after at least the part of the suture lock fits into the holding zone, the holding arm supports at least the part of the suture lock in the holding zone.

Example 230. The system according to any one of examples 225–229, wherein the shuttle is configured to compress the holding zone around the suture lock by sliding distally over and along the multi-shaft system while the tool is in the ready state.

Example 231. The system according to example 230, wherein: the multi-shaft system further comprises an outer shaft; and/or the tool is configured such that, while: the tool is in the ready state, and/or the shuttle is engaged with the outer shaft, sliding the shuttle distally over and along the distal portion of the tool compresses the holding zone around at least the part of the unlocked suture lock by passing the outer shaft over and along the holding zone.

Example 232. A system for locking a suture, the system comprising: a suture lock comprising: a plug, and/or a tube defining a tube-lumen; and/or a tool comprising: a lock shaft: extending from a proximal portion of the tool to a distal portion of the tool, and/or defining a holding zone at a distal portion of the lock shaft, and/or a shuttle, configured to: slide the suture lock from a proximal portion of the lock shaft to the distal portion of the lock shaft, and/or unlock the suture lock by sliding the tube away from the plug and into the holding zone.

Example 233. The system according to example 232, wherein at least one of the plug, the tube, and the tool is sterile.

Example 234. The system according to any one of examples 232–233, wherein the tool is configured to thread a suture through the suture lock by pulling the suture proximally through the tube-lumen while the tube is seated at the holding zone.

Example 235. The system according to any one of examples 232–234, wherein the tool further comprises an outer shaft slidably housing the lock shaft, and the shuttle is configured to compress the holding zone by drawing the outer shaft over the holding zone.

Example 236. The system according to any one of examples 232–235, wherein: the tool further comprises a snare, housed within the shuttle; and/or the tool is configured such that, while the suture lock is unlocked and the tube is seated in the holding zone, the shuttle is operatable to: release the snare from the shuttle and distally through the tube-lumen, and/or retract the snare proximally through the tube and into the shuttle.

Example 237. The system according to any one of examples 232–236, wherein the tool is configured such that distally sliding the shuttle over and along the holding zone fits the tube into the holding zone.

Example 238. The system according to example 237, wherein: the distal portion of the lock shaft defines a holding arm bordering the holding zone, and/or the tool is configured such that distally sliding the shuttle over and along the holding zone fits the tube into the holding zone by changing an orientation of the holding arm.

Example 239. The system according to example 238, wherein the distal portion of the lock shaft is configured such that, after the tube fits into the holding zone, the holding arm supports the tube in the holding zone.

Example 240. The system according to any one of examples 232–239, wherein the tool is configured such that distally sliding the shuttle over and along the holding zone compresses the holding zone around the tube.

Example 241. The system according to example 240, wherein: the tool further comprises an outer shaft, and/or the tool is configured such that, while the shuttle is engaged with the outer shaft, sliding the shuttle distally over and along the distal portion of the tool compresses the holding zone around the tube by passing the outer shaft over and along the holding zone.

Example 242. The system according to any one of examples 232–241, wherein: the tool further comprises a snare, housed within the shuttle; and/or the tool is configured such that, while the suture lock is unlocked and the tube is disposed at the holding zone, the shuttle is operatable to: advance the snare from the shuttle and distally through the tube, and/or retract the snare proximally out of the tube and into the shuttle.

Example 243. The system according to example 242, wherein the tool is configured to thread a suture through the tube by pulling the suture proximally through the tube while at least a part of the unlocked suture lock is disposed at the holding zone.

Example 244. The system according to any one of examples 232–243, wherein the tool comprises an inner shaft, configured to longitudinally restrain the plug while the shuttle unlocks the suture lock.

Example 245. The system according to example 244, wherein: the plug is shaped to define a plug-lumen, and/or the shuttle is configured to slide the suture lock from the proximal portion of the lock shaft to the distal portion of the lock shaft by sliding the suture lock over and along the inner shaft, while the inner shaft is disposed in the plug-lumen.

Example 246. The system according to example 245, wherein the tool comprises a plug shaft, configured to lock the suture lock by: pushing the plug distally into the tube-lumen, and/or sliding the inner shaft distally.

Example 247. A system for locking a suture, the system comprising: multiple suture locks; and/or a tool comprising: at a proximal portion of the tool, a storage zone in which the multiple suture locks are disposed, a lock shaft: extending from the proximal portion of the tool to a distal portion of the tool, and/or defining a holding zone at a distal portion of the lock shaft, and/or a shuttle, mounted such that sliding of the shuttle distally over and along the lock shaft slides one of the suture locks distally from the storage zone into the holding zone, and compresses the holding zone in a manner that traps the suture lock in the holding zone.

Example 248. The system according to example 247, wherein at least one of the tool and the suture locks is sterile.

Example 249. The system according to any one of examples 247–248, wherein the tool further comprises an outer shaft slidably housing the lock shaft, and the shuttle is configured to compress the holding zone by drawing the outer shaft over the holding zone.

Example 250. A system for locking a suture, the system comprising: a suture lock; and/or a tool comprising: at a proximal portion of the tool, a storage zone in which the suture lock are disposed, a lock shaft, extending from the proximal portion of the tool to a distal portion of the tool, at the distal portion of the tool, a holding zone, and/or a shuttle, mounted such that sliding of the shuttle distally over and along the lock shaft: slides the suture lock distally out of the storage zone, unlocks the suture lock, and/or advances at least a part of the unlocked suture lock into the holding zone.

Example 251. The system according to example 250, wherein at least one of the suture lock and the tool is sterile.

Example 252. The system according to any one of examples 250–251, wherein: the tool further comprises a snare, housed within the shuttle; and/or the tool is configured such that, while the suture lock is unlocked and at least the part of the unlocked the suture lock is disposed at the holding zone, the shuttle is operatable to: advance the snare from the shuttle and distally through at least the part of the unlocked suture lock, and/or retract the snare proximally out of at least the part of the unlocked suture lock and into the shuttle.

Example 253. The system according to example 252, wherein the tool is configured to thread a suture through at least the part of the unlocked suture lock by pulling the suture proximally through at least the part of the unlocked suture lock while at least the part of the unlocked suture lock is disposed at the holding zone.

Example 254. The system according to any one of examples 252–253, wherein: the suture lock is shaped to define a lumen, and/or the shuttle is configured to thread the suture through at least the part of the unlocked suture lock within the holding zone by pulling the snare proximally through the lumen while at least the part of the unlocked suture lock is seated at the holding zone.

Example 255. The system according to example 254, wherein the shuttle comprises: a snare, housed within the shuttle; a snare pump, operatable to: release the snare from the shuttle, and/or thread the suture through at least the part of the suture lock by pulling the suture proximally through the lumen while at least the part of the suture lock is seated at the holding zone; and/or a pusher, reversibly engageable to at least the part of the suture lock and configured to: support at least the part of the suture lock while at least the part of the suture lock is seated at the holding zone, and/or inhibit operation of the snare pump while the pusher is disengaged from the suture lock.

Example 256. The system according to example 255, wherein: the snare pump is operatable by longitudinally sliding the snare pump with respect to the holding zone; and/or the pusher is configured such that: while the pusher engages at least the part of the suture lock, the pusher assumes a first conformation in which the snare pump is slidable with respect to the pusher, and/or while the pusher is disengaged from the suture lock, the pusher assumes a second conformation in which the pusher inhibits sliding the snare pump with respect to the holding zone.

Example 257. The system according to any one of examples 254–256, wherein the shuttle comprises: a snare, housed within a snare tube; a snare pump, operatable to: release the snare from the snare tube, and/or thread the suture through at least the part of the suture lock by pulling the suture proximally through the lumen while at least the part of the suture lock is seated at the holding zone; and/or a snare interlock coupled to the snare tube, the snare interlock configured to: facilitate release of the snare from the snare tube while a distal opening of the snare tube faces the holding zone, and/or inhibit release of the snare from the snare tube while the distal opening of the snare tube faces away from the holding zone.

Example 258. The system according to example 257, wherein: the snare pump is operatable by longitudinally sliding the snare pump with respect to the holding zone; and/or the snare interlock comprises a snare interlock lever, the snare interlock lever: coupled to the snare tube at a first portion of the snare interlock lever, shaped to define a brake at a second portion of the snare interlock lever, and/or pivotable such that: while the distal opening of the snare tube faces away from the holding zone, the brake inhibits sliding the snare pump with respect to the holding zone, and/or while the distal opening of the snare tube faces the holding zone, the brake allows sliding the snare pump with respect to the holding zone.

Example 259. A system, comprising: multiple suture locks, each suture lock comprising: a plug, and/or a tube; and/or a tool comprising: at a proximal portion of the tool, a storage zone in which the multiple suture locks are disposed, a multi-shaft system, extending from the proximal portion of the tool to a distal portion of the tool, a shuttle: mounted to be repeatedly shuttled, each shuttling including (i) an outbound leg in which the shuttle is slid distally over and along the multi-shaft system from the proximal portion of the tool to the distal portion of the tool, and (ii) a return leg in which the shuttle is subsequently slid proximally over and along the multi-shaft system to return from the distal portion of the tool to the proximal portion of the tool, and/or configured such that, during each shuttling: the outbound leg slides one of the suture locks distally from the storage zone to the distal portion, and/or the return leg leaves the suture lock at the distal portion, and/or a trigger, operable to lock the suture lock within the distal portion by actuating the multi-shaft system to push the plug into the tube.

Example 260. A system for securing a suture, the system comprising a suture lock that comprises: a plug; and/or a tube, configured to receive the plug, and comprising a wall that: circumscribes and defines a tube axis and a tube-lumen along the tube axis, and/or has a set of axial slits therein, distributed circumferentially around the tube axis to provide the wall with compliance that facilitates insertion of the plug into the tube and retention of the plug by the tube.

Example 261. The system according to example 260, wherein the suture lock is sterile.

Example 262. The system according to any one of examples 260–261, wherein the plug is configured to be inserted into the tube by being advanced along the tube axis and into the tube-lumen by a locking force that is lesser than a locking force that would be required in absence of the set of axial slits.

Example 263. The system according to any one of examples 260–262, wherein the plug is configured to be retained within the tube-lumen by interference fit.

Example 264. The system according to example 263, wherein a proximal portion of the tube has a compliance that is greater than a compliance of a distal portion of the tube.

Example 265. The system according to any one of examples 260–264, wherein the set of axial slits is configured to provide the wall with compliance by each axial slit changing shape upon insertion of the plug into the tube.

Example 266. The system according to example 265, wherein the set of axial slits is configured to provide the wall with compliance by each axial slit expanding upon insertion of the plug into the tube.

Example 267. The system according to any one of examples 260–266, wherein the set of axial slits are a set of proximal slits cut from a proximal end of the tube such that the proximal slits define a set of proximal tabs therebetween.

Example 268. The system according to example 267, wherein the proximal tabs are configured to provide the wall with compliance by expanding radially outward as the plug enters the tube-lumen.

Example 269. The system according to any one of examples 260–268, wherein: the set of slits is a first set of axial slits, and/or the tube has a second set of axial slits therein, distributed circumferentially around the tube axis and extending further distally than the first set of slits.

Example 270. The system according to example 269, wherein the second set of axial slits are circumferentially offset from the first set of axial slits.

Example 271. The system according to any one of examples 269–270, wherein the first set of axial slits are configured to provide a proximal portion of the wall with a greater compliance than the second set of axial slits provide to a distal portion of the wall.

Example 272. The system according to example 271, wherein the first set of axial slits are configured to provide the proximal portion of the wall with the greater compliance by changing shape, upon insertion of the plug into the tube, to a greater degree than the second set of axial slits.

Example 273. The system according to any one of examples 260–272, wherein the tube has a distal slit therein, the distal slit configured to resist advancing the plug distally through the tube, past the distal slit.

Example 274. The system according to example 273, wherein the distal slit is perpendicular to the tube axis.

Example 275. The system according to any one of examples 260–274, wherein the plug is shaped to define a ridge that facilitates retention of the plug by the tube.

Example 276. The system according to example 275, wherein the ridge is a proximal ridge, configured to inhibit advancing the plug distally through the tube such that the proximal ridge enters the tube.

Example 277. The system according to any one of examples 275–276, wherein: the ridge is a first ridge, the plug is shaped to further define a second ridge, and/or the tube is configured to snap into place between the first ridge and the second ridge.

Example 278. The system according to any one of examples 275–277, wherein the ridge is a distal ridge, configured to, after insertion of the plug into the tube, inhibit removing the plug from the tube.

Example 279. The system according to example 278, wherein the distal ridge has: a distal shoulder that facilitates insertion of the plug into the tube, and/or a proximal step that inhibits withdrawing the plug through the tube.

Example 280. The system according to any one of examples 260–279, wherein the suture lock has: an unlocked state in which the plug is disposed outside of the tube-lumen, and/or a locked state in which the plug is at least partially disposed within the tube-lumen.

Example 281. The system according to example 280, wherein, while the suture lock is in the unlocked state, the plug has an external diameter that is greater than an internal diameter of the tube.

Example 282. The system according to any one of examples 260–281, wherein the tube has a greater elasticity than the plug.

Example 283. The system according to example 282, wherein the tube comprises nitinol.

Example 284. The system according to example 282, wherein the plug comprises cobalt-chrome.

Example 285. A system for securing a suture, the system comprising a suture lock that comprises: a plug; and/or a tube, configured to receive the plug, and comprising a wall that: circumscribes and defines a tube axis and a tube-lumen along the tube axis, and/or has a set of slits therein, distributed circumferentially around the tube axis and aligned with the tube axis.

Example 286. The system according to example 285, wherein the suture lock is sterile.

Example 287. The system according to any one of examples 285–286, wherein the plug is configured to be inserted into the tube by being advanced along the tube axis and into the tube-lumen by a locking force that is lesser than a locking force that would be required in absence of the set of slits.

Example 288. The system according to any one of examples 285–287, wherein the plug is configured to be retained within the tube-lumen by interference fit.

Example 289. The system according to example 288, wherein a proximal portion of the tube has a compliance that is greater than a compliance of a distal portion of the tube.

Example 290. The system according to any one of examples 285–289, wherein the set of slits is configured to provide the wall with compliance by each slit changing shape upon insertion of the plug into the tube.

Example 291. The system according to example 290, wherein the set of slits is configured to provide the wall with compliance by each slit expanding upon insertion of the plug into the tube.

Example 292. The system according to any one of examples 290–291, wherein the set of slits are a set of proximal slits cut from a proximal end of the tube such that the proximal slits define a set of proximal tabs therebetween.

Example 293. The system according to example 292, wherein the proximal tabs are configured to provide the wall with compliance by expanding radially outward as the plug enters the tube-lumen.

Example 294. The system according to any one of examples 285–293, wherein: the set of slits is a first set of slits, and/or the tube has a second set of slits therein, distributed circumferentially around the tube axis and extending further distally than the first set of slits.

Example 295. The system according to example 294, wherein the second set of slits are circumferentially offset from the first set of slits.

Example 296. The system according to any one of examples 294–295, wherein the first set of slits are configured to provide a proximal portion of the wall with a greater compliance than the second set of slits provide to a distal portion of the wall.

Example 297. The system according to example 296, wherein the first set of slits are configured to provide the proximal portion of the wall with the greater compliance by changing shape, upon insertion of the plug into the tube, to a greater degree than the second set of slits.

Example 298. The system according to any one of examples 285–297, wherein the tube has a distal slit therein, the distal slit configured to resist advancing the plug distally through the tube, past the distal slip.

Example 299. The system according to example 298, wherein the distal slit is perpendicular to the tube axis.

Example 300. The system according to any one of examples 285–299, wherein the plug is shaped to define a ridge that facilitates retention of the plug by the tube.

Example 301. The system according to example 300, wherein the ridge is a proximal ridge, configured to inhibit advancing the plug distally through the tube such that the proximal ridge enters the tube.

Example 302. The system according to any one of examples 300–301, wherein: the ridge is a first ridge, the plug is shaped to further define a second ridge, and/or the tube is configured to snap into place between the first ridge and the second ridge.

Example 303. The system according to any one of examples 300–302, wherein the ridge is a distal ridge, configured to, after insertion of the plug into the tube, inhibit removing the plug from the tube.

Example 304. The system according to example 303, wherein the distal ridge has: a distal shoulder that facilitates insertion of the plug into the tube, and/or a proximal step that inhibits withdrawing the plug through the tube.

Example 305. The system according to any one of examples 285–304, wherein the suture lock has: an unlocked state in which the plug is disposed outside of the tube-lumen, and/or a locked state in which the plug is at least partially disposed within the tube-lumen.

Example 306. The system according to example 305, wherein, while the suture lock is in the unlocked state, the plug has an external diameter that is greater than an internal diameter of the tube.

Example 307. The system according to any one of examples 285–306, wherein the tube has a greater elasticity than the plug.

Example 308. The system according to example 307, wherein the tube comprises nitinol.

Example 309. The system according to any one of examples 307–308, wherein the plug comprises cobalt-chrome.

Example 310. A system for securing a suture, the system comprising a suture lock that comprises: a plug, formed from cobalt-chrome; and/or a tube, formed from nitinol, and configured to receive the plug.

Example 311. The system according to example 310, wherein the suture lock is sterile.

Example 312. A system for use with a suture, the system comprising: multiple suture locks, each suture lock comprising: a first lock piece, and/or a second lock piece; and/or a tool: comprising: at a proximal portion of the tool, a storage zone in which the multiple suture locks are disposed, and/or operable to, for each of the multiple suture locks, sequentially: advance the first lock piece and the second lock piece distally from the storage zone to a distal portion of the tool, thread the suture through the first lock piece, and/or while the suture remains disposed through the first lock piece: secure the suture lock to the suture by engaging the first lock piece with the second lock piece, and/or trim the suture.

Example 313. The system according to example 312, wherein at least one of the tool and the suture locks is sterile.

Example 314. The system according to any one of examples 312–313, wherein, for each of the suture locks disposed in the storage zone, the second lock piece is engaged with the first lock piece.

Example 315. The system according to any one of examples 312–314, wherein the tool is configured to advance each of the suture locks distally from the storage zone while the second lock piece is engaged with the first lock piece.

Example 316. The system according to any one of examples 312–315, wherein the tool is operable to secure each of the suture locks to the suture by sandwiching the suture between first lock piece and the second lock piece.

Example 317. The system according to any one of examples 312–316, wherein the tool is configured to separate the first lock piece from the second lock piece, prior to engaging the first lock piece with the second lock piece.

Example 318. The system according to example 317, wherein the tool is configured to: advance each of the suture locks from the storage zone to the distal portion while the second lock piece is engaged with the first lock piece, and/or within the distal portion of the tool, separate the first lock piece from the second lock piece.

Example 319. The system according to example 318, wherein the tool is configured to separate the first lock piece from the second lock piece by advancing the first lock piece, within the distal portion of the tool, distally from the second lock piece.

Example 320. The system according to any one of examples 312–319, wherein the tool further comprises a trigger, configured to, upon application of an actuation force to the trigger, actuate the tool to trim the suture while engaging the first lock piece with the second lock piece.

Example 321. The system according to example 320, wherein: the first lock piece and the second lock piece comprise a first suture lock of the multiple suture locks, the trigger is operable to, upon application of the actuation force to the trigger, cyclically actuate the tool such that trimming the suture advances the tool toward a ready state in which the tool is actuatable to advance a first lock piece and a second lock piece of a second suture lock to the distal portion of the tool.

Example 322. The system according to any one of examples 320–322, wherein, subsequently to securing the suture lock to the suture by engaging the first lock piece with the second lock piece, and prior to actuation of the tool to trim the suture, the tool continues to engage the first lock piece with the second lock piece, in absence of the actuation force.

Example 323. A system for use with a suture, the system comprising: multiple suture locks, each suture lock comprising: a first lock piece, and/or a second lock piece; and/or a tool, comprising: at a proximal portion of the tool, a storage zone in which the multiple suture locks are disposed, a snare, operable to, for each of the multiple suture locks, sequentially thread the suture through the first lock piece and into a distal portion of the tool, and/or a multi-shaft system: extending from the proximal portion of the tool to the distal portion of the tool, and/or operable to, for each of the multiple suture locks, sequentially: advance the first lock piece and the second lock piece distally from the storage zone to the distal portion, and/or while the suture remains disposed through the first lock piece, secure the suture lock to the suture by engaging the first lock piece with the second lock piece, and/or a suture-exposing mechanism, operable to expose part of the suture laterally from the tool.

Example 324. The system according to example 323, wherein at least one of the tool and the suture locks is sterile.

Example 325. The system according to any one of examples 323–324, wherein the snare is operable to, for each of the multiple suture locks, sequentially thread the suture through the first lock piece and into a distal portion of the multi-shaft system.

Example 326. The system according to any one of examples 323–325, wherein the suture-exposing mechanism is operable to expose part of the suture laterally from the multi-shaft system and into the distal portion of the multi-shaft system while the suture remains disposed through the first lock piece.

Example 327. The system according to any one of examples 323–326, wherein the snare is operable to: extend distally through the first lock piece, while the snare is extended distally from the first lock piece, snare a length of the suture and while holding the snared suture, retract proximally, thereby threading the suture through the first lock piece.

Example 328. The system according to any one of examples 323–327, wherein the suture-exposing mechanism comprises a cantilever that is pivotable between: an aligned state in which the cantilever is aligned along the multi-shaft system, and/or an oblique state in which the cantilever is angled laterally away from the multi-shaft system.

Example 329. The system according to example 328, wherein: the snare is operable to thread the suture through the first lock piece and through a portion of the cantilever, while the cantilever is in the aligned state, and/or the cantilever is pivotable toward the oblique state while the suture remains threaded through the portion of the cantilever, thereby exposing part of the suture from the multi-shaft system, while the suture remains threaded through the first lock piece.

What is claimed is:

1. A system for use with a suture, the system comprising:
multiple suture locks, each suture lock comprising a plug and a tube; and
a tool, the tool comprising:
at a proximal portion of the tool, a storage zone in which the multiple suture locks are disposed, and
a multi-shaft system, extending from the proximal portion of the tool to a distal portion of the tool, and
operable to, for each of the multiple suture locks, sequentially:
advance the suture lock distally from the storage zone to the distal portion, and
within the distal portion, secure the suture lock to the suture by pushing the plug into the tube.

2. The system according to claim 1, wherein, for each of the suture locks disposed in the storage zone, the plug is disposed in the tube.

3. The system according to claim 1, wherein the tool is configured to advance each of the suture locks distally from the storage zone while the plug is disposed at least partly within the tube.

4. The system according to claim 1, wherein the tool is configured to separate the tube from the plug prior to pushing the plug into the tube.

5. The system according to claim 1, wherein the tool is configured such that, following the securing of the suture lock to the suture, the suture lock remains within the distal portion of the tool, until an external force is applied to remove the suture lock from the tool.

6. The system according to claim 1,
the multi-shaft system defines a holding zone at the distal portion of the tool; and
the tool is configured to, while holding the tube in the holding zone:
thread the suture through the tube,
secure the suture lock to the suture by pushing the plug into the tube such that a portion of the suture becomes sandwiched between an outer surface of the plug and an inner surface of the tube, and
trim the suture.

7. The system according to claim 1, wherein the tool further comprises a shuttle:
mounted to be repeatedly shuttled, each shuttling including (i) an outbound leg in which the shuttle is slid distally over and along the multi-shaft system from the proximal portion of the tool to the distal portion of the tool, and (ii) a return leg in which the shuttle is subsequently slid proximally over and along the multi-shaft system to return from the distal portion of the tool to the proximal portion of the tool; and
configured such that, during each shuttling:
the outbound leg slides one of the suture locks distally from the storage zone to the distal portion, and
the return leg leaves the suture lock at the distal portion.

8. The system according to claim 1, wherein the tool further comprises:
a force applicator at the distal portion of the tool; and
a force regulator, proximal from the force applicator, the force regulator defining a force threshold, and being configured to, upon application of an actuation force to the force regulator, transfer the actuation force to the force applicator in a manner that:
secures the suture lock to the suture by pushing the plug into the tube while the actuation force remains below the force threshold, and
upon the actuation force reaching the force threshold, causes the tool to trim the suture.

9. A system for use with a suture, the system comprising:
multiple suture locks, each suture lock comprising:
a first lock piece, and
a second lock piece; and
a tool, the tool comprising:
at a proximal portion of the tool, a storage zone in which the multiple suture locks are disposed, and
operable to, for each of the multiple suture locks, sequentially:
advance the first lock piece and the second lock piece distally from the storage zone to a distal portion of the tool,
thread the suture through the first lock piece, and
while the suture remains disposed through the first lock piece:
secure the suture lock to the suture by engaging the first lock piece with the second lock piece, and
trim the suture.

10. The system according to claim 9, wherein, for each of the suture locks disposed in the storage zone, the second lock piece is engaged with the first lock piece.

11. The system according to claim 9, wherein the tool is configured to advance each of the suture locks distally from the storage zone while the second lock piece is engaged with the first lock piece.

12. The system according to claim 9, wherein the tool further comprises a trigger, configured to, upon application of an actuation force to the trigger, actuate the tool to trim the suture while engaging the first lock piece with the second lock piece.

13. The system according to claim 9, wherein the tool is configured to separate the first lock piece from the second lock piece, prior to engaging the first lock piece with the second lock piece.

14. The system according to claim 13, wherein the tool is configured to:
advance each of the suture locks from the storage zone to the distal portion while the second lock piece is engaged with the first lock piece, and
within the distal portion of the tool, separate the first lock piece from the second lock piece.

15. A system for use with a suture, the system comprising:
multiple suture locks, each suture lock comprising:
a first lock piece, and
a second lock piece; and
a tool, comprising:
at a proximal portion of the tool, a storage zone in which the multiple suture locks are disposed,
a snare, operable to, for each of the multiple suture locks sequentially, thread the suture through the first lock piece and into a distal portion of the tool, and
a multi-shaft system:
extending from the proximal portion of the tool to the distal portion of the tool, and
operable to, for each of the multiple suture locks, sequentially:
advance the first lock piece and the second lock piece distally from the storage zone to the distal portion, and
while the suture remains disposed through the first lock piece, secure the suture lock to the suture by engaging the first lock piece with the second lock piece, and
a suture-exposing mechanism, operable to expose part of the suture laterally from the tool.

16. The system according to claim 15, wherein, the snare is operable to, for each of the multiple suture locks sequentially, thread the suture through the first lock piece and into a distal portion of the multi-shaft system.

17. The system according to claim 15, wherein, for each of the locks, the first lock piece comprises a tube, configured to receive the second lock piece, and comprising a wall that:
circumscribes and defines a tube axis and a tube-lumen along the tube axis, and
has a set of axial slits therein, distributed circumferentially around the tube axis to provide the wall with compliance that facilitates insertion of the second lock piece into the tube and retention of the second lock piece by the tube.

18. The system according to claim 15, wherein the snare is operable to:
extend distally through the first lock piece,
while the snare is extended distally from the first lock piece, snare a length of the suture and
while holding the snared suture, retract proximally, thereby threading the suture through the first lock piece.

19. The system according to claim 15, wherein the suture-exposing mechanism comprises a cantilever that is pivotable between:
an aligned state in which the cantilever is aligned along the multi-shaft system, and
an oblique state in which the cantilever is angled laterally away from the multi-shaft system.

20. The system according to claim 19, wherein:
the snare is operable to thread the suture through the first lock piece and through a portion of the cantilever, while the cantilever is in the aligned state, and
the cantilever is pivotable toward the oblique state while the suture remains threaded through the portion of the cantilever, thereby exposing part of the suture from the multi-shaft system, while the suture remains threaded through the first lock piece.
